(19) United States
(12) Patent Application Publication
(10) Pub. No.: US 2022/0105166 A1
(43) Pub. Date: Apr. 7, 2022

(54) ANUCLEATE CELL-DERIVED VACCINES

(71) Applicant: SQZ Biotechnologies Company, Watertown, MA (US)

(72) Inventors: Armon R. SHAREI, Watertown, MA (US); Howard BERNSTEIN, Cambridge, MA (US); Jonathan B. GILBERT, Somerville, MA (US); Finola MOORE, Watertown, MA (US); Devin BRIDGEN, Watertown, MA (US); Luke CASSEREAU, San Bruno, CA (US)

(73) Assignee: SQZ Biotechnologies Company, Watertown, MA (US)

(21) Appl. No.: 17/425,709

(22) PCT Filed: Jan. 24, 2020
(86) PCT No.: PCT/US2020/015098

§ 371 Date: Jul. 23, 2021

Related U.S. Application Data
(60) Provisional application No. 62/797,185, filed on Jan. 25, 2019.
(60) Provisional application No. 62/797,187, filed on Jan. 25, 2019.
(60) Provisional application No. 62/933,301, filed on Nov. 8, 2019.
(60) Provisional application No. 62/933,302, filed on Nov. 8, 2019.

(51) Int. Cl. A61K 39/00; A61K 38/21; A61K 38/20; A61K 39/39; A61P 37/04

(52) U.S. Cl. CPC A61K 39/0011; A61K 38/212; A61K 38/217; A61K 38/2013; A61K 38/2086; A61K 39/39; A61K 2039/6031; A61K 2039/545; A61K 2039/5156; A61K 2039/55561; A61K 2039/55522; A61K 2039/55572; A61P 37/04

(57) ABSTRACT

The present invention provides methods for stimulating an immune response to an antigen comprising administering to an individual, an anucleate cell-derived vesicle comprising an antigen and/or an adjuvant. In some embodiments, the anucleate cell-derived vesicle comprising the antigen and/or adjuvant is generated by passing a cell suspension containing an input anucleate cell through a constriction, wherein the constriction deforms the input anucleate cell thereby causing a perturbation of the cell to form an anucleate cell-derived vesicle such that an antigen and/or an adjuvant enters the anucleate cell-derived vesicle. In some embodiments, the anucleate cell-derived vesicle comprising the antigen and/or adjuvant is delivered to an individual and the antigen is delivered to and processed in an immunogenic environment to treat a disease, prevent a disease, and/or vaccinate an individual against an antigen.

### CROSS REFERENCE TO RELATED APPLICATIONS

This application claims the benefit of U.S. Provisional Application No. 62/797,185, filed on Jan. 25, 2019, U.S. Provisional Application No. 62/797,187, filed on Jan. 25, 2019, U.S. Provisional Application No. 62/933,301, filed on Nov. 8, 2019, and U.S. Provisional Application No. 62/933,302, filed on Nov. 8, 2019, the entire contents of each of which are incorporated herein by reference.

### FIELD OF THE INVENTION

The present disclosure relates generally to methods for stimulating an immune response or methods of treating cancer, infectious diseases or viral-associated disease by delivering an anucleate cell-derived vesicle to an individual, wherein the anucleate cell-derived vesicles are loaded with an antigen and/or adjuvant. In some embodiments, the antigen and/or adjuvant is delivered to an anucleate cell by passing a cell suspension through a cell-deforming constriction.

### BACKGROUND

The complexity of the immune system and immune response to foreign matter makes challenging the development of efficacious approaches for triggering an in vivo antigen-specific immune response. In addition to the continued development of agents, such as small molecules and polypeptide- and/or nucleotide-based vaccines, capable of triggering antigen-specific immune responses, carrier strategies for use with such agents are in need of further development to optimize delivery and immune response. Carriers known in the art, including polymer-based carriers, particle carriers, liposomes, and cell-based vesicles, such as those derived from red blood cells, still face challenges limiting their use for triggering an in vivo antigen-specific immune response. For example, use of red blood cells as a carrier is difficult due to challenges associated with manipulation of red blood cells to associate antigenic material given that red blood cells are irregularly shaped (biconcave), anucleate, and transcriptionally inactive. As a result, standard transfection techniques do not work. To overcome these challenges, methods of using red blood cells as a carrier for triggering an immune response have focused on conjugating materials to the surface of erythrocytes. See, e.g., Lorentz et al., Sci. Adv, 1:e15001122015; Grimm et al., Sci Rep, 5, 2015; and Kontos et al., Proc Natl Acad Sci USA, 110, 2013. Initial work using surface conjugation has shown promising results with model antigens and mouse models of Type 1 diabetes but has some significant drawbacks including: (a) the need for chemically modified antigens for attachment; (b) the limited surface area for loading; and (c) immunogenicity.

References that describe methods of using microfluidic constrictions to deliver compounds to cells include WO2013059343, WO2015023982, WO2016070136, WO2016077761, and WO/2017/192785.

All references cited herein, including patent applications and publications, are incorporated by reference in their entirety.

### BRIEF SUMMARY OF THE INVENTION

In some aspects, the invention provides methods for delivering an antigen into an anucleate cell-derived vesicle, the method comprising: a) passing a cell suspension comprising an input (e.g., parent) anucleate cell through a cell-deforming constriction, wherein a diameter of the constriction is a function of a diameter of the input anucleate cell in the suspension, thereby causing perturbations of the input anucleate cell large enough for the antigen to pass through to form an anucleate cell-derived vesicle; and b) incubating the anucleate cell-derived vesicle with the antigen for a sufficient time to allow the antigen to enter the anucleate cell-derived vesicle. In some embodiments, the input anucleate cell further comprises an adjuvant.

In some aspects, the invention provides methods for delivering an adjuvant into an anucleate cell-derived vesicle, the method comprising: a) passing a cell suspension comprising an input anucleate cell through a cell-deforming constriction, wherein a diameter of the constriction is a function of a diameter of the input anucleate cell in the suspension, thereby causing perturbations of the input anucleate cell large enough for the adjuvant to pass through to form an anucleate cell-derived vesicle; and b) incubating the anucleate cell-derived vesicle with the adjuvant for a sufficient time to allow the adjuvant to enter the anucleate cell-derived vesicle. In some embodiments, input anucleate cell further comprises an antigen.

In some aspects, the invention provides methods for delivering an antigen and an adjuvant into an anucleate cell-derived vesicle, the method comprising: a) passing a cell suspension comprising an input anucleate cell through a cell-deforming constriction, wherein a diameter of the constriction is a function of a diameter of the input anucleate cell in the suspension, thereby causing perturbations of the input anucleate cell large enough for the antigen and the adjuvant to pass through to form an anucleate cell-derived vesicle; and b) incubating the anucleate cell-derived vesicle with the antigen and the adjuvant for a sufficient time to allow the antigen and the adjuvant to enter the anucleate cell-derived vesicle.

In some embodiments, the invention provides methods for stimulating an immune response to an antigen in an individual, the method comprising administering to the individual an effective amount of an anucleate cell-derived vesicle comprising an antigen, wherein the anucleate cell-derived vesicle comprising the antigen is prepared by a process comprising the steps of: a) passing a cell suspension comprising an input anucleate cell through a cell-deforming constriction, wherein a diameter of the constriction is a function of a diameter of the input anucleate cell in the suspension, thereby causing perturbations of the input anucleate cell large enough for the antigen to pass through to form an anucleate cell-derived vesicle; and b) incubating the anucleate cell-derived vesicle with the antigen for a sufficient time to allow the antigen to enter the anucleate cell-derived vesicle. In some embodiments, the method further comprises administering an adjuvant systemically to the individual. In some embodiments, the adjuvant is administered systemically before, after or at the same time as the anucleate cell derived vesicle. In some embodiments, the input anucleate cell comprises an adjuvant.

In some aspects, the invention provides methods for stimulating an immune response to an antigen in an individual, the method comprising administering to the individual an effective amount of an anucleate cell-derived vesicle comprising an antigen and an adjuvant, wherein the anucleate cell-derived vesicle comprising the antigen and the adjuvant is prepared by a process comprising the steps of: a) passing a cell suspension comprising an input anucleate cell through a cell-deforming constriction, wherein a diameter of the constriction is a function of a diameter of the input anucleate cell in the suspension, thereby causing perturbations of the input anucleate cell large enough for the antigen and the adjuvant to pass through to form an anucleate cell-derived vesicle; and b) incubating the anucleate cell-derived vesicle with the antigen and the adjuvant for a sufficient time to allow the antigen and the adjuvant to enter the anucleate cell-derived vesicle. In some embodiments, the method further comprises administering an adjuvant systemically to the individual. In some embodiments, the adjuvant is administered systemically before, after or at the same time as the anucleate cell-derived vesicle. In some aspects, the invention provides methods for treating a disease in an individual, comprising administering to the individual an anucleate cell-derived vesicle comprising a disease-associated antigen, wherein an immune response against the antigen ameliorates conditions of the disease, and wherein the anucleate cell-derived vesicle comprising the disease-associated antigen is prepared by a process comprising the steps of: a) passing a cell suspension comprising an input anucleate cell through a cell-deforming constriction, wherein a diameter of the constriction is a function of a diameter of the input anucleate cell in the suspension, thereby causing perturbations of the input anucleate cell large enough for the antigen to pass through to form an anucleate cell-derived vesicle; and b) incubating the anucleate cell-derived vesicle with the antigen for a sufficient time to allow the antigen to enter the anucleate cell-derived vesicle.

In some aspects, the invention provides methods for preventing a disease in an individual, comprising administering to the individual an anucleate cell-derived vesicle comprising a disease-associated antigen, wherein an immune response against the antigen prevents development of the disease, and wherein the anucleate cell-derived vesicle comprising the disease-associated antigen is prepared by a process comprising the steps of: a) passing a cell suspension comprising an input anucleate cell through a cell-deforming constriction, wherein a diameter of the constriction is a function of a diameter of the input anucleate cell in the suspension, thereby causing perturbations of the input anucleate cell large enough for the antigen to pass through to form an anucleate cell-derived vesicle; and b) incubating the anucleate cell-derived vesicle with the antigen for a sufficient time to allow the antigen to enter the anucleate cell-derived vesicle. In some embodiments, the invention provides methods for vaccinating an individual against an antigen, comprising administering to the individual an anucleate cell-derived vesicle comprising the antigen, wherein the anucleate cell-derived vesicle comprising the antigen is prepared by a process comprising the steps of: a) passing a cell suspension comprising an input anucleate cell through a cell-deforming constriction, wherein a diameter of the constriction is a function of a diameter of the input anucleate cell in the suspension, thereby causing perturbations of the input anucleate cell large enough for the antigen to pass through to form an anucleate cell-derived vesicle; and b) incubating the anucleate cell-derived vesicle with the antigen for a sufficient time to allow the antigen to enter the anucleate cell-derived vesicle. In some embodiments, the method further comprises administering an adjuvant systemically to the individual. In some embodiments, the adjuvant is administered systemically before, after or at the same time as the anucleate cell derived vesicle. In some embodiments, the input anucleate cell comprises an adjuvant.

In some aspects, the invention provides methods for treating a disease in an individual, comprising administering to the individual an anucleate cell-derived vesicle comprising a disease-associated antigen and an adjuvant, wherein an immune response against the antigen ameliorates conditions of the disease, and wherein the anucleate cell-derived vesicle comprising the disease-associated antigen and the adjuvant is prepared by a process comprising the steps of: a) passing a cell suspension comprising an input anucleate cell through a cell-deforming constriction, wherein a diameter of the constriction is a function of a diameter of the input anucleate cell in the suspension, thereby causing perturbations of the input anucleate cell large enough for the antigen and an adjuvant to pass through to form an anucleate cell-derived vesicle; and b) incubating the anucleate cell-derived vesicle with the antigen and the adjuvant for a sufficient time to allow the antigen and the adjuvant to enter the anucleate cell-derived vesicle. In some aspects, the invention provides methods for preventing a disease in an individual, comprising administering to the individual an anucleate cell-derived vesicle comprising a disease-associated antigen and an adjuvant, wherein an immune response against the antigen prevents development of the disease, and wherein the anucleate cell-derived vesicle comprising a disease-associated antigen and an adjuvant is prepared by a process comprising the steps of: a) passing a cell suspension comprising an input anucleate cell through a cell-deforming constriction, wherein a diameter of the constriction is a function of a diameter of the input anucleate cell in the suspension, thereby causing perturbations of the input anucleate cell large enough for the antigen and an adjuvant to pass through to form an anucleate cell-derived vesicle; and b) incubating the anucleate cell-derived vesicle with the antigen and the adjuvant for a sufficient time to allow the antigen and the adjuvant to enter the anucleate cell-derived vesicle. In some aspects, the invention provides methods for vaccinating an individual against an antigen, comprising administering to the individual an anucleate cell-derived vesicle comprising the antigen and an adjuvant, wherein the anucleate cell-derived vesicle comprising the antigen and the adjuvant is prepared by a process comprising the steps of: a) passing a cell suspension comprising an input anucleate cell through a cell-deforming constriction, wherein a diameter of the constriction is a function of a diameter of the input anucleate cell in the suspension, thereby causing perturbations of the input anucleate cell large enough for the antigen and an adjuvant to pass through to form an anucleate cell-derived vesicle; and b) incubating the anucleate cell-derived vesicle with the antigen and the adjuvant for a sufficient time to allow the antigen and the adjuvant to enter the anucleate cell-derived vesicle.

In some aspects, the invention provides methods for treating a disease in an individual, wherein an immune response against a disease-associated antigen ameliorates conditions of the disease, the method comprising a) passing a cell suspension comprising an input anucleate cell through a cell-deforming constriction, wherein a diameter of the constriction is a function of a diameter of the input anucleate cell in the suspension, thereby causing perturbations of the input anucleate cell large enough for the antigen to pass through to form an anucleate cell-derived vesicle; b) incubating the anucleate cell-derived vesicle with the antigen for a sufficient time to allow the antigen to enter the anucleate cell-derived vesicle, thereby generating an anucleate cell-derived vesicle comprising the antigen; and c) administering the anucleate cell-derived vesicle comprising the antigen to the individual. In some aspects, the invention provides methods for preventing a disease in an individual, wherein an immune response against a disease-associated antigen prevents development of the disease, the method comprising a) passing a cell suspension comprising an input anucleate cell through a cell-deforming constriction, wherein a diameter of the constriction is a function of a diameter of the input anucleate cell in the suspension, thereby causing perturbations of the input anucleate cell large enough for the antigen to pass through to form an anucleate cell-derived vesicle; b) incubating the anucleate cell-derived vesicle with the antigen for a sufficient time to allow the antigen to enter the anucleate cell-derived vesicle, thereby generating an anucleate cell-derived vesicle comprising the antigen; and c) administering the anucleate cell-derived vesicle comprising the antigen to the individual. In some aspects, the invention provides methods for vaccinating an individual against an antigen, the method comprising, a) passing a cell suspension comprising an input anucleate cell through a cell-deforming constriction, wherein a diameter of the constriction is a function of a diameter of the input anucleate cell in the suspension, thereby causing perturbations of the input anucleate cell large enough for the antigen to pass through to form an anucleate cell-derived vesicle; b) incubating the anucleate cell-derived vesicle with the antigen for a sufficient time to allow the antigen to enter the anucleate cell-derived vesicle, thereby generating an anucleate cell-derived vesicle comprising the antigen; and c) administering the anucleate cell-derived vesicle comprising the antigen to the individual. In some embodiments, the method further comprises administering an extravesicular adjuvant systemically to the individual. In some embodiments, the extravesicular adjuvant is administered before, after or at the same time as the anucleate cell-derived vesicle. In some embodiments, the input anucleate cell comprises an adjuvant.

In some aspects, the invention provides methods for treating a disease in an individual, wherein an immune response against a disease-associated antigen ameliorates conditions of the disease, the method comprising a) passing a cell suspension comprising an input anucleate cell through a cell-deforming constriction, wherein a diameter of the constriction is a function of a diameter of the input anucleate cell in the suspension, thereby causing perturbations of the input anucleate cell large enough for the disease-associated antigen and an adjuvant to pass through to form an anucleate cell-derived vesicle; b) incubating the anucleate cell-derived vesicle with the antigen and the adjuvant for a sufficient time to allow the antigen and the adjuvant to enter the anucleate cell-derived vesicle, thereby generating an anucleate cell-derived vesicle comprising the antigen and the adjuvant; and c) administering the anucleate cell-derived vesicle comprising the antigen and the adjuvant to the individual. In some aspects, the invention provides methods for preventing a disease in an individual, wherein an immune response against a disease-associated antigen prevents development of the disease, the method comprising a) passing a cell suspension comprising an input anucleate cell through a cell-deforming constriction, wherein a diameter of the constriction is a function of a diameter of the input anucleate cell in the suspension, thereby causing perturbations of the input anucleate cell large enough for the antigen and an adjuvant to pass through to form an anucleate cell-derived vesicle; b) incubating the anucleate cell-derived vesicle with the antigen and the adjuvant for a sufficient time to allow the antigen and the adjuvant to enter the anucleate cell-derived vesicle, thereby generating an anucleate cell-derived vesicle comprising the antigen and the adjuvant; and c) administering the anucleate cell-derived vesicle comprising the antigen and the adjuvant to the individual. In some embodiments, the invention provides methods for vaccinating an individual against an antigen, the method comprising, a) passing a cell suspension comprising an input anucleate cell through a cell-deforming constriction, wherein a diameter of the constriction is a function of a diameter of the input anucleate cell in the suspension, thereby causing perturbations of the input anucleate cell large enough for the antigen and an adjuvant to pass through to form an anucleate cell-derived vesicle; b) incubating the anucleate cell-derived vesicle with the antigen and the adjuvant for a sufficient time to allow the antigen and the adjuvant to enter the anucleate cell-derived vesicle, thereby generating an anucleate cell-derived vesicle comprising the antigen and the adjuvant; and c) administering the anucleate cell-derived vesicle comprising the antigen and the adjuvant to the individual. In some embodiments, the method further comprises administering an extravesicular adjuvant systemically to the individual. In some embodiments, the extravesicular adjuvant is administered before, after or at the same time as the anucleate cell derived vesicle.

In some embodiments of the above aspects, the disease is cancer, an infectious disease or a viral-associated disease. In some embodiments, the anucleate cell-derived vesicle is autologous to the individual. In some embodiments, the anucleate cell-derived vesicle is allogeneic to the individual. In some embodiments, the anucleate cell-derived vesicle is in a pharmaceutical formulation. In some embodiments, the anucleate cell-derived vesicle is administered systemically. In some embodiments, the anucleate cell-derived vesicle is administered intravenously, intraarterially, subcutaneously, intramuscularly, or intraperitoneally.

In some embodiments of the above aspects, the anucleate cell-derived vesicle is administered to the individual in combination with a therapeutic agent. In some embodiments, the therapeutic agent is administered before, after or at the same time as the anucleate cell-derived vesicle. In some embodiments, the therapeutic agent is an immune checkpoint inhibitor and/or a cytokine. In some embodiments, the cytokine is one or more of IFN-α, IFN-γ, IL-2, IL-10, or IL-15. In some embodiments, the immune checkpoint inhibitor is targeted to any one of PD-1, PD-L1, CTLA-4, TIM-3, LAGS, TIGIT, VISTA, TIM1, B7-H4 (VTCN1) and BTLA. In some embodiments, the therapeutic agent is a bispecific agent; for example, a bispecific agent comprising a cytokine component and a targeting component. In some embodiments, the bispecific agent comprises a targeting component and a trap for a molecule such as TGFb. In some embodiments, the anucleate cell-derived vesicle is administered to the individual in combination with a chemotherapy or a radiation therapy. In some embodiments, the anucleate cell-derived vesicle is administered to the individual in combination with one or more agents that improve antigen presentation (e.g., CD40 or Ox40L), improve T cell proliferation, and/or improve tumor microenvironments (e.g., ICOS).

In some embodiments of the above aspects, the antigen is capable of being processed into an MHC class I-restricted peptide and/or an MHC class II-restricted peptide. In some embodiments, the antigen is a CD-1 restricted antigen. In some embodiments, the antigen is a disease-associated antigen. In some embodiments, the antigen is a tumor antigen. In some embodiments, the antigen is derived from a lysate. In some embodiments, the lysate is a tumor lysate. In some embodiments, the antigen is a viral antigen, a bacterial antigen or a fungal antigen. In some embodiments, the antigen is a microorganism. In some embodiments, the antigen is a polypeptide. In some embodiments, the antigen is a lipid antigen. In some embodiments, the antigen is a carbohydrate antigen. In some embodiments, a nucleic acid encoding the antigen is delivered to the cell. In some embodiments, the antigen is a modified antigen. In some embodiments, the modified antigen comprises an antigen fused with a polypeptide. In some embodiments, the modified antigen comprises an antigen fused with a targeting peptide. In some embodiments, the modified antigen comprises an antigen fused with a lipid. In some embodiments, the modified antigen comprises an antigen fused with a carbohydrate. In some embodiments, the modified antigen comprises an antigen fused with a nanoparticle. In some embodiments, a nucleic acid encoding the antigen is delivered to the cell. In some embodiments, a plurality of antigens is delivered to the anucleate cell-derived vesicle.

In some embodiments of the above aspects, the adjuvant is a CpG ODN, IFN-α, STING agonists, RIG-I agonists, poly I:C, polyinosinic-polycytidylic acid stabilized with polylysine and carboxymethylcellulose (HILTONOL®), imiquimod, resiquimod, and/or lipopolysaccharide (LPS). In some embodiments, the adjuvant is low molecular weight poly I:C.

In some embodiments of the above aspects, the input anucleate cell is a red blood cell. In some embodiments, the red blood cell is an erythrocyte. In some embodiments, the red blood cell is a reticulocyte. In some embodiments, the input anucleate cell is a platelet. In some embodiments, the input anucleate cell is a mammalian cell. In some embodiments, the input anucleate cell is a monkey, mouse, dog, cat, horse, rat, sheep, goat, pig, or rabbit cell. In some embodiments, the input anucleate cell is a human cell.

In some embodiments of the above aspects, the constriction is contained within a microfluidic channel. In some embodiments, the microfluidic channel comprises a plurality of constrictions. In some embodiments, the plurality of constrictions is arranged in series and/or in parallel. In some embodiments, the constriction is between a plurality of micropillars; between a plurality of micropillars configured in an array; or between one or more movable plates. In some embodiments, the constriction is a pore or contained within a pore. In some embodiments, the pore is contained in a surface. In some embodiments, the surface is a filter. In some embodiments, the surface is a membrane. In some embodiments, the constriction size is a function of the diameter of the input anucleate cell in suspension. In some embodiments, the constriction size is about 10%, about 20%, about 30%, about 40%, about 50%, about 60%, or about 70% of the diameter of the input anucleate cell in suspension. In some embodiments, the constriction has a width of about 0.25 μm to about 4 μm. In some embodiments, the constriction has a width of about 4 μm, about 3.5 μm, about 3 μm, about 2.5 μm, about 2 μm, about 1.5 μm, about 1 μm, about 0.5 μm, or about 0.25 μm. In some embodiments, the constriction has a width of about 2.2 μm. In some embodiments, the input anucleate cells are passed through the constriction under a pressure ranging from about 10 psi to about 90 psi. In some embodiments, said cell suspension is contacted with the antigen before, concurrently, or after passing through the constriction.

In some aspects, the invention provides an anucleate cell-derived vesicle comprising an antigen, wherein the anucleate cell-derived vesicle comprising the antigen is prepared by a process comprising the steps of: a) passing a cell suspension comprising an input anucleate cell through a cell-deforming constriction, wherein a diameter of the constriction is a function of a diameter of the input anucleate cell in the suspension, thereby causing perturbations of the input anucleate cell large enough for the antigen to pass through to form an anucleate cell-derived vesicle; and b) incubating the anucleate cell-derived vesicle with the antigen for a sufficient time to allow the antigen to enter the anucleate cell-derived vesicle; thereby generating the anucleate cell-derived vesicle comprising the antigen. In some embodiments, the input anucleate cell comprises an adjuvant. In some aspects, the invention provides an anucleate cell-derived vesicle comprising an adjuvant, wherein the anucleate cell-derived vesicle comprising the adjuvant is prepared by a process comprising the steps of: a) passing a cell suspension comprising an input anucleate cell through a cell-deforming constriction, wherein a diameter of the constriction is a function of a diameter of the input anucleate cell in the suspension, thereby causing perturbations of the input anucleate cell large enough for the adjuvant to pass through to form an anucleate cell-derived vesicle; and b) incubating the anucleate cell-derived vesicle with the adjuvant for a sufficient time to allow the adjuvant to enter the anucleate cell-derived vesicle; thereby generating the anucleate cell-derived vesicle comprising the adjuvant. In some embodiments, the input anucleate cell comprises an antigen. In some aspects, the invention provides an anucleate cell-derived vesicle comprising an antigen and an adjuvant, wherein the anucleate cell-derived vesicle comprising the antigen and the adjuvant is prepared by a process comprising the steps of: a) passing a cell suspension comprising an input anucleate cell through a cell-deforming constriction, wherein a diameter of the constriction is a function of a diameter of the input anucleate cell in the suspension, thereby causing perturbations of the input anucleate cell large enough for the antigen and the adjuvant to pass through to form an anucleate cell-derived vesicle; and b) incubating the anucleate cell-derived vesicle with the antigen and the adjuvant for a sufficient time to allow the antigen and the adjuvant to enter the anucleate cell-derived vesicle; thereby generating the anucleate cell-derived vesicle comprising the antigen and the adjuvant. In some embodiments, the anucleate cell-derived vesicle is a red blood cell-derived vesicle or a platelet-derived vesicle. In some embodiments, the red blood cell-derived vesicle is an erythrocyte-derived vesicle, or a reticulocyte-derived vesicle.

In some embodiments of the above anucleate cell-derived vesicles, the antigen is capable of being processed into an MHC class I-restricted peptide and/or an MHC class II-restricted peptide. In some embodiments, the antigen is a CD-1 restricted antigen. In some embodiments, the antigen is a disease-associated antigen. In some embodiments, the antigen is a tumor antigen. In some embodiments, the antigen is derived from a lysate. In some embodiments, the lysate is a tumor lysate. In some embodiments, the antigen is a viral antigen, a bacterial antigen or a fungal antigen. In some embodiments, the antigen is a microorganism. In some embodiments, the antigen is a polypeptide. In some embodiments, the antigen is a lipid antigen. In some embodiments, the antigen is a carbohydrate antigen. In some embodiments, a nucleic acid encoding the antigen is delivered to the cell. In some embodiments, the antigen is a modified antigen. In some embodiments, the modified antigen comprises an antigen fused with a polypeptide. In some embodiments, the modified antigen comprises an antigen fused with a targeting peptide. In some embodiments, the modified antigen comprises an antigen fused with a lipid. In some embodiments, the modified antigen comprises an antigen fused with a carbohydrate. In some embodiments, the modified antigen comprises an antigen fused with a nanoparticle. In some embodiments, a plurality of antigens is delivered to the anucleate cell-derived vesicle.

In some embodiments of the above anucleate cell-derived vesicles, the adjuvant is a CpG ODN, IFN-α, STING agonists, RIG-I agonists, poly I:C, polyinosinic-polycytidylic acid stabilized with polylysine and carboxymethylcellulose (HILTONOL®), imiquimod, resiquimod and/or LPS. In some embodiments, the adjuvant is low molecular weight poly I:C.

In some embodiments of the above anucleate cell-derived vesicles, the input anucleate cell is a red blood cell. In some embodiments, the input anucleate cell is an erythrocyte. In some embodiments, the input anucleate cell is a reticulocyte. In some embodiments, the input anucleate cell is a platelet. In some embodiments, the input anucleate cell is a mammalian cell. In some embodiments, the input anucleate cell is a monkey, mouse, dog, cat, horse, rat, sheep, goat, pig, or rabbit cell. In some embodiments, the input anucleate cell is a human cell.

In some embodiments of the above anucleate cell-derived vesicles, the half-life of the anucleate cell-derived vesicle following administration to a mammal is decreased compared to a half-life of the input anucleate cell following administration to the mammal. In some embodiments, the hemoglobin content of the anucleate cell-derived vesicle is decreased compared to the hemoglobin content of the input anucleate cell. In some embodiments, ATP production of the anucleate cell-derived vesicle is decreased compared to ATP production of the input anucleate cell. In some embodiments, the anucleate cell-derived vesicle exhibits a spherical morphology. In some embodiments, the input anucleate cell is an erythrocyte and wherein the anucleate cell-derived vesicle has a reduced biconcave shape compared to the input anucleate cell. In some embodiments, the anucleate cell-derived vesicle is a red blood cell ghost. In some embodiments, the anucleate cell-derived vesicles prepared by the process have greater than about 1.5 fold more phosphatidylserine on its surface compared to the input anucleate cell. In some embodiments, a population profile of anucleate cell-derived vesicles prepared by the process exhibits higher average phosphatidylserine levels on the surface compared to the input anucleate cells. In some embodiments, at least 50% of the population profile of anucleate cell-derived vesicles prepared by the process exhibits higher phosphatidylserine levels on the surface compared to the input anucleate cells.

In some embodiments, the anucleate cell-derived vesicle exhibits enhanced uptake in a tissue or cell compared to the input anucleate cell. In some embodiments, the anucleate cell-derived vesicle exhibits enhanced uptake in phagocytic cells and/or antigen presenting cells compared to the input anucleate cell. In some embodiments, the anucleate cell-derived vesicle is modified to enhance uptake in a tissue or cell compared to an unmodified anucleate cell-derived vesicle. In some embodiments, the anucleate cell-derived vesicle is modified to enhance uptake in phagocytic cells and/or antigen presenting cells compared to an unmodified anucleate cell-derived vesicle. In some embodiments, the phagocytic cells and/or antigen presenting cells comprise one or more of a dendritic cell or macrophage. In some embodiments, the tissue or cell comprises one or more of liver or spleen. In some embodiments, the anucleate cell-derived vesicle comprises CD47 on its surface.

In some embodiments, the anucleate cell-derived vesicle is not (a) heat processed, (b) chemically treated, and/or (c) subjected to hypotonic or hypertonic conditions during the preparation of the anucleate cell-derived vesicles. In some embodiments, the osmolarity of the cell suspension is maintained throughout the process. In some embodiments, the osmolarity of the cell suspension is maintained between 200 mOsm and 400 mOsm throughout the process.

In some embodiments of the above anucleate cell-derived vesicles, the constriction is contained within a microfluidic channel. In some embodiments, the microfluidic channel comprises a plurality of constrictions. In some embodiments, the plurality of constrictions is arranged in series and/or in parallel. In some embodiments, the constriction is between a plurality of micropillars; between a plurality of micropillars configured in an array; or between one or more movable plates. In some embodiments, the constriction is a pore or contained within a pore. In some embodiments, the pore is contained in a surface. In some embodiments, the surface is a membrane. In some embodiments, the constriction size is a function of the diameter of the input anucleate cell in suspension. In some embodiments, the constriction size is about 10%, about 20%, about 30%, about 40%, about 50%, about 60%, or about 70% of the diameter of the input anucleate cell in suspension. In some embodiments, the constriction has a width of about 0.25 μm to about 4 μm. In some embodiments, the constriction has a width of about 4 μm, 3.5 μm, about 3 μm, about 2.5 μm, about 2 μm, about 1.5 μm, about 1 μm, about 0.5 μm, or about 0.25 μm. In some embodiments, the constriction has a width of about 2.2 μm. In some embodiments, the input anucleate cells are passed through the constriction under a pressure ranging from about 10 psi to about 90 psi. In some embodiments, said cell suspension is contacted with the antigen before, concurrently, or after passing through the constriction.

In some aspects, the invention provides compositions comprising a plurality of anucleate cell-derived vesicles as described herein. In some embodiments, the composition further comprises a pharmaceutically acceptable excipient.

In some aspects, the invention provides methods for generating an anucleate cell-derived vesicle comprising an antigen, the method comprising: a) passing a cell suspension comprising an input anucleate cell through a cell-deforming constriction, wherein a diameter of the constriction is a function of a diameter of the input anucleate cell in the suspension, thereby causing perturbations of the input anucleate cell large enough for the antigen to pass through to form an anucleate cell-derived vesicle; b) incubating the anucleate cell-derived vesicle with the antigen for a sufficient time to allow the antigen to enter the anucleate cell-derived vesicle, thereby generating an anucleate cell-derived vesicle comprising the antigen. In some embodiments, the input anucleate cell comprises an adjuvant.

In some aspects, the invention provides methods for generating an anucleate cell-derived vesicle comprising an adjuvant, the method comprising: a) passing a cell suspension comprising an input anucleate cell through a cell-deforming constriction, wherein a diameter of the constriction is a function of a diameter of the input anucleate cell in the suspension, thereby causing perturbations of the input anucleate cell large enough for the adjuvant to pass through to form an anucleate cell-derived vesicle; b) incubating the anucleate cell-derived vesicle with the adjuvant for a sufficient time to allow the adjuvant to enter the anucleate cell-derived vesicle, thereby generating an anucleate cell-derived vesicle comprising the adjuvant. In some embodiments, the input anucleate cell comprises an antigen.

In some aspects, the invention provides methods for generating an anucleate cell-derived vesicle comprising an antigen and an adjuvant, the method comprising: a) passing a cell suspension comprising an input anucleate cell through a cell-deforming constriction, wherein a diameter of the constriction is a function of a diameter of the input anucleate cell in the suspension, thereby causing perturbations of the input anucleate cell large enough for the antigen and the adjuvant to pass through to form an anucleate cell-derived vesicle; b) incubating the anucleate cell-derived vesicle with the antigen and the adjuvant for a sufficient time to allow the antigen and the adjuvant to enter the anucleate cell-derived vesicle, thereby generating an anucleate cell-derived vesicle comprising the antigen and the adjuvant.

In some embodiments of the above method for generating an anucleate cell-derived vesicle, the anucleate cell-derived vesicle is a red blood cell-derived vesicle or a platelet derived vesicle. In some embodiments, the red blood cell-derived vesicle is an erythrocyte-derived vesicle or a reticulocyte-derived vesicle.

In some embodiments of the above method for generating an anucleate cell-derived vesicle, the antigen is capable of being processed into an MHC class I-restricted peptide and/or an MHC class II-restricted peptide. In some embodiments, the antigen is a CD-1 restricted antigen. In some embodiments, the antigen is a disease-associated antigen. In some embodiments, the antigen is a tumor antigen. In some embodiments, the antigen is derived from a lysate. In some embodiments, the lysate is a tumor lysate. In some embodiments, the antigen is a viral antigen, a bacterial antigen or a fungal antigen. In some embodiments, the antigen is a microorganism. In some embodiments, the antigen is a polypeptide. In some embodiments, the antigen is a lipid antigen. In some embodiments, the antigen is a carbohydrate antigen. In some embodiments, a nucleic acid encoding the antigen is delivered to the cell. In some embodiments, the antigen is a modified antigen. In some embodiments, the modified antigen comprises an antigen fused with a polypeptide. In some embodiments, the modified antigen comprises an antigen fused with a targeting peptide. In some embodiments, the modified antigen comprises an antigen fused with a lipid. In some embodiments, the modified antigen comprises an antigen fused with a carbohydrate. In some embodiments, the modified antigen comprises an antigen fused with a nanoparticle. In some embodiments, a plurality of antigens is delivered to the anucleate cell-derived vesicle.

In some embodiments of the above method for generating an anucleate cell-derived vesicle, the adjuvant is a CpG ODN, IFN-α, STING agonists, RIG-I agonists, poly I:C, polyinosinic-polycytidylic acid stabilized with polylysine and carboxymethylcellulose (HILTONOL®), imiquimod, resiquimod, and/or LPS. In some embodiments, the adjuvant is a low molecular weight poly I:C.

In some embodiments of the above method for generating an anucleate cell-derived vesicle, the input anucleate cell is a red blood cell. In some embodiments, the input anucleate cell is an erythrocyte. In some embodiments, the input anucleate cell is a reticulocyte. In some embodiments, the input anucleate cell is a platelet. In some embodiments, the input anucleate cell is a mammalian cell. In some embodiments, the input anucleate cell is a monkey, mouse, dog, cat, horse, rat, sheep, goat, pig, or rabbit cell. In some embodiments, the input anucleate cell is a human cell.

In some embodiments of the above method for generating an anucleate cell-derived vesicle, the half-life of the anucleate cell-derived vesicle following administration to a mammal is decreased compared to a half-life of the input anucleate cell following administration to the mammal. In some embodiments, the hemoglobin content of the anucleate cell-derived vesicle is decreased compared to the hemoglobin content of the input anucleate cell. In some embodiments, ATP production of the anucleate cell-derived vesicle is decreased compared to ATP production of the input anucleate cell. In some embodiments, the anucleate cell-derived vesicle exhibits a spherical morphology. In some embodiments, the input anucleate cell is an erythrocyte and wherein the anucleate cell-derived vesicle has a reduced biconcave shape compared to the input anucleate cell. In some embodiments, the anucleate cell-derived vesicle is a red blood cell ghost. In some embodiments, the anucleate cell-derived vesicles prepared by the process have greater than about 1.5 fold more phosphatidylserine on its surface compared to the input anucleate cell. In some embodiments, a population profile of anucleate cell-derived vesicles prepared by the process exhibits higher average phosphatidylserine levels on the surface compared to the input anucleate cells. In some embodiments, at least 50% of the population profile of anucleate cell-derived vesicles prepared by the process exhibits higher phosphatidylserine levels on the surface compared to the input anucleate cells. In some embodiments, the anucleate cell-derived vesicle exhibits enhanced uptake in a tissue or cell compared to the input anucleate cell. In some embodiments, the anucleate cell-derived vesicle exhibits enhanced uptake in phagocytic cells and/or antigen presenting cells compared to the input anucleate cell. In some embodiments, the anucleate cell-derived vesicle is modified to enhance uptake in a tissue or cell compared to the input anucleate cell. In some embodiments, the anucleate cell-derived vesicle is modified to enhance uptake in phagocytic cells and/or antigen presenting cells compared to an unmodified anucleate cell-derived vesicle. In some embodiments, the phagocytic cells and/or antigen presenting cells comprise one or more of a dendritic cell or macrophage. In some embodiments, the tissue or cell comprises one or more of liver or spleen. In some embodiments, the anucleate cell-derived vesicle comprises CD47 on its surface.

In some embodiments of the above method for generating an anucleate cell-derived vesicle, the anucleate cell-derived vesicle is not (a) heat processed, (b) chemically treated, and/or (c) subjected to hypotonic or hypertonic conditions during the preparation of the anucleate cell-derived vesicles. In some embodiments, the osmolarity of the cell suspension is maintained throughout the process. In some embodiments, the osmolarity of the cell suspension is maintained between about 200 mOsm and about 400 mOsm throughout the process.

In some embodiments of the above method for generating an anucleate cell-derived vesicle, the constriction is contained within a microfluidic channel. In some embodiments, the microfluidic channel comprises a plurality of constrictions. In some embodiments, the plurality of constrictions is arranged in series and/or in parallel. In some embodiments, the constriction is between a plurality of micropillars; between a plurality of micropillars configured in an array; or between one or more movable plates. In some embodiments, the constriction is a pore or contained within a pore. In some embodiments, the pore is contained in a surface. In some embodiments, the surface is a filter. In some embodiments, the surface is a membrane. In some embodiments, the constriction size is a function of the diameter of the input anucleate cell in suspension. In some embodiments, the constriction size is about 10%, about 20%, about 30%, about 40%, about 50%, about 60%, or about 70% of the diameter of the input anucleate cell in suspension. In some embodiments, the constriction has a width of about 0.25 μm to about 4 μm. In some embodiments, the constriction has a width of about 4 μm, 3.5 μm, about 3 μm, about 2.5 μm, about 2 μm, about 1.5 μm, about 1 μm, about 0.5 μm, or about 0.25 μm. In some embodiments, the constriction has a width of about 2.2 μm. In some embodiments, the input anucleate cells are passed through the constriction under a pressure ranging from about 10 psi to about 90 psi. In some embodiments, said cell suspension is contacted with the antigen before, concurrently, or after passing through the constriction.

The present disclosure provides, in one aspect, an anucleate cell-derived vesicle prepared from a parent anucleate cell, the anucleate cell-derived vesicle having one or more of the following properties: (a) a circulating half-life in a mammal is decreased compared to the parent anucleate cell, (b) decreased hemoglobin levels compared to the parent anucleate cell, (c) spherical morphology, (d) increased surface phosphatidylserine levels compared to the parent anucleate cell, or (e) reduced ATP production compared to the parent anucleate cell.

In some embodiments, the parent anucleate cell is a mammalian cell. In some embodiments, the parent anucleate cell is human cell. In some embodiments, the parent anucleate cell is a red blood cell or a platelet. In some embodiments, the red blood cell is an erythrocyte or a reticulocyte.

In some embodiments, the circulating half-life of the anucleate cell-derived vesicle in a mammal is decreased compared to the parent anucleate cell. In some embodiments, the circulating half-life in the mammal is decreased by more than about 50%, about 60%, about 70%, about 80%, about 90%, about 95%, about 96%, about 97%, about 98%, or about 99% compared to the parent anucleate cell.

In some embodiments, the parent anucleate cell is a human cell and wherein the circulating half-life of the anucleate cell-derived vesicle is less than about 1 minute, about 2 minutes, about 5 minutes, about 10 minutes, about 15 minutes, about 30 minutes, about 1 hour, about 6 hours, about 12 hours, about 1 day, about 2 days, about 3 days, about 4 days, about 5 days, about 10 days, about 25 days, about 50 days, about 75 days, about 100 days, about 120 days.

In some embodiments, the parent anucleate cell is a red blood cell, wherein the hemoglobin levels in the anucleate cell-derived vesicle are decreased compared to the parent anucleate cell. In some embodiments, the hemoglobin levels in the anucleate cell-derived vesicle are decreased by at least about 10%, about 20%, about 30%, about 40%, about 50%, about 60%, about 70%, about 80%, about 90%, about 99% or about 100% compared to the parent anucleate cell. In some embodiments, the hemoglobin levels in the anucleate cell-derived vesicle are about 1%, about 5%, about 10%, about 20%, about 30%, about 40%, or about 50% the level of hemoglobin in the parent anucleate cell.

In some embodiments, the parent anucleate cell is an erythrocyte and wherein the anucleate cell-derived vesicle is spherical in morphology. In some embodiments, the parent anucleate cell is an erythrocyte and wherein the anucleate cell-derived vesicle has a reduced biconcave shape compared to the parent anucleate cell.

In some embodiments, the parent anucleate cell is a red blood cell or an erythrocyte and wherein the anucleate cell-derived vesicle is a red blood cell ghost (RBC ghost).

In some embodiments, the anucleate cell-derived vesicle has increased surface phosphatidylserine levels compared to the parent anucleate cell. In some embodiments, the anucleate cell-derived vesicles prepared by the process has greater than about 1.5 fold more phosphatidylserine on its surface compared to the parent anucleate cell. In some embodiments, the anucleate cell-derived vesicle has about 10%, about 20%, about 30%, about 40%, about 50%, about 60%, about 70%, about 80%, about 90%, about 99%, about 100% or more than about 100% more phosphatidylserine on its surface compared to the parent anucleate cell.

In some embodiments, the anucleate cell-derived vesicle has reduced ATP production compared to the parent anucleate cell. In some embodiments, the anucleate cell-derived vesicle produces ATP at less than about 1%, about 5%, about 10%, about 20%, about 30%, about 40%, or about 50% the level of ATP produced by the parent anucleate cell. In some embodiments, the anucleate cell-derived vesicle does not produce ATP.

In some embodiments, the anucleate cell-derived vesicle is modified to enhance uptake in a tissue or cell compared to the parent anucleate cell. In some embodiments, the anucleate cell-derived vesicle is modified to enhance uptake in liver or spleen or by a phagocytic cell or an antigen-presenting cell compared to the uptake of the parent anucleate cell.

In some embodiments, the anucleate cell-derived vesicle comprises CD47 on its surface.

In some embodiments, the parent anucleate cell was not (a) heat processed, (b) chemically treated, and/or (c) subjected to hypotonic or hypertonic conditions during the preparation of the anucleate cell-derived vesicles. In some embodiments, osmolarity was maintained during preparation of the anucleate cell-derived vesicle from the parent anucleate cell. In some embodiments, the osmolarity was maintained between about 200 mOsm and about 600 mOsm. In some embodiments, the osmolarity was maintained between about 200 mOsm and about 400 mOsm.

In some embodiments, the anucleate cell-derived vesicle was prepared by a process comprising: passing a suspension comprising the input parent anucleate cells through a cell deforming constriction, wherein a diameter of the constriction is a function of a diameter of the input parent anucleate cell in the suspension, thereby causing perturbations of the anucleate cell large enough for a payload to pass through; thereby producing an anucleate cell-derived vesicle.

In some embodiments, the anucleate cell-derived vesicle comprises a payload. In some embodiments, the payload is a polypeptide, a nucleic acid, a lipid, a carbohydrate, a small molecule, a complex, a nanoparticle.

In some embodiments, the anucleate cell-derived vesicle was prepared by a process comprising: (a) passing a cell suspension comprising the input parent anucleate cell through a cell-deforming constriction, wherein a diameter of the constriction is a function of a diameter of the input parent anucleate cell in the suspension, thereby causing perturbations of the input parent anucleate cell large enough for the payload to pass through to form an anucleate cell-derived vesicle; and (b) incubating the anucleate cell-derived vesicle with the payload for a sufficient time to allow the payload to enter the anucleate cell-derived vesicle; thereby producing an anucleate cell-derived vesicle comprising the payload.

In some embodiments, the anucleate cell-derived vesicle comprises an antigen. In some embodiments, the anucleate cell-derived vesicle comprises adjuvant. In some embodiments, the anucleate cell-derived vesicle comprises an antigen and/or a tolerogenic factor.

In some embodiments, the anucleate cell-derived vesicle was prepared by a process comprising: (a) passing a cell suspension comprising the input parent anucleate cell through a cell-deforming constriction, wherein a diameter of the constriction is a function of a diameter of the input parent anucleate cell in the suspension, thereby causing perturbations of the input parent anucleate cell large enough for the antigen to pass through to form an anucleate cell-derived vesicle; and (b) incubating the anucleate cell-derived vesicle with the antigen for a sufficient time to allow the antigen to enter the anucleate cell-derived vesicle; thereby producing an anucleate cell-derived vesicle comprising an antigen.

In some embodiments, the anucleate cell-derived vesicle was prepared by a process comprising: (a) passing a cell suspension comprising the input parent anucleate cell through a cell-deforming constriction, wherein a diameter of the constriction is a function of a diameter of the input parent anucleate cell in the suspension, thereby causing perturbations of the input parent anucleate cell large enough for the adjuvant to pass through to form an anucleate cell-derived vesicle; and (b) incubating the anucleate cell-derived vesicle with the adjuvant for a sufficient time to allow the adjuvant to enter the anucleate cell-derived vesicle; thereby producing an anucleate cell-derived vesicle comprising an adjuvant.

In some embodiments, the anucleate cell-derived vesicle comprises an antigen and an adjuvant, wherein the anucleate cell-derived vesicle was prepared by a process comprising: (a) passing a cell suspension comprising the input parent anucleate cell through a cell-deforming constriction, wherein a diameter of the constriction is a function of a diameter of the input parent anucleate cell in the suspension, thereby causing perturbations of the input parent anucleate cell large enough for the antigen and the adjuvant to pass through to form an anucleate cell-derived vesicle; and (b) incubating the anucleate cell-derived vesicle with the antigen and adjuvant for a sufficient time to allow the antigen and the adjuvant to enter the anucleate cell-derived vesicle; thereby producing an anucleate cell-derived vesicle comprising an antigen and an adjuvant.

In some embodiments, the anucleate cell-derived vesicle comprises an antigen and a tolerogenic factor, wherein the anucleate cell-derived vesicle was prepared by a process comprising: (a) passing a cell suspension comprising the input parent anucleate cell through a cell-deforming constriction, wherein a diameter of the constriction is a function of a diameter of the input parent anucleate cell in the suspension, thereby causing perturbations of the input parent anucleate cell large enough for the antigen and the tolerogenic factor to pass through to form an anucleate cell-derived vesicle; and (b) incubating the anucleate cell-derived vesicle with the antigen and the tolerogenic factor for a sufficient time to allow the antigen and the tolerogenic factor to enter the anucleate cell-derived vesicle; thereby producing an anucleate cell-derived vesicle comprising an antigen and an tolerogenic factor.

In some embodiments, the constriction is contained within a microfluidic channel. In some embodiments, the microfluidic channel comprises a plurality of constrictions. In some embodiments, the plurality of constrictions are arranged in series and/or in parallel. In some embodiments, the constriction is between a plurality of micropillars, between a plurality of micropillars configured in an array, or between one or more movable plates. In some embodiments, the constriction is a pore or contained within a pore. In some embodiments, the pore is contained in a surface. In some embodiments, the surface is a filter. In some embodiments, the surface is a membrane. In some embodiments, the constriction size is about 10%, about 20%, about 30%, about 40%, about 50%, about 60%, or about 70% of the cell diameter. In some embodiments, the constriction has a width of about 0.25 μm to about 4 μm. In some embodiments, the constriction has a width of about 4 μm, 3.5 μm, about 3 μm, about 2.5 μm, about 2 μm, about 1.5 μm, about 1 μm, about 0.5 μm, or about 0.25 μm. In some embodiments, the constriction has a width of about 2.2 μm. In some embodiments, the input parent anucleate cells are passed through the constriction under a pressure ranging from about 10 psi to about 150 psi. In some embodiments, the cell suspension is contacted with the payload before, concurrently, or after passing through the constriction.

In some embodiments, the antigen is capable of being processed into an MHC class I-restricted peptide and/or an MHC class II-restricted peptide.

In some embodiments, the antigen is a disease-associated antigen. In some embodiments, the antigen is a tumor antigen. In some embodiments, the antigen is derived from a lysate. In some embodiments, the antigen is derived from a transplant lysate. In some embodiments, the lysate is a tumor lysate. In some embodiments, the antigen is a viral antigen, a bacterial antigen or a fungal antigen. In some embodiments, the viral antigen is a virus, a viral particle, or a viral capsid. In some embodiments, the antigen is a microorganism. In some embodiments, the antigen is a polypeptide. In some embodiments, the antigen is a lipid antigen. In some embodiments, the antigen is a carbohydrate antigen. In some embodiments, a nucleic acid encoding the antigen is delivered to the cell.

In some embodiments, the antigen is a modified antigen. In some embodiments, the modified antigen comprises an antigen fused with a polypeptide. In some embodiments, the modified antigen comprises an antigen fused with a targeting peptide. In some embodiments, the modified antigen comprises an antigen fused with a lipid. In some embodiments, the modified antigen comprises an antigen fused with a carbohydrate. In some embodiments, the modified antigen comprises an antigen fused with a nanoparticle.

In some embodiments, a plurality of antigens is delivered to the anucleate cell.

In some embodiments, the adjuvant is a CpG ODN, IFN-α, STING agonists, RIG-I agonists, poly I:C, imiquimod, resiquimod, and/or lipopolysaccharide (LPS).

The present disclosure provides, in another aspect, a composition comprising a plurality of anucleate cell-derived vesicles according to the description herein.

The present disclosure provides, in another aspect, a composition comprising a plurality of anucleate cell-derived vesicles prepared from parent anucleate cells, the composition having one or more of the following properties: (a) greater than about 20% of the anucleate cell-derived vesicles in the composition have a circulating half-life in a mammal that is decreased compared to the parent anucleate cell, (b) greater than 20% of the anucleate cell-derived vesicles in the composition have decreased hemoglobin levels compared to the parent anucleate cell, (c) greater than 20% of the anucleate cell-derived vesicles in the composition have spherical morphology, (d) greater than 20% of the anucleate cell-derived vesicles in the composition are RBC ghosts, (e) greater than 20% of the anucleate cell-derived vesicles in the composition vesicles in the composition have higher levels of phosphatidylserine compared to the population of parent anucleate cells, or (f) greater than 20% of the anucleate cell-derived vesicles in the composition have reduced ATP production compared to the parent anucleate cell.

The present disclosure provides, in another aspect, a composition comprising a plurality of anucleate cell-derived vesicles prepared from a population of a parent anucleate cell, the composition having one or more of the following properties: (a) greater than about 20% of the anucleate cell-derived vesicles in the composition have a circulating half-life in a mammal that is decreased compared to the average of the population of the parent anucleate cell, (b) greater than 20% of the anucleate cell-derived vesicles in the composition have decreased hemoglobin levels compared to the average of the population of the parent anucleate cell, (c) greater than 20% of the anucleate cell-derived vesicles in the composition have spherical morphology, (d) greater than 20% of the anucleate cell-derived vesicles in the composition are RBC ghosts, (e) greater than 20% of the anucleate cell-derived vesicles in the composition vesicles in the composition have higher levels of phosphatidylserine compared to the average of the population of the parent anucleate cell, or (f) greater than 20% of the anucleate cell-derived vesicles in the composition have reduced ATP production compared to the average of the population of the parent anucleate cell.

In some embodiments, the parent anucleate cell used to prepare the composition is a mammalian cell. In some embodiments, the parent anucleate cell used to prepare the composition is a human cell. In some embodiments, the parent anucleate cell used to prepare the composition is a red blood cell or a platelet. In some embodiments, the red blood cell is an erythrocyte or a reticulocyte.

In some embodiments, the circulating half-life of 20% of the anucleate cell-derived vesicles in the composition in a mammal is decreased compared to the parent anucleate cell or the average of the population of the parent anucleate cell. In some embodiments, the circulating half-life of 20% of the anucleate cell-derived vesicles in the composition in the mammal is decreased by more than about 50%, about 60%, about 70%, about 80% or about 90% compared to the parent anucleate cell or the average of the population of the parent anucleate cell. In some embodiments, the parent anucleate cell used to prepare the composition is a human cell and wherein the circulating half-life of 20% of the anucleate cell-derived vesicles in the composition is less than about 5 minutes, about 10 minutes, about 15 minutes, about 30 minutes, about 1 hour, about 6 hours, about 12 hours, about 1 day, about 2 days, about 3 days, about 4 days, about 5 days, about 10 days.

In some embodiments, the parent anucleate cell used to prepare the composition is a red blood cell and wherein the hemoglobin levels of 20% of the anucleate cell-derived vesicles in the composition are decreased compared to the parent anucleate cell or the average of the population of the parent anucleate cell.

In some embodiments, the hemoglobin levels of 20% of the anucleate cell-derived vesicles in the composition of the anucleate cell-derived vesicle are decreased by at least about 10%, about 20%, about 30%, about 40%, about 50%, about 60%, about 70%, about 80%, about 90%, about 99% or about 100% compared to the parent anucleate cell or the average of the population of the parent anucleate cell. In some embodiments, the hemoglobin levels of 20% of the anucleate cell-derived vesicles in the composition are about 1%, about 5%, about 10%, about 20%, about 30%, about 40%, or about 50% the level of hemoglobin in the parent anucleate cell or the average of the population of the parent anucleate cell.

In some embodiments, the parent anucleate cell used to prepare the composition is an erythrocyte and wherein greater than 20% of the anucleate cell-derived vesicles in the composition are spherical in morphology. In some embodiments, the parent anucleate cell used to prepare the composition is an erythrocyte and wherein greater than 20% of the anucleate cell-derived vesicles in the composition have a reduced biconcave shape compared to the parent anucleate cell.

In some embodiments, the parent anucleate cell used to prepare the composition is a red blood cell or an erythrocyte and wherein greater than 20% of the anucleate cell-derived vesicles in the composition are red blood cell ghosts.

In some embodiments, the anucleate cell-derived vesicles in the composition comprise surface phosphatidylserine. In some embodiments, 20% of the anucleate cell-derived vesicles in the composition comprise increased surface phosphatidylserine levels compared to the parent anucleate cells or the average of the population of the parent anucleate cell. In some embodiments, 20% of the anucleate cell-derived vesicles in the composition have about 10%, about 20%, about 30%, about 40%, about 50%, about 60%, about 70%, about 80%, about 90%, about 99%, about 100% or more than about 100% higher surface phosphatidylserine levels compared to a composition comprising a plurality of parent anucleate cells.

In some embodiments, 20% of the anucleate cell-derived vesicles in the composition have reduced ATP production compared to the parent anucleate cell or the average of the population of the parent anucleate cell. In some embodiments, 20% of the anucleate cell-derived vesicles in the composition produce ATP at less than about 1%, about 5%, about 10%, about 20%, about 30%, about 40%, or about 50% the level of ATP produced by the parent anucleate cell or the average of the population of the parent anucleate cell. In some embodiments, the anucleate cell-derived vesicle in the composition does not produce ATP.

In some embodiments, the parent anucleate cell used to prepare the composition was not (a) heat processed, (b) chemically treated, and/or (c) subjected to hypotonic or hypertonic conditions during the preparation of the compositions. In some embodiments, osmolarity was maintained during preparation of the anucleate cell-derived vesicles from the parent anucleate cell. In some embodiments, the osmolarity was maintained between about 200 mOsm and about 600 mOsm. In some embodiments, the osmolarity was maintained between about 200 mOsm and about 400 mOsm.

In some embodiments, the anucleate cell-derived vesicles of the composition were prepared by a process comprising: passing a suspension comprising the input parent anucleate cells through a cell deforming constriction, wherein a diameter of the constriction is a function of a diameter of the input parent anucleate cells in the suspension, thereby causing perturbations of the anucleate cells large enough for a payload to pass through; thereby producing the anucleate cell-derived vesicles.

In some embodiments, the anucleate cell-derived vesicles of the composition comprise a payload. In some embodiments, the payload is a therapeutic payload. In some embodiments, the payload is a polypeptide, a nucleic acid, a lipid, a carbohydrate a small molecule, a complex, a nanoparticle.

In some embodiments, the anucleate cell-derived vesicles of the composition were prepared by a process comprising: (a) passing a cell suspension comprising the input parent anucleate cells through a cell-deforming constriction, wherein a diameter of the constriction is a function of a diameter of the input parent anucleate cells in the suspension, thereby causing perturbations of the input parent anucleate cells large enough for the payload to pass through to form an anucleate cell-derived vesicles; and (b) incubating the anucleate cell-derived vesicles with the payload for a sufficient time to allow the payload to enter the anucleate cell-derived vesicles; thereby producing an anucleate cell-derived vesicles comprising the payload.

In some embodiments, the anucleate cell-derived vesicles of the composition comprise an antigen. In some embodiments, the anucleate cell-derived vesicles of the composition comprise an adjuvant. In some embodiments, the anucleate cell-derived vesicles of the composition comprise an antigen and a tolerogenic factor.

In some embodiments, the anucleate cell-derived vesicles of the composition were prepared by a process comprising: (a) passing a cell suspension comprising the input parent anucleate cell through a cell-deforming constriction, wherein a diameter of the constriction is a function of a diameter of the input parent anucleate cell in the suspension, thereby causing perturbations of the input parent anucleate cell large enough for the antigen to pass through to form an anucleate cell-derived vesicle; and (b) incubating the anucleate cell-derived vesicle with the antigen for a sufficient time to allow the antigen to enter the anucleate cell-derived vesicle; thereby producing an anucleate cell-derived vesicle comprising an antigen.

In some embodiments, the anucleate cell-derived vesicles of the composition were prepared by a process comprising: (a) passing a cell suspension comprising the input parent anucleate cell through a cell-deforming constriction, wherein a diameter of the constriction is a function of a diameter of the input parent anucleate cell in the suspension, thereby causing perturbations of the input parent anucleate cell large enough for the adjuvant to pass through to form an anucleate cell-derived vesicle; and (b) incubating the anucleate cell-derived vesicle with the adjuvant for a sufficient time to allow the adjuvant to enter the anucleate cell-derived vesicle; thereby producing an anucleate cell-derived vesicle comprising an adjuvant.

In some embodiments, the anucleate cell-derived vesicles of the composition comprises an antigen and an adjuvant, wherein the anucleate cell-derived vesicles of the composition were prepared by a process comprising: (a) passing a cell suspension comprising the input parent anucleate cell through a cell-deforming constriction, wherein a diameter of the constriction is a function of a diameter of the input parent anucleate cell in the suspension, thereby causing perturbations of the input parent anucleate cell large enough for the antigen and the adjuvant to pass through to form an anucleate cell-derived vesicle; and (b) incubating the anucleate cell-derived vesicle with the antigen and the adjuvant for a sufficient time to allow the antigen and the adjuvant to enter the anucleate cell-derived vesicle; thereby producing an anucleate cell-derived vesicle comprising an antigen and/or an adjuvant.

In some embodiments, the anucleate cell-derived vesicle of the composition comprises an antigen and a tolerogenic factor, wherein the anucleate cell-derived vesicles of the composition were prepared by a process comprising: (a) passing a cell suspension comprising the input parent anucleate cell through a cell-deforming constriction, wherein a diameter of the constriction is a function of a diameter of the input parent anucleate cell in the suspension, thereby causing perturbations of the input parent anucleate cell large enough for the antigen and the tolerogenic factor to pass through to form an anucleate cell-derived vesicle; and (b) incubating the anucleate cell-derived vesicle with the antigen and the tolerogenic factor for a sufficient time to allow the antigen and the tolerogenic factor to enter the anucleate cell-derived vesicle; thereby producing an anucleate cell-derived vesicle comprising an antigen and/or an tolerogenic factor.

In some embodiments, the constriction used to prepare the composition is contained within a microfluidic channel. In some embodiments, the microfluidic channel used to prepare the composition comprises a plurality of constrictions. In some embodiments, the plurality of constrictions are arranged in series and/or in parallel. In some embodiments, the constriction used to prepare the composition is between a plurality of micropillars, between a plurality of micropillars configured in an array, or between one or more movable plates. In some embodiments, the constriction used to prepare the composition is a pore or contained within a pore. In some embodiments, the pore used to prepare the composition is contained in a surface. In some embodiments, the surface used to prepare the composition is a filter. In some embodiments, the surface used to prepare the composition is a membrane. In some embodiments, the constriction size used to prepare the composition is about 10%, about 20%, about 30%, about 40%, about 50%, about 60%, or about 70% of the cell diameter. In some embodiments, the constriction used to prepare the composition has a width of about 0.25 μm to about 4 μm. In some embodiments, the constriction used to prepare the composition has a width of about 4 μm, 3.5 μm, about 3 μm, about 2.5 μm, about 2 μm, about 1.5 μm, about 1 μm, about 0.5 μm, or about 0.25 μm. In some embodiments, the constriction used to prepare the composition has a width of about 2.2 μm. In some embodiments, the input parent anucleate cells used to prepare the composition are passed through the constriction under a pressure ranging from about 10 psi to about 150 psi. In some embodiments, the cell suspension used to prepare the composition is contacted with the antigen before, concurrently, or after passing through the constriction.

In some embodiments, the antigen of the composition is capable of being processed into an MHC class I-restricted peptide and/or an MHC class II-restricted peptide. In some embodiments, the antigen is a disease-associated antigen. In some embodiments, the antigen is a tumor antigen. In some embodiments, the antigen is derived from a lysate. In some embodiments, the lysate is a transplant lysate. In some embodiments, the lysate is a tumor lysate. In some embodiments, the antigen is a viral antigen, a bacterial antigen or a fungal antigen. In some embodiments, the antigen is a microorganism. In some embodiments, the antigen is a polypeptide. In some embodiments, the antigen is a lipid antigen. In some embodiments, the antigen is a carbohydrate antigen. In some embodiments, a nucleic acid encoding the antigen is delivered to the cell.

In some embodiments, the antigen of the composition is a modified antigen. In some embodiments, the modified antigen comprises an antigen fused with a polypeptide. In some embodiments, the modified antigen comprises an antigen fused with a targeting peptide. In some embodiments, the modified antigen comprises an antigen fused with a lipid. In some embodiments, the modified antigen comprises an antigen fused with a carbohydrate. In some embodiments, the modified antigen comprises an antigen fused with a nanoparticle.

In some embodiments, the anucleate cell-derived vesicle of the composition comprises a plurality of antigens, wherein the plurality of antigens is delivered to the anucleate cell.

In some embodiments, the adjuvant of the composition is a CpG ODN, IFN-α, STING agonists, RIG-I agonists, poly I:C, imiquimod, resiquimod, and/or LPS.

In some embodiments, the composition is a pharmaceutical composition.

The present disclosure provides, in another aspect, a method of making a composition comprising a plurality of anucleate cell-derived vesicles prepared from parent anucleate cells, the composition having one or more of the following properties: (a) greater than 20% of the anucleate cell-derived vesicles in the composition have a circulating half-life in a mammal that is decreased compared to the parent anucleate cell, (b) greater than 20% of the anucleate cell-derived vesicles in the composition have decreased hemoglobin levels compared to the parent anucleate cell, (c) greater than 20% of the anucleate cell-derived vesicles in the composition have spherical morphology, (d) greater than 20% of the anucleate cell-derived vesicles in the composition are RBC ghosts, (e) greater than 20% of the anucleate cell-derived vesicles in the composition have higher levels of phosphatidylserine, or (f) greater than 20% of the anucleate cell-derived vesicles in the composition have reduced ATP production compared to the parent anucleate cell; the method comprising passing a cell suspension comprising the parent anucleate cell through a cell-deforming constriction, wherein a diameter of the constriction is a function of a diameter of the parent anucleate cell in the suspension, thereby causing perturbations of the parent anucleate cell large enough for a payload to pass through to form an anucleate cell-derived vesicle, thereby producing an anucleate cell-derived vesicle.

In some embodiments, the constriction used in the methods of making described herein is contained within a microfluidic channel. In some embodiments, the microfluidic channel used in the methods of making described herein comprises a plurality of constrictions. In some embodiments, the plurality of constrictions used in the methods of making described herein are arranged in series and/or in parallel. In some embodiments, the constriction used in the methods of making described herein is between a plurality of micropillars, between a plurality of micropillars configured in an array, or between one or more movable plates. In some embodiments, the constriction used in the methods of making described herein is a pore or contained within a pore. In some embodiments, the pore used in the methods of making described herein is contained in a surface. In some embodiments, the surface used in the methods of making described herein is a filter. In some embodiments, the surface used in the methods of making described herein is a membrane. In some embodiments, the constriction size used in the methods of making described herein is about 10%, about 20%, about 30%, about 40%, about 50%, about 60%, or about 70% of the cell diameter. In some embodiments, the constriction used in the methods of making described herein has a width of about 0.25 μm to about 4 μm. In some embodiments, the constriction used in the methods of making described herein has a width of about 4 μm, 3.5 μm, about 3 μm, about 2.5 μm, about 2 μm, about 1.5 μm, about 1 μm, about 0.5 μm, or about 0.25 μm. In some embodiments, the constriction used in the methods of making described herein has a width of about 2.2 μm. In some embodiments, the input parent anucleate cells used in the methods of making described herein are passed through the constriction under a pressure ranging from about 10 psi to about 150 psi. In some embodiments, the cell suspension used in the methods of making described herein is contacted with a payload before, concurrently, or after passing through the constriction such that the payload enters the cell.

In some embodiments, the payload used in the methods of making described herein is a therapeutic payload. In some embodiments, the payload is a polypeptide, a nucleic acid, a lipid, a carbohydrate a small molecule, a complex, or a nanoparticle. In some embodiments, the payload is an antigen and/or an adjuvant. In some embodiments, the payload is an antigen and/or a tolerogenic factor.

The present disclosure provides, in another aspect, a method for treating a disease or disorder in an individual in need thereof, the method comprising administering a anucleate cell-derived vesicle described herein. The present disclosure provides, in another aspect, a method for treating a disease or disorder in an individual in need thereof, the method comprising administering a composition described herein. In some embodiments, the anucleate cell-derived vesicles used in the methods for treating described herein comprise a therapeutic payload. In some embodiments, the individual has cancer and wherein the payload comprises an antigen. In some embodiments, the individual has cancer and wherein the payload comprises an antigen and an adjuvant. In some embodiments, the antigen is a tumor antigen. In some embodiments, the individual has an infectious disease or a viral-associated disease and wherein the payload comprises an antigen. In some embodiments, the individual has an infectious disease or a viral-associated disease and wherein the payload comprises an antigen and an adjuvant. In some embodiments, the antigen is a viral antigen, a bacterial antigen or a fungal antigen. In some embodiments, the individual has an autoimmune disease and wherein the payload comprises an antigen. In some embodiments, the individual has an autoimmune disease and wherein the payload comprises an antigen and/or a tolerogenic factor.

The present disclosure provides, in another aspect, a method for preventing a disease or disorder in an individual in need thereof, the method comprising administering a anucleate cell-derived vesicle described herein. The present disclosure provides, in another aspect, a method for preventing a disease or disorder in an individual in need thereof, the method comprising administering a composition described herein. In some embodiments, the anucleate cell-derived vesicles used in the methods for preventing described herein comprise an antigen. In some embodiments, the individual has cancer and wherein the payload comprises an antigen and an adjuvant. In some embodiments, the disease or disorder is cancer and the antigen is a tumor antigen. In some embodiments, the individual has an infectious disease and wherein the payload comprises an antigen. In some embodiments, the individual has an infectious disease and wherein the payload comprises an antigen and an adjuvant In some embodiments, the antigen is a viral antigen, a bacterial antigen or a fungal antigen.

### BRIEF DESCRIPTION OF THE DRAWINGS

FIG. 1A shows the percentage of antigen-specific T cells as measured by tetramer staining for each condition. FIG. 1B shows the percentage of IFN-γ positive cells as measured by intracellular cytokine staining (ICS) for each condition after re-stimulation with OVA epitope SIINFEKL (circular dots). Stimulation with anti-CD28 alone (without SIINFEKL) is used as a negative control (square dots), while unspecific stimulation by PMA/Ionomycin is used as a positive control (triangular dots). FIG. 1C shows the amounts of IFN-γ in each cell as measured by the mean fluorescence intensity (MFI) of each cell in ICS for each condition. FIG. 1D shows the percentage of IL-2 positive cells as measured by intracellular cytokine staining (ICS) for each condition after re-stimulation with OVA epitope SIINFEKL (circular dots). Stimulation with anti-CD28 alone (without SIINFEKL) is used as a negative control (square dots), while unspecific stimulation by PMA/Ionomycin is used as a positive control (triangular dots). FIG. 1E shows the amounts of IL-2 in each cell as measured by the mean fluorescence intensity (MFI) of each cell in ICS for each condition.

FIG. 2A shows the percentage of antigen-specific T cells as measured by tetramer staining for each condition. FIG. 2B shows the percentage of IFN-γ positive cells as measured by intracellular cytokine staining (ICS) for each condition after re-stimulation with OVA epitope SIINFEKL (circular dots). FIG. 2C shows the percentage of IL-2 positive cells as measured by intracellular cytokine staining (ICS) for each condition after re-stimulation with OVA epitope SIINFEKL (circular dots). For both FIGS. 2B and 2C, Stimulation with anti-CD28 alone (without SIINFEKL) is used as a negative control (square dots), while unspecific stimulation by PMA/Ionomycin is used as a positive control (triangular dots).

FIG. 3A shows the percentage of antigen-specific T cells as measured by tetramer staining for each condition. FIG. 3B shows the percentage of IFN-γ positive cells as measured by intracellular cytokine staining (ICS) for each condition after re-stimulation with OVA epitope SIINFEKL (circular dots). FIG. 3C shows the percentage of IL-2 positive cells as measured by intracellular cytokine staining (ICS) for each condition after re-stimulation with OVA epitope SIINFEKL (circular dots). For both FIGS. 3B and 3C, Stimulation with anti-CD28 alone (without SIINFEKL) is used as a negative control (square dots), while unspecific stimulation by PMA/Ionomycin is used as a positive control (triangular dots).

FIG. 4 shows lactate levels of red blood cell derived vesicles that has been processed by constriction mediated delivery (SQZ) versus the unprocessed input red blood cells.

FIG. 5A shows the images from brightfield microscopy, fluorescent microscopy for CellTrace Violet staining (CT) as well as fluorescent microscopy for FITC labeled Dextran (D-FITC), for untreated RBCs (Untrtd), RBCs incubated with D-FITC (No SQZ), as well as RBC-derived vesicles with D-FITC loaded using SQZ (SQZ). FIG. 5B shows the levels of phosphatidylserine staining for untreated RBCs (Untrt), RBCs incubated with D-FITC (No SQZ), as well as RBC-derived vesicles with D-FITC loaded using SQZ (SQZ).

FIG. 6A shows the representative schematics of an experiment to determine the circulating half-life of anucleate cell-derived vesicles generated by SQZ-processing. FIG. 6B shows the circulating levels of the separately labeled RBCs and SQZ-loaded RBC-derived vesicles over time. FIG. 6C shows the forward and side scatter in the flow plot of the mixture of RBCs and SQZ-loaded RBC-derived vesicles that were injected into mice.

FIG. 7A shows the appearance of cell pellet and supernatant after centrifugation of untreated RBCs (NC), and RBC-derived vesicles that were SQZ-processed at pressure of 10 psi and 12 psi, respectively. FIG. 7B shows the loss of hemoglobin (hemolysis) as measured by HemoCue® system for untreated RBCs (NC), RBC-derived vesicles that were SQZ-processed at pressure of 10 psi and 12 psi, and RBCs diluted in water (Lysis Control).

FIGS. 8A and 8B show the loss of hemoglobin (hemolysis) as quantified by liquid chromatography/mass spectrometry of 2 hemoglobin peptide, respectively, in RBCs incubated with B9-23 (Endo Control) and RBC-derived vesicles that were SQZ-loaded with B9-23 (SQZ).

FIG. 9 shows the percentage of ghost formation in SQZ-mediated derivation of RBC-derived vesicles under various constriction widths and driving pressures in SQZ-processing.

FIG. 10 shows the in vivo persistence of unprocessed murine RBCs and SQZ-processed murine RBC vesicles in recipient mice.

FIG. 11A shows the organs involved in internalization of SQZ-processed RBC-derived vesicles. FIG. 11B shows the cell types within liver and spleen that are involved in internalization of SQZ-processed RBC-derived vesicles.

FIG. 12A shows the proliferation of OVA-specific CD4+ T cell proliferation induced by RBC-derived vesicles SQZ-loaded with OVA and Poly I:C. FIG. 12B shows the proliferation of OVA-specific CD8+ T cell proliferation induced by RBC-derived vesicles SQZ-loaded with OVA and Poly I:C.

FIG. 13 shows the endogenous CD8+ T cell response upon ex vivo SIINFEKL re-simulation for mice administered with induced by RBC-derived vesicles SQZ-loaded with (i) Poly I:C only, (ii) OVA only, or (iii) OVA and Poly I:C.

FIG. 14 shows the endogenous CD8+ T cell response upon ex vivo E7 re-stimulation for mice induced by RBC-derived vesicles SQZ-loaded with (i) Poly I:C only, (ii) E7 only, or (iii) E7 and Poly I:C.

FIG. 15 shows the quantification of E7-specific CD8+ T cells for mice treated with different priming and boosting dosing regimens of RBC-derived vesicles SQZ-loaded with E7 and Poly I:C.

FIGS. 16A and 16B show the effect of prophylactic administration of RBC-derived vesicles SQZ-loaded with E7 and Poly I:C on the tumor growth and survival respectively in a murine model receiving E7-positive tumor.

FIGS. 17A and 17B show the effect of therapeutic administration of RBC-derived vesicles SQZ-loaded with E7 and Poly I:C at different dosages on the tumor growth and survival respectively in a murine model carrying E7-positive tumor.

FIGS. 18A and 18B show the effect of therapeutic administration of RBC-derived vesicles SQZ-loaded with E7 and Poly I:C with different dosing regimens on the tumor growth and survival respectively in a murine model carrying E7-positive tumor.

FIGS. 19A-19D show the antigen-specific immune response induced by RBC-derived vesicles SQZ-loaded with E7 and Poly I:C, specifically the recruitment of CD8+ T cells into an E7 positive tumor (FIG. 19A), the percentage of CD8+ T cells within the tumor that is specific to E7 (FIG. 19B), the ratio of E7-specific CD8+ T cells versus regulatory T cells in the tumor (FIG. 19C), and correlation of E7-specific CD8+ T cells versus tumor weight (FIG. 19D), when a murine model carrying a E7-positive tumor was administered with RBC-derived vesicles SQZ-loaded with E7 and Poly I:C.

FIGS. 20A-20C show the ghost formation, the efficiency of payload delivery, and the surface phosphatidylserine levels respectively when human RBC-derived vesicles were generated by SQZ-processing in the presence of E7-SLP (payload).

FIG. 21 shows the internalization of human RBC-derived vesicles by human monocyte-derived dendritic cells at 37° C. and at 0° C.

FIG. 22 shows the IFN-γ production and secretion by CMV antigen-specific CD8+ T cells when co-cultured with human RBC-derived vesicles loaded with CMV antigen, and exogenous adjuvant.

FIGS. 23A-23C show the efficiency of payload delivery, the ghost formation, and the surface phosphatidylserine levels in ghost and non-ghost populations, respectively, when murine RBC-derived vesicles were generated by SQZ-processing.

FIG. 24A shows the representative schematics of an experiment to determine if in vivo antigen-dependent tolerance to a viral capsid is induced by anucleate cell-derived vesicles with SQZ-loaded antigen. FIG. 24B shows the percentage of IFN-γ positive cells as measured by intracellular cytokine staining (ICS) in splenocytes of naïve mice, mice treated with RBC incubated with SNYNKSVNV (Peptide), or mice treated with SNYNKSVNV-loaded RBC-derived vesicles (SQZ). FIG. 24C shows the luciferase levels in serum for mice in Peptide group and SQZ group over the course of 43 days.

FIG. 25A shows the representative schematics of an experiment to determine if in vivo antigen-dependent tolerance to an antibody is induced by anucleate cell-derived vesicles with SQZ-loaded antigen. FIG. 25B shows the levels of circulating rat IgG2b in serum for control mice, mice injected with free rat IgG2b, and mice injected with RBC-derived vesicles SQZ-loaded with rat IgG2b (SQZ) on Day 20, as determined by ELISA. FIG. 25C shows the levels of circulating rat IgG2b in serum for mice in control, free rat IgG2b, and SQZ group on Day 76.

FIG. 26A shows the representative schematics of an experiment to determine if in vivo antigen-dependent tolerance to B9-23 is induced by anucleate cell-derived vesicles with SQZ-loaded antigen. FIG. 26B shows the percentage of IFN-γ or IL-2 positive cells as measured by intracellular cytokine staining (ICS) after re-stimulation with AAV-NL virus in splenocytes of control mice, mice treated with HEL-loaded RBC-derived vesicles (SQZ HEL), or mice treated Ins B9-23-loaded RBC-derived vesicles (SQZ FAM). FIG. 26C shows the representative schematics of an experiment to determine if in vivo antigen-dependent tolerance to 1040-p31 is induced by anucleate cell-derived vesicles with SQZ-loaded antigen. FIG. 26D shows the levels of serum blood glucose measured in control mice and mice treated with 1040-31-loaded RBC-derived vesicles (SQZ). FIG. 26E shows the disease onset for control mice and SQZ mice, as determined from serum blood glucose measurements.

### DETAILED DESCRIPTION

The present application provides anucleate cells, including anucleate cell-derived vesicles (such as those prepared from an input anucleate cell), and compositions thereof, wherein the anucleate cells and/or anucleate cell-derived vesicles are loaded and/or admixed with one or more of an antigen, adjuvant, or therapeutic agent. The present application also provides methods of generating anucleate cell-derived vesicles via constriction-mediated delivery (SQZ) described herein and methods of use thereof. The present application further provides methods of stimulating an immune response and of treating and/or preventing diseases in individuals using anucleate cell-derived vesicles generated via constriction-mediated delivery (SQZ) described herein.

The disclosure of the present application is based, at least in part, on the finding that input anucleate cells can be processed by constriction-mediated delivery (SQZ) to generate anucleate cell-derived vesicles. The disclosure of the present application is also based, at least in part, on the finding that anucleate cell-derived vesicles with antigen(s) and/or adjuvant(s) (whether or not encapsulated within the anucleate cell-derived vesicle) can induce an in vivo antigen-specific immune response.

The invention provides methods for delivering an antigen and/or an adjuvant into an anucleate cell-derived vesicle, the method comprising: a) passing a cell suspension comprising an input anucleate cell through a cell-deforming constriction, wherein a diameter of the constriction is a function of a diameter of the input anucleate cell in the suspension, thereby causing perturbations of the input anucleate cell large enough for the antigen and/or adjuvant to pass through to form an anucleate cell-derived vesicle; and b) incubating the anucleate cell-derived vesicle with the antigen and/or the adjuvant for a sufficient time to allow the antigen and/or adjuvant to enter the anucleate cell-derived vesicle.

Certain aspects of the present disclosure relate to methods for stimulating an immune response to an antigen in an individual, the method comprising administering to the individual an effective amount of an anucleate cell-derived vesicle comprising an antigen, wherein the anucleate cell-derived vesicle comprising the antigen is prepared by a process comprising the steps of: a) passing a cell suspension comprising an input anucleate cell through a cell-deforming constriction, wherein a diameter of the constriction is a function of a diameter of the input anucleate cell in the suspension, thereby causing perturbations of the input anucleate cell large enough for the antigen to pass through to form an anucleate cell-derived vesicle; and b) incubating the perturbed input anucleate cell with the antigen for a sufficient time to allow the antigen to enter the anucleate cell-derived vesicle. In some embodiments, an adjuvant is also delivered to the anucleate cell-derived vesicle. In other embodiments, an adjuvant is administered systemically to the individual in combination with the anucleate cell-derived vesicle comprising the antigen.

In certain aspects, the invention provides an anucleate cell-derived vesicle comprising an antigen and/or an adjuvant, wherein the anucleate cell-derived vesicle comprising the antigen and/or adjuvant is prepared by a process comprising the steps of: a) passing a cell suspension comprising an input anucleate cell through a cell-deforming constriction, wherein a diameter of the constriction is a function of a diameter of the input anucleate cell in the suspension, thereby causing perturbations of the input anucleate cell large enough for the antigen and/or adjuvant to pass through to form an anucleate cell-derived vesicle; and b) incubating the anucleate cell-derived vesicle with the antigen and/or adjuvant for a sufficient time to allow the antigen and/or adjuvant to enter the anucleate cell-derived vesicle; thereby generating the anucleate cell-derived vesicle comprising the antigen and/or adjuvant.

In certain aspects, the invention provides methods for generating an anucleate cell-derived vesicle comprising an antigen and/or an adjuvant, the method comprising: a) passing a cell suspension comprising an input anucleate cell through a cell-deforming constriction, wherein a diameter of the constriction is a function of a diameter of the input anucleate cell in the suspension, thereby causing perturbations of the input anucleate cell large enough for the antigen and/or adjuvant to pass through to form an anucleate cell-derived vesicle; b) incubating the anucleate cell-derived vesicle with the antigen and/or adjuvant for a sufficient time to allow the antigen and/or adjuvant to enter the anucleate cell-derived vesicle, thereby generating an anucleate cell-derived vesicle comprising the antigen and/or adjuvant.

In some aspects, the present application provides anucleate cell-derived vesicles (such as those prepared from a parent anucleate cell), and compositions thereof, wherein the anucleate cell-derived vesicles are loaded with a payload, such as any one or more of an antigen, adjuvant, or tolerogenic factor. The present application also provides methods of making compositions of anucleate cell-derived vesicles described herein and methods of use thereof.

The disclosure of the present application is also based, at least in part, on the finding that compositions comprising anucleate cell-derived vesicles comprising a payload, such as an antigen(s) and/or adjuvant(s), can induce an in vivo antigen-specific immune response. The disclosure of the present application is also based, at least in part, on the finding that higher doses of compositions comprising anucleate cell-derived vesicles loaded with an antigen(s) and/or an adjuvant(s) can induce a greater in vivo antigen-specific immune response. Furthermore, the disclosure of the present application is based, at least in part, on the finding that the in vivo antigen-specific immune response can be modulated based on: the adjuvant of the composition; the amount of payload, such as an antigen, encapsulated in an anucleate cell-derived vesicle; and/or the dosing strategy used for administration of the composition comprising anucleate cell-derived vesicles. The disclosure of the present application is also based, at least in part, on the finding that a composition comprising a plurality of anucleate cell-derived vesicles can be actively tuned to generate anucleate cell-derived vesicles, such as a population of anucleate cell-derived vesicles, within the composition having one or more select properties. Generation of a composition of anucleate cell-derived vesicles having desired amounts and/or properties of the anucleate cell-derived vesicle therein is achieved, e.g., by adjusting one or more of the preparation parameters when the anucleate cell-derived vesicles are prepared from parent anucleate cells.

Thus, in some aspects, provided herein are anucleate cell-derived vesicles prepared from a parent anucleate cell, the anucleate cell-derived vesicles having one or more of the following properties: (a) a circulating half-life in a mammal is decreased compared to the parent anucleate cell, (b) decreased hemoglobin levels compared to the parent anucleate cell, (c) spherical morphology, (d) increased surface phosphatidylserine levels compared to the parent anucleate cell, or (e) reduced ATP production compared to the parent anucleate cell.

In another aspect, provided herein are compositions comprising a plurality of any anucleate cell-derived vesicles described herein. In some embodiments, the composition has one or more of the following properties: (a) greater than 20% of the anucleate cell-derived vesicles in the composition have a circulating half-life in a mammal that is decreased compared to the parent anucleate cell, (b) greater than 20% of the anucleate cell-derived vesicles in the composition have decreased hemoglobin levels compared to the parent anucleate cell, (c) greater than 20% of the anucleate cell-derived vesicles in the composition have spherical morphology, (d) greater than 20% of the anucleate cell-derived vesicles in the composition are RBC ghosts, (e) greater than 20% of the anucleate cell-derived vesicles in the composition have higher levels of phosphatidylserine, or (f) greater than 20% of the anucleate cell-derived vesicles in the composition have reduced ATP production compared to the parent anucleate cell.

In another aspect, provided herein are compositions comprising a plurality of any anucleate cells admixed with an adjuvant, as described herein.

In another aspect, provided herein are methods of making a composition disclosed herein, e.g., a method of making a composition comprising a plurality of anucleate cell-derived vesicles prepared from parent anucleate cells, the composition having one or more of the following properties: (a) greater than 20% of the anucleate cell-derived vesicles in the composition have a circulating half-life in a mammal that is decreased compared to the parent anucleate cell, (b) greater than 20% of the anucleate cell-derived vesicles in the composition have decreased hemoglobin levels compared to the parent anucleate cell, (c) greater than 20% of the anucleate cell-derived vesicles in the composition have spherical morphology, (d) greater than 20% of the anucleate cell-derived vesicles in the composition are RBC ghosts, (e) greater than 20% of the anucleate cell-derived vesicles in the composition have higher levels of phosphatidylserine, or (f) greater than 20% of the anucleate cell-derived vesicles in the composition have reduced ATP production compared to the parent anucleate cell; the method comprising passing a cell suspension comprising the parent anucleate cell through a cell-deforming constriction, wherein a diameter of the constriction is a function of a diameter of the parent anucleate cell in the suspension, thereby causing perturbations of the parent anucleate cell large enough for a payload to pass through to form an anucleate cell-derived vesicle, thereby producing an anucleate cell-derived vesicle.

In another aspect, provided herein are methods for using any of the compositions described herein. In some embodiments, the method for use is a method for treating a disease or disorder an individual in need thereof, the method comprising administering any of the anucleate cell-derived vesicles described herein. In some embodiments, the method for use is a method for preventing a disease or disorder an individual in need thereof, the method comprising administering any of the anucleate cell-derived vesicles described herein.

### Definitions

For purposes of interpreting this specification, the following definitions will apply and whenever appropriate, terms used in the singular will also include the plural and vice versa. In the event that any definition set forth below conflicts with any document incorporated herein by reference, the definition set forth shall control.

As used herein, the singular form “a”, “an”, and “the” includes plural references unless indicated otherwise.

The terms “comprising,” “having,” “containing,” and “including,” and other similar forms, and grammatical equivalents thereof, as used herein, are intended to be equivalent in meaning and to be open ended in that an item or items following any one of these words is not meant to be an exhaustive listing of such item or items, or meant to be limited to only the listed item or items. For example, an article “comprising” components A, B, and C can consist of (i.e., contain only) components A, B, and C, or can contain not only components A, B, and C but also one or more other components. As such, it is intended and understood that “comprises” and similar forms thereof, and grammatical equivalents thereof, include disclosure of embodiments of “consisting essentially of” or “consisting of.”

Where a range of values is provided, it is understood that each intervening value, to the tenth of the unit of the lower limit, unless the context clearly dictates otherwise, between the upper and lower limit of that range and any other stated or intervening value in that stated range, is encompassed within the disclosure, subject to any specifically excluded limit in the stated range. Where the stated range includes one or both of the limits, ranges excluding either or both of those included limits are also included in the disclosure.

The term “about” as used herein refers to the usual error range for the respective value readily known to the skilled person in this technical field. Reference to “about” a value or parameter herein includes (and describes) embodiments that are directed to that value or parameter per se. For example, description referring to “about X” includes description of “X”.

As used herein, “anucleate cell” refers to a cell lacking a nucleus. Such cells can include, but are not limited to, platelets, red blood cells (RBCs) such as erythrocytes and reticulocytes. Reticulocytes are immature (e.g., not yet biconcave) red blood cells, typically comprising about 1% of the red blood cells in the human body. Reticulocytes are also anucleate. In certain embodiments, the systems and methods described herein are used the treatment and/or processing of enriched (e.g., comprising a greater percentage of the total cellular population than would be found in nature), purified, or isolated (e.g., from their natural environment, in substantially pure or homogeneous form) populations of anucleate cells (e.g., RBCs, reticulocytes, and/or platelets). In certain embodiments, the systems and methods described herein are used for the treatment and/or processing of whole blood containing RBCs (e.g., erythrocytes or reticulocytes), platelets as well as other blood cells. Purification or enrichment of these cell types is accomplished using known methods such as density gradient systems (e.g., Ficoll-Hypaque), fluorescence activated cell sorting (FACS), magnetic cell sorting, or in vitro differentiation of erythroblasts and erythroid precursors.

The term “vesicle” as used herein refers to a structure comprising liquid enclosed by a lipid bilayer. In some examples, the lipid bilayer is sourced from naturally existing lipid composition. In some examples, the lipid bilayer can be sourced from a cellular membrane. In some examples, vesicles can be derived from various kinds of entities, such as cells. In such examples, a vesicle can retain molecules (such as intracellular proteins or membrane components) from the originating entity. For example, a vesicle derived from a red blood cell may contain any number of intracellular proteins that were in the red blood cell and/or membrane components of the red blood cell. In some examples, a vesicle can contain any number of molecules intracellularly in addition to the desired payload.

As used herein “payload” refers to the material that is being delivered into, such as loaded in, the anucleate cell-derived vesicle (e.g., an RBC-derived vesicle). “Payload,” “cargo,” “delivery material,” and “compound” are used interchangeably herein. In some embodiments, a payload may refer to a protein, a small molecule, a nucleic acid (e.g., RNA and/or DNA), a lipid, a carbohydrate, a macromolecule, a vitamin, a polymer, fluorescent dyes and fluorophores, carbon nanotubes, quantum dots, nanoparticles, and steroids. In some embodiments, the payload may refer to a protein or small molecule drug. In some embodiments, the payload may comprise one or more compounds.

The term “pore” as used herein refers to an opening, including without limitation, a hole, tear, cavity, aperture, break, gap, or perforation within a material. In some examples, (where indicated) the term refers to a pore within a surface of the present disclosure. In other examples, (where indicated) a pore can refer to a pore in a cell membrane.

The term “membrane” as used herein refers to a selective barrier or sheet containing pores. The term includes a pliable sheet-like structure that acts as a boundary or lining. In some examples, the term refers to a surface or filter containing pores. This term is distinct from the term “cell membrane”.

The term “filter” as used herein refers to a porous article that allows selective passage through the pores. In some examples the term refers to a surface or membrane containing pores.

The term “heterologous” as it relates to nucleic acid sequences such as coding sequences and control sequences, denotes sequences that are not normally joined together, and/or are not normally associated with a particular cell. Thus, a “heterologous” region of a nucleic acid construct or a vector is a segment of nucleic acid within or attached to another nucleic acid molecule that is not found in association with the other molecule in nature. For example, a heterologous region of a nucleic acid construct could include a coding sequence flanked by sequences not found in association with the coding sequence in nature. Another example of a heterologous coding sequence is a construct where the coding sequence itself is not found in nature (e.g., synthetic sequences having codons different from the native gene). Similarly, a cell transformed with a construct which is not normally present in the cell would be considered heterologous for purposes of this invention. Allelic variation or naturally occurring mutational events do not give rise to heterologous DNA, as used herein.

The term “heterologous” as it relates to amino acid sequences such as peptide sequences and polypeptide sequences, denotes sequences that are not normally joined together, and/or are not normally associated with a particular cell. Thus, a “heterologous” region of a peptide sequence is a segment of amino acids within or attached to another amino acid molecule that is not found in association with the other molecule in nature. For example, a heterologous region of a peptide construct could include the amino acid sequence of the peptide flanked by sequences not found in association with the amino acid sequence of the peptide in nature. Another example of a heterologous peptide sequence is a construct where the peptide sequence itself is not found in nature (e.g., synthetic sequences having amino acids different as coded from the native gene). Similarly, a cell transformed with a vector that expresses an amino acid construct which is not normally present in the cell would be considered heterologous for purposes of this invention. Allelic variation or naturally occurring mutational events do not give rise to heterologous peptides, as used herein.

The term “exogenous” when used in reference to an agent, such as an antigen or an adjuvant, with relation to a cell refers to an agent delivered from the extracellular space (that is, from outside the cell). The cell may or may not have the agent already present, and may or may not produce the agent after the exogenous agent has been delivered.

The term “homologous” as used herein refers to a molecule which is derived from the same organism. In some examples the term refers to a nucleic acid or protein which is normally found or expressed within the given organism.

As used herein, “treatment” or “treating” is an approach for obtaining beneficial or desired results, including clinical results. For purposes of this invention, beneficial or desired clinical results include, but are not limited to, one or more of the following: alleviating one or more symptoms resulting from the disease, diminishing the extent of the disease, stabilizing the disease (e.g., preventing or delaying the worsening of the disease), preventing or delaying the spread (e.g., metastasis) of the disease, preventing or delaying the recurrence of the disease, delay or slowing the progression of the disease, ameliorating the disease state, providing a remission (partial or total) of the disease, decreasing the dose of one or more other medications required to treat the disease, delaying the progression of the disease, increasing or improving the quality of life, increasing weight gain, and/or prolonging survival. Also encompassed by “treatment” is a reduction of pathological consequence of cancer (such as, for example, tumor volume). The methods of the invention contemplate any one or more of these aspects of treatment.

As used herein, the term “modulate” may refer to the act of changing, altering, varying, or otherwise modifying the presence, or an activity of, a particular target. For example, modulating an immune response may refer to any act leading to changing, altering, varying, or otherwise modifying an immune response. In other examples, modulating the expression of a nucleic acid may include, but not limited to a change in the transcription of a nucleic acid, a change in mRNA abundance (e.g., increasing mRNA transcription), a corresponding change in degradation of mRNA, a change in mRNA translation, and so forth.

As used herein, the term “inhibit” may refer to the act of blocking, reducing, eliminating, or otherwise antagonizing the presence, or an activity of, a particular target. Inhibition may refer to partial inhibition or complete inhibition. For example, inhibiting an immune response may refer to any act leading to a blockade, reduction, elimination, or any other antagonism of an immune response. In other examples, inhibition of the expression of a nucleic acid may include, but not limited to reduction in the transcription of a nucleic acid, reduction of mRNA abundance (e.g., silencing mRNA transcription), degradation of mRNA, inhibition of mRNA translation, gene editing and so forth. In other examples, inhibition of the expression of a protein may include, but not be limited to, reduction in the transcription of a nucleic acid encoding the protein, reduction in the stability of mRNA encoding the protein, inhibition of translation of the protein, reduction in stability of the protein, and so forth.

As used herein, the term “suppress” may refer to the act of decreasing, reducing, prohibiting, limiting, lessening, or otherwise diminishing the presence, or an activity of, a particular target. Suppression may refer to partial suppression or complete suppression. For example, suppressing an immune response may refer to any act leading to decreasing, reducing, prohibiting, limiting, lessening, or otherwise diminishing an immune response. In other examples, suppression of the expression of a nucleic acid may include, but not limited to reduction in the transcription of a nucleic acid, reduction of mRNA abundance (e.g., silencing mRNA transcription), degradation of mRNA, inhibition of mRNA translation, and so forth. In other examples, suppression of the expression of a protein may include, but not be limited to, reduction in the transcription of a nucleic acid encoding the protein, reduction in the stability of mRNA encoding the protein, inhibition of translation of the protein, reduction in stability of the protein, and so forth.

As used herein, the term “enhance” may refer to the act of improving, boosting, heightening, or otherwise increasing the presence, or an activity of, a particular target. For example, enhancing an immune response may refer to any act leading to improving, boosting, heightening, or otherwise increasing an immune response. In other examples, enhancing the expression of a nucleic acid may include, but not limited to increase in the transcription of a nucleic acid, increase in mRNA abundance (e.g., increasing mRNA transcription), decrease in degradation of mRNA, increase in mRNA translation, and so forth. In other examples, enhancing the expression of a protein may include, but not be limited to, increase in the transcription of a nucleic acid encoding the protein, increase in the stability of mRNA encoding the protein, increase in translation of the protein, increase in the stability of the protein, and so forth.

As used herein, the term “induce” may refer to the act of initiating, prompting, stimulating, establishing, or otherwise producing a result. For example, inducing an immune response may refer to any act leading to initiating, prompting, stimulating, establishing, or otherwise producing a desired immune response. In other examples, inducing the expression of a nucleic acid may include, but not limited to initiation of the transcription of a nucleic acid, initiation of mRNA translation, and so forth. In other examples, inducing the expression of a protein may include, but not be limited to, increase in the transcription of a nucleic acid encoding the protein, increase in the stability of mRNA encoding the protein, increase in translation of the protein, increase in the stability of the protein, and so forth.

The term “polynucleotide” or “nucleic acid” as used herein refers to a polymeric form of nucleotides of any length, including ribonucleotides and deoxyribonucleotides. Thus, this term includes, but is not limited to, single-, double- or multi-stranded DNA or RNA, genomic DNA, cDNA, DNA-RNA hybrids, or a polymer comprising purine and pyrimidine bases, or other natural, chemically or biochemically modified, non-natural, or derivatized nucleotide bases. The backbone of the polynucleotide can comprise sugars and phosphate groups (as may typically be found in RNA or DNA), or modified or substituted sugar or phosphate groups. The backbone of the polynucleotide can comprise repeating units, such as N-(2-aminoethyl)-glycine, linked by peptide bonds (i.e., peptide nucleic acid). Alternatively, the backbone of the polynucleotide can comprise a polymer of synthetic subunits such as phosphoramidates and thus can be an oligodeoxynucleotide phosphoramidate (P—NH2) or a mixed phosphoramidate-phosphodiester oligomer. In addition, a double-stranded polynucleotide can be obtained from the single stranded polynucleotide product of chemical synthesis either by synthesizing the complementary strand and annealing the strands under appropriate conditions, or by synthesizing the complementary strand de novo using a DNA polymerase with an appropriate primer.

The terms “polypeptide” and “protein” are used interchangeably to refer to a polymer of amino acid residues, and are not limited to a minimum length. Therefore as used herein, polypeptide includes short peptides. Such polymers of amino acid residues may contain natural or non-natural amino acid residues, and include, but are not limited to, peptides, oligopeptides, dimers, trimers, and multimers of amino acid residues. Both full-length proteins and fragments thereof are encompassed by the definition. The terms also include post-translational modifications of the polypeptide, for example, glycosylation, sialylation, acetylation, phosphorylation, and the like. Furthermore, for purposes of the present invention, a “polypeptide” refers to a protein which includes modifications, such as deletions, additions, and substitutions (generally conservative in nature), to the native sequence, as long as the protein maintains the desired activity. These modifications may be deliberate, as through site-directed mutagenesis, or may be accidental, such as through mutations of hosts which produce the proteins or errors due to PCR amplification.

As used herein, the term “adjuvant” refers to a substance which modulates and/or engenders an immune response. Generally, the adjuvant is administered in conjunction with an antigen to effect enhancement of an immune response to the antigen as compared to antigen alone. Various adjuvants are described herein.

The terms “CpG oligodeoxynucleotide” and “CpG ODN” refer to DNA molecules containing a dinucleotide of cytosine and guanine separated by a phosphate (also referred to herein as a “CpG” dinucleotide, or “CpG”). The CpG ODNs of the present disclosure contain at least one unmethylated CpG dinucleotide. That is, the cytosine in the CpG dinucleotide is not methylated (i.e., is not 5-methylcytosine). CpG ODNs may have a partial or complete phosphorothioate (PS) backbone.

As used herein, by “pharmaceutically acceptable” or “pharmacologically compatible” is meant a material that is not biologically or otherwise undesirable, e.g., the material may be incorporated into a pharmaceutical composition administered to a patient without causing any significant undesirable biological effects or interacting in a deleterious manner with any of the other components of the composition in which it is contained. Pharmaceutically acceptable carriers or excipients have preferably met the required standards of toxicological and manufacturing testing and/or are included on the Inactive Ingredient Guide prepared by the U.S. Food and Drug Administration.

For any of the structural and functional characteristics described herein, methods of determining these characteristics are known in the art.

As used herein, “microfluidic systems” refers to systems in which low volumes (e.g., m\L, nL, pL, fL) of fluids are processed to achieve the discrete treatment of small volumes of liquids. Certain implementations described herein include multiplexing, automation, and high throughput screening. The fluids (e.g., a buffer, a solution, a payload-containing solution, or a cell suspension) can be moved, mixed, separated, or otherwise processed. In certain embodiments described herein, microfluidic systems are used to apply mechanical constriction to a cell suspended in a buffer, inducing perturbations in the cell (e.g., holes) that allow a payload or compound to enter the cytosol of the cell.

As used herein, a “constriction” may refer to a portion of a microfluidic channel defined by an entrance portion, a centerpoint, and an exit portion, wherein the centerpoint is defined by a width, a length, and a depth. In other examples, a constriction may refer to a pore or may be a portion of a pore. The pore may be contained on a surface (e.g., a filter and/or membrane).

As used herein, “width of constriction” refers to the width of the microfluidic channel at the centerpoint. In some embodiments, the constriction has a width of less than about 6 μm. For example, in some embodiments the constriction may be less than about any of 0.6 μm, 0.7 μm, 0.8 μm, 0.9 μm, 1.0 μm, 1.5 μm, or 2 μm. In some embodiments, the constriction has a width of less than about 4 μm. In certain aspects of the invention, the constriction has a width between about 0.5 μm and about 4 μm. In further embodiments, the constriction has a width between about 3 μm and about 4 μm. In further embodiments, the constriction has a width between about 2 μm and about 4 μm. In further aspects, the constriction has a width of about 3.9 μm or less. In further aspects, the constriction has a width of about 3.9 μm or less. In further aspects, the constriction has a width of about 2.2 μm. In certain embodiments, the constriction is configured such that a single cell passes through the constriction at a time.

As used herein “length of constriction” refers to the length of the microfluidic channel at the centerpoint. In certain aspects of the invention, the length of the constriction is about 30 μm or less. In some embodiments, the length of the constriction is between about 10 μm and about 30 μm. In certain embodiments, the length of the constriction is between about 10 μm and about 20 μm. For example, the length of the constriction may be about any of 11 μm, 12 μm, 13 μm, 14 μm, 15 μm, 20 μm, or 25 μm including all integers, decimals, and fractions between about 10 μm and about 30 μm. The length of the constriction can vary to increase the length of time a cell is under constriction (e.g., greater lengths result in longer constrictions times at a given flow rate). The length of the constriction can vary to decrease the length of time a cell is under constriction (e.g., shorter lengths result in shorter constriction times at a given flow rate).

As used herein, “depth of constriction” refers to the depth of the microfluidic channel at the centerpoint. The depth of constriction can be adjusted to provide a tighter constriction and thereby enhance delivery, similar to adjustments of the constriction width. In some embodiments, the depth of the constriction is between about 1 μm and about 1 mm, including all integers, decimals, and fractions between about 1 μm and about 1 mm. In some embodiments, the depth is about 20 μm. In some embodiments the depth is uniform throughout the channel. In certain embodiments, the depth is decreased at the point of constriction to result in a greater constriction of the cell. In some embodiments, the depth is increased at the point of constriction to result in a lesser constriction of the cell. In some embodiment, the depth of the constriction is greater than the width of the constriction. In certain embodiments, the depth of constriction is less than the width of the constriction. In some embodiments, the depth of constriction and the width of the constriction are equal.

In some embodiments, the dimensions of the microfluidic device are denoted by length of constriction, width of constriction, and number of constrictions in series. For example, a microfluidic device with a constriction length of 30 μm, a width of 5 μm, and 5 constrictions in series is represented herein as 30×5×5 (L×W×# of constrictions).

In some embodiments, the microfluidic system comprises at least one microfluidic channel comprising at least one constriction. In some embodiments, the microfluidic system comprises multiple microfluidic channels each comprising at least one constriction. For example, the microfluidic system may comprise 2, 3, 4, 5, 6, 7, 8, 9, 10, 50, 100, 500, 1000, 10,000, 20,000 or greater microfluidic channels, including all integers from 10 to 50, 50 to 100, 100 to 500, 500, 1000, 10000 to 20,000, and the like. In certain aspects, the multiple microfluidic channels each comprising one constriction are arranged in parallel. In certain aspects, the multiple microfluidic channels each comprising one constriction are arranged linearly in series. In certain aspects of the invention, the microfluidic system comprises one microfluidic channel comprising multiple constrictions. For example, one microfluidic channel may comprise 2, 3, 4, 5, 10, 20, or greater constrictions. In some embodiments, the microfluidic system comprises multiple microfluidic channels comprising multiple constrictions. In some aspects of the invention, the multiple microfluidic channels comprising multiple constrictions are arranged in parallel. In some aspects of the invention, the multiple microfluidic channels comprising multiple constrictions are arranged linearly in series.

The entrance portion may comprise a “constriction angle” that can vary to increase or decrease how quickly the diameter of the channel decreases towards the centerpoint of the constriction. The constriction angle can vary to minimize clogging of the microfluidic system while cells are passing therethrough. For example, the constriction angle may be between 1 and 140 degrees. In certain embodiments, the constriction angle may be between 1 and 90 degrees. The exit portion may also comprise an angle to reduce the likelihood of turbulence/eddies that can result in non-laminar flow. For example, the angle of the exit portion may be between 1 and 140 degrees. In certain embodiments, the angle of the exit portion may be between 1 and 90 degrees.

The cross-section of the microfluidic channel, the entrance portion, the centerpoint, and the exit portion may vary. Non-limiting examples of various cross-sections include circular, elliptical, an elongated slit, square, hexagonal, or triangular cross-sections.

The velocity at which the anucleate cells (e.g., RBCs) pass through the microfluidic channels described herein can also be varied to control delivery of the delivery material to the cells. For example, adjusting the velocity of the cells through the microfluidic channel can vary the amount of time that a deforming force is applied to the cells, and can vary how rapidly the deforming force is applied to the cell. In some embodiments, adjusting the velocity of the cells through the microfluidic channel can vary the amount of time that a pressure is applied to the cells, and can vary how rapidly the pressure is applied to the cell. In some embodiments, the cells can pass through the microfluidic system at a rate of at least 0.1 mm/s. In further embodiments, the cells can pass through the microfluidic system at a rate between 0.1 mm/s and 5 m/s, including all integers and decimals therein. In still further embodiments, the cells can pass through the microfluidic system at a rate between 10 mm/s and 500 mm/s, including all integers and decimals therein. In some embodiments, the cells can pass through the system at a rate greater than 5 m/s.

Cells are moved (e.g., pushed) through the constriction by application of pressure. In some embodiments, said pressure is applied by a cell driver. As used herein, a cell driver is a device or component that applies a pressure or force to the buffer or solution in order to drive a cell through a constriction. In some embodiments, a pressure can be applied by a cell driver at the inlet. In some embodiments, a vacuum pressure can be applied by a cell driver at the outlet. In certain embodiments, the cell driver is adapted to supply a pressure about 10 to about 150 psi, such as about 10 to about 90 psi. In further embodiments, the cell driver is adapted to apply a pressure of 120 psi. In certain embodiments, the cell driver is selected from a group consisting of a pressure pump, a gas cylinder, a compressor, a vacuum pump, a syringe pump, a peristaltic pump, a pipette, a piston, a capillary actor, a human heart, human muscle, gravity, a microfluidic pumps, and a syringe. Modifications to the pressure applied by the cell driver also affect the velocity at which the cells pass through the microfluidic channel (e.g., increases in the amount of pressure will result in increased cell velocities).When a cell (e.g., an anucleate cell) passes through the constriction, its membrane is perturbed causing temporary disruptions in the membrane and resulting in the uptake of the payload that is present in the surrounding medium. As used herein, these temporary disruptions are referred to as “perturbations.” Perturbations created by the methods described herein are breaches in a cell that allow material from outside the cell to move into the cell. Non-limiting examples of perturbations include a hole, a tear, a cavity, an aperture, a pore, a break, a gap, or a perforation. The perturbations (e.g., pores or holes) created by the methods described herein are not formed as a result of assembly of protein subunits to form a multimeric pore structure such as that created by complement or bacterial hemolysins.

### Methods for Stimulating an Immune Response to an Antigen in an Individual

Methods for Delivering an Antigen into an Anucleate Cell-Derived Vesicle

In certain aspects, there is provided a method for delivering an antigen into an anucleate cell-derived vesicle, the method comprising passing a cell suspension comprising an input anucleate cell through a cell-deforming constriction, wherein a diameter of the constriction is a function of a diameter of the input anucleate cell in the suspension, thereby causing perturbations of the input anucleate cell large enough for the antigen to pass through to form an anucleate cell-derived vesicle; and incubating the anucleate cell-derived vesicle with the antigen for a sufficient time to allow the antigen to enter the anucleate cell-derived vesicle. In some embodiments, the anucleate cell-derived vesicle further comprises an adjuvant. In some embodiments, the input anucleate cell further comprises an adjuvant.

In certain aspects, there is provided a method for delivering an adjuvant into an anucleate cell-derived vesicle, the method comprising passing a cell suspension comprising an input anucleate cell through a cell-deforming constriction, wherein a diameter of the constriction is a function of a diameter of the input anucleate cell in the suspension, thereby causing perturbations of the input anucleate cell large enough for the adjuvant to pass through to form an anucleate cell-derived vesicle; and incubating the anucleate cell-derived vesicle with the adjuvant for a sufficient time to allow the adjuvant to enter the anucleate cell-derived vesicle. In some embodiments, the anucleate cell-derived vesicle further comprises an antigen. In some embodiments, the input anucleate cell further comprises an antigen.

In certain aspects, there is provided a method for delivering an antigen and an adjuvant into an anucleate cell-derived vesicle, the method comprising passing a cell suspension comprising an input anucleate cell through a cell-deforming constriction, wherein a diameter of the constriction is a function of a diameter of the input anucleate cell in the suspension, thereby causing perturbations of the input anucleate cell large enough for the antigen and the adjuvant to pass through to form an anucleate cell-derived vesicle; and incubating the anucleate cell-derived vesicle with the antigen and the adjuvant for a sufficient time to allow the antigen and the adjuvant to enter the anucleate cell-derived vesicle.

### Methods for Stimulating an Immune Response

In certain aspects, there is provided a method for stimulating an immune response to an antigen in an individual, the method comprising administering to the individual an effective amount of an anucleate cell-derived vesicle comprising an antigen, wherein the anucleate cell-derived vesicle comprising the antigen is prepared by a process comprising the steps of: a) passing a cell suspension comprising an input anucleate cell through a cell-deforming constriction, wherein a diameter of the constriction is a function of a diameter of the input anucleate cell in the suspension, thereby causing perturbations of the input anucleate cell large enough for the antigen to pass through to form an anucleate cell-derived vesicle; and b) incubating the anucleate cell-derived vesicle with the antigen for a sufficient time to allow the antigen to enter the anucleate cell-derived vesicle. In some embodiments, the method further comprises administering an adjuvant systemically to the individual. In some embodiments, the systemic adjuvant is administered before, after or at the same time as the anucleate cell-derived vesicle. In some embodiments, the input anucleate cell comprises an adjuvant. In some embodiments, the systemic adjuvant is an extracellular adjuvant. In some embodiment, the systemic adjuvant is an extravesicular adjuvant. In some embodiments, the method of stimulating an immune response to the antigen in the individual enhances a pre-existing immune response to the antigen.

In certain aspects, there is provided a method for stimulating an immune response to an antigen in an individual, the method comprising administering to the individual an effective amount of an anucleate cell-derived vesicle comprising an antigen and an adjuvant, wherein the anucleate cell-derived vesicle comprising the antigen and the adjuvant is prepared by a process comprising the steps of: a) passing a cell suspension comprising an input anucleate cell through a cell-deforming constriction, wherein a diameter of the constriction is a function of a diameter of the input anucleate cell in the suspension, thereby causing perturbations of the input anucleate cell large enough for the antigen and the adjuvant to pass through to form an anucleate cell-derived vesicle; and b) incubating the anucleate cell-derived vesicle with the antigen for a sufficient time to allow the antigen and the adjuvant to enter the anucleate cell-derived vesicle. In some embodiments, the method further comprises administering an adjuvant systemically to the individual. In some embodiments, the systemic adjuvant is administered before, after or at the same time as the anucleate cell-derived vesicle. In some embodiments, the input anucleate cell comprises an adjuvant. In some embodiments, the systemic adjuvant is an extracellular adjuvant. In some embodiment, the systemic adjuvant is an extravesicular adjuvant. In some embodiments, the method of stimulating a pre-existing immune response to the antigen in the individual enhances an immune response to the antigen.

### Methods for Treating or Preventing a Disease in an Individual

In certain aspects, there is provided a method for treating a disease in an individual, comprising administering to the individual an anucleate cell-derived vesicle comprising a disease-associated antigen, wherein an immune response against the antigen ameliorates conditions of the disease, and wherein the anucleate cell-derived vesicle comprising the disease-associated antigen is prepared by a process comprising the steps of: a) passing a cell suspension comprising an input anucleate cell through a cell-deforming constriction, wherein a diameter of the constriction is a function of a diameter of the input anucleate cell in the suspension, thereby causing perturbations of the input anucleate cell large enough for the antigen to pass through to form an anucleate cell-derived vesicle; and b) incubating the anucleate cell-derived vesicle with the antigen for a sufficient time to allow the antigen to enter the anucleate cell-derived vesicle.

In certain aspects, there is provided a method for preventing a disease in an individual, comprising administering to the individual an anucleate cell-derived vesicle comprising a disease-associated antigen, wherein an immune response against the antigen prevents development of the disease, and wherein the anucleate cell-derived vesicle comprising the disease-associated antigen is prepared by a process comprising the steps of: a) passing a cell suspension comprising an input anucleate cell through a cell-deforming constriction, wherein a diameter of the constriction is a function of a diameter of the input anucleate cell in the suspension, thereby causing perturbations of the input anucleate cell large enough for the antigen to pass through to form an anucleate cell-derived vesicle; and b) incubating the anucleate cell-derived vesicle with the antigen for a sufficient time to allow the antigen to enter the anucleate cell-derived vesicle.

In certain aspects, there is provided a method for vaccinating an individual against an antigen, comprising administering to the individual an anucleate cell-derived vesicle comprising the antigen, wherein the anucleate cell-derived vesicle comprising the antigen is prepared by a process comprising the steps of: a) passing a cell suspension comprising an input anucleate cell through a cell-deforming constriction, wherein a diameter of the constriction is a function of a diameter of the input anucleate cell in the suspension, thereby causing perturbations of the input anucleate cell large enough for the antigen to pass through to form an anucleate cell-derived vesicle; and b) incubating the anucleate cell-derived vesicle with the antigen for a sufficient time to allow the antigen to enter the anucleate cell-derived vesicle.

In some embodiments according to any of the methods described herein, the method further comprises administering an adjuvant systemically to the individual. In some embodiments, the systemic adjuvant is administered before, after or at the same time as the anucleate cell-derived vesicle. In some embodiments, the input anucleate cell comprises an adjuvant. In some embodiments, the systemic adjuvant is an extracellular adjuvant. In some embodiment, the systemic adjuvant is an extravesicular adjuvant.

In other aspects, there is provided a method for treating a disease in an individual, comprising administering to the individual an anucleate cell-derived vesicle comprising a disease-associated antigen and an adjuvant, wherein an immune response against the antigen ameliorates conditions of the disease, and wherein the anucleate cell-derived vesicle comprising the disease-associated antigen and the adjuvant is prepared by a process comprising the steps of: a) passing a cell suspension comprising an input anucleate cell through a cell-deforming constriction, wherein a diameter of the constriction is a function of a diameter of the input anucleate cell in the suspension, thereby causing perturbations of the input anucleate cell large enough for the antigen and an adjuvant to pass through to form an anucleate cell-derived vesicle; and b) incubating the anucleate cell-derived vesicle with the antigen and the adjuvant for a sufficient time to allow the antigen and the adjuvant to enter the anucleate cell-derived vesicle.

In certain aspects, there is provided a method for preventing a disease in an individual, comprising administering to the individual an anucleate cell-derived vesicle comprising a disease-associated antigen and an adjuvant, wherein an immune response against the antigen prevents development of the disease, and wherein the anucleate cell-derived vesicle comprising the disease-associated antigen and the adjuvant is prepared by a process comprising the steps of: a) passing a cell suspension comprising an input anucleate cell through a cell-deforming constriction, wherein a diameter of the constriction is a function of a diameter of the input anucleate cell in the suspension, thereby causing perturbations of the input anucleate cell large enough for the antigen and an adjuvant to pass through to form an anucleate cell-derived vesicle; and b) incubating the anucleate cell-derived vesicle with the antigen and the adjuvant for a sufficient time to allow the antigen and the adjuvant to enter the anucleate cell-derived vesicle.

In certain aspects, there is a provided a method for vaccinating an individual against an antigen, comprising administering to the individual an anucleate cell-derived vesicle comprising the antigen and an adjuvant, wherein the anucleate cell-derived vesicle comprising the antigen and the adjuvant is prepared by a process comprising the steps of: a) passing a cell suspension comprising an input anucleate cell through a cell-deforming constriction, wherein a diameter of the constriction is a function of a diameter of the input anucleate cell in the suspension, thereby causing perturbations of the input anucleate cell large enough for the antigen and an adjuvant to pass through to form an anucleate cell-derived vesicle; and b) incubating the anucleate cell-derived vesicle with the antigen and the adjuvant for a sufficient time to allow the antigen and the adjuvant to enter the anucleate cell-derived vesicle.

In certain aspects, there is provided a method for treating a disease in an individual, wherein an immune response against a disease-associated antigen ameliorates conditions of the disease, the method comprising: a) passing a cell suspension comprising an input anucleate cell through a cell-deforming constriction, wherein a diameter of the constriction is a function of a diameter of the input anucleate cell in the suspension, thereby causing perturbations of the input anucleate cell large enough for the antigen to pass through to form an anucleate cell-derived vesicle; b) incubating the anucleate cell-derived vesicle with the antigen for a sufficient time to allow the antigen to enter the anucleate cell-derived vesicle, thereby generating an anucleate cell-derived vesicle comprising the antigen; and c) administering the anucleate cell-derived vesicle comprising the antigen to the individual.

In certain aspects, there is provided a method for preventing a disease in an individual, wherein an immune response against a disease-associated antigen prevents development of the disease, the method comprising: a) passing a cell suspension comprising an input anucleate cell through a cell-deforming constriction, wherein a diameter of the constriction is a function of a diameter of the input anucleate cell in the suspension, thereby causing perturbations of the input anucleate cell large enough for the antigen to pass through to form an anucleate cell-derived vesicle; b) incubating the anucleate cell-derived vesicle with the antigen for a sufficient time to allow the antigen to enter the anucleate cell-derived vesicle, thereby generating an anucleate cell-derived vesicle comprising the antigen; and c) administering the anucleate cell-derived vesicle to the individual.

In certain aspects, there is provided a method for vaccinating an individual against an antigen, the method comprising: a) passing a cell suspension comprising an input anucleate cell through a cell-deforming constriction, wherein a diameter of the constriction is a function of a diameter of the input anucleate cell in the suspension, thereby causing perturbations of the input anucleate cell large enough for the antigen to pass through to form an anucleate cell-derived vesicle; b) incubating the anucleate cell-derived vesicle with the antigen for a sufficient time to allow the antigen to enter the anucleate cell-derived vesicle, thereby generating an anucleate cell-derived vesicle comprising the antigen; and c) administering the anucleate cell-derived vesicle comprising the antigen to the individual.

In some embodiments, the method further comprises administering an adjuvant systemically to the individual. In some embodiments, the systemic adjuvant is administered before, after or at the same time as the anucleate cell-derived vesicle. In some embodiments, the input anucleate cell comprises an adjuvant. In some embodiments, the systemic adjuvant is an extracellular adjuvant. In some embodiment, the systemic adjuvant is an extravesicular adjuvant.

In other aspects, there is provided a method for treating a disease in an individual, wherein an immune response against a disease-associated antigen ameliorates conditions of the disease, the method comprising: a) passing a cell suspension comprising an input anucleate cell through a cell-deforming constriction, wherein a diameter of the constriction is a function of a diameter of the input anucleate cell in the suspension, thereby causing perturbations of the input anucleate cell large enough for the disease-associated antigen and an adjuvant to pass through to form an anucleate cell-derived vesicle; b) incubating the anucleate cell-derived vesicle with the antigen and the adjuvant for a sufficient time to allow the antigen and the adjuvant to enter the anucleate cell-derived vesicle, thereby generating an anucleate cell-derived vesicle comprising the antigen and the adjuvant; and c) administering the anucleate cell-derived vesicle comprising the antigen and the adjuvant to the individual.

In some aspects, there is provided a method for preventing a disease in an individual, wherein an immune response against a disease-associated antigen prevents development of the disease, the method comprising: a) passing a cell suspension comprising an input anucleate cell through a cell-deforming constriction, wherein a diameter of the constriction is a function of a diameter of the input anucleate cell in the suspension, thereby causing perturbations of the input anucleate cell large enough for the antigen and an adjuvant to pass through to form an anucleate cell-derived vesicle; b) incubating the anucleate cell-derived vesicle with the antigen and the adjuvant for a sufficient time to allow the antigen and the adjuvant to enter the anucleate cell-derived vesicle, thereby generating an anucleate cell-derived vesicle comprising the antigen and the adjuvant; and c) administering the anucleate cell-derived vesicle comprising the antigen and the adjuvant to the individual.

In certain aspects, there is provided a method for vaccinating an individual against an antigen, the method comprising: a) passing a cell suspension comprising an input anucleate cell through a cell-deforming constriction, wherein a diameter of the constriction is a function of a diameter of the input anucleate cell in the suspension, thereby causing perturbations of the input anucleate cell large enough for the antigen and an adjuvant to pass through to form an anucleate cell-derived vesicle; b) incubating the anucleate cell-derived vesicle with the antigen and the adjuvant for a sufficient time to allow the antigen and the adjuvant to enter the anucleate cell-derived vesicle, thereby generating an anucleate cell-derived vesicle comprising the antigen and the adjuvant; and c) administering the anucleate cell-derived vesicle comprising the antigen and the adjuvant to the individual.

In some embodiments, the method further comprises administering an adjuvant systemically to the individual. In some embodiments, the systemic adjuvant is administered before, after or at the same time as the anucleate cell-derived vesicle. In some embodiments, the input anucleate cell comprises an adjuvant. In some embodiments, the systemic adjuvant is an extracellular adjuvant. In some embodiment, the systemic adjuvant is an extravesicular adjuvant.

In some embodiments according to any of the methods described herein, the disease is cancer, an infectious disease or a viral-associated disease. In some embodiment, the cancer comprises one or more of head and neck, cervical, uterine, rectal, penile, ovarian, testicular, bone, soft tissue, skin (melanoma), gastric, intestinal, colon, prostate, breast, esophageal, liver, lung, pancreatic, brain, or blood cancers. In some embodiments, the infectious disease or the viral-associated disease is associated with one or more of HPV, EBV, HIV, HBV, RSV, or KSHV. In some embodiments, the disease-associated antigen is an HPV antigen or an HPV-associated antigen. In some embodiments, the HPV antigen is an HPV-16 or an HPV-18 antigen. In some embodiments, the HPV antigen is an HPV E6 antigen or an HPV E7 antigen. In some embodiments, the HPV-associated disease is an HPV-associated cancer. In some embodiments, the HPV-associated cancer is cervical cancer, anal cancer, oropharyngeal cancer, vaginal cancer, vulvar cancer, penile cancer, skin cancer or head and neck cancer. In some embodiments, the HPV-associated disease is an HPV-associated infectious disease. In some embodiments, the HPV-associated diseases can include common warts, plantar warts, flat warts, anogenital warts, anal lesions, epidermodysplasia, focal epithelial hyperplasia, mouth papillomas, verrucous cysts, laryngeal papillomatosis, squamous intraepithelial lesions (SILs), cervical intraepithelial neoplasia (CIN), vulvar intraepithelial neoplasia (VIN) and vaginal intraepithelial neoplasia (VAIN). In certain embodiments, the disease-associated antigen is an EBV antigen or an EBV-associated antigen. In some embodiments, the EBV antigen or EBV-associated antigen is one or more of EBNA-1, EBNA-2, EBNA-3A, EBNA-3B, EBNA-3C, EBNA-LP, LMP-1, LMP-2A, LMP-2B or EBER. In some embodiments, the viral associated disease is an EBV-associated disease. In some embodiments, the EBV-associated disease is multiple sclerosis (MS). In some embodiments, the disease-associated antigen is a human CMV (HCMV) antigen or an HCMV-associated antigen. In some embodiments, the antigen is derived from any of strains Merlin, Toledo, Davis, Esp, Kerr, Smith, TB40E, TB40F, AD169 or Towne HCMV. In some embodiments, the HCMV antigen or HCMV-associated antigen is derived from one or more of pUL48, pUL47, pUL32, pUL82, pUL83, and pUL99, pUL69, pUL25, pUL56, pUL94, pUL97, pUL144 or pUL128. In some embodiments, the viral associated disease is an HCMV-associated disease. In other embodiments, the disease-associated antigen is an HIV antigen or an HIV-associated antigen. In some embodiments, the viral-associated disease is an HIV-associated disease. Opportunistic infections are infections that occur more frequently and are more severe in individuals with weakened immune systems, including people with HIV. In some embodiments, the HIV-associated disease are opportunistic infections, which may include but are not limited to: candidiasis of bronchi, trachea, esophagus, or lungs; invasive cervical cancer; coccidioidomycosis; cryptococcosis; chronic intestinal cryptosporidiosis, Cytomegalovirus diseases; HIV-related encephalopathy; HSV-related chronic ulcers or bronchitis, pneumonitis, or esophagitis; histoplasmosis; chronic intestinal isosporiasis; Kaposi's sarcoma; lymphoma; tuberculosis; Mycobacterium avium complex (MAC); Pneumocystis carinii pneumonia (PCP); recurrent pneumonia; progressive multifocal leukoencephalopathy; recurrent Salmonella septicemia; Toxoplasmosis of brain; and wasting syndrome due to HIV.

In some embodiments, provided are methods of treating an individual by introducing an anucleate cell-derived vesicle comprising an antigen and/or an adjuvant, generated by passing an input anucleate cell through a constriction to form an anucleate cell-derived vesicle such that the antigen and/or adjuvant enters the anucleate cell-derived vesicle, to the individual. In some embodiments, the input anucleate cell is an autologous cell. For example, the input anucleate cell is isolated from an individual (e.g., a patient), processed according to the methods disclosed, and the resulting anucleate cell-derived vesicle is introduced back into the same individual. Therefore, in some embodiments, the anucleate cell-derived vesicle is autologous to the individual. In other embodiments, the input anucleate cell is an allogeneic cell. For example, the anucleate cell is isolated from a different individual (e.g., the donor), processed according to the methods disclosed, and the resulting anucleate cell-derived vesicle is introduced into the first individual (e.g., the patient). In some embodiments, the anucleate cell-derived vesicle is allogeneic to the individual. In some embodiments, a pool of input anucleate cells from multiple individuals is processed according to the methods disclosed, and a pool of anucleate cell-derived vesicles is introduced into the first individual (e.g., the patient). In some embodiments, the input anucleate cell is isolated from an individual, processed according to the disclosed methods, and the anucleate cell-derived vesicle is introduced into a different individual. In some embodiments, a population of input anucleate cells is isolated from an individual (the patient) or a different individual, passed through the constriction to achieve delivery of an antigen and/or an adjuvant, and then a population of anucleate cell-derived vesicles is re-infused into the patient to augment a therapeutic response.

In some aspects, the invention provides methods of treating a patient by introducing an anucleate cell-derived vesicle comprising an antigen and/or an adjuvant, generated by passing an input anucleate cell through a constriction to form an anucleate cell-derived vesicle such that the antigen and/or adjuvant enters the anucleate cell-derived vesicle, to the individual. In some embodiments, the treatment comprises multiple (such as any of 2, 3, 4, 5, 6, or more) steps of introducing such anucleate cell-derived vesicles to the individual. For example, in some embodiments, there is provided a method of treating an individual by administering an anucleate cell-derived vesicle comprising an antigen and/or an adjuvant, generated by passing an input anucleate cell through a constriction to form an anucleate cell-derived vesicle such that the antigen and/or adjuvant enters the anucleate cell-derived vesicle, to the individual 2, 3, 4, 5, 6, or more times. In some embodiments, the duration of time between any two consecutive administrations of the cell is at least about 1 day (such at least about any of 2 days, 3 days, 4 days, 5 days, 6 days, 1 week, 2 weeks, 3 weeks, 1 month, 2 months, 3 months, 4 months, 5 months, 6 months, 7 months, 8 months, 9 months, 10 months, 11 months, 1 year, or longer, including any ranges between these values).

In some embodiments, the input anucleate cell is isolated from an individual, processed according to the methods disclosed, and the resulting anucleate cell-derived vesicle comprising an antigen and/or an adjuvant is introduced back into the same individual (e.g. the patient). For example, a population of input anucleate cells is isolated from an individual, passed through the constriction to achieve delivery of an antigen and/or an adjuvant, and the resulting anucleate cell derived vesicles are then re-infused into the individual to augment a therapeutic immune response. In some embodiments, the input anucleate cell is isolated from an individual, processed according to the disclosed methods, and the resulting anucleate cell derived vesicle is introduced back into the individual. For example, a population of input anucleate cells is isolated from an individual, passed through the constriction to achieve delivery of an antigen and/or an adjuvant, and the resulting anucleate cell-derived vesicles are then re-infused into the individual to stimulate and/or enhance an immune response in the individual.

In some embodiments, an input anucleate cell is isolated from a universal blood donor (e.g. an O− blood donor) and then stored and/or frozen for later constriction-mediated delivery. In some embodiments, an antigen is isolated from an individual and delivered to an input anucleate cell isolated from a universal donor. In some embodiments, an input anucleate cell is isolated from a blood donor and then stored and/or frozen for later constriction mediated delivery (SQZ). In some embodiments, an antigen is isolated from an individual and delivered to an input anucleate cell isolated from a blood donor. In some embodiments, an anucleate cell-derived vesicle comprising an antigen and/or an adjuvant is generated by constriction-mediated delivery described above. In some embodiments, the anucleate cell-derived vesicle comprising the antigen and/or the adjuvant is introduced into an individual. In some embodiments, the anucleate cell-derived vesicle comprising the antigen and/or the adjuvant is stored and/or frozen (e.g., for later treatments). In some embodiments, the individual has a matched blood type to the blood donor. In some embodiments, an anucleate cell-derived vesicle comprising an antigen and/or an adjuvant is generated by constriction-mediated delivery described above. In some embodiments, the anucleate cell-derived vesicle comprising the antigen and/or the adjuvant is introduced into an individual. In some embodiments, the individual has a matched blood type to the blood donor. In some embodiments, the individual has a mismatched blood type to the blood donor.

In some embodiments, provided are methods of preventing a disease in an individual by introducing an anucleate cell-derived vesicle comprising the antigen and/or an adjuvant, generated by passing an input anucleate cell through a constriction to form an anucleate cell-derived vesicle such that the antigen and/or adjuvant enters the anucleate cell-derived vesicle, to the individual. In some embodiments, the input anucleate cell is an autologous cell. For example, the input anucleate cell is isolated from an individual (e.g., a patient), processed according to the methods disclosed, and the resulting anucleate cell-derived vesicle is introduced back into the same individual. Therefore, in some embodiments, the anucleate cell-derived vesicle is autologous to the individual. In other embodiments, the input anucleate cell is an allogeneic cell. For example, the anucleate cell is isolated from a different individual (e.g., the donor), processed according to the methods disclosed, and the resulting anucleate cell-derived vesicle is introduced into the first individual (e.g., the patient). In some embodiments, the anucleate cell-derived vesicle is allogeneic to the individual. In some embodiments, a pool of input anucleate cells from multiple individuals is processed according to the methods disclosed, and a pool of anucleate cell-derived vesicles is introduced into the first individual (e.g., the patient). In some embodiments, the input anucleate cell is isolated from an individual, processed according to the disclosed methods, and the anucleate cell-derived vesicle is introduced into a different individual. In some embodiments, a population of input anucleate cells is isolated from an individual (the patient) or a different individual, passed through the constriction to achieve delivery of an antigen and/or an adjuvant, and then a population of anucleate cell-derived vesicles is re-infused into the patient to augment a prophylactic response.

In some embodiments, the method of prevention comprises multiple (such as any of 2, 3, 4, 5, 6, or more) steps of administering the anucleate cell-derived vesicles as described herein to the individual. For example, in some embodiments, there is provided a method of vaccinating an individual against an antigen by administering an anucleate cell-derived vesicle comprising an antigen and/or an adjuvant, generated by passing an input anucleate cell through a constriction to form an anucleate cell-derived vesicle such that the antigen and/or adjuvant enters the anucleate cell-derived vesicle, to the individual 2, 3, 4, 5, 6, or more times. In some embodiments, the duration of time between any two consecutive administrations of the cell is at least about 1 day (such at least about any of 2 days, 3 days, 4 days, 5 days, 6 days, 1 week, 2 weeks, 3 weeks, 1 month, 2 months, 3 months, 4 months, 5 months, 6 months, 7 months, 8 months, 9 months, 10 months, 11 months, 1 year, or longer, including any ranges between these values).

In some embodiments, provided are methods of vaccinating an individual against an antigen by introducing an anucleate cell-derived vesicle comprising the antigen and/or an adjuvant, generated by passing an input anucleate cell through a constriction to form an anucleate cell-derived vesicle such that the antigen and/or adjuvant enters the anucleate cell-derived vesicle, to the individual. In some embodiments, the input anucleate cell is an autologous cell. For example, the input anucleate cell is isolated from an individual (e.g., a patient), processed according to the methods disclosed, and the resulting anucleate cell-derived vesicle is introduced back into the same individual. Therefore, in some embodiments, the anucleate cell-derived vesicle is autologous to the individual. In other embodiments, the input anucleate cell is an allogeneic cell. For example, the anucleate cell is isolated from a different individual (e.g., the donor), processed according to the methods disclosed, and the resulting anucleate cell-derived vesicle is introduced into the first individual (e.g., the patient). In some embodiments, the anucleate cell-derived vesicle is allogeneic to the individual. In some embodiments, a pool of input anucleate cells from multiple individuals is processed according to the methods disclosed, and a pool of anucleate cell-derived vesicles is introduced into the first individual (e.g., the patient). In some embodiments, the input anucleate cell is isolated from an individual, processed according to the disclosed methods, and the anucleate cell-derived vesicle is introduced into a different individual. In some embodiments, a population of input anucleate cells is isolated from an individual (the patient) or a different individual, passed through the constriction to achieve delivery of an antigen and/or an adjuvant, and then a population of anucleate cell-derived vesicles is re-infused into the patient to induce a prophylactic response.

In some embodiments, the vaccination comprises multiple (such as any of 2, 3, 4, 5, 6, or more) steps of administering the anucleate cell-derived vesicles as described herein to the individual. For example, in some embodiments, there is provided a method of vaccinating an individual against an antigen by administering an anucleate cell-derived vesicle comprising an antigen and/or an adjuvant, generated by passing an input anucleate cell through a constriction to form an anucleate cell-derived vesicle such that the antigen and/or adjuvant enters the anucleate cell-derived vesicle, to the individual 2, 3, 4, 5, 6, or more times. In some embodiments, the duration of time between any two consecutive administrations of the cell is at least about 1 day (such at least about any of 2 days, 3 days, 4 days, 5 days, 6 days, 1 week, 2 weeks, 3 weeks, 1 month, 2 months, 3 months, 4 months, 5 months, 6 months, 7 months, 8 months, 9 months, 10 months, 11 months, 1 year, or longer, including any ranges between these values).

Any of the methods described above are carried out in vitro, ex vivo, or in vivo. For in vivo applications, the device may be implanted in a vascular lumen, e.g., an in-line stent in an artery or vein. In some embodiments, the methods are used as part of a bedside system for ex vivo treatment of patient cells and immediate reintroduction of the cells into the patient. Such methods could be employed as a means of enhancing and/or stimulating an immune response in an individual. In some embodiments, the method can be implemented in a typical hospital laboratory with a minimally trained technician. In some embodiments, a patient operated treatment system can be used. In some embodiments, the method is implemented using an in-line blood treatment system, in which blood is directly diverted from a patient, passed through the constriction, resulting in formation of vesicles derived from anucleate cells in the blood and delivery of antigen and/or adjuvant to the anucleate cell-derived vesicles in blood, and directly transfused back into the patient after treatment.

In some embodiments according to any of the methods described herein, the anucleate cell-derived vesicle is in a pharmaceutical formulation. In some embodiments, the anucleate cell-derived vesicle is administered systemically. In some embodiments, the anucleate cell-derived vesicle is administered intravenously, intraarterially, subcutaneously, intramuscularly, or intraperitoneally. In certain embodiments, the anucleate cell-derived vesicle is administered to the individual in combination with a therapeutic agent. In some embodiments, the therapeutic agent is administered before, after or at the same time as the anucleate cell-derived vesicle.

In some embodiments, the therapeutic agent is an immune checkpoint inhibitor, and/or a cytokine. In some embodiments, the therapeutic agent comprises one or more of: IFN-α, IFN-γ, IL-2 (any of its natural or modified forms), IL-10, or IL-15. In some embodiments, the therapeutic agent is one or more forms of immunotherapy. Immunotherapy includes but is not limited to: monoclonal antibodies, immune checkpoint inhibitors, cytokines, vaccines to treat cancers, and adoptive cell transfer. In some embodiments according to any of the methods described herein, the method further comprises administration of immunotherapy. In some embodiments, the method further comprises administering one or more therapeutic agents. In some embodiments, the immune checkpoint inhibitor is targeted to any one of PD-1, PD-L1, CTLA-4, TIM-3, LAGS, TIGIT, VISTA, TIM1, B7-H4 (VTCN1) and BTLA. In some embodiments, the therapeutic agent is a bispecific agent; for example, a bispecific agent comprising a cytokine component and a targeting component. In some embodiments, the anucleate cell-derived vesicle is administered to the individual in combination with a chemotherapy or a radiation therapy. In some embodiments, the anucleate cell-derived vesicle is administered to the individual in combination with one or more agents that improve antigen presentation, improve T cell proliferation, and/or improve tumor microenvironments.

### Anucleate Cells

In some embodiments according to any of the methods described herein, the input anucleate cell is a mammalian cell. Anucleate cells lack a nucleus. In some embodiments, the anucleate cell is a monkey, mouse, dog, cat, horse, rat, sheep, goat, pig, or rabbit cell. In some embodiments, the anucleate cell is a human cell. In some embodiments, the anucleate cell is a non-mammalian cell. In some embodiments, the anucleate cell is a chicken, frog, insect, fish, or nematode cell.

In some embodiments, the anucleate cell is a red blood cell. Red blood cells (RBCs) are flexible and oval biconcave discs with cytoplasm rich in the oxygen-carrier biomolecule hemoglobin. RBCs serve as the primary means for oxygen delivery and carbon dioxide removal throughout the human body. RBCs can stay in circulation for up to 120 days, after which they are removed from the body via clearance in the liver and spleen. In some embodiments, the anucleate cell is a precursor to RBCs. In some embodiments, the anucleate cell is a reticulocyte. Reticulocytes are anucleate immature (not yet biconcave) red blood cells and typically comprise about 1% of the red blood cells in the human body. Mature red blood cells are also referred to as erythrocytes. In some embodiments, the anucleate cell is an erythrocyte. In some embodiments, the anucleate cell is a platelet. Platelets, also called thrombocytes, are a component of blood whose function involves blood clotting. Platelets are biconvex discoid (lens-shaped) structures 2-3 μm in diameter.

In some embodiments according to any of the methods described herein, presentation of antigen in an immunogenic environment enhances an immune response to the antigen and/or stimulates an immune response to the antigen. Antigens derived from apoptotic bodies, such as anucleate cell-derived vesicles, which can be cleared in the immunogenic environment of the liver and spleen, may stimulate and/or enhance an immune response to the antigens via activation of T cells. In some embodiments, the immune response is antigen-specific. Anucleate cell-derived vesicles, such as red blood cell-derived vesicles have a limited life span and are unable to self-repair, causing eryptosis, a process analogous to apoptosis, that leads to subsequent removal of the anucleate cell-derived vesicles from the bloodstream. In some embodiments, the antigen may be released upon eryptosis of the anucleate cell-derived vesicles within the immunogenic environment, where it is subsequently engulfed, processed, and presented by an antigen-presenting cell. In some embodiments, the anucleate cell-derived vesicle comprising the antigen is phagocytosed by an antigen-presenting cell, and the antigen is subsequently processed and presented by the antigen-presenting cell. In some embodiments, the anucleate cell-derived vesicle comprising the antigen is phagocytosed by a resident macrophage, and the antigen is subsequently processed and presented by the resident macrophage.

In some embodiments, the antigen contained in the anucleate cell-derived vesicle is subsequently presented. In some embodiments, presentation of the antigen in an immunogenic environment stimulates an immune response to the antigen. In some embodiments, the antigen is processed in an immunogenic environment. In some embodiments, the immune response is antigen-specific.

In some embodiments, the anucleate cell-derived vesicle comprises an adjuvant. In some embodiments, the adjuvant generates or promotes an immunogenic environment, wherein presentation of an antigen in said immunogenic environment stimulates an immune response to the antigen. In some embodiments, the immune stimulation is multi-specific, including stimulation of an immune response to a plurality of antigens.

In some embodiments according to any of the methods described herein, the method comprises passing a cell suspension comprising an input anucleate cell through a constriction, wherein said constriction deforms the input anucleate cell thereby causing a perturbation of the input anucleate cell to form an anucleate cell-derived vesicle such that an antigen and/or an adjuvant enter the anucleate cell-derived vesicle. In some embodiments, the antigen is presented in an immunogenic environment. In some embodiments, the adjuvant generates or promotes an immunogenic environment, wherein presentation of the antigen in the immunogenic environment stimulates an immune response to the antigen. In some embodiments, the antigen is processed in an immunogenic environment. In some embodiments, the immune stimulation is antigen-specific. In some embodiments, the immune stimulation is multi-specific, including stimulation of an immune response to a plurality of antigens.

In certain embodiments according to any of the methods described herein, the method comprises passing a first cell suspension comprising a first input anucleate cell through a constriction, wherein said constriction deforms the cell thereby causing a perturbation of the first input anucleate cell such that an antigen enters a vesicle derived from perturbing the first input anucleate cell, passing a second cell suspension comprising a second input anucleate cell through a constriction, wherein said constriction deforms the second input anucleate cell thereby causing a perturbation of the second input anucleate cell such that an adjuvant enters a vesicle derived from perturbing the second input anucleate cell, and introducing a vesicle derived from the first input anucleate cell and a vesicle derived from the second input anucleate cell into the individual, thereby stimulating an immune response to the antigen. Therefore, in some embodiments, the vesicle derived from the first input anucleate cell comprises an antigen and the vesicle derived from the second input anucleate cell comprises an adjuvant. In some embodiments, the antigen is presented in an immunogenic environment. In some embodiments, the adjuvant generates or promotes an immunogenic environment, wherein presentation of the antigen in the immunogenic environment stimulates an immune response to the antigen. In some embodiments, the antigen is processed in an immunogenic environment. In some embodiments, the vesicle derived from the first input anucleate cell and the vesicle derived from the second input anucleate cell are introduced simultaneously. In some embodiments, the vesicle derived from the first input anucleate cell and the vesicle derived from the second input anucleate cell are introduced sequentially. In some embodiments, the vesicle derived from the first input anucleate cell is introduced to the individual before introduction of the vesicle derived from the second input anucleate cell. In some embodiments, the vesicle derived from the first input anucleate cell is introduced to the individual more than any of about 1 minute, 5 minutes, 10 minutes, 15 minutes, 30 minutes, 1 hour, 2 hours, 3 hours, 4 hours, 5 hours, 6 hours, 12 hours, or 24 hours before introduction of the vesicle derived from the second input anucleate cell. In some embodiments, the vesicle derived from the first input anucleate cell is introduced to the individual more than any of about 1 day, 2 days, 3 days, 4 days, 5 days, 6 days, or 7 days before introduction of the vesicle derived from the second input anucleate cell. In some embodiments, the vesicle derived from the second input anucleate cell is introduced to the individual before introduction of the vesicle derived from the first input anucleate cell. In some embodiments, the vesicle derived from the second input anucleate cell is introduced to the individual more than any of about 1 minute, 5 minutes, 10 minutes, 15 minutes, 30 minutes, 1 hour, 2 hours, 3 hours, 4 hours, 5 hours, 6 hours, 12 hours, or 24 hours before introduction of the vesicle derived from the first input anucleate cell. In some embodiments, the vesicle derived from the second input anucleate cell is introduced to the individual more than any of about 1 day, 2 days, 3 days, 4 days, 5 days, 6 days, or 7 days before introduction of the vesicle derived from the first input anucleate cell. In some embodiments, the immune stimulation is antigen-specific. In some embodiments, the immune stimulation is multi-specific, including stimulation of an immune response to a plurality of antigens.

In some embodiments, the stimulated and/or enhanced immune response comprises an increased T cell response. For example, an increased T cell response may include, without limitation, increased T cell activation or proliferation, increased T cell survival, or increased cell functionality. In some embodiments, the increased T cell response comprises increased T cell activation. In some embodiments, the increased T cell response comprises increased T cell survival. In some embodiments, the increased T cell response comprises increased T cell proliferation. In some embodiments, the increased T cell response comprises increased T cell functionality. For example, increased T cell functionality can include, without limitation, modulated cytokine secretion, increased T cell migration to sites of inflammation, and increased T cell cytotoxic activity. In some embodiments, the stimulated and/or enhanced immune response comprises increased inflammatory cytokine production and/or secretion, and/or decreased anti-inflammatory cytokine production and/or secretion. In some embodiments, the stimulated and/or enhanced immune response comprises increased production and/or secretion of one or more inflammatory cytokines selected from interleukin-1 (IL-1), IL-2, IL-12, and IL-18, tumor necrosis factor (TNF), interferon gamma (IFN-γ), and granulocyte-macrophage colony stimulating factor (GM-CSF). In some embodiments, the stimulated and/or enhanced immune response comprises decreased production and/or secretion of one or more anti-inflammatory cytokines selected from IL-4, IL-10, IL-13, IL-35, IFN-α and transforming growth factor-beta (TGF-β). In some embodiments, the stimulated and/or enhanced immune response comprises a change in T cell phenotype. For example, the T cell state may change from a regulatory (Treg) or anti-inflammatory phenotype to a pro-inflammatory phenotype. In some embodiments, the stimulated and/or enhanced immune response suppresses non-specific activation of a T cell, which otherwise may subsequently lead to cell death. In some embodiments, the stimulated and/or enhanced immune response comprises a suppressed Treg response. In some embodiments, the stimulated and/or enhanced immune response comprises an increased B cell response. In some embodiments, the increased B cell response comprises increased antibody production.

### Anucleate Cell-Derived Vesicles

In some aspects, the present application provides anucleate cell-derived vesicles prepared from a parent anucleate cell, the anucleate cell-derived vesicle having one or more of the following properties: (a) a circulating half-life in a mammal that is decreased compared to the parent anucleate cell, (b) decreased hemoglobin levels compared to the parent anucleate cell, (c) spherical morphology, (d) increased surface phosphatidylserine levels compared to the parent anucleate cell, or (e) reduced ATP production compared to the parent anucleate cell.

In certain aspects, there is provided an anucleate cell-derived vesicle comprising an antigen, wherein the anucleate cell-derived vesicle comprising the antigen is prepared by a process comprising the steps of: a) passing a cell suspension comprising an input anucleate cell through a cell-deforming constriction, wherein a diameter of the constriction is a function of a diameter of the input anucleate cell in the suspension, thereby causing perturbations of the input anucleate cell large enough for the antigen to pass through to form an anucleate cell-derived vesicle; and b) incubating the anucleate cell-derived vesicle with the antigen for a sufficient time to allow the antigen to enter the anucleate cell-derived vesicle; thereby generating the anucleate cell-derived vesicle comprising the antigen. In some embodiments, the input anucleate cell comprises an adjuvant.

In certain aspects, there is provided an anucleate cell-derived vesicle comprising an adjuvant, wherein the anucleate cell-derived vesicle comprising the adjuvant is prepared by a process comprising the steps of: a) passing a cell suspension comprising an input anucleate cell through a cell-deforming constriction, wherein a diameter of the constriction is a function of a diameter of the input anucleate cell in the suspension, thereby causing perturbations of the input anucleate cell large enough for the adjuvant to pass through to form an anucleate cell-derived vesicle; and b) incubating the anucleate cell-derived vesicle with the adjuvant for a sufficient time to allow the antigen to enter the anucleate cell-derived vesicle; thereby generating the anucleate cell-derived vesicle comprising the adjuvant. In some embodiments, the input anucleate cell comprises an antigen.

In certain aspects, there is provided an anucleate cell-derived vesicle comprising an antigen and an adjuvant, wherein the anucleate cell-derived vesicle comprising the antigen and the adjuvant is prepared by a process comprising the steps of: a) passing a cell suspension comprising an input anucleate cell through a cell-deforming constriction, wherein a diameter of the constriction is a function of a diameter of the input anucleate cell in the suspension, thereby causing perturbations of the input anucleate cell large enough for the antigen and the adjuvant to pass through to form an anucleate cell-derived vesicle; and b) incubating the anucleate cell-derived vesicle with the antigen and the adjuvant for a sufficient time to allow the antigen and the adjuvant to enter the anucleate cell-derived vesicle; thereby generating the anucleate cell-derived vesicle comprising the antigen and the adjuvant.

In some embodiments, the anucleate cell-derived vesicle is a red blood cell-derived vesicle or a platelet-derived vesicle. In some embodiments, the anucleate cell-derived vesicle is an erythrocyte-derived vesicle or a reticulocyte-derived vesicle

In some embodiments according to any of the anucleate cell-derived vesicles described herein, the input or parent anucleate cell is a mammalian cell. Anucleate cells lack a nucleus. In some embodiments, the input anucleate cell is a monkey, mouse, dog, cat, horse, rat, sheep, goat, pig, or rabbit cell. In some embodiments, the input anucleate cell is a human cell. In some embodiments, the input anucleate cell is a non-mammalian cell. In some embodiments, the input anucleate cell is a chicken, frog, insect, fish, or nematode cell. In some embodiments, the input anucleate cell is an erythrocyte. In some embodiments, the input anucleate cell is a red blood cell. In some embodiments, the input anucleate cell is a precursor to red blood cells. In some embodiments, the input anucleate cell is a reticulocyte. In some embodiments, the input anucleate cell is a platelet.

In some embodiments, presentation of antigen in an immunogenic environment enhances an immune response to the antigen or induces an immune response to the antigen. Antigens derived from eryptotic bodies, such as anucleate cell-derived vesicles, which can be cleared in the immunogenic environment of the liver and spleen, may stimulate and/or enhance an immune response to the antigens via activation of T cells. In some embodiments, the immune response is antigen-specific. Anucleate cell-derived vesicles, such as RBC-derived vesicles have a limited life span and are unable to self-repair, causing eryptosis, a process analogous to apoptosis, that leads to removal of the anucleate cell-derived vesicle from the bloodstream. In some embodiments, the antigen may be released upon eryptosis of the anucleate cell-derived vesicles within the immunogenic environment, where it is subsequently engulfed, processed, and presented by an antigen-presenting cell. In some embodiments, the anucleate cell-derived vesicle containing the antigen is phagocytosed by an antigen-presenting cell, such as a macrophage, and the antigen is subsequently processed and presented by the antigen-presenting cell. In some embodiments, the antigen-presenting cell is a resident macrophage.

In some embodiments, the input or parent anucleate cell is a red blood cell. In some embodiments, the input or parent anucleate cell is a platelet. In some embodiments, the red blood cell is an erythrocyte. In some embodiments, the red blood cell is a reticulocyte.

In some embodiments, the circulating half-life of an anucleate cell-derived vesicle in a mammal is decreased compared to an input or parent anucleate cell. Methods for measuring the half-life of a cell, such as an anucleate cell, e.g., red blood cell, or an anucleate cell-derived vesicle are known in the art. See, e.g., Franco, R. S., Transfus Med Hemother, 39, 2012. For example, in some embodiments, the method for measuring the half-life of an anucleate cell or an anucleate cell-derived vesicle comprises a cohort labeling technique or a random labeling technique. In some embodiments, the method for measuring the half-life of an anucleate cell or an anucleate cell-derived vesicle comprises labeling, reinfusing the cell or vesicle, and measuring the disappearance upon reinfusion. In some embodiments, the method for measuring the half-life of an anucleate cell or an anucleate cell-derived vesicle encompassed in the present application comprises measuring the half-life of an appropriate reference control(s), such as a control comprising an input or parent anucleate cell or a population of input or parent anucleate cells.

In some embodiments, the circulating half-life in the mammal is decreased by more than about 50%, such as more than about any of 55%, 60%, 65%, 70%, 75%, 80%, 85%, 90%, 95%, 96%, 97%, 98%, 99%, or 99.9% as compared to the input or parent anucleate cell. In some embodiments, the circulating half-life in the mammal is decreased by about 50% to about 99.9%, such as any of about 70% to about 99.9%, about 85% to about 99.9%, or about 95% to about 99.9%, as compared to the input or parent anucleate cell. In some embodiments, the circulating half-life in the mammal is decreased by about any of 50%, 55%, 60%, 65%, 70%, 75%, 80%, 85%, 90%, 95%, 96%, 97%, 98%, 99%, or 99.9%, as compared to the input or parent anucleate cell.

In some embodiments, the circulating half-life of the anucleate cell-derived vesicle is less than about any of 5 minutes, 10 minutes, 15 minutes, 30 minutes, 1 hour, 6 hours, 12 hours, 1 day, 2 days, 3 days, 4 days, 5 days, or 10 days. In some embodiments, the circulating half-life of the anucleate cell-derived vesicle is about any of 0.5 minute, 1 minute, 2 minutes, 3 minutes, 4 minutes, 5 minutes, 6 minutes, 7 minutes, 8 minutes, 9 minutes, 10 minutes, 15 minutes, 30 minutes, 45 minutes, 1 hour, 3 hours, 6 hours, 12 hours, 1 day, 2 days, 3 days, 4 days, 5 days, 10 days, 20 days, 30 days, 40 days, 50 days, 60 days, 70 days, 80 days, 90 days, or 100 days.

In some embodiments, the input or parent anucleate cell is a human cell and wherein the circulating half-life of the anucleate cell-derived vesicle is less than about any of 5 minutes, 10 minutes, 15 minutes, 30 minutes, 1 hour, 6 hours, 12 hours, 1 day, 2 days, 3 days, 4 days, 5 days, or 10 days. In some embodiments, the input or parent anucleate cell is a human cell and wherein the circulating half-life of the anucleate cell-derived vesicle is less than about any of 0.5 minute, 1 minute, 2 minutes, 3 minutes, 4 minutes, 5 minutes, 6 minutes, 7 minutes, 8 minutes, 9 minutes, 10 minutes, 15 minutes, 30 minutes, 45 minutes, 1 hour, 3 hours, 6 hours, 12 hours, 1 day, 2 days, 3 days, 4 days, 5 days, 10 days, 20 days, 30 days, 40 days, 50 days, 60 days, 70 days, 80 days, 90 days, or 100 days.

In some embodiments, the input or parent anucleate cell is a red blood cell, wherein the hemoglobin level in the anucleate cell-derived vesicle is decreased compared to the input or parent anucleate cell. Methods of measuring the hemoglobin level of a cell, such as an anucleate cell, e.g., red blood cell, or an anucleate cell-derived vesicle, e.g., a red blood cell-derived vesicle, is known in the art. See, e.g., Chaudhary, R., J Blood Med, 8, 2017. For example, in some embodiments, the method comprises measuring a metabolic precursor or product to determine the turnover of hemoglobin. In some embodiments, the method comprises measuring one or more hemoglobin-derived (Hb) peptides. In some embodiments, the method for measuring the hemoglobin level of an anucleate cell or an anucleate cell-derived vesicle encompassed in the present application comprises measuring the levels of hemoglobin of an appropriate reference control(s), such as a control comprising an input or parent anucleate cell or a population of input or parent anucleate cell.

In some embodiments, the anucleate cell is characterized by loss, such as a reduction in the level, of an intracellular component compared to a parent anucleate cell.

In some embodiments, the hemoglobin level in the anucleate cell-derived vesicle is decreased by at least about any of 10%, 15%, 20%, 25%, 30%, 35%, 40%, 45%, 50%, 55% 60%, 65%, 70%, 75%, 80%, 85%, 90%, 95%, 96%, 97%, 98%, 99%, or 99.9%, as compared to the input or parent anucleate cell. In some embodiments, the hemoglobin level in the anucleate cell-derived vesicle is decreased by about 50% to about 99.9%, such as any of about 70% to about 99.9%, about 85% to about 99.9%, or about 95% to about 99.9%, as compared to the input or parent anucleate cell. In some embodiments, the hemoglobin level in the anucleate cell-derived vesicle is decreased by about any of 50%, 55%, 60%, 65%, 70%, 75%, 80%, 85%, 90%, 95%, 96%, 97%, 98%, 99%, or 99.9%, as compared to the input or parent anucleate cell. In some embodiments, the anucleate cell-derived vesicle is devoid of hemoglobin. In some embodiments, the hemoglobin level in the anucleate cell-derived vesicle is about any of 0.01%, 0.1%, 0.5%, 1%, 2%, 3%, 4%, 5%, 10%, 20%, 30%, 40%, or 50% of the hemoglobin level in the input or parent anucleate cell.

In some embodiments, the level of one or more hemoglobin (Hb) peptides in the anucleate cell-derive vesicle is decreased by at least about any of 10%, 15%, 20%, 25%, 30%, 35%, 40%, 45%, 50%, 55% 60%, 65%, 70%, 75%, 80%, 85%, 90%, 95%, 96%, 97%, 98%, 99%, 99.9%, or 100%, as compared to the input or parent anucleate cell. In some embodiments, the level of one or more Hb peptides in the anucleate cell-derived vesicle is decreased by about 50% to about 99.9%, such as any of about 70% to about 99.9%, about 85% to about 99.9%, or about 95% to about 99.9%, as compared to the input anucleate cell. In some embodiments, the level of one or more Hb peptides in the anucleate cell-derived vesicle is decreased by about any of 50%, 55%, 60%, 65%, 70%, 75%, 80%, 85%, 90%, 95%, 96%, 97%, 98%, 99%, or 99.9%, as compared to the input or parent anucleate cell.

In some embodiments, the input or parent anucleate cell is an erythrocyte and wherein the morphology of the anucleate cell-derived vesicle is modulated from that of the input or parent anucleate cell. Morphology concerns the classification of, e.g., the shape, structure, geometry, intensity, form, smoothness, roughness, circularity, volume, surface area, and/or size of a cell or a cell-derived vesicle. Methods for determining (such as measuring) morphology are known in the art. See, e.g., Boutros et al., Cell, 163, 2015; Girasole, M. et al., Biochim Biophys Acta Biomembr, 1768, 2007; and Chen et al., Comput Math Methods Med, 2012. In some embodiments, the method for determining morphology comprises high-content imaging. For example, the morphology of the cell can be assessed by staining with Hoechst dye followed by automated high-content image analysis. In other examples, the morphology can be determined through a shift in the forward and side scatter plots from flow cytometry. In some embodiments, the input or parent anucleate cell is an erythrocyte and wherein the anucleate cell-derived vesicle is spherical in morphology. In some embodiments, the input or parent anucleate cell is an erythrocyte and wherein the anucleate cell-derived vesicle has a reduced biconcave shape compared to the input or parent anucleate cell. In some embodiments, the method for measuring morphology of an anucleate cell or an anucleate cell-derived vesicle encompassed in the present application comprises measuring the morphology of an appropriate reference control(s), such as a control comprising an input or parent anucleate cell or a population of input or parent anucleate cell.

In some embodiments, the input or parent anucleate cell is an erythrocyte and wherein the anucleate cell-derived vesicle has a reduced biconcave shape, such as reduced by more than about any of 5%, 10%, 15%, 20%, 25%, 30%, 35%, 40%, 45%, 50%, 55%, 60%, 65%, 70%, 75%, 80%, 85%, 90% 95%, 96%, 97%, 98%, 99%, or 99.9% as compared to the input or parent anucleate cell.

In some embodiments, the anucleate cell-derived vesicle is characterized by spherical morphology, including a substantially spherical morphology. In some embodiments, the spherical morphology of an anucleate cell-derived vesicle is assessed qualitatively. In some embodiments, the spherical morphology of an anucleate cell-derived vesicle is assessed quantitatively.

In some embodiments, the anucleate cell-derived vesicle has a reduced surface area to volume ratio, such as reduced by more than about any of 5%, 10%, 15%, 20%, 25%, 30%, 35%, 40%, 45%, 50%, 55%, 60%, 65%, 70%, 75%, 80%, 85%, 90%, 95%, or 99% as compared to the input or parent anucleate cell.

In some embodiments, the variation between each diameter measurement of a plurality of diameter measurements of an anucleate cell-derived vesicle is less than about 50%, such as less than about any of 45%, 40%, 35%, 30%, 25%, 20%, 15%, 10% or 5%, wherein the plurality of diameter measurements comprises at least two diameter measurements that measure the diameter at different points of the anucleate cell-derived vesicle.

In some embodiments, the smallest dimension, such as diameter, of an anucleate cell-derived vesicle in suspension is about 5 μm to about 7.25 μm, such as any of about 6 μm to about 7 μm, or about 6.25 μm to about 6.75 μm. In some embodiments, the smallest dimension, such as diameter, of an anucleate cell-derived vesicle in suspension is at least about 5 μm, such as at least about any of 5.25 μm, 5.5 μm, 5.75 μm, 6 μm, 6.25 μm, 6.5 μm, 6.75 μm, 7 μm, or 7.25 μm. In some embodiments, the largest dimension, such as diameter, of an anucleate cell-derived vesicle in suspension is about 5 μm to about 7.25 μm, such as any of about 6 μm to about 7 μm, or about 6.25 μm to about 6.75 μm. In some embodiments, the largest dimension, such as diameter, of an anucleate cell-derived vesicle in suspension is no greater than about 7.25 μm, such as no greater than about any of 7 μm, 6.75 μm, 6.5 μm, 6.25 μm, 6 μm, 5.75 μm, 5.5 μm, 5.25 μm, or 5 μm.

In some embodiments, the anucleate cell-derived vesicle exhibits one or more of the following properties: (a) a circulating half-life in a mammal is decreased compared to the parent anucleate cell, (b) decreased hemoglobin levels compared to the parent anucleate cell, (c) spherical morphology, (d) increased surface phosphatidylserine levels compared to the parent anucleate cell, or (e) reduced ATP production compared to the parent anucleate cell.

In some embodiments, the input or parent anucleate cell is a red blood cell or an erythrocyte and the anucleate cell-derived vesicle is a red blood cell ghost (RBC ghost).

In some embodiments, the anucleate cell is characterized by acquisition, such as an increase in the level, of a property compared to an input or parent anucleate cell.

In some embodiments, the anucleate cell-derived vesicle has increased surface phosphatidylserine levels compared to the input or parent anucleate cell. Phosphatidylserine exposure on the outer cell membrane is a hallmark of apoptosis and is recognized by receptors on phagocytes in a manner that promotes engulfment. Methods of measuring the phosphatidylserine level (such as surface phosphatidylserine level) of a cell, such as an anucleate cell, e.g., red blood cell, or an anucleate cell-derived vesicle are known in the art. See, e.g., Morita, S., et al., J Lipid Res, 53, 2012; Kay, J. G. et al., Sensors (Basel), 11, 2011; and Fabisiak J P et al., Methods Mol Biol, 1105, 2014. In some embodiments, the method for measuring the phosphatidylserine level of an anucleate cell or an anucleate cell-derived vesicle encompassed in the present application comprises measuring the phosphatidylserine level of an appropriate reference control(s), such as a control comprising an input or parent anucleate cell or a population of input or parent anucleate cell.

In some embodiments, the anucleate cell-derived vesicles prepared by the process have greater than about 1.5 fold more, such as greater than about any of 2 fold more, 2.5 fold more, 3 fold more, 3.5 fold more, 4 fold more, 4.5 fold more, 5 fold more, 10 fold more, 15 fold more, 20 fold more, or 25 fold more phosphatidylserine on its surface compared to the input or parent anucleate cell. In some embodiments, the anucleate cell-derived vesicles have greater than about 1.5 fold more, such as greater than about any of 2 fold more, 2.5 fold more, 3 fold more, 3.5 fold more, 4 fold more, 4.5 fold more, 5 fold more, 10 fold more, 15 fold more, 20 fold more, or 25 fold more phosphatidylserine on its surface as compared to the input or parent anucleate cell.

In some embodiments, the anucleate cell-derived vesicle prepared by the process has greater than about 1.5 fold more, such as greater than about any of 2 fold more, 2.5 fold more, 3 fold more, 3.5 fold more, 4 fold more, 4.5 fold more, 5 fold more, 10 fold more, 15 fold more, 20 fold more, or 25 fold more, phosphatidylserine on its surface per unit volume compared to the input or parent anucleate cell. In some embodiments, the anucleate cell-derived vesicles have greater than about 1.5 fold more, such as greater than about any of 2 fold more, 2.5 fold more, 3 fold more, 3.5 fold more, 4 fold more, 4.5 fold more, 5 fold more, 10 fold more, 15 fold more, 20 fold more, or 25 fold more, phosphatidylserine per unit volume on its surface as compared to the input or parent anucleate cell.

In some embodiments, the anucleate cell-derived vesicle prepared by the process has have greater than about 1.5 fold more, such as greater than about any of 2 fold more, 2.5 fold more, 3 fold more, 3.5 fold more, 4 fold more, 4.5 fold more, 5 fold more, 10 fold more, 15 fold more, 20 fold more, or 25 fold more, phosphatidylserine on its surface per unit surface area compared to the input or parent anucleate cell. In some embodiments, the anucleate cell-derived vesicles have greater than about 1.5 fold more, such as greater than about any of 2 fold more, 2.5 fold more, 3 fold more, 3.5 fold more, 4 fold more, 4.5 fold more, 5 fold more, 10 fold more, 15 fold more, 20 fold more, or 25 fold more, phosphatidylserine per unit surface area on its surface as compared to the input or parent anucleate cell.

In some embodiments, the anucleate cell-derived vesicle prepared by the process has have greater than about 1.5 fold more, such as greater than about any of 2 fold more, 2.5 fold more, 3 fold more, 3.5 fold more, 4 fold more, 4.5 fold more, 5 fold more, 10 fold more, 15 fold more, 20 fold more, or 25 fold more, phosphatidylserine on its surface per unit of membrane phospholipid compared to the input or parent anucleate cell. In some embodiments, the anucleate cell-derived vesicles have greater than about 1.5 fold more, such as greater than about any of 2 fold more, 2.5 fold more, 3 fold more, 3.5 fold more, 4 fold more, 4.5 fold more, 5 fold more, 10 fold more, 15 fold more, 20 fold more, or 25 fold more, phosphatidylserine per unit of membrane phospholipid on its surface as compared to the input or parent anucleate cell.

In some embodiments, the anucleate cell-derived vesicle has about any of 10%, 15%, 20%, 25%, 30%, 35%, 40%, 45%, 50%, 55% 60%, 65%, 70%, 75%, 80%, 85%, 90%, 95%, 100%, 125%, 150%, 175%, or 200% or more phosphatidylserine on its surface as compared to the input or parent anucleate cell. In some embodiments, the anucleate cell-derived vesicle has about 50% to about 200% more, such as any of about 50% to about 100%, about 100% to about 200%, or about 75% to about 125% more phosphatidylserine on its surface, as compared to the input or parent anucleate cell.

In some embodiments, the anucleate cell-derived vesicle has about any of 10%, 15%, 20%, 25%, 30%, 35%, 40%, 45%, 50%, 55% 60%, 65%, 70%, 75%, 80%, 85%, 90%, 95%, 100%, 125%, 150%, 175%, or 200% or more phosphatidylserine on its surface per unit volume as compared to the input or parent anucleate cell. In some embodiments, the anucleate cell-derived vesicle has about 50% to about 200% more, such as any of about 50% to about 100%, about 100% to about 200%, or about 75% to about 125% more phosphatidylserine on its surface per unit volume, as compared to the input or parent anucleate cell.

In some embodiments, the anucleate cell-derived vesicle has about any of 10%, 15%, 20%, 25%, 30%, 35%, 40%, 45%, 50%, 55% 60%, 65%, 70%, 75%, 80%, 85%, 90%, 95%, 100%, 125%, 150%, 175%, or 200% or more phosphatidylserine on its surface per unit surface area as compared to the input or parent anucleate cell. In some embodiments, the anucleate cell-derived vesicle has about 50% to about 200% more, such as any of about 50% to about 100%, about 100% to about 200%, or about 75% to about 125% more phosphatidylserine on its surface per unit surface area, as compared to the input or parent anucleate cell.

In some embodiments, the anucleate cell-derived vesicle has about any of 10%, 15%, 20%, 25%, 30%, 35%, 40%, 45%, 50%, 55% 60%, 65%, 70%, 75%, 80%, 85%, 90%, 95%, 100%, 125%, 150%, 175%, or 200% or more phosphatidylserine on its surface per unit of membrane phospholipid as compared to the input or parent anucleate cell. In some embodiments, the anucleate cell-derived vesicle has about 50% to about 200% more, such as any of about 50% to about 100%, about 100% to about 200%, or about 75% to about 125% more phosphatidylserine on its surface per unit of membrane phospholipid, as compared to the input or parent anucleate cell.

In some embodiments according to any of the anucleate cell-derived vesicles described herein, more than any of 5%, 10%, 20%, 30%, 40%, 50%, 60%, 70%, 80%, 90%, 95%, 98% or 99.5% of total membrane phosphatidylserine are localized on the external membrane leaflet in the anucleate cell-derived vesicles. In some embodiments, more than 50% of total membrane phosphatidylserine are localized on the external membrane leaflet in the anucleate cell-derived vesicles.

In some embodiments according to any of the anucleate cell-derived vesicles described herein, a population profile of anucleate cell-derived vesicles prepared by the process exhibits higher average phosphatidylserine levels on the surface compared to the input or parent anucleate cells. In some embodiments, a population profile of anucleate cell-derived vesicles prepared by the process exhibits about any of 10%, 15%, 20%, 25%, 30%, 35%, 40%, 45%, 50%, 55% 60%, 65%, 70%, 75%, 80%, 85%, 90%, 95%, 100%, 125%, 150%, 175%, or 200% higher average phosphatidylserine levels on the surface compared to the input or parent anucleate cells. In some embodiments, a population profile of anucleate cell-derived vesicles prepared by the process exhibits about any of 1.5 fold, 2 fold, 2.5 fold, 3 fold, 3.5 fold, 4 fold, 4.5 fold, 5 fold, 10 fold, 15 fold, 20 fold, 25 fold, 50 fold, or 100 fold higher average phosphatidylserine levels on the surface compared to the input or parent anucleate cells.

In some embodiments according to any of the anucleate cell-derived vesicles described herein, at least any one of 10%, 20%, 30%, 40%, 50%, 60%, 70%, 80%, 90%, 95% or 99% of the population profile of anucleate cell-derived vesicles prepared by the process exhibits higher phosphatidylserine levels on the surface compared to the input or parent anucleate cells. In some embodiments, at least 50% of the population profile of anucleate cell-derived vesicles prepared by the process exhibits higher phosphatidylserine levels on the surface compared to the input or parent anucleate cells.

In some embodiments, the half-life of the anucleate cell-derived vesicle can be further modified. In some embodiments, the half-life of the anucleate cell-derived vesicle is increased by the further modification. For example, the anucleate cell-derived vesicle may be modified to increase the time the anucleate cell-derived vesicle circulates in the blood stream before clearance in the liver and spleen. In some embodiments, the half-life of the anucleate cell-derived vesicle is further decreased by the modification. For example, the anucleate cell-derived vesicle may be modified to decrease the time the anucleate cell circulates in the blood stream before clearance in the liver and spleen. In some embodiments, an altered ratio of phospholipids, on the surface of the anucleate cell-derived vesicle decreases the half-life of the anucleate cell-derived vesicle. In some embodiments, an increased ratio of phosphatidylserine to other phospholipids on the surface of the anucleate cell-derived vesicle decreases the half-life of the anucleate cell-derived vesicle. For example, the presence of phosphatidylserine on the surface of the anucleate cell-derived vesicle can be further increased to decrease the half-life of the anucleate cell-derived vesicle, such as by using any method known in the art for increasing surface phosphatidylserine (See, Hamidi et al., J. Control. Release, 2007, 118(2): 145-60). In some embodiments, the anucleate cell-derived vesicle is incubated with lipids or phospholipids prior to delivery to an individual. In some embodiments, the anucleate cell-derived vesicle is treated by chemicals such as bis(sulfosuccinimidyl)suberate or other cross-linking agents, prior to delivery to an individual. In other embodiments, the surface phosphatidylserine of the anucleate cell-derived vesicle can be decreased to increase the half-life of the anucleate cell-derived vesicle. Flippases are enzymes that transport phospholipids from the external surface to the cytosolic surface in the plasma membrane. In some embodiments, the anucleate cell-derived vesicle is treated with flippase prior to delivery to an individual. In some embodiments, the anucleate cell-derived vesicle is treated with an enzyme that cleaves phosphatidylserines prior to delivery to an individual. A non-limiting example of an enzyme that cleaves phosphatidylserine is phosphatidylserine carboxylase.

In some embodiments, the anucleate cell-derived vesicle has reduced ATP production compared to the input or parent anucleate cell. In some embodiments, the anucleate cell-derived vesicle has reduced ATP production, or levels of intracellular ATP, over time. Methods of measuring the ATP (such as reduced ATP production, or levels of intracellular ATP, over time) of a cell, such as an anucleate cell, e.g., red blood cell, or an anucleate cell-derived vesicle are known in the art. See, e.g., Morciano, G. et al., Nat Protoc, 12, 2017. In some embodiments, the ATP production is measured via a surrogate or a marker, such as lactate production. In some embodiments, the method for measuring ATP production of an anucleate cell or an anucleate cell-derived vesicle encompassed in the present application comprises measuring the ATP production of an appropriate reference control(s), such as a control comprising an input or parent anucleate cell or a population of input or parent anucleate cell. In some embodiments, the method for measuring ATP production, which allow for comparisons of ATP production between a sample and a control, comprises measuring ATP production of the sample and the control under similar conditions. In some embodiments, the method for measuring ATP production or intracellular ATP levels of an anucleate cell-derived vesicle encompassed in the present application comprises measuring the ATP production or intracellular ATP level of anucleate cell-derived vesicles of a population of the anucleate cell-derived vesicles at a first time and a second time, wherein the first time is before the second time, and comparing the results from the first time and the second time.

In some embodiments, the anucleate cell-derived vesicle produces ATP at less than about any of 0.01%, 0.1%, 0.5%, 1%, 2%, 3%, 4%, 5%, 10%, 15%, 20%, 25%, 30%, 35%, 40%, 45%, or 50% of the level of ATP produced by the input or parent anucleate cell. In some embodiments, the anucleate cell-derived vesicle does not produce ATP.

In some embodiments, ATP production is determined by a lactate assay. In some embodiments, to measure the metabolic activity, such as ATP production, of the input anucleate cells versus anucleate cell-derived vesicles, the level of glycolysis can be indirectly measured over time by monitoring the level of lactate production using a fluorescent enzymatic assay. For example, the input anucleate cells are resuspended in citrate-phosphate-dextrose with adenine (dCPDA-1) buffer at 1 billion cells/mL and model antigen and/or adjuvant (at 20 μg/mL) is delivered at room temperature via SQZ (2.2 μm constriction width at 50 psi) to generate anucleate cell-derived vesicles. The anucleate cell-derived vesicles, as well as the unprocessed input anucleate cells are then incubated at 37° C. for the indicated time points and supernatant is collected. To quantify the levels of lactate produced by the input anucleate cells versus anucleate cell-derived vesicles, the Lactate-Glo assay (Promega) can be used employed to assay supernatant from the respective time points. Briefly, the supernatants are subjected to inactivation and neutralization steps, prior to the addition of the fluorescent lactate detection reagent. Fluorescence is normalized to a blank control and the absolute lactate levels in the supernatant are determined using a lactate standard curve (0.1-10 μM). In some embodiments, the absolute lactate level is about 0 μM to about 200 μM, such as any of about 0.01 μM to about 10 μM, about 0.01 μM to about

In some aspects, the present application provides anucleate cell-derived vesicles prepared from a parent anucleate cell, the anucleate cell-derived vesicle having any one or more of the following properties, as further described herein, of: (a) a circulating half-life in a mammal that is decreased compared to the parent anucleate cell, (b) decreased hemoglobin levels compared to the parent anucleate cell, (c) spherical morphology, (d) increased surface phosphatidylserine levels compared to the parent anucleate cell, or (e) reduced ATP production compared to the parent anucleate cell.

In some aspects, the present application provides anucleate cell-derived vesicles prepared from a parent anucleate cell, the anucleate cell-derived vesicle having any two or more of the following properties, as further described herein, of: (a) a circulating half-life in a mammal that is decreased compared to the parent anucleate cell, (b) decreased hemoglobin levels compared to the parent anucleate cell, (c) spherical morphology, (d) increased surface phosphatidylserine levels compared to the parent anucleate cell, or (e) reduced ATP production compared to the parent anucleate cell.

In some aspects, the present application provides anucleate cell-derived vesicles prepared from a parent anucleate cell, the anucleate cell-derived vesicle having any three or more of the following properties, as further described herein, of: (a) a circulating half-life in a mammal that is decreased compared to the parent anucleate cell, (b) decreased hemoglobin levels compared to the parent anucleate cell, (c) spherical morphology, (d) increased surface phosphatidylserine levels compared to the parent anucleate cell, or (e) reduced ATP production compared to the parent anucleate cell.

In some aspects, the present application provides anucleate cell-derived vesicles prepared from a parent anucleate cell, the anucleate cell-derived vesicle having any four or more of the following properties, as further described herein, of: (a) a circulating half-life in a mammal that is decreased compared to the parent anucleate cell, (b) decreased hemoglobin levels compared to the parent anucleate cell, (c) spherical morphology, (d) increased surface phosphatidylserine levels compared to the parent anucleate cell, or (e) reduced ATP production compared to the parent anucleate cell.

In some aspects, the present application provides anucleate cell-derived vesicles prepared from a parent anucleate cell, the anucleate cell-derived vesicle having the following properties, as further described herein, of: (a) a circulating half-life in a mammal that is decreased compared to the parent anucleate cell, (b) decreased hemoglobin levels compared to the parent anucleate cell, (c) spherical morphology, (d) increased surface phosphatidylserine levels compared to the parent anucleate cell, or (e) reduced ATP production compared to the parent anucleate cell.

In some embodiments, the anucleate cell-derived vesicle is modified to enhance uptake, such as increase uptake, in a tissue or cell compared to the uptake of the parent anucleate cell. In some embodiments, the anucleate cell-derived vesicle is modified to enhance uptake, such as increase uptake, in liver and/or spleen compared to the uptake of the parent anucleate cell in the respective tissue. In some embodiments, the anucleate cell-derived vesicle is modified to enhance uptake, such as increase uptake, in a phagocytic cell or an antigen-presenting cell, such as a macrophage or a dendritic cell, compared to the uptake of the parent anucleate cell in the respective phagocytic cell. In some embodiments, the macrophage is an adipose tissue macrophage, monocyte, Kupffer cell, sinus histiocyte, alveolar macrophage, tissue macrophage, microglia, Hofbauer cell, intraglomerular mesangial cell, osteoclast, epitheloid cell, red pulp macrophage, peritoneal macrophage, or LysoMac. In some embodiments, the antigen-presenting cell is a professional antigen-presenting cell. In some embodiments, the antigen-presenting cell is a non-professional antigen-presenting cell. In some embodiments, the antigen-presenting cell is a dendritic cell, or macrophage. In some embodiments, the anucleate cell-derived vesicle is cleared by a phagocytic cell and/or an antigen-presenting cell in the liver and/or spleen, thereby leading to antigen presentation including via CD8+ and CD4+ T cell responses.

In some embodiments according to any of the anucleate cell-derived vesicles described herein, the anucleate cell-derived vesicle exhibits enhanced uptake in a tissue or cell compared to the input or parent anucleate cell. In some embodiments, the modified anucleate cell-derived vesicle exhibits a rate of uptake in tissue or cell that is enhanced by more than any one of about 1.5-fold, about 2 fold, about 3 fold, about 4 fold, about 5 fold, about 10 fold, about 20 fold, about 30 fold, about 50 fold, about 100 fold, about 200 fold, about 500 fold, or about 1000 fold compared to the input or parent anucleate cell. In some embodiments, the anucleate cell-derived vesicle exhibits enhanced uptake in phagocytic cells and/or antigen presenting cells compared to the input or parent anucleate cell. In some embodiments, phagocytic cells and/or antigen presenting cells comprise macrophages and/or dendritic cells. In some embodiments, the anucleate cell-derived vesicle exhibits enhanced uptake in liver, spleen or macrophages compared to the input or parent anucleate cell. In some embodiments, the anucleate cell-derived vesicle exhibits enhanced uptake in liver and/or spleen or by a phagocytic cell and/or an antigen-presenting cell compared to the uptake of the input or parent anucleate cell. In some embodiments, the anucleate cell-derived vesicle is not cleared in the lungs. In some embodiments, the anucleate cell-derived vesicle is cleared by macrophages in the liver and/or spleen, thereby leading to antigen presentation including via CD8+ and CD4+ T cell responses.

In some embodiments according to any of the anucleate cell-derived vesicles described herein, the anucleate cell-derived vesicle is modified to enhance uptake in a tissue or cell compared to an unmodified anucleate cell-derived vesicle. In some embodiments, the modified anucleate cell-derived vesicle exhibits a rate of uptake in tissue or cell that is enhanced by more than any one of about 1.5-fold, about 2 fold, about 3 fold, about 4 fold, about 5 fold, about 10 fold, about 20 fold, about 30 fold, about 50 fold, about 100 fold, about 200 fold, about 500 fold, or about 1000 fold compared to an unmodified anucleate cell-derived vesicle. In some embodiments, the anucleate cell-derived vesicle is modified to enhance uptake in phagocytic cells and/or antigen presenting cells compared to an unmodified anucleate cell-derived vesicle. In some embodiments, phagocytic cells and/or antigen presenting cells comprise macrophages and/or dendritic cells. In some embodiments, the anucleate cell-derived vesicle is modified to enhance uptake in liver, spleen or macrophages compared to an unmodified anucleate cell-derived vesicle. In some embodiments, the anucleate cell-derived vesicle is modified to enhance uptake in liver and/or spleen or by a phagocytic cell and/or an antigen-presenting cell compared to the uptake of the input or parent anucleate cell. In some embodiments, the anucleate cell-derived vesicle is not cleared in the lungs. In some embodiments, the anucleate cell-derived vesicle is cleared by macrophages in the liver and/or spleen, thereby leading to antigen presentation including via CD8+ and CD4+ T cell responses.

In some embodiments, the anucleate cell-derived vesicle comprises CD47 on its surface.

In some embodiments, the anucleate cell-derived vesicle is not (a) heat processed, (b) chemically treated, and/or (c) subjected to hypotonic or hypertonic conditions during the preparation of the anucleate cell-derived vesicles. In certain embodiments, the anucleate cell-derived vesicle is not heat-treated or heat-shocked. In certain embodiments, the anucleate cell-derived vesicle is not treated by chemicals such as bis(sulfosuccinimidyl)suberate or other cross-linking agents. In certain embodiments, the anucleate cell-derived vesicle is not further modified to express or contain ionophores or other ion transporters. In certain embodiments, the anucleate cell-derived vesicle is not associated with antibodies such as anti-TER119.

In some embodiments according to any of the anucleate cell-derived vesicles described herein, the osmolarity of the cell suspension is maintained throughout the process. In further embodiments, the osmolarity of the cell suspension is maintained between about 200 mOsm and about 400 mOsm throughout the process. In some embodiments, the osmolarity of the cell suspension is maintained between about 200 mOsm and about 600 mOsm throughout the process. In further embodiments, the osmolarity of the cell suspension is maintained between about 200 mOsm and about 800 mOsm throughout the process. In some embodiments, the osmolarity of the cell suspension is maintained between any one of: about 200 mOsm and about 300 mOsm, about 300 mOsm and about 400 mOsm, about 400 mOsm and about 500 mOsm, about 500 mOsm and about 600 mOsm, about 600 mOsm and about 700 mOsm, about 700 mOsm and about 800 mOsm, about 200 mOsm and about 400 mOsm, about 400 mOsm and about 600 mOsm, or about 600 mOsm and about 800 mOsm. In some embodiments, the osmolarity was maintained during preparation of the anucleate cell-derived vesicle from the input or parent anucleate cell. In some embodiments, the osmolarity was maintained between about 200 mOsm and about 600 mOsm, such as between any of about 200 mOsm and about 300 mOsm, about 200 mOsm and about 400 mOsm, about 200 mOsm and about 500 mOsm, about 300 mOsm and about 500 mOsm or about 350 mOsm and about 450 mOsm. In some embodiments, the osmolarity was maintained between about 200 mOsm and about 400 mOsm.

In some embodiments, cell suspension is contacted with the antigen before, concurrently, and/or after passing through the constriction.

In some embodiments according to any of the anucleate cell-derived vesicles described herein, there is provided a composition comprising a plurality of anucleate cell-derived vesicle. In some embodiments, the composition further comprises a pharmaceutically acceptable excipient.

### Antigens and Adjuvants

In some embodiments according to any of methods or anucleate cell-derived vesicles described herein, the antigen is a disease-associated antigen. In further embodiments, the antigen is a tumor antigen. In some embodiments, the antigen is derived from a lysate. In some embodiments, the lysate is derived from a biopsy of an individual. In some embodiments, the lysate is derived from a biopsy of an individual being infected by a pathogen, such as a bacterium or a virus. In some embodiments, the lysate is derived from a biopsy of an individual bearing tumors (i.e. tumor biopsy lysates). Thus, in some embodiments, the lysate is a tumor lysate. In some embodiments, the antigen is derived from a transplant lysate. In some embodiments, the lysate is derived from a biopsy of a transplanted organ. In some embodiments, the antigen is a viral antigen, a bacterial antigen or a fungal antigen. In some embodiments, the antigen is a microorganism.

In some embodiments, according to any of the methods or anucleate cell-derived vesicles described herein, the anucleate cell-derived vesicle comprises an antigen comprising an immunogenic epitope. In some embodiments, the antigen is a disease-associated antigen. In some embodiments, the antigen is derived from peptides or mRNA isolated from a diseased cell. In some embodiments, the antigen is derived from a protein ectopically expressed or overexpressed in a diseased cell. In some embodiments, the antigen is derived from a neoantigen, e.g., a cancer-associated neoantigen. In some embodiments, the antigen comprises a neoepitope, e.g., a cancer-associated neoepitope. In some embodiments, the antigen is a non-self antigen. In some embodiments, the antigen is a mutated or otherwise altered self antigen. In some embodiments, the antigen is a tumor antigen, viral antigen, bacterial antigen, or fungal antigen. In some embodiments, the antigen comprises an immunogenic epitope fused to heterologous peptide sequences. In some embodiments, the antigen comprises a plurality of immunogenic epitopes. In some embodiments, some of the plurality of immunogenic epitopes are derived from the same source. For example, in some embodiments, some of the plurality of immunogenic epitope are derived from the same viral antigen. In some embodiments, all of the plurality of immunogenic epitopes are derived from the same source. In some embodiments, none of the plurality of immunogenic epitopes are derived from the same source. In some embodiments, the anucleate cell-derived vesicle comprises a plurality of different antigens. In some embodiments, a plurality of antigens, such as any of 2, 3, 4, 5, 6, 7, 8, 9, or 10 different types of antigens, are delivered to the anucleate cell.

In some embodiments according to any of the methods or anucleate cell-derived vesicles described herein, the antigen is a polypeptide antigen. In some embodiments, the antigen is a non-protein antigen. For example, in some embodiments, the antigen is a lipid antigen. In some embodiments, the antigen is carbohydrate antigen, such as a polysaccharide. In some embodiments, the antigen is a glycolipid. In some embodiments, a nucleic acid encoding the antigen is delivered to the cell. In some embodiments, the antigen is a whole microorganism, such as an intact bacterium. In some embodiments, the antigen is a disease-associated antigen. In some embodiments, antigens are derived from foreign sources, such as bacteria, fungi, viruses, or allergens. In some embodiments, the antigen is a modified antigen. For example, antigens may be fused with therapeutic agents or targeting peptides. In some embodiments, the modified antigen comprises an antigen fused with a polypeptide. In some embodiments, the modified antigen comprises an antigen fused with a targeting peptide. In some embodiments, the modified antigen comprises an antigen fused with a lipid. In some embodiments, the modified antigen comprises an antigen fused with a carbohydrate. In some embodiments, the modified antigen comprises an antigen fused with a nanoparticle. In some embodiments, a plurality of antigens is delivered to the anucleate cell.

In some embodiments, according to any of the methods or anucleate cell-derived vesicles described herein, the anucleate cell-derived vesicle comprises an antigen, wherein the antigen comprises an immunogenic epitope. In some embodiments, the antigen is a polypeptide and the immunogenic epitope is an immunogenic peptide epitope. In some embodiments, the immunogenic peptide epitope is fused to an N-terminal flanking polypeptide and/or a C-terminal flanking polypeptide. In some embodiments, the immunogenic peptide epitope fused to the N-terminal flanking polypeptide and/or the C-terminal flanking polypeptide is a non-naturally occurring sequence. In some embodiments, the N-terminal and/or C-terminal flanking polypeptides are non-natural. In some embodiments, the immunogenic peptide epitope fused to the N-terminal flanking polypeptide and/or the C-terminal flanking polypeptide is synthetic. In some embodiments, the N-terminal and/or C-terminal flanking polypeptides are derived from an immunogenic synthetic long peptide (SLP). In some embodiments, the N-terminal and/or C-terminal flanking polypeptides are derived from a disease-associated immunogenic SLP.

In some embodiments, according to any of the methods or anucleate cell-derived vesicle described herein, the anucleate cell-derived vesicle comprises an antigen, wherein the antigen is capable of being processed into an MHC class I-restricted peptide and/or an MHC class II-restricted peptide. In some embodiments, the antigen is capable of being processed into an MHC class I-restricted peptide. In some embodiments, the antigen is capable of being processed into an MHC class II-restricted peptide. In some embodiments, the antigen comprises a plurality of immunogenic epitopes, and is capable of being processed into an MHC class I-restricted peptide and an MHC class II-restricted peptide. In some embodiments, the anucleate cell-derived vesicle comprising the antigen is taken up by an antigen presenting cell, and the antigen is processed into one or more MHC class I-restricted peptide and/or one or more MHC class II-restricted peptide by the antigen presenting cell. In some embodiments, the antigen is a CD-1 restricted antigen. In some embodiments, the CD-1 restricted antigen is a lipid antigen. In some embodiments, the antigen comprises a plurality of immunogenic epitopes, and is capable of being processed into a plurality of CD-1 restricted antigens. In some embodiments, the anucleate cell-derived vesicle comprising the antigen is taken up by an antigen presenting cell, and the antigen is processed into one or more CD-1 restricted antigens by the antigen presenting cell. In some embodiments, the antigen comprises a plurality of immunogenic epitopes, and is capable of being processed into one or more of (a) a MHC class I-restricted peptide; (b) an MHC class II-restricted peptide; or (c) a CD-1 restricted antigen. In some embodiments, some of the plurality of immunogenic epitopes are derived from the same source. In some embodiments, all of the plurality of immunogenic epitopes are derived from the same source. In some embodiments, none of the plurality of immunogenic epitopes are derived from the same source.

In some embodiments, according to any of the methods or anucleate cell-derived vesicles described herein, the anucleate cell-derived vesicle comprises a plurality of antigens that comprise a plurality of immunogenic epitopes. In some embodiments, following administration to an individual of the anucleate cell-derived vesicle comprising the plurality of antigens that comprise the plurality of immunogenic epitopes, none of the plurality of immunogenic epitopes decreases an immune response in the individual to any of the other immunogenic epitopes.

In some embodiments, according to any of the methods or any of the anucleate cell-derived vesicles described herein, the anucleate cell-derived vesicle comprises an adjuvant. In some embodiments, the adjuvant is a CpG oligodeoxynucleotide (ODN), IFN-α, STING agonists, RIG-I agonists, poly I: C (low and/or high molecular weight), polyinosinic-polycytidylic acid stabilized with polylysine and carboxymethylcellulose (HILTONOL®), imiquimod, resiquimod and/or lipopolysaccharide (LPS). In some embodiments, the adjuvant is a CpG ODN. In some embodiments, the adjuvant is low molecular weight poly I:C. In some embodiments, the CpG ODN is no greater than about 50 (such as no greater than about any of 45, 40, 35, 30, 25, 20, or fewer) nucleotides in length. In some embodiments, the CpG ODN is a Class A CpG ODN, a Class B CpG ODN, or a Class C CpG ODN. In some embodiments, the CpG ODN comprises the nucleotide sequences as disclosed in US provisional application U.S. 62/641,987. In some embodiments, the anucleate cell-derived vesicle comprises a plurality of different CpG ODNs. For example, in some embodiments, the anucleate cell-derived vesicle comprises a plurality of different CpG ODNs selected from among Class A, Class B, and Class C CpG ODNs.). In some embodiments, a plurality of adjuvants, such as any of 2, 3, 4, 5, 6, 7, 8, 9, or 10 different types of adjuvants, is delivered to the anucleate cell.

In some embodiments, the anucleate cell-derived vesicle comprises an antigen and/or an adjuvant. In some embodiments, the anucleate cell-derived vesicle comprises the antigen at a concentration between about 1 pM and about 10 mM. In some embodiments, the anucleate cell-derived vesicle comprises the adjuvant at a concentration between about 1 pM and about 10 mM. In some embodiments, the anucleate cell-derived vesicle comprises the antigen at a concentration between about 0.1 μM and about 10 mM. In some embodiments, the anucleate cell-derived vesicle comprises the adjuvant at a concentration between about 0.1 μM and about 10 mM. For example, in some embodiments, the concentration of adjuvant in the anucleate cell-derived vesicle is any of less than about 1 pM, about 10 pM, about 100 pM, about 1 nM, about 10 nM, about 100 nM, about 1 μM, about 10 μM, about 100 μM, about 1 mM or about 10 mM. In some embodiments, the concentration of adjuvant in the anucleate cell-derived vesicle is greater than about 10 mM. In some embodiments, the concentration of antigen in the anucleate cell-derived vesicle is any of less than about 1 pM, about 10 pM, about 100 pM, about 1 nM, about 10 nM, about 100 nM, about 1 μM, about 10 μM, about 100 μM, about 1 mM or about 10 mM. In some embodiments, the concentration of antigen in the anucleate cell-derived vesicle is greater than about 10 mM. In some embodiments, the concentration of antigen in the anucleate cell-derived vesicle is any of between about 1 pM and about 10 pM, between about 10 pM and about 100 pM, between about 100 pM and about 1 nM, between about 1 nM and about 10 nM, between about 10 nM and about 100 nM, between about 100 nM and about 1 μM, between about 1 μM and about 10 μM, between about 10 μM and about 100 μM, between about 100 μM and about 1 mM, or between 1 mM and about 10 mM. In some embodiments, the concentration of adjuvant in the anucleate cell-derived vesicle is any of between about 1 pM and about 10 pM, between about 10 pM and about 100 pM, between about 100 pM and about 1 nM, between about 1 nM and about 10 nM, between about 10 nM and about 100 nM, between about 100 nM and about 1 μM, between about 1 μM and about 10 μM, between about 10 μM and about 100 μM, between about 100 μM and about 1 mM, or between 1 mM and about 10 mM.

In some embodiments, the molar ratio of adjuvant to antigen in the anucleate cell-derived vesicle is any of between about 10000:1 to about 1:10000. For example, in some embodiments, the molar ratio of adjuvant to antigen in the anucleate cell-derived vesicle is about any of 10000:1, about 1000:1, about 100:1, about 10:1, about 1:1, about 1:10, about 1:100, about 1:1000, or about 1:10000. In some embodiments, the anucleate cell-derived vesicle comprises a complex comprising: a) the antigen, b) the adjuvant, and/or c) the antigen and the adjuvant.

In some embodiments, according to any of the methods or anucleate cell-derived vesicles described herein, the anucleate cell-derived vesicle further comprises an additional agent that enhances the function of the anucleate cell-derived vesicle as compared to a corresponding anucleate cell-derived vesicle that does not comprise the additional agent. In some embodiments, the additional agent is a stabilizing agent or a co-factor. In some embodiments, the agent is albumin. In some embodiments, the albumin is mouse, bovine, or human albumin. In some embodiments, the additional agent is a divalent metal cation, glucose, ATP, potassium, glycerol, trehalose, D-sucrose, PEG1500, L-arginine, L-glutamine, or EDTA.

In some embodiments, according to any of the methods or anucleate cell-derived vesicles described herein, the anucleate cell-derived vesicle further comprises one or more therapeutic agents.

### Other Payloads

In some embodiments, the payload is a tolerogenic factor. In some embodiments, the payload is a polypeptide, a nucleic acid, a lipid, a carbohydrate, a small molecule, a complex (such as a protein-based complex, a nucleic acid complex, a protein-protein complex, nucleic acid-nucleic acid complex, or a protein-nucleic acid complex), a nanoparticle, a virus, or a viral particle.

In some embodiments, the payload is selected from the group consisting of an uricase (including semi-synthetic forms, e.g., Pegloticase) glucocerebrosidase (e.g., Imiglucerase, velaglucerase alfa, β-glucosidase), tissue non-specific alkaline phosphatase (TNSALP) (e.g., Asfotase alfa), lysosomal acid lipase (e.g., Sebelipase alfa), alpha-glucosidase (e.g., alglucosidase alfa), α-L-iduronidase (e.g., laronidase), Iduronate sulfatase (e.g., Idursulfase), heparan sulfate, keratin sulfate, chondroitin 6-sulfate (e.g., elosulfase alfa), N-acetylgalactosamine-4-sulfatase (e.g., galsulfase), β-glucuronidase, hyaluronidase, α-galactosidase A (e.g., agalsidase beta), phenylalanine hydroxylase, medium-chain acyl-CoA dehydrogenase, gliadin, acetylcholine receptor and receptor-associated proteins, thyroid stimulating hormone receptor (TSHR), desmoglein 1 and 3, aquaporin 4, GADD65, insulin, pro-insulin, and pre-pro-insulin.

In some embodiments, the anucleate cell-derived vesicle was prepared by a process comprising: (a) passing a cell suspension comprising the input parent anucleate cell through a cell-deforming constriction, wherein a diameter of the constriction is a function of a diameter of the input parent anucleate cell in the suspension, thereby causing perturbations of the input parent anucleate cell large enough for the payload to pass through to form an anucleate cell-derived vesicle; and (b) incubating the anucleate cell-derived vesicle with the payload for a sufficient time to allow the payload to enter the anucleate cell-derived vesicle; thereby producing an anucleate cell-derived vesicle comprising the payload.

In some embodiments, the anucleate cell-derived vesicle comprises an antigen and a tolerogenic factor. In some embodiments, the tolerogenic factor enhances suppression of an immune response to an antigen and/or enhances the induction of tolerance to an antigen. In some embodiments, the tolerogenic factor may promote tolerogenic presentation of the antigen by an antigen-presenting cell. In some embodiments, the tolerogenic factor comprises a polypeptide. In some embodiments, the polypeptide is IL-4, IL-10, IL-13, IL-35, IFN-α, or TGF-β. In some embodiments, the polypeptide is a therapeutic polypeptide. In some embodiments, the polypeptide is a fragment of a therapeutic polypeptide. In some embodiments, the polypeptide is conjugated to a carbohydrate. In some embodiments, the tolerogenic factor is a nucleic acid. In some embodiments, the nucleic acid can include, without limitation, mRNA, DNA, miRNA, or siRNA. For example, the tolerogenic factor can include siRNA to knock down expression of inflammatory genes. In some embodiments, the tolerogenic factor is a DNA sequence that binds NF-κB and prevents NF-κB activation and downstream signaling. In some embodiments, the tolerogenic factor is a small molecule.

In some embodiments, the tolerogenic factor modulates expression and/or activity of an immunomodulatory agent (such as an immunostimulatory agent (e.g., a costimulatory molecule), an immunosuppressive agent, or an inflammatory or anti-inflammatory molecule). In some embodiments, the tolerogenic factor inhibits expression and/or activity of an immunostimulatory agent (e.g., a costimulatory molecule), enhances expression and/or activity of an immunosuppressive molecule, inhibits expression and/or activity of an inflammatory molecule, and/or enhances expression and/or activity of an anti-inflammatory molecule. In some embodiments, the tolerogenic factor inhibits the activity of a costimulatory molecule. Interaction between costimulatory molecules and their ligands is important to sustain and integrate TCR signaling to stimulate optimal T cell proliferation and differentiation. In some embodiments, the tolerogenic factor decreases expression of a costimulatory molecule. Exemplary costimulatory molecules expressed on antigen-presenting cells include, without limitation, CD40, CD80, CD86, CD54, CD83, CD79, Ox40 or ICOS Ligand. In some embodiments, the costimulatory molecule is CD80 or CD86. In some embodiments, the tolerogenic factor inhibits the expression of a nucleic acid that expresses or modulates expression of the costimulatory molecule. In some embodiments, the tolerogenic factor deletes a nucleic acid that expresses or modulates expression of the costimulatory molecule. In some embodiments, deletion of the nucleic acid that expresses or modulates expression of the costimulatory molecule is achieved via gene editing. In some embodiments, the tolerogenic factor inhibits the costimulatory molecule. In some embodiments, the tolerogenic factor is a siRNA that inhibits the costimulatory molecule. In some embodiments, the tolerogenic factor increases the activity of a transcriptional regulator that suppresses expression of the costimulatory molecule. In some embodiments, the tolerogenic factor increases the activity of a protein inhibitor that suppresses expression of the costimulatory molecule. In some embodiments, the tolerogenic factor comprises nucleic acid encoding a suppressor of the costimulatory molecule. In some embodiments, the tolerogenic factor degrades the costimulatory molecule. In some embodiments, the tolerogenic factor labels the costimulatory molecule for destruction. For example, the tolerogenic factor may enhance ubiquitination of the costimulatory molecule, thereby targeting it for destruction.

In some embodiments, the tolerogenic factor enhances the expression and/or activity of an immunosuppressive molecule. In some embodiments, the immunosuppressive molecule is a co-inhibitory molecule, a transcriptional regulator, or an immunosuppressive molecule. Co-inhibitory molecules negatively regulate the activation of lymphocytes. Exemplary co-inhibitory molecules include, without limitation, PD-L1, PD-L2, HVEM, B7-H3, TRAIL, immunoglobulin-like transcripts (ILT) receptors (ILT2, ILT3, ILT4), FasL, CTLA4, CD39, CD73, and B7-H4. In some embodiments, the co-inhibitory molecule is PD-L1 or PD-L2. In some embodiments, the tolerogenic factor increases the activity of the co-inhibitory molecule. In some embodiments, the tolerogenic factor increases expression of a co-inhibitory molecule. In some embodiments, the tolerogenic factor encodes the co-inhibitory molecule. In some embodiments, the tolerogenic factor increases the activity of the co-inhibitory molecule. In some embodiments, the tolerogenic factor increases the activity of a transcriptional regulator that may enhance expression of the co-inhibitory molecule. In some embodiments, the tolerogenic factor increases the activity of a polypeptide that increases expression of the co-inhibitory molecule. In some embodiments, the tolerogenic factor comprises nucleic acid encoding an enhancer of the co-inhibitory molecule. In some embodiments, the tolerogenic factor inhibits an inhibitor of a co-inhibitory molecule.

In some embodiments, the tolerogenic factor increases expression and/or activity of an immunosuppressive molecule. Exemplary immunosuppressive molecules include, without limitation, arginase-1 (ARG1), indoleamine 2,3-dioxygenase (IDO), Prostaglandin E2 (PGE2), inducible nitric-oxide synthase (iNOS), nitric oxide (NO), nitric-oxide synthase 2 (NOS2), thymic stromal lymphopoietin (TSLP), vascular intestinal peptide (VIP), hepatocyte growth factor (HGF), transforming growth factor-β (TGF-β), IFN-α, IL-4, IL-10, IL-13, and IL-35. In some embodiments, the immunosuppressive molecule is NO or IDO. In some embodiments, the tolerogenic factor encodes the immunosuppressive molecule. In some embodiments, the tolerogenic factor increases the activity of the immunosuppressive molecule. In some embodiments, the tolerogenic factor increases the activity of a transcriptional regulator that enhances expression of the immunosuppressive molecule. In some embodiments, the tolerogenic factor increases the activity of a polypeptide that enhances expression of the immunosuppressive molecule. In some embodiments, the tolerogenic factor comprises nucleic acid encoding an enhancer of the immunosuppressive molecule. In some embodiments, the tolerogenic factor inhibits a negative regulator of an immunosuppressive molecule.

In some embodiments, the tolerogenic factor inhibits expression and/or activity of an inflammatory molecule. In some embodiments, the inflammatory molecule is an inflammatory transcription factor. In some embodiments, the tolerogenic factor inhibits the inflammatory transcription factor. In some embodiments, the tolerogenic factor decreases expression of an inflammatory transcription factor. In some embodiments, the inflammatory transcription factor is NF-κB, an interferon regulatory factor (IRF), or a molecule associated with the JAK-STAT signaling pathway. The NF-κB pathway is a prototypical proinflammatory signaling pathway that mediates the expression of proinflammatory genes including cytokines, chemokines, and adhesion molecules. Interferon regulatory factors (IRFs) constitute a family of transcription factors that can regulate the expression of proinflammatory genes. The JAK-STAT signaling pathway transmits information from extracellular cytokine signals to the nucleus, resulting in DNA transcription and expression of genes involved in immune cell proliferation and differentiation. The JAK-STAT system, consists of a cell surface receptor, Janus kinases (JAKs), and Signal Transducer and Activator of Transcription (STAT) proteins. Exemplary JAK-STAT molecules include, without limitation, JAK1, JAK2, JAK 3, Tyk2, STAT1, STAT2, STAT3, STAT4, STATS (STAT5A and STAT5B), and STAT6. In some embodiments, the tolerogenic factor enhances expression of a suppressor of cytokine signaling (SOCS) protein. SOCS proteins may inhibit signaling through the JAK-STAT pathway. In some embodiments, the tolerogenic factor inhibits the expression of a nucleic acid encoding the inflammatory transcription factor. In some embodiments, the tolerogenic factor deletes a nucleic acid encoding the inflammatory transcription factor. In some embodiments, the tolerogenic factor increases the activity of a transcriptional regulator that suppresses expression of the inflammatory transcription factor. In some embodiments, the tolerogenic factor increases the activity of a protein inhibitor that suppresses expression of the inflammatory transcription factor. In some embodiments, the tolerogenic factor comprises nucleic acid encoding a suppressor of the inflammatory transcription factor.

In some embodiments, the tolerogenic factor enhances expression and/or activity of an anti-inflammatory molecule. In some embodiments, the anti-inflammatory molecule is an anti-inflammatory transcription factor. In some embodiments, the tolerogenic factor enhances the anti-inflammatory transcription factor. In some embodiments, the tolerogenic factor increases expression of an anti-inflammatory transcription factor. In some embodiments, the tolerogenic factor enhances expression of nucleic acid encoding the anti-inflammatory transcription factor. In some embodiments, the tolerogenic factor decreases the activity of a transcriptional regulator that suppresses expression of the anti-inflammatory transcription factor. In some embodiments, the tolerogenic factor decreases the activity of a protein inhibitor that suppresses expression of the anti-inflammatory transcription factor. In some embodiments, the tolerogenic factor comprises nucleic acid encoding an enhancer of the anti-inflammatory transcription factor.

In some embodiments, the tolerogenic factor comprises a nucleic acid. In some embodiments, the tolerogenic factor is a nucleic acid. Exemplary nucleic acids include, without limitation, recombinant nucleic acids, DNA, recombinant DNA, cDNA, genomic DNA, RNA, siRNA, mRNA, saRNA, miRNA, lncRNA, tRNA, gRNA, and shRNA. In some embodiments, the nucleic acid is homologous to a nucleic acid in the cell. In some embodiments, the nucleic acid is heterologous to a nucleic acid in the cell. In some embodiments, the tolerogenic factor is a plasmid. In some embodiments, the nucleic acid is a therapeutic nucleic acid. In some embodiments, the nucleic acid encodes a therapeutic polypeptide. In some embodiments, the tolerogenic factor comprises a nucleic acid encoding an siRNA, mRNA, miRNA, lncRNA, tRNA, or shRNA. For example, the tolerogenic factor can include siRNA to knock down expression of inflammatory genes. In some embodiments, the tolerogenic factor is a DNA sequence that binds NF-κB and prevents NF-κB activation and downstream signaling.

In some embodiments, the tolerogenic factor comprises a polypeptide. In some embodiments, the tolerogenic factor is a polypeptide. In some embodiments, the protein or polypeptide is a therapeutic protein, antibody, fusion protein, antigen, synthetic protein, reporter marker, or selectable marker. In some embodiments, the protein is a gene-editing protein or nuclease such as a zinc-finger nuclease (ZFN), transcription activator-like effector nuclease (TALEN), mega nuclease, CRE recombinase, transposase, RNA-guided endonuclease (e.g., CAS9 enzyme), DNA-guided endonuclease, or integrase enzyme. In some embodiments, the fusion proteins can include, without limitation, chimeric protein drags such as antibody drug conjugates or recombinant fusion proteins such as proteins tagged with GST or streptavidin. In some embodiments, the compound is a transcription factor. Exemplary transcription factors include, without limitation, Oct4, Sox2, c-Myc, Klf-4, T-bet, GATA3, FoxP3, and RORγt. In some embodiments, the polypeptide is IL-4, IL-10, IL-13, IL-35, IFN-α, or TGFβ. In some embodiments, the polypeptide is a therapeutic polypeptide. In some embodiments, the polypeptide is a fragment of a therapeutic polypeptide. In some embodiments, the polypeptide is a peptide nucleic acid (PNA).

In some embodiments, the tolerogenic factor comprises a protein-nucleic acid complex. In some embodiments, the tolerogenic factor is a protein-nucleic acid complex. In some embodiments, protein-nucleic acid complexes, such as clustered regularly interspaced short palindromic repeats (CRISPR)-Cas9, are used in genome editing applications. These complexes contain sequence-specific DNA-binding domains in combination with nonspecific DNA cleavage nucleases. These complexes enable targeted genome editing, including adding, disrupting, or changing the sequence of a specific gene. In some embodiments, a disabled Cas9 (dCas9) is used to block or induce transcription of a target gene. In some embodiments, the tolerogenic factor contains a Cas9 protein and a guide RNA and donor DNA. In some embodiments, the tolerogenic factor includes a nucleic acid encoding for a Cas9 protein and a guide RNA or donor DNA. In some embodiments, the gene editing complex targets expression of a costimulatory molecule (e.g., CD80 and/or CD86).

In some embodiments, the tolerogenic factor comprises a small molecule. In some embodiments, the tolerogenic factor is a small molecule. In some embodiments, the small molecule inhibits the activity of a costimulatory molecule, enhances the activity of a co-inhibitory molecule, and/or inhibits the activity of an inflammatory molecule. Exemplary small molecules include, without limitation, pharmaceutical agents, metabolites, or radionuclides. In some embodiments, the pharmaceutical agent is a therapeutic drug and/or cytotoxic agent. In some embodiments, the compound comprises a nanoparticle. Examples of nanoparticles include gold nanoparticles, quantum dots, carbon nanotubes, nanoshells, dendrimers, and liposomes. In some embodiments, the nanoparticle contains or is linked (covalently or noncovalently) to a therapeutic molecule. In some embodiments, the nanoparticle contains a nucleic acid, such as mRNA or cDNA.

In some embodiments, the anucleate cell-derived vesicle comprises a cytokine. In some embodiments, the anucleate cell-derived vesicle comprises an agent for modulating genetic material, such as DNA. In some embodiments, the anucleate cell-derived vesicle comprises a gene editing component, such as a CRISPR component. In some embodiments, the anucleate cell-derived vesicle comprises an agent for modulating RNA, such as decreasing the presence of an RNA species. In some embodiments, the anucleate cell-derived vesicle comprises a siRNA.

### Methods for Generating Anucleate Cell-Derived Vesicles

In certain aspects, there is provided a method for generating an anucleate cell-derived vesicle comprising an antigen, the method comprising: a) passing a cell suspension comprising an input (e.g., parent) anucleate cell through a cell-deforming constriction, wherein a diameter of the constriction is a function of a diameter of the input anucleate cell in the suspension, thereby causing perturbations of the input anucleate cell large enough for the antigen to pass through to form an anucleate cell-derived vesicle; b) incubating the anucleate cell-derived vesicle with the antigen for a sufficient time to allow the antigen to enter the anucleate cell-derived vesicle, thereby generating an anucleate cell-derived vesicle comprising the antigen. In some embodiments, the input anucleate cell comprises an adjuvant.

In certain aspects, there is provided a method for generating an anucleate cell-derived vesicle comprising an adjuvant, the method comprising: a) passing a cell suspension comprising an input (e.g., parent) anucleate cell through a cell-deforming constriction, wherein a diameter of the constriction is a function of a diameter of the input anucleate cell in the suspension, thereby causing perturbations of the input anucleate cell large enough for the adjuvant to pass through to form an anucleate cell-derived vesicle; b) incubating the anucleate cell-derived vesicle with the adjuvant for a sufficient time to allow the antigen to enter the anucleate cell-derived vesicle, thereby generating an anucleate cell-derived vesicle comprising the adjuvant. In some embodiments, the input anucleate cell comprises an adjuvant.

In certain aspect, there is provided a method for generating an anucleate cell-derived vesicle comprising an antigen and an adjuvant, the method comprising: a) passing a cell suspension comprising an input (e.g., parent) anucleate cell through a cell-deforming constriction, wherein a diameter of the constriction is a function of a diameter of the input anucleate cell in the suspension, thereby causing perturbations of the input anucleate cell large enough for the antigen and the adjuvant to pass through to form an anucleate cell-derived vesicle; b) incubating the anucleate cell-derived vesicle with the antigen and the adjuvant for a sufficient time to allow the antigen and the adjuvant to enter the anucleate cell-derived vesicle, thereby generating an anucleate cell-derived vesicle comprising the antigen and the adjuvant.

In some embodiments, the anucleate cell-derived vesicle is a red blood cell-derived vesicle, or a platelet-derived vesicle. In some embodiments, the anucleate cell-derived vesicle is an erythrocyte-derived vesicle or a reticulocyte-derived vesicle.

In some embodiments according to any of the methods described herein, the input (e.g., parent) anucleate cell is a mammalian cell. In some embodiments, the input anucleate cell is a monkey, mouse, dog, cat, horse, rat, sheep, goat, pig, or rabbit cell. In some embodiments, the input anucleate cell is a human cell. In some embodiments, the input anucleate cell is a non-mammalian cell. In some embodiments, the input anucleate cell is a chicken, frog, insect, fish, or nematode cell. In some embodiments, the input anucleate cell is an erythrocyte. In some embodiments, the input anucleate cell is a red blood cell. In some embodiments, the input anucleate cell is a precursor to RBCs. In some embodiments, the input anucleate cell is a reticulocyte. In some embodiments, the input anucleate cell is a platelet.

In some embodiments, presentation of antigen in an immunogenic environment enhances an immune response to the antigen or induces an immune response to the antigen. Antigens derived from eryptotic bodies, such as anucleate cell-derived vesicles, which can be cleared in the immunogenic environment of the liver and spleen, may stimulate or enhance an immune response to the antigens via activation of T cells. In some embodiments, the immune response is antigen-specific. Anucleate cell-derived vesicles, such as RBC-derived vesicles have a limited life-span and are unable to self-repair, causing eryptosis, a process analogous to apoptosis, that leads to removal of the anucleate cell-derived vesicles from the bloodstream. In some embodiments, the antigen may be released upon eryptosis of the anucleate cell-derived vesicles within the immunogenic environment, where it is subsequently engulfed, processed, and presented by an antigen-presenting cell. In some embodiments, the anucleate cell-derived vesicle containing the antigen is phagocytosed by an antigen-presenting cell, such as a macrophage, and the antigen is subsequently processed and presented by the antigen presenting cell. In some embodiments, the antigen presenting cell is a resident macrophage.

In some embodiments, the circulating half-life of an anucleate cell-derived vesicle in a mammal is decreased compared to an input (e.g., parent) anucleate cell. Methods for measuring the half-life of a cell, such as an anucleate cell, e.g., red blood cell, or an anucleate cell-derived vesicle are known in the art. See, e.g., Franco, R. S., Transfus Med Hemother, 39, 2012. For example, in some embodiments, the method for measuring the half-life of an anucleate cell or an anucleate cell-derived vesicle comprises a cohort labeling technique or a random labeling technique. In some embodiments, the method for measuring the half-life of an anucleate cell or an anucleate cell-derived vesicle comprises labeling, reinfusing the cell or vesicle, and measuring the disappearance upon reinfusion. In some embodiments, the method for measuring the half-life of an anucleate cell or an anucleate cell-derived vesicle encompassed in the present application comprises measuring the half-life of an appropriate reference control(s), such as a control comprising an input anucleate cell or a population of input anucleate cells.

In some embodiments, the circulating half-life in the mammal is decreased by more than about 50%, such as more than about any of 55%, 60%, 65%, 70%, 75%, 80%, 85%, 90%, 95%, 96%, 97%, 98%, or 99%, as compared to the input (e.g., parent) anucleate cell. In some embodiments, the circulating half-life in the mammal is decreased by about 50% to about 99.9%, such as any of about 70% to about 99.9%, about 85% to about 99.9%, or about 95% to about 99.9%, as compared to the input anucleate cell. In some embodiments, the circulating half-life in the mammal is decreased by about any of 50%, 55%, 60%, 65%, 70%, 75%, 80%, 85%, 90%, 95%, 96%, 97%, 98%, 99%, or 99.9%, as compared to the input anucleate cell.

In some embodiments, the circulating half-life of the anucleate cell-derived vesicle is less than about any of 5 minutes, 10 minutes, 15 minutes, 30 minutes, 1 hour, 6 hours, 12 hours, 1 day, 2 days, 3 days, 4 days, 5 days, 10 days, 20 days, 30 days, 40 days, 50 days, 60 days, 70 days, 80 days, 90 days, or 100 days. In some embodiments, the circulating half-life of the anucleate cell-derived vesicle is about any of 0.5 minute, 1 minute, 2 minutes, 3 minutes, 4 minutes, 5 minutes, 6 minutes, 7 minutes, 8 minutes, 9 minutes, 10 minutes, 15 minutes, 30 minutes, 45 minutes, 1 hour, 3 hours, 6 hours, 12 hours, 1 day, 2 days, 3 days, 4 days, 5 days, 10 days, 20 days, 30 days, 40 days, 50 days, 60 days, 70 days, 80 days, 90 days, or 100 days.

In some embodiments, the input (e.g., parent) anucleate cell is a human cell and wherein the circulating half-life of the anucleate cell-derived vesicle is less than about any of 5 minutes, 10 minutes, 15 minutes, 30 minutes, 1 hour, 6 hours, 12 hours, 1 day, 2 days, 3 days, 4 days, 5 days, 10 day, 20 days, 30 days, 40 days, 50 days, 60 days, 70 days, 80 days, 90 days, or 100 days s. In some embodiments, the input anucleate cell is a human cell and wherein the circulating half-life of the anucleate cell-derived vesicle is about any of 0.5 minute, 1 minute, 2 minutes, 3 minutes, 4 minutes, 5 minutes, 6 minutes, 7 minutes, 8 minutes, 9 minutes, 10 minutes, 15 minutes, 30 minutes, 45 minutes, 1 hour, 3 hours, 6 hours, 12 hours, 1 day, 2 days, 3 days, 4 days, 5 days, 10 days, 20 days, 30 days, 40 days, 50 days, 60 days, 70 days, 80 days, 90 days, or 100 days.

In some embodiments, the input (e.g., parent) anucleate cell is a red blood cell, wherein the hemoglobin level in the anucleate cell-derived vesicle is decreased compared to the input anucleate cell. Methods of measuring the hemoglobin level of a cell, such as an anucleate cell, e.g., red blood cell, or an anucleate cell-derived vesicle is known in the art. See, e.g., Chaudhary, R., J Blood Med, 8, 2017. For example, in some embodiments, the method comprises measuring a metabolic precursor or product to determine the turnover of hemoglobin. In some embodiments, the method for measuring the hemoglobin level of an anucleate cell or an anucleate cell-derived vesicle encompassed in the present application comprises measuring the hemoglobin levels of an appropriate reference control(s), such as a control comprising an input anucleate cell or a population of input anucleate cell.

In some embodiments, the hemoglobin level in the anucleate cell-derived vesicle is decreased by at least about any of 10%, 15%, 20%, 25%, 30%, 35%, 40%, 45%, 50%, 55% 60%, 65%, 70%, 75%, 80%, 85%, 90%, 95%, 96%, 97%, 98%, 99%, 99.9%, or 100%, as compared to the input (e.g., parent) anucleate cell. In some embodiments, the hemoglobin level in the anucleate cell-derived vesicle is decreased by about 50% to about 99.9%, such as any of about 70% to about 99.9%, about 85% to about 99.9%, or about 95% to about 99.9%, as compared to the input anucleate cell. In some embodiments, the hemoglobin level in the anucleate cell-derived vesicle is decreased by about any of 50%, 55%, 60%, 65%, 70%, 75%, 80%, 85%, 90%, 95%, 96%, 97%, 98%, 99%, or 99.9%, as compared to the input anucleate cell.

In some embodiments, the hemoglobin level in the anucleate cell-derived vesicle is about any of 0.01%, 0.1%, 0.5%, 1%, 2%, 3%, 4%, 5%, 10%, 20%, 30%, 40%, or 50% of the hemoglobin level in the input (e.g., parent) anucleate cell.

In some embodiments, the input (e.g., parent) anucleate cell is an erythrocyte and wherein the morphology of the anucleate cell-derived vesicle is modulated from that of the input anucleate cell. Morphology concerns the classification of, e.g., the shape, structure, geometry, intensity, form, smoothness, roughness, circularity, volume, surface area and/or size of a cell or a cell-derived vesicle. Methods for determining (such as measuring) morphology are known in the art. See, e.g., Boutros et al., Cell, 163, 2015; Girasole, M. et al., Biochim Biophys Acta Biomembr, 1768, 2007; and Chen et al., Comput Math Methods Med, 2012. In some embodiments, the method for determining morphology comprises high-content imaging. For example, the morphology of the cell can be assessed by staining with Hoechst dye followed by automated high-content image analysis. In other examples, the morphology can be determined through a shift in the forward and side scatter plots from flow cytometry. In some embodiments, the input anucleate cell is an erythrocyte and wherein the anucleate cell-derived vesicle is spherical in morphology. In some embodiments, the input anucleate cell is an erythrocyte and wherein the anucleate cell-derived vesicle has a reduced biconcave shape compared to the input anucleate cell. In some embodiments, the method for measuring morphology of an anucleate cell or an anucleate cell-derived vesicle encompassed in the present application comprises measuring the morphology of an appropriate reference control(s), such as a control comprising an input anucleate cell or a population of input anucleate cell.

In some embodiments, the input (e.g., parent) anucleate cell is an erythrocyte and wherein the anucleate cell-derived vesicle has a reduced biconcave shape, such as reduced by more than about any of 5%, 10%, 15%, 20%, 25%, 30%, 35%, 40%, 45%, 50%, 55%, 60%, 65%, 70%, 75%, 80%, 85%, 90%, 95%, 96%, 97%, 98%, 99%, or 99.9%, as compared to the input anucleate cell.

In some embodiments, the input (e.g., parent) anucleate cell is a red blood cell or an erythrocyte and wherein the anucleate cell-derived vesicle is a red blood cell ghost (RBC ghost).

In some embodiments, the half-life of the anucleate cell-derived vesicle can be further modified. In some embodiments, the half-life of the anucleate cell-derived vesicle is increased by the further modification. For example, the anucleate cell-derived vesicle may be modified to increase the time the anucleate cell-derived vesicle circulates in the blood stream before clearance in the liver and spleen. In some embodiments, the half-life of the anucleate cell-derived vesicle is further decreased by the modification. For example, the anucleate cell-derived vesicle may be modified to decrease the time the anucleate cell circulates in the blood stream before clearance in the spleen. In some embodiments, an altered ratio of phospholipids on the surface of the anucleate cell-derived vesicle decreases the half-life of the anucleate cell-derived vesicle. In some embodiments, an increased ratio of phosphatidylserine to other phospholipids on the surface of the anucleate cell-derived vesicle decreases the half-life of the anucleate cell-derived vesicle. For example, the presence of phosphatidylserine on the surface of the anucleate cell-derived vesicle can be further increased to decrease the half-life of the anucleate cell, such as by using any method known in the art for increasing surface phosphatidylserine (see Hamidi et al., J. Control. Release, 2007, 118(2): 145-60). In some embodiments, the anucleate cell-derived vesicle is incubated with lipids or phospholipids prior to delivery to an individual. In some embodiments, the anucleate cell-derived vesicle is treated by chemicals such as bis(sulfosuccinimidyl)suberate or other cross-linking agents, prior to delivery to an individual. In other embodiments, the surface phosphatidylserine of the anucleate cell-derived vesicle can be decreased to increase the half-life of the anucleate cell-derived vesicle. In some embodiments, the anucleate cell-derived vesicle is treated with flippase prior to delivery to an individual. Generally, flippases are enzymes that transport phospholipids from the external leaflet to the cytosolic leaflet in the plasma membrane. In some embodiments, the anucleate cell-derived vesicle is treated with an enzyme that cleaves phosphatidylserines, prior to delivery to an individual. A non-limiting example of an enzyme that cleaves phosphatidylserine is phosphatidylserine carboxylase.

In some embodiments, the anucleate cell-derive vesicle exhibits one or more of the following properties: (a) a circulating half-life in a mammal is decreased compared to the parent anucleate cell, (b) decreased hemoglobin levels compared to the parent anucleate cell, (c) spherical morphology, (d) increased surface phosphatidylserine levels compared to the parent anucleate cell, or (e) reduced ATP production compared to the parent anucleate cell.

In some embodiments according to any of the methods described herein, the osmolarity of the cell suspension is maintained throughout the process. In further embodiments, the osmolarity of the cell suspension is maintained between 200 mOsm and 400 mOsm throughout the process. In some embodiments, the osmolarity of the cell suspension is maintained between 200 mOsm and 600 mOsm throughout the process. In further embodiments, the osmolarity of the cell suspension is maintained between 200 mOsm and 800 mOsm throughout the process. In some embodiments, the osmolarity of the cell suspension is maintained between any one of: 200 mOsm and 300 mOsm, 300 mOsm and 400 mOsm, 400 mOsm and 500 mOsm, 500 mOsm and 600 mOsm, 600 mOsm and 700 mOsm, 700 mOsm and 800 mOsm.

In some embodiments, according to any of the methods or anucleate cell-derived vesicles described herein, the anucleate cell-derived vesicle further comprises an additional agent that enhances the function of the anucleate cell-derived vesicle as compared to a corresponding anucleate cell-derived vesicle that does not comprise the additional agent. In some embodiments, the additional agent is a stabilizing agent or a co-factor. In some embodiments, the agent is albumin. In some embodiments, the albumin is mouse, bovine, or human albumin. In some embodiments, the additional agent is a divalent metal cation, glucose, ATP, potassium, glycerol, trehalose, D-sucrose, PEG1500, L-arginine, L-glutamine, or EDTA. In some embodiments, the anucleate cell-derived vesicles further comprise one or more therapeutic agents.

In some embodiments, the anucleate cell-derived vesicle comprises an antigen and a tolerogenic factor, wherein the anucleate cell-derived vesicle was prepared by a process comprising: (a) passing a cell suspension comprising the input parent anucleate cell through a cell-deforming constriction, wherein a diameter of the constriction is a function of a diameter of the input parent anucleate cell in the suspension, thereby causing perturbations of the input parent anucleate cell large enough for the antigen and the tolerogenic factor to pass through to form an anucleate cell-derived vesicle; and (b) incubating the anucleate cell-derived vesicle with the antigen and the tolerogenic factor for a sufficient time to allow the antigen and the tolerogenic factor to enter the anucleate cell-derived vesicle; thereby producing an anucleate cell-derived vesicle comprising an antigen and an tolerogenic factor.

In some embodiments, the constriction is contained within a microfluidic channel. In some embodiments, the microfluidic channel comprises a plurality of constrictions. In some embodiments, the plurality of constrictions are arranged in series and/or in parallel. In some embodiments, the constriction is between a plurality of micropillars; between a plurality of micropillars configured in an array; or between one or more movable plates. In some embodiments, the constriction is formed by a plurality of micropillars. In some embodiments, the constriction is formed between a plurality of micropillars configured in an array. In some embodiments, the constriction is formed by one or more movable plates.

In some embodiments, the constriction is a pore or contained within a pore. In some embodiments, the pore is contained in a surface. In some embodiments, the surface is a filter. In some embodiments, the surface is a membrane.

In some embodiments, the constriction size is about any of 10%, 15%, 20%, 25%, 30%, 35%, 40%, 45%, 50%, 55%, 60%, 65%, 70%, 75%, or 80% of a diameter of the cell, such as the largest diameter of an anucleate cell in suspension. In some embodiments, the constriction size is less than about any of 80%, 75%, 70%, 65%, 60%, 55%, 50%, 45%, 40%, 35%, 30%, 25%, 20%, 15%, or 10% of a diameter of the cell, such as the largest diameter of an anucleate cell in suspension. In some embodiments, the constriction has a width of about 0.1 μm to about 4 μm, such as any of about 1 μm to about 3 μm, about 1.75 μm to about 2.5 μm, or about 2 μm to about 2.5 μm. In some embodiments, the constriction has a width of about any of 4 μm, 3.5 μm, 3 μm, 2.5 μm, 2 μm, 1.5 μm, 1 μm, 0.5 μm, or about 0.25 μm. In some embodiments, the constriction has a width of about any of 1.8 μm, 1.9 μm, 2 μm, 2.1 μm, 2.2 μm, 2.3 μm, 2.4 μm, 2.5 μm or 2.6 μm. In some embodiments, the constriction has a width of about 2.2 μm.

In some embodiments, the input parent anucleate cells are passed through the constriction under a pressure ranging from about 10 psi to about 150 psi, such as any of about 30 psi to about 60 psi, about 10 psi to about 40 psi, about 50 psi to about 90 psi. In some embodiments, the input parent anucleate cells are passed through the constriction under a pressure of at least about any of 5 psi, 10 psi, 15 psi, 20 psi, 25 psi, 30 psi, 35 psi, 40 psi, 45 psi, 50 psi, 55 psi, 60 psi, 65 psi, 70 psi, 75 psi, 80 psi, 85 psi, 90 psi, 95 psi, 100 psi, 105 psi, 110 psi, 115 psi, 120 psi, 125 psi, 130 psi, 135 psi, 140 psi, 145 psi, or 150 psi. In some embodiments, the input parent anucleate cells are passed through the constriction under a pressure of at least about 5 psi and less than about any of 150 psi, 145 psi, 140 psi, 135 psi, 130 psi, 125 psi, 120 psi, 115 psi, 110 psi, 105 psi, 100 psi, 95 psi, 90 psi, 85 psi, 80 psi, 75 psi, 70 psi, 65 psi, 60 psi, 55 psi, 50 psi, 45 psi, 40 psi, 35 psi, 30 psi, 25 psi, 20 psi, or 15 psi.

In some embodiments, the cell suspension is contacted (such as first contacted) with the payload before passing through the constriction. In some embodiments, the cell suspension is contacted (such as first contacted) with the payload concurrently with passing through the constriction. In some embodiments, the cell suspension is contacted (such as first contacted) with the payload after passing through the constriction. In some embodiments, the cell suspension is at least contacted with the payload concurrently with passing through the constriction and after passing through the constriction. In some embodiments, the cell suspension is contacted with the payload before passing through the constriction, concurrently with passing through the constriction, and after passing through the constriction.

In some embodiments, an anucleate cell-derived vesicle comprising an antigen and an adjuvant as described herein is an activating antigen carrier (AAC).

In some embodiments, an anucleate cell-derived vesicle comprising an antigen for tolearization as described herein is an tolerizing antigen carrier (TAC).

### Compositions

In some aspects, the present application provides compositions comprising a plurality of any of the anucleate cell-derived vesicles described herein.

In some embodiments, provided is a composition comprising a plurality of anucleate cell-derived vesicles prepared from parent anucleate cells, the composition having any one or more of the following properties: (a) greater than about 20%, such as greater than about any of 25%, 30%, 35%, 40%, 45%, 50%, 55%, 60%, 65%, 70%, 75%, 80%, 85%, 90% or 95%, of the anucleate cell-derived vesicles in the composition have a circulating half-life in a mammal that is decreased compared to the parent anucleate cell, (b) greater than about 20%, such as greater than about any of 25%, 30%, 35%, 40%, 45%, 50%, 55%, 60%, 65%, 70%, 75%, 80%, 85%, 90% or 95%, of the anucleate cell-derived vesicles in the composition have decreased hemoglobin levels compared to the parent anucleate cell, (c) greater than about 20%, such as greater than about any of 25%, 30%, 35%, 40%, 45%, 50%, 55%, 60%, 65%, 70%, 75%, 80%, 85%, 90% or 95%, of the anucleate cell-derived vesicles in the composition have spherical morphology, (d) greater than about 20%, such as greater than about any of 25%, 30%, 35%, 40%, 45%, 50%, 55%, 60%, 65%, 70%, 75%, 80%, 85%, 90% or 95%, of the anucleate cell-derived vesicles in the composition are RBC ghosts, (e) greater than about 20%, such as greater than about any of 25%, 30%, 35%, 40%, 45%, 50%, 55%, 60%, 65%, 70%, 75%, 80%, 85%, 90% or 95%, of the anucleate cell-derived vesicles in the composition vesicles in the composition have higher levels of phosphatidylserine, or (f) greater than about 20%, such as greater than about any of 25%, 30%, 35%, 40%, 45%, 50%, 55%, 60%, 65%, 70%, 75%, 80%, 85%, 90% or 95%, of the anucleate cell-derived vesicles in the composition have reduced ATP production compared to the parent anucleate cell.

In some embodiments, the composition comprising a plurality of anucleate cell-derived vesicles may be actively tuned to generate a desired profile of anucleate cell-derived vesicles within the composition having one or more select properties, including one or more of: (a) greater than about 20%, such as greater than about any of 25%, 30%, 35%, 40%, 45%, 50%, 55%, 60%, 65%, 70%, 75%, 80%, 85%, 90% or 95%, of the anucleate cell-derived vesicles in the composition have a circulating half-life in a mammal that is decreased compared to the parent anucleate cell, (b) greater than about 20%, such as greater than about any of 25%, 30%, 35%, 40%, 45%, 50%, 55%, 60%, 65%, 70%, 75%, 80%, 85%, 90% or 95%, of the anucleate cell-derived vesicles in the composition have decreased hemoglobin levels compared to the parent anucleate cell, (c) greater than about 20%, such as greater than about any of 25%, 30%, 35%, 40%, 45%, 50%, 55%, 60%, 65%, 70%, 75%, 80%, 85%, 90% or 95%, of the anucleate cell-derived vesicles in the composition have spherical morphology, (d) greater than about 20%, such as greater than about any of 25%, 30%, 35%, 40%, 45%, 50%, 55%, 60%, 65%, 70%, 75%, 80%, 85%, 90% or 95%, of the anucleate cell-derived vesicles in the composition are RBC ghosts, (e) greater than about 20%, such as greater than about any of 25%, 30%, 35%, 40%, 45%, 50%, 55%, 60%, 65%, 70%, 75%, 80%, 85%, 90% or 95%, of the anucleate cell-derived vesicles in the composition vesicles in the composition have higher levels of phosphatidylserine, or (f) greater than about 20%, such as greater than about any of 25%, 30%, 35%, 40%, 45%, 50%, 55%, 60%, 65%, 70%, 75%, 80%, 85%, 90% or 95%, of the anucleate cell-derived vesicles in the composition have reduced ATP production compared to the parent anucleate cell.

In some embodiments, the composition comprising a plurality of anucleate cell-derived vesicles having a desired profile of select properties is prepared from parent anucleate cells using the methods of making described herein, including use of microfluidic constrictions, wherein parameters of the methods of making, including constriction dimension, speed of passing a parent anucleate cell through the constriction, constriction architecture (e.g., Weir structure and size), processing time, pressure, and buffer composition, are selected to produce the composition comprising a plurality of anucleate cell-derived vesicles having the desired profile of select properties. In some embodiments, the method of making a composition comprising a plurality of anucleate cell-derived vesicles having a desired profile of select properties comprises selecting a set of parameters, including constriction dimension, speed of passing a parent anucleate cell through the constriction, constriction architecture (e.g., Weir structure and size), processing time, pressure, and buffer composition, to produce the composition. In some embodiments, the method of making a composition comprising a plurality of anucleate cell-derived vesicles having a desired profile of select properties comprises using a set of parameters, including constriction dimension, speed of passing a parent anucleate cell through the constriction, constriction architecture (e.g., Weir structure and size), processing time, pressure, and buffer composition, to produce the composition.

For example, in some embodiments, the composition comprising a plurality of anucleate cell-derived vesicles having a desired profile of select properties is prepared using a set of parameters comprising a constriction dimension, e.g., about 2.2 μm or about 2.5 μm, and a pressure, e.g., about 30 psi or about 50 psi. In some embodiments, the composition comprising a plurality of anucleate cell-derived vesicles having a desired profile of select properties is prepared using a set of parameters comprising a constriction dimension of about 2.2 μm and a pressure of about 30 psi. In some embodiments, the composition comprising a plurality of anucleate cell-derived vesicles having a desired profile of select properties is prepared using a set of parameters comprising a constriction dimension of about 2.2 μm and a pressure of about 50 psi. In some embodiments, the composition comprising a plurality of anucleate cell-derived vesicles having a desired profile of select properties is prepared using a set of parameters comprising a constriction dimension of about 2.5 μm and a pressure of about 30 psi. In some embodiments, the composition comprising a plurality of anucleate cell-derived vesicles having a desired profile of select properties is prepared using a set of parameters comprising a constriction dimension of about 2.5 μm and a pressure of about 50 psi.

In some embodiments, the parent anucleate cell is a mammalian cell, which includes, but is not limited to, a cell from a human, bovine, horse, feline, canine, rodent, or primate. In some embodiments, the parent anucleate cell is a human cell. In some embodiments, the parent anucleate cell is an anucleate cell from a mammal, which includes, but is not limited to, a human, bovine, horse, feline, canine, rodent, or primate.

In some embodiments, the parent anucleate cell is a red blood cell. In some embodiments, the parent anucleate cell is a platelet. In some embodiments, the red blood cell is an erythrocyte. In some embodiments, the red blood cell is a reticulocyte.

In some embodiments, the circulating half-life of at least about 20%, such as at least about any of 25%, 30%, 35%, 40%, 45%, 50%, 55%, 60%, 65%, 70%, 75%, 80%, 85%, 90% or 95%, of the anucleate cell-derived vesicles in the composition in a mammal is decreased compared to the parent anucleate cell. In some embodiments, the circulating half-life of at least about 75%, such as at least about any of 80%, 85%, 90% or 95%, of the anucleate cell-derived vesicles in the composition in a mammal is decreased compared to the parent anucleate cell. In some embodiments, the circulating half-life of about any of 20%, 25%, 30%, 35%, 40%, 45%, 50%, 55%, 60%, 65%, 70%, 75%, 80%, 85%, 90% or 95% of the anucleate cell-derived vesicles in the composition in a mammal is decreased compared to the parent anucleate cell. In some embodiments, the circulating half-life of at least about 20%, such as at least about any of 25%, 30%, 35%, 40%, 45%, 50%, 55%, 60%, 65%, 70%, 75%, 80%, 85%, 90% or 95%, of the anucleate cell-derived vesicles in the composition in the mammal is decreased by more than about any of 50%, 55%, 60%, 65%, 70%, 75%, 80%, 85%, 90%, or 90% compared to the parent anucleate cell. In some embodiments, the parent anucleate cell is a human cell, and the circulating half-life of at least about 20%, such as at least about any of 25%, 30%, 35%, 40%, 45%, 50%, 55%, 60%, 65%, 70%, 75%, 80%, 85%, 90% or 95%, of the anucleate cell-derived vesicles in the composition is less than about any of 5 minutes, 10 minutes, 15 minutes, 30 minutes, 1 hour, 6 hours, 12 hours, 1 day, 2 days, 3 days, 4 days, 5 days, or 10 days. In some embodiments, the parent anucleate cell is a human cell, and the circulating half-life of at least about 20%, such as at least about any of 25%, 30%, 35%, 40%, 45%, 50%, 55%, 60%, 65%, 70%, 75%, 80%, 85%, 90% or 95%, of the anucleate cell-derived vesicles in the composition is about any of 1 minute, 2 minutes, 3 minutes, 4 minutes, 5 minutes, 6 minutes, 7 minutes, 8 minutes, 9 minutes, 10 minutes, 15 minutes, 30 minutes, 45 minutes, 1 hour, 3 hours, 6 hours, 12 hours, 1 day, 2 days, 3 days, 4 days, 5 days, or 10 days.

In some embodiments, the parent anucleate cell is a red blood cell, and the hemoglobin levels of at least about 20%, such as at least about any of 25%, 30%, 35%, 40%, 45%, 50%, 55%, 60%, 65%, 70%, 75%, 80%, 85%, 90% or 95%, of the anucleate cell-derived vesicles in the composition are decreased compared to the parent anucleate cell. In some embodiments, the hemoglobin levels of at least about 75%, such as at least about any of 80%, 85%, 90% or 95%, of the anucleate cell-derived vesicles in the composition in a mammal is decreased compared to the parent anucleate cell. In some embodiments, the hemoglobin levels of 20%, such as at least about any of 25%, 30%, 35%, 40%, 45%, 50%, 55%, 60%, 65%, 70%, 75%, 80%, 85%, 90% or 95%, of the anucleate cell-derived vesicles in the composition of the anucleate cell-derived vesicle are decreased by at least about any of 10%, 15%, 20%, 25%, 30%, 35%, 40%, 45%, 50%, 55%, 60%, 65% 70%, 75%, 80%, 85%, 90%, 95%, 99%, or 100% compared to the parent anucleate cell. In some embodiments, the hemoglobin levels of at least about 20%, such as at least about any of 25%, 30%, 35%, 40%, 45%, 50%, 55%, 60%, 65%, 70%, 75%, 80%, 85%, 90% or 95%, of the anucleate cell-derived vesicles in the composition are about any of 1%, 2%, 3%, 4%, 5%, 10%, 15%, 20%, 25%, 30%, 35%, 40%, 45%, or 50% the level of hemoglobin in the parent anucleate cell.

In some embodiments, the parent anucleate cell is an erythrocyte, and at least about 20%, such as at least about any of 25%, 30%, 35%, 40%, 45%, 50%, 55%, 60%, 65%, 70%, 75%, 80%, 85%, 90% or 95%, of the anucleate cell-derived vesicles in the composition have a modulated morphology as compared to the parent anucleate cell. In some embodiments, the parent anucleate cell is an erythrocyte, and at least about 20%, such as at least about any of 25%, 30%, 35%, 40%, 45%, 50%, 55%, 60%, 65%, 70%, 75%, 80%, 85%, 90% or 95%, of the anucleate cell-derived vesicles in the composition are spherical in morphology. In some embodiments, the parent anucleate cell is an erythrocyte, and at least about 20%, such as at least about any of 25%, 30%, 35%, 40%, 45%, 50%, 55%, 60%, 65%, 70%, 75%, 80%, 85%, 90% or 95%, of the anucleate cell-derived vesicles in the composition have a reduced, such as reduced by more than about any of 5%, 10%, 15%, 20%, 25%, 30%, 35%, 40%, 45%, 50%, 55%, 60%, 65%, 70%, 75%, 80%, 85%, 90% or 95%, bioconcave shape compared to the parent anucleate cell.

In some embodiments, the parent anucleate cell is a red blood cell or an erythrocyte, and at least about 20%, such as at least about any of 25%, 30%, 35%, 40%, 45%, 50%, 55%, 60%, 65%, 70%, 75%, 80%, 85%, 90% or 95%, of the anucleate cell-derived vesicles in the composition are red blood cell ghosts.

In some embodiments, at least about 20%, such as at least about any of 25%, 30%, 35%, 40%, 45%, 50%, 55%, 60%, 65%, 70%, 75%, 80%, 85%, 90% or 95%, of the anucleate cell-derived vesicles in the composition comprise surface phosphatidylserine. In some embodiments, at least about 20%, such as at least about any of 25%, 30%, 35%, 40%, 45%, 50%, 55%, 60%, 65%, 70%, 75%, 80%, 85%, 90% or 95%, of the anucleate cell-derived vesicles in the composition comprise increased surface phosphatidylserine levels compared to the parent anucleate cells. In some embodiments, at least about 20%, such as at least about any of 25%, 30%, 35%, 40%, 45%, 50%, 55%, 60%, 65%, 70%, 75%, 80%, 85%, 90% or 95%, of the anucleate cell-derived vesicles in the composition have greater than about any of 5%, 10%, 15%, 20%, 25%, 30%, 35%, 40%, 45%, 50%, 55%, 60%, 65%, 70%, 75%, 80%, 85%, 90%, 95%, 100%, 125%, 150%, 175%, or 200% higher surface phosphatidylserine levels compared to a composition comprising a plurality of parent anucleate cells.

In some embodiments, at least about 20%, such as at least about any of 25%, 30%, 35%, 40%, 45%, 50%, 55%, 60%, 65%, 70%, 75%, 80%, 85%, 90% or 95%, of the anucleate cell-derived vesicles in the composition have reduced ATP production compared to the parent anucleate cell. In some embodiments, at least about 20%, such as at least about any of 25%, 30%, 35%, 40%, 45%, 50%, 55%, 60%, 65%, 70%, 75%, 80%, 85%, 90% or 95%, of the anucleate cell-derived vesicles in the composition produce ATP at less than about any of 1%, 2%, 3%, 4%, 5%, 10%, 15%, 20%, 25%, 30%, 35%, 40%, 45%, or 50% the level of ATP produced by the parent anucleate cell. In some embodiments, ATP production of a sample and a control is measured under similar conditions. In some embodiments, at least about 20%, such as at least about any of 25%, 30%, 35%, 40%, 45%, 50%, 55%, 60%, 65%, 70%, 75%, 80%, 85%, 90% or 95%, do not produce ATP.

In some embodiments, provided is a composition comprising a plurality of anucleate cell-derived vesicles prepared from parent anucleate cells, the composition comprising the following property, as further described herein, of greater than about 20%, such as greater than about any of 25%, 30%, 35%, 40%, 45%, 50%, 55%, 60%, 65%, 70%, 75%, 80%, 85%, 90% or 95%, of the anucleate cell-derived vesicles in the composition have a circulating half-life in a mammal that is decreased compared to the parent anucleate cell.

In some embodiments, provided is a composition comprising a plurality of anucleate cell-derived vesicles prepared from parent anucleate cells, the composition comprising the following property, as further described herein, of greater than about 20%, such as greater than about any of 25%, 30%, 35%, 40%, 45%, 50%, 55%, 60%, 65%, 70%, 75%, 80%, 85%, 90% or 95%, of the anucleate cell-derived vesicles in the composition have decreased hemoglobin levels compared to the parent anucleate cell.

In some embodiments, provided is a composition comprising a plurality of anucleate cell-derived vesicles prepared from parent anucleate cells, the composition comprising the following property, as further described herein, of greater than about 20%, such as greater than about any of 25%, 30%, 35%, 40%, 45%, 50%, 55%, 60%, 65%, 70%, 75%, 80%, 85%, 90% or 95%, of the anucleate cell-derived vesicles in the composition have spherical morphology.

In some embodiments, provided is a composition comprising a plurality of anucleate cell-derived vesicles prepared from parent anucleate cells, the composition comprising the following property, as further described herein, of greater than about 20%, such as greater than about any of 25%, 30%, 35%, 40%, 45%, 50%, 55%, 60%, 65%, 70%, 75%, 80%, 85%, 90% or 95%, of the anucleate cell-derived vesicles in the composition are RBC ghosts.

In some embodiments, provided is a composition comprising a plurality of anucleate cell-derived vesicles prepared from parent anucleate cells, the composition comprising the following property, as further described herein, of greater than about 20%, such as greater than about any of 25%, 30%, 35%, 40%, 45%, 50%, 55%, 60%, 65%, 70%, 75%, 80%, 85%, 90% or 95%, of the anucleate cell-derived vesicles in the composition vesicles in the composition have higher levels of phosphatidylserine.

In some embodiments, provided is a composition comprising a plurality of anucleate cell-derived vesicles prepared from parent anucleate cells, the composition comprising the following property, as further described herein, of greater than about 20%, such as greater than about any of 25%, 30%, 35%, 40%, 45%, 50%, 55%, 60%, 65%, 70%, 75%, 80%, 85%, 90% or 95%, of the anucleate cell-derived vesicles in the composition have reduced ATP production compared to the parent anucleate cell.

In some embodiments, provided is a composition comprising a plurality of anucleate cell-derived vesicles prepared from parent anucleate cells, the composition comprising any two of the following properties, as further described herein, of (a) greater than about 20%, such as greater than about any of 25%, 30%, 35%, 40%, 45%, 50%, 55%, 60%, 65%, 70%, 75%, 80%, 85%, 90% or 95%, of the anucleate cell-derived vesicles in the composition have a circulating half-life in a mammal that is decreased compared to the parent anucleate cell, (b) greater than about 20%, such as greater than about any of 25%, 30%, 35%, 40%, 45%, 50%, 55%, 60%, 65%, 70%, 75%, 80%, 85%, 90% or 95%, of the anucleate cell-derived vesicles in the composition have decreased hemoglobin levels compared to the parent anucleate cell, (c) greater than about 20%, such as greater than about any of 25%, 30%, 35%, 40%, 45%, 50%, 55%, 60%, 65%, 70%, 75%, 80%, 85%, 90% or 95%, of the anucleate cell-derived vesicles in the composition have spherical morphology, (d) greater than about 20%, such as greater than about any of 25%, 30%, 35%, 40%, 45%, 50%, 55%, 60%, 65%, 70%, 75%, 80%, 85%, 90% or 95%, of the anucleate cell-derived vesicles in the composition are RBC ghosts, (e) greater than about 20%, such as greater than about any of 25%, 30%, 35%, 40%, 45%, 50%, 55%, 60%, 65%, 70%, 75%, 80%, 85%, 90% or 95%, of the anucleate cell-derived vesicles in the composition vesicles in the composition have higher levels of phosphatidylserine, or (f) greater than about 20%, such as greater than about any of 25%, 30%, 35%, 40%, 45%, 50%, 55%, 60%, 65%, 70%, 75%, 80%, 85%, 90% or 95%, of the anucleate cell-derived vesicles in the composition have reduced ATP production compared to the parent anucleate cell.

In some embodiments, provided is a composition comprising a plurality of anucleate cell-derived vesicles prepared from parent anucleate cells, the composition comprising any three of the following properties, as further described herein, of (a) greater than about 20%, such as greater than about any of 25%, 30%, 35%, 40%, 45%, 50%, 55%, 60%, 65%, 70%, 75%, 80%, 85%, 90% or 95%, of the anucleate cell-derived vesicles in the composition have a circulating half-life in a mammal that is decreased compared to the parent anucleate cell, (b) greater than about 20%, such as greater than about any of 25%, 30%, 35%, 40%, 45%, 50%, 55%, 60%, 65%, 70%, 75%, 80%, 85%, 90% or 95%, of the anucleate cell-derived vesicles in the composition have decreased hemoglobin levels compared to the parent anucleate cell, (c) greater than about 20%, such as greater than about any of 25%, 30%, 35%, 40%, 45%, 50%, 55%, 60%, 65%, 70%, 75%, 80%, 85%, 90% or 95%, of the anucleate cell-derived vesicles in the composition have spherical morphology, (d) greater than about 20%, such as greater than about any of 25%, 30%, 35%, 40%, 45%, 50%, 55%, 60%, 65%, 70%, 75%, 80%, 85%, 90% or 95%, of the anucleate cell-derived vesicles in the composition are RBC ghosts, (e) greater than about 20%, such as greater than about any of 25%, 30%, 35%, 40%, 45%, 50%, 55%, 60%, 65%, 70%, 75%, 80%, 85%, 90% or 95%, of the anucleate cell-derived vesicles in the composition vesicles in the composition have higher levels of phosphatidylserine, or (f) greater than about 20%, such as greater than about any of 25%, 30%, 35%, 40%, 45%, 50%, 55%, 60%, 65%, 70%, 75%, 80%, 85%, 90% or 95%, of the anucleate cell-derived vesicles in the composition have reduced ATP production compared to the parent anucleate cell.

In some embodiments, provided is a composition comprising a plurality of anucleate cell-derived vesicles prepared from parent anucleate cells, the composition comprising any four of the following properties, as further described herein, of (a) greater than about 20%, such as greater than about any of 25%, 30%, 35%, 40%, 45%, 50%, 55%, 60%, 65%, 70%, 75%, 80%, 85%, 90% or 95%, of the anucleate cell-derived vesicles in the composition have a circulating half-life in a mammal that is decreased compared to the parent anucleate cell, (b) greater than about 20%, such as greater than about any of 25%, 30%, 35%, 40%, 45%, 50%, 55%, 60%, 65%, 70%, 75%, 80%, 85%, 90% or 95%, of the anucleate cell-derived vesicles in the composition have decreased hemoglobin levels compared to the parent anucleate cell, (c) greater than about 20%, such as greater than about any of 25%, 30%, 35%, 40%, 45%, 50%, 55%, 60%, 65%, 70%, 75%, 80%, 85%, 90% or 95%, of the anucleate cell-derived vesicles in the composition have spherical morphology, (d) greater than about 20%, such as greater than about any of 25%, 30%, 35%, 40%, 45%, 50%, 55%, 60%, 65%, 70%, 75%, 80%, 85%, 90% or 95%, of the anucleate cell-derived vesicles in the composition are RBC ghosts, (e) greater than about 20%, such as greater than about any of 25%, 30%, 35%, 40%, 45%, 50%, 55%, 60%, 65%, 70%, 75%, 80%, 85%, 90% or 95%, of the anucleate cell-derived vesicles in the composition vesicles in the composition have higher levels of phosphatidylserine, or (f) greater than about 20%, such as greater than about any of 25%, 30%, 35%, 40%, 45%, 50%, 55%, 60%, 65%, 70%, 75%, 80%, 85%, 90% or 95%, of the anucleate cell-derived vesicles in the composition have reduced ATP production compared to the parent anucleate cell.

In some embodiments, provided is a composition comprising a plurality of anucleate cell-derived vesicles prepared from parent anucleate cells, the composition comprising any five of the following properties, as further described herein, of (a) greater than about 20%, such as greater than about any of 25%, 30%, 35%, 40%, 45%, 50%, 55%, 60%, 65%, 70%, 75%, 80%, 85%, 90% or 95%, of the anucleate cell-derived vesicles in the composition have a circulating half-life in a mammal that is decreased compared to the parent anucleate cell, (b) greater than about 20%, such as greater than about any of 25%, 30%, 35%, 40%, 45%, 50%, 55%, 60%, 65%, 70%, 75%, 80%, 85%, 90% or 95%, of the anucleate cell-derived vesicles in the composition have decreased hemoglobin levels compared to the parent anucleate cell, (c) greater than about 20%, such as greater than about any of 25%, 30%, 35%, 40%, 45%, 50%, 55%, 60%, 65%, 70%, 75%, 80%, 85%, 90% or 95%, of the anucleate cell-derived vesicles in the composition have spherical morphology, (d) greater than about 20%, such as greater than about any of 25%, 30%, 35%, 40%, 45%, 50%, 55%, 60%, 65%, 70%, 75%, 80%, 85%, 90% or 95%, of the anucleate cell-derived vesicles in the composition are RBC ghosts, (e) greater than about 20%, such as greater than about any of 25%, 30%, 35%, 40%, 45%, 50%, 55%, 60%, 65%, 70%, 75%, 80%, 85%, 90% or 95%, of the anucleate cell-derived vesicles in the composition vesicles in the composition have higher levels of phosphatidylserine, or (f) greater than about 20%, such as greater than about any of 25%, 30%, 35%, 40%, 45%, 50%, 55%, 60%, 65%, 70%, 75%, 80%, 85%, 90% or 95%, of the anucleate cell-derived vesicles in the composition have reduced ATP production compared to the parent anucleate cell.

In some embodiments, provided is a composition comprising a plurality of anucleate cell-derived vesicles prepared from parent anucleate cells, the composition comprising the following properties, as further described herein, of (a) greater than about 20%, such as greater than about any of 25%, 30%, 35%, 40%, 45%, 50%, 55%, 60%, 65%, 70%, 75%, 80%, 85%, 90% or 95%, of the anucleate cell-derived vesicles in the composition have a circulating half-life in a mammal that is decreased compared to the parent anucleate cell, (b) greater than about 20%, such as greater than about any of 25%, 30%, 35%, 40%, 45%, 50%, 55%, 60%, 65%, 70%, 75%, 80%, 85%, 90% or 95%, of the anucleate cell-derived vesicles in the composition have decreased hemoglobin levels compared to the parent anucleate cell, (c) greater than about 20%, such as greater than about any of 25%, 30%, 35%, 40%, 45%, 50%, 55%, 60%, 65%, 70%, 75%, 80%, 85%, 90% or 95%, of the anucleate cell-derived vesicles in the composition have spherical morphology, (d) greater than about 20%, such as greater than about any of 25%, 30%, 35%, 40%, 45%, 50%, 55%, 60%, 65%, 70%, 75%, 80%, 85%, 90% or 95%, of the anucleate cell-derived vesicles in the composition are RBC ghosts, (e) greater than about 20%, such as greater than about any of 25%, 30%, 35%, 40%, 45%, 50%, 55%, 60%, 65%, 70%, 75%, 80%, 85%, 90% or 95%, of the anucleate cell-derived vesicles in the composition vesicles in the composition have higher levels of phosphatidylserine, and (f) greater than about 20%, such as greater than about any of 25%, 30%, 35%, 40%, 45%, 50%, 55%, 60%, 65%, 70%, 75%, 80%, 85%, 90% or 95%, of the anucleate cell-derived vesicles in the composition have reduced ATP production compared to the parent anucleate cell.

In some embodiments described herein, one or more properties of a composition comprising a plurality of anucleate cell-derived vesicles are based on comparison to a population of a parent anucleate cell, from which the anucleate cell-derived vesicles were prepared. In some embodiments, the comparison is based on the average value measured for the population of the parent anucleate cell. In some embodiments, the comparison is based on a range of values measured for the population of the parent anucleate cell. In some embodiments, provided is a composition comprising a plurality of anucleate cell-derived vesicles prepared from a population of a parent anucleate cell, the composition having one or more of the following properties: (a) greater than about 20%, such as greater than about any of 25%, 30%, 35%, 40%, 45%, 50%, 55%, 60%, 65%, 70%, 75%, 80%, 85%, 90% or 95%, of the anucleate cell-derived vesicles in the composition have a circulating half-life in a mammal that is decreased compared to the average of the population of the parent anucleate cell, (b) greater than 20%, such as greater than about any of 25%, 30%, 35%, 40%, 45%, 50%, 55%, 60%, 65%, 70%, 75%, 80%, 85%, 90% or 95%, of the anucleate cell-derived vesicles in the composition have decreased hemoglobin levels compared to the average of the population of the parent anucleate cell, (c) greater than 20%, such as greater than about any of 25%, 30%, 35%, 40%, 45%, 50%, 55%, 60%, 65%, 70%, 75%, 80%, 85%, 90% or 95%, of the anucleate cell-derived vesicles in the composition have spherical morphology, (d) greater than 20%, such as greater than about any of 25%, 30%, 35%, 40%, 45%, 50%, 55%, 60%, 65%, 70%, 75%, 80%, 85%, 90% or 95%, of the anucleate cell-derived vesicles in the composition are RBC ghosts, (e) greater than 20%, such as greater than about any of 25%, 30%, 35%, 40%, 45%, 50%, 55%, 60%, 65%, 70%, 75%, 80%, 85%, 90% or 95%, of the anucleate cell-derived vesicles in the composition vesicles in the composition have higher levels of phosphatidylserine compared to the average of the population of the parent anucleate cell, or (f) greater than 20%, such as greater than about any of 25%, 30%, 35%, 40%, 45%, 50%, 55%, 60%, 65%, 70%, 75%, 80%, 85%, 90% or 95%, of the anucleate cell-derived vesicles in the composition have reduced ATP production compared to the average of the population of the parent anucleate cell.

In some embodiments, the parent anucleate cell was not subjected to one or more, such as all, of the following (a) heat processed, such as heat-treated or heat-shocked, (b) chemically treated, and (c) subjected to hypotonic or hypertonic conditions during the preparation of the anucleate cell-derived vesicles.

In some embodiments, the osmolarity was maintained during preparation of the anucleate cell-derived vesicle from the parent anucleate cell. In some embodiments, the osmolarity was maintained between about 200 mOsm and about 600 mOsm, such as between any of about 200 mOsm and about 300 mOsm, about 200 mOsm and about 400 mOsm, about 200 mOsm and about 500 mOsm, about 300 mOsm and about 500 mOsm or about 350 mOsm and about 450 mOsm. In some embodiments, the osmolarity was maintained between about 200 mOsm and about 400 mOsm.

In some embodiments, the anucleate cell-derived vesicles of the composition were prepared by a process comprising: passing a suspension comprising the input parent anucleate cells through a cell deforming constriction, wherein a diameter of the constriction is a function of a diameter of the input parent anucleate cells in the suspension, thereby causing perturbations of the anucleate cells large enough for a payload to pass through; thereby producing the anucleate cell-derived vesicles. In some embodiments, the anucleate cell-derived vesicles of the composition comprise a payload. In some embodiments, the payload is a therapeutic payload. In some embodiments, the payload is an antigen. In some embodiments, the payload is an adjuvant. In some embodiments, the payload is a tolerogenic factor. In some embodiments, the payload is a polypeptide, a nucleic acid, a lipid, a carbohydrate, a small molecule, a complex (such as a protein-based complex, a nucleic acid complex, a protein-protein complex, nucleic acid-nucleic acid complex, or a protein-nucleic acid complex), or a nanoparticle.

In some embodiments, the anucleate cell-derived vesicles of the composition were prepared by a process comprising: (a) passing a cell suspension comprising the input parent anucleate cells through a cell-deforming constriction, wherein a diameter of the constriction is a function of a diameter of the input parent anucleate cells in the suspension, thereby causing perturbations of the input parent anucleate cells large enough for the payload to pass through to form an anucleate cell-derived vesicles; and (b) incubating the anucleate cell-derived vesicles with the payload for a sufficient time to allow the payload to enter the anucleate cell-derived vesicles; thereby producing an anucleate cell-derived vesicles comprising the payload.

In some embodiments, the anucleate cell-derived vesicles comprise an antigen, such as any antigen described herein. In some embodiments, the anucleate cell-derived vesicles comprise a plurality of different types of antigens (such as 2, 3, 4, or 5 different types of antigens), such as selected from any antigens described herein. In some embodiments, the anucleate cell-derived vesicles comprise an adjuvant, such as any adjuvant described herein. In some embodiments, the anucleate cell-derived vesicles comprise a plurality of different types of adjuvants (such as 2, 3, 4, or 5 different types of adjuvants), such as selected from any adjuvants described herein. In some embodiments, the anucleate cell-derived vesicles comprise a tolerogenic factor, such as any tolerogenic factor described herein. In some embodiments, the anucleate cell-derived vesicles comprise a plurality of different types of tolerogenic factors (such as 2, 3, 4, or 5 different types of tolerogenic factors), such as selected from any tolerogenic factors described herein. In some embodiments, the anucleate cell-derived vesicles comprise an antigen and an adjuvant. In some embodiments, the anucleate cell-derived vesicles comprise an adjuvant and a tolerogenic factor. In some embodiments, the anucleate cell-derived vesicles comprise an antigen and a tolerogenic factor. In some embodiments, the anucleate cell-derived vesicles comprise an antigen, an adjuvant, and a tolerogenic factor.

In some embodiments, an anucleate cell-derived vesicle comprising an antigen and an adjuvant as described herein is an activating antigen carrier (AAC).

For example, in some embodiments, the antigen is capable of being processed into an MHC class I-restricted peptide. In some embodiments, the antigen is capable of being processed into an MHC class II-restricted peptide. In some embodiments, the antigen is capable of being processed into an MHC class I-restricted peptide and an MHC class II-restricted peptide. In some embodiments, the antigen is a disease-associated antigen. In some embodiments, the antigen is a tumor antigen. In some embodiments, the antigen is derived from a lysate. In some embodiments, the lysate is a transplanted tissue lysate. In some embodiments, the lysate is a tumor lysate. In some embodiments, the antigen is a viral antigen, a bacterial antigen, or a fungal antigen. In some embodiments, the antigen is a microorganism. In some embodiments, the antigen is a polypeptide. In some embodiments, the antigen is a lipid antigen. In some embodiments, the antigen is a carbohydrate antigen. In some embodiments, a nucleic acid encoding the antigen is delivered to the cell. In some embodiments, the antigen is a modified antigen. In some embodiments, the modified antigen comprises an antigen fused with a polypeptide. In some embodiments, the modified antigen comprises an antigen fused with a targeting peptide. In some embodiments, the modified antigen comprises an antigen fused with a lipid. In some embodiments, the modified antigen comprises an antigen fused with a carbohydrate. In some embodiments, the modified antigen comprises an antigen fused with a nanoparticle. In some embodiments, a plurality of antigens is delivered to the anucleate cell. In some embodiments, the adjuvant is a CpG ODN, IFN-α, STING agonists, RIG-I agonists, poly I:C, imiquimod, resiquimod, and/or LPS.

In some embodiments, the anucleate cell-derived vesicles of the composition were prepared by a process comprising: (a) passing a cell suspension comprising the input parent anucleate cell through a cell-deforming constriction, wherein a diameter of the constriction is a function of a diameter of the input parent anucleate cell in the suspension, thereby causing perturbations of the input parent anucleate cell large enough for the antigen to pass through to form an anucleate cell-derived vesicle; and (b) incubating the anucleate cell-derived vesicle with the antigen for a sufficient time to allow the antigen to enter the anucleate cell-derived vesicle; thereby producing an anucleate cell-derived vesicle comprising an antigen.

In some embodiments, the anucleate cell-derived vesicles of the composition were prepared by a process comprising: (a) passing a cell suspension comprising the input parent anucleate cell through a cell-deforming constriction, wherein a diameter of the constriction is a function of a diameter of the input parent anucleate cell in the suspension, thereby causing perturbations of the input parent anucleate cell large enough for the adjuvant to pass through to form an anucleate cell-derived vesicle; and (b) incubating the anucleate cell-derived vesicle with the adjuvant for a sufficient time to allow the adjuvant to enter the anucleate cell-derived vesicle; thereby producing an anucleate cell-derived vesicle comprising an adjuvant.

In some embodiments, the anucleate cell-derived vesicles of the composition comprises an antigen and an adjuvant, wherein the anucleate cell-derived vesicles of the composition were prepared by a process comprising: (a) passing a cell suspension comprising the input parent anucleate cell through a cell-deforming constriction, wherein a diameter of the constriction is a function of a diameter of the input parent anucleate cell in the suspension, thereby causing perturbations of the input parent anucleate cell large enough for the antigen and the adjuvant to pass through to form an anucleate cell-derived vesicle; and (b) incubating the anucleate cell-derived vesicle with the antigen and the adjuvant for a sufficient time to allow the antigen and the adjuvant to enter the anucleate cell-derived vesicle; thereby producing an anucleate cell-derived vesicle comprising an antigen and an adjuvant.

In some embodiments, the anucleate cell-derived vesicle of the composition comprises an antigen and a tolerogenic factor, wherein the anucleate cell-derived vesicles of the composition were prepared by a process comprising: (a) passing a cell suspension comprising the input parent anucleate cell through a cell-deforming constriction, wherein a diameter of the constriction is a function of a diameter of the input parent anucleate cell in the suspension, thereby causing perturbations of the input parent anucleate cell large enough for the antigen and the tolerogenic factor to pass through to form an anucleate cell-derived vesicle; and (b) incubating the anucleate cell-derived vesicle with the antigen and the tolerogenic factor for a sufficient time to allow the antigen and the tolerogenic factor to enter the anucleate cell-derived vesicle; thereby producing an anucleate cell-derived vesicle comprising an antigen and the tolerogenic factor.

In some embodiments, the constriction is contained within a microfluidic channel. In some embodiments, the microfluidic channel comprises a plurality of constrictions. In some embodiments, the plurality of constrictions are arranged in series and/or in parallel. In some embodiments, the constriction is between a plurality of micropillars; between a plurality of micropillars configured in an array; or between one or more movable plates. In some embodiments, the constriction is formed by a plurality of micropillars. In some embodiments, the constriction is formed between a plurality of micropillars configured in an array. In some embodiments, the constriction is formed by one or more movable plates.

In some embodiments, the constriction is a pore or contained within a pore. In some embodiments, the pore is contained in a surface. In some embodiments, the surface is a filter. In some embodiments, the surface is a membrane.

In some embodiments, the constriction size is about any of 10%, 15%, 20%, 25%, 30%, 35%, 40%, 45%, 50%, 55%, 60%, 65%, 70%, 75%, or 80% of a diameter of the cell, such as the largest diameter of an anucleate cell. In some embodiments, the constriction size is less than about any of 80%, 75%, 70%, 65%, 60%, 55%, 50%, 45%, 40%, 35%, 30%, 25%, 20%, 15%, or 10% of a diameter of the cell, such as the largest diameter of an anucleate cell. In some embodiments, the constriction has a width of about 0.1 μm to about 4 μm, such as any of about 1 μm to about 3 μm, about 1.75 μm to about 2.5 μm, or about 2 μm to about 2.5 μm. In some embodiments, the constriction has a width of about any of 4 μm, 3.5 μm, 3 μm, 2.5 μm, 2 μm, 1.5 μm, 1 μm, 0.5 μm, or about 0.25 μm. In some embodiments, the constriction has a width of about any of 1.8 μm, 1.9 μm, 2 μm, 2.1 μm, 2.2 μm, 2.3 μm, 2.4 μm, 2.5 μm or 2.6 μm. In some embodiments, the constriction has a width of about 2.2 μm.

In some embodiments, the input parent anucleate cells are passed through the constriction under a pressure ranging from about 10 psi to about 150 psi, such as any of about 30 psi to about 60 psi, about 10 psi to about 40 psi, about 50 psi to about 90 psi. In some embodiments, the input parent anucleate cells are passed through the constriction under a pressure of at least about any of 5 psi, 10 psi, 15 psi, 20 psi, 25 psi, 30 psi, 35 psi, 40 psi, 45 psi, 50 psi, 55 psi, 60 psi, 65 psi, 70 psi, 75 psi, 80 psi, 85 psi, 90 psi, 95 psi, 100 psi, 105 psi 110 psi, 115 psi, 120 psi, 125 psi, 130 psi, 135 psi, 140 psi, 145 psi, or 150 psi. In some embodiments, the input parent anucleate cells are passed through the constriction under a pressure of at least about 5 psi and less than about any of 150 psi, 145 psi, 140 psi, 135 psi, 130 psi, 125 psi, 120 psi, 115 psi, 110 psi, 105 psi, 100 psi, 95 psi, 90 psi, 85 psi, 80 psi, 75 psi, 70 psi, 65 psi, 60 psi, 55 psi, 50 psi, 45 psi, 40 psi, 35 psi, 30 psi, 25 psi, 20 psi, or 15 psi.

In some embodiments, the cell suspension is contacted (such as first contacted) with the payload before passing through the constriction. In some embodiments, the cell suspension is contacted (such as first contacted) with the payload concurrently with passing through the constriction. In some embodiments, the cell suspension is contacted (such as first contacted) with the payload after passing through the constriction. In some embodiments, the cell suspension is at least contacted with the payload concurrently with passing through the constriction and after passing through the constriction. In some embodiments, the cell suspension is contacted with the payload before passing through the constriction, concurrently with passing through the constriction, and after passing through the constriction.

In some embodiments, the composition comprises at least about 500,000, such as at least about any of 1 million (M), 1.5 M, 2 M, 2.5 M, 3 M, 3.5 M, 4 M, 4.5 M, 5 M, 5.5 M, 6 M, 6.5 M, 7 M, 7.5 M, 8 M, 8.5 M, 9 M, 9.5 M, 1 billion (B), 1.1 B, 1.2 B, 1.3 B, 1.4 B, 1.5 B, 10 B, 100 B, or 1 trillion (T) anucleate cell-derived vesicles.

In some embodiments, the composition comprises at least about 500,000, such as at least about any of 1 million (M), 1.5 M, 2 M, 2.5 M, 3 M, 3.5 M, 4 M, 4.5 M, 5 M, 5.5 M, 6 M, 6.5 M, 7 M, 7.5 M, 8 M, 8.5 M, 9 M, 9.5 M, 1 billion (B), 1.1 B, 1.2 B, 1.3 B, 1.4 B, or 1.5 B, 10 B, 100 B, or 1 trillion (T) anucleate cells and an adjuvant.

In some embodiments, the composition has a hematocrit (Ht) level of about 25% to about 80%, such as any of about 25% to about 45%, about 35% to about 55%, about 35% to about 65%, or about 45% to about 70. In some embodiments, the composition has a hematocrit (Ht) level of greater than about 20%, such as greater than about any of 25%, 30%, 35%, 40%, 45%, 50%, 55%, 60%, 65% 70%, 75%, or 80%. In some embodiments, the composition has a hematocrit (Ht) level of less than about 80%, such as less than about any of 75%, 70%, 65%, 60%, 55%, 50%, 45%, 40%, 35%, 30%, 25%, or 20%. In some embodiments, the composition has a hematocrit (Ht) level of about any of 20%, 25%, 30%, 35%, 40%, 45%, 50%, 55%, 60%, 65% 70%, 75%, or 80%.

In some embodiments, the composition is a pharmaceutical composition. In some embodiments, the composition is a sterile pharmaceutical composition. In some embodiments, according to any of the compositions described herein, the composition further comprises one or more additional agents that enhance the ghost formation and/or viability and/or function and/or provide utility, such as for administration, to the anucleate cells and/or anucleate cell-derived vesicles as compared to a corresponding composition comprising anucleate cells and/or anucleate cell-derived vesicles not having the one more additional agents. In some embodiments, the additional agent is a stabilizing agent or a co-factor. In some embodiments, the additional agent is a buffer. In some embodiments, the additional agent is a buffer suitable for administration to a mammal. In some embodiments, the agent is albumin. In some embodiments, the albumin is mouse, bovine, or human albumin. In some embodiments, the additional agent is a divalent metal cation, glucose, ATP, potassium, glycerol, trehalose, D-sucrose, PEG1500, L-arginine, L-glutamine, or EDTA.

### Cell-Deforming Constrictions

In some embodiments according to any of the methods or any of the anucleate cell-derived vesicles described herein, the constriction is contained within a microfluidic channel. In some embodiments, the microfluidic channel comprises a plurality of constrictions. Multiple constrictions can be placed in parallel and/or in series within the microfluidic channel. Therefore in some embodiments, the plurality of constrictions is arranged in series and/or in parallel. In some embodiments, the constriction is between a plurality of micropillars; between a plurality of micropillars configured in an array; or between one or more movable plates. In some embodiments, the constriction is formed by a plurality of micropillars; between a plurality of micropillars configured in an array; or by one or more movable plates. Exemplary microfluidic channels containing cell-deforming constrictions for use in the methods disclosed herein are described in WO2013059343. In some embodiments, the constriction is a pore or contained within a pore. Exemplary surfaces having pores for use in the methods disclosed herein are described in WO2017041050.

In some embodiments, the microfluidic channel includes a lumen and is configured such that an input anucleate cell suspended in a buffer can pass through, wherein the microfluidic channel includes a constriction. The microfluidic channel can be made of any one of a number of materials, including silicon, metal (e.g., stainless steel), plastic (e.g., polystyrene, PET, PETG), ceramics, glass, crystalline substrates, amorphous substrates, or polymers (e.g., Poly-methyl methacrylate (PMMA), PDMS, Cyclic Olefin Copolymer (COC), etc.). Fabrication of the microfluidic channel can be performed by any method known in the art, including dry etching, wet etching, photolithography, injection molding, laser ablation, or SU-8 masks.

In some embodiments, the constriction within the microfluidic channel includes an entrance portion, a centerpoint, and an exit portion. In some embodiments, the length, depth, and width of the constriction within the microfluidic channel can vary. In some embodiments, the diameter of the constriction is a function of the diameter of the input anucleate cell or cluster of input anucleate cells in suspension. In some embodiments, the diameter of the constriction within the microfluidic channel is from about 10% to about 99% of the diameter of the input anucleate cell in suspension. In some embodiments, the constriction size is about 10%, about 15%, about 20%, about 30%, about 40%, about 50%, about 60%, about 70%, about 80%, about 90%, or about 99% of the diameter of the input anucleate cell in suspension. In some embodiments, the constriction size is about 10%, about 15%, about 20%, about 30%, about 40%, about 50%, about 60%, about 70%, about 80%, about 90%, or about 99% of the minimum cross-sectional distance of the input anucleate cell (e.g., an RBC) in suspension. In some embodiments, the constriction size is about any of 10%, 15%, 20%, 25%, 30%, 35%, 40%, 45%, 50%, 55%, 60%, 65%, 70%, 75%, or 80% of a diameter of the cell, such as the largest diameter of an anucleate cell in suspension. In some embodiments, the constriction size is less than about any of 80%, 75%, 70%, 65%, 60%, 55%, 50%, 45%, 40%, 35%, 30%, 25%, 20%, 15%, or 10% of a diameter of the cell, such as the largest diameter of an anucleate cell in suspension. In some embodiments, the constriction has a width of about 0.25 μm to about 4 μm. In some embodiments, the constriction has a width of about 7 μm, about 6 μm, about 5 μm, about 4 μm, about 3.5 μm, about 3 μm, about 2.5 μm, about 2 μm, about 1 μm, about 0.5 μm, or about 0.25 μm (including any ranges between these values). In some embodiments, the constriction has a width of any one of about 1.6 μm, about 1.8 μm, about 2.0 μm, about 2.2 μm, about 2.4 μm, about 2.6 μm, about 2.8 μm, or about 3.0 μm. In some embodiments, the constriction has a width of about 2.2 μm. In some embodiments, the constriction has a width of about 0.1 μm to about 4 μm, such as any of about 1 μm to about 3 μm, about 1.75 μm to about 2.5 μm, or about 2 μm to about 2.5 μm. In some embodiments, the constriction has a width of about any of 4 μm, 3.5 μm, 3 μm, 2.5 μm, 2 μm, 1.5 μm, 1 μm, 0.5 μm, or about 0.25 μm. In some embodiments, the constriction has a width of about any of 1.8 μm, 1.9 μm, 2 μm, 2.1 μm, 2.2 μm, 2.3 μm, 2.4 μm, 2.5 μm or 2.6 μm. In some embodiments, the constriction has a width of about 2.2 μm.

In some applications, the constriction width may be varied to modulate the relative amount of ghost formation from input anucleate cells. In some applications, the constriction width may be reduced to increase the relative amount of ghost formation from input anucleate cells. In some applications, the constriction length may be varied to modulate the relative amount of ghost formation from input anucleate cells. In some applications, the constriction length may be increased to increase the relative amount of ghost formation from input anucleate cells.

In certain embodiments, the input anucleate cells are passed through the constriction under a pressure ranging from about 10 psi to about 90 psi. In certain embodiments, the input anucleate cells are passed through the constriction under a pressure ranging from about 5 psi to about 150 psi. In certain embodiments, the input anucleate cells are passed through the constriction under a pressure ranging from any one of about 5 psi to about 10 psi, about 10 psi to about 20 psi, about 20 psi to about 30 psi, about 30 psi to about 40 psi, about 40 psi to about 50 psi, about 50 psi to about 60 psi, about 60 psi to about 70 psi, about 70 psi to about 80 psi, about 80 psi to about 90 psi, about 90 psi to about 100 psi, about 100 psi to about 110 psi, about 110 psi to about 120 psi, about 120 psi to about 130 psi, about 130 psi to about 140 psi, about 140 psi to about 150 psi, or about 150 psi to about 200 psi. In some embodiments, the parent anucleate cells are passed through the constriction under a pressure ranging from about 10 psi to about 150 psi, such as any of about 30 psi to about 60 psi, about 10 psi to about 40 psi, about 50 psi to about 90 psi. In some embodiments, the parent anucleate cells are passed through the constriction under a pressure of at least about any of 5 psi, 10 psi, 15 psi, 20 psi, 25 psi, 30 psi, 35 psi, 40 psi, 45 psi, 50 psi, 55 psi, 60 psi, 65 psi, 70 psi, 75 psi, 80 psi, 85 psi, 90 psi, 95 psi, 100 psi, 105 psi, 110 psi, 115 psi, 120 psi, 125 psi, 130 psi, 135 psi, 140 psi, 145 psi, or 150 psi. In some embodiments, the parent anucleate cells are passed through the constriction under a pressure of at least about 5 psi and less than about any of 150 psi, 145 psi, 140 psi, 135 psi, 130 psi, 125 psi, 120 psi, 115 psi, 105 psi, 100 psi, 95 psi, 90 psi, 85 psi, 80 psi, 75 psi, 70 psi, 65 psi, 60 psi, 55 psi, 50 psi, 45 psi, 40 psi, 35 psi, 30 psi, 25 psi, 20 psi, or 15 psi. In some applications, the pressure may be varied to modulate the relative amount of ghost formation from input anucleate cells. In some embodiments, the pressure may be increased to increase the relative amount of ghost formation from input anucleate cells. The cross-section of the channel, the entrance portion, the centerpoint, and the exit portion can also vary. For example, the cross-sections can be circular, elliptical, an elongated slit, square, hexagonal, or triangular in shape. The entrance portion defines a constriction angle, wherein the constriction angle is optimized to reduce clogging of the channel and optimized for enhanced delivery of an antigen and/or an adjuvant into the cell. The angle of the exit portion can vary as well. For example, the angle of the exit portion is configured to reduce the likelihood of turbulence that can result in non-laminar flow. In some embodiments, the walls of the entrance portion and/or the exit portion are linear. In other embodiments, the walls of the entrance portion and/or the exit portion are curved. In some embodiments, the cell suspension is contacted with the antigen before, concurrently, and/or after passing through the constriction.

In some embodiments according to any of the methods or anucleate cell-derived vesicles described herein, a cell suspension comprising an input anucleate cell is passed through a constriction, wherein the constriction deforms the input anucleate cell thereby causing a perturbation of the input anucleate cell such that an antigen and/or an adjuvant enters the input anucleate cell. In some embodiments, the constriction is a pore or contained within a pore. In some embodiments, the pore is contained in a surface. Exemplary surfaces having pores for use in the methods disclosed herein are described in WO2017041050.

In some embodiments, the constriction size is a function of the anucleate cell. In some embodiments, the constriction size is about 10% to about 99% of the diameter of the input anucleate cell in suspension. In some embodiments, the constriction size is about 10% to about 70% of the diameter of the input anucleate cell in suspension. In some embodiments, the constriction size is about 10%, about 15%, about 20%, about 30%, about 40%, about 50%, about 60%, about 70%, about 80%, about 90%, or about 99% of the diameter of the input anucleate cell in suspension. In some embodiments, the constriction size is about 10%, about 15%, about 20%, about 30%, about 40%, about 50%, about 60%, about 70%, about 80%, about 90%, or about 99% of the minimum cross-sectional distance of the input anucleate cell (e.g., an anucleate cell such as an RBC) in suspension. Optimal constriction size or constriction width can vary based upon the application and/or cell type. In some embodiments, the constriction has a width of about 0.25 μm to about 4 μm. In some embodiments, the constriction has a width of about 7 μm, about 6 μm, about 5 μm, about 4 μm, about 3.5 μm, about 3 μm, about 2.5 μm, about 2 μm, about 1 μm, about 0.5 μm, or about 0.25 μm (including any ranges between these values). In some embodiments, the constriction has a width of less than any of about 7 μm, about 6 μm, about 5 μm, about 4 μm, about 3.5 μm, about 3 μm, about 2.5 μm, about 2 μm, about 1.5 μm, about 1 μm, about 0.5 μm, about 0.25 μm, or about 0.1 μm. In some embodiments, the constriction has a width of any one of about 1.6 μm, about 1.8 μm, about 2.0 μm, about 2.2 μm, about 2.4 μm, about 2.6 μm, about 2.8 μm, or about 3.0 μm. In some embodiments, the constriction has a width of about 2.2 μm. In some applications, the constriction width may be varied to modulate the relative amount of ghost formation from input anucleate cells. In some applications, the constriction width may be reduced to increase the relative amount of ghost formation from input anucleate cells. In some applications, the constriction length may be varied to modulate the relative amount of ghost formation from input anucleate cells. In some applications, the constriction length may be increased to increase the relative amount of ghost formation from input anucleate cells. In certain embodiments, the input anucleate cells are passed through the constriction under a pressure ranging from about 10 psi to about 90 psi. In certain embodiments, the input anucleate cells are passed through the constriction under a pressure ranging from about 5 psi to about 150 psi. In certain embodiments, the input anucleate cells are passed through the constriction under a pressure ranging from any one of about 5 psi to about 10 psi, about 10 psi to about 20 psi, about 20 psi to about 30 psi, about 30 psi to about 40 psi, about 40 psi to about 50 psi, about 50 psi to about 60 psi, about 60 psi to about 70 psi, about 70 psi to about 80 psi, about 80 psi to about 90 psi, about 90 psi to about 100 psi, about 100 psi to about 110 psi, about 110 psi to about 120 psi, about 120 psi to about 130 psi, about 130 psi to about 140 psi, about 140 psi to about 150 psi, or about 150 psi to about 200 psi. In some embodiments, the pressure may be varied to modulate the relative amount of ghost formation from input anucleate cells. In some embodiments, the pressure may be increased to increase the relative amount of ghost formation from input anucleate cells. In some embodiments, the cell suspension is contacted with the antigen before, concurrently, and/or after passing through the constriction.

The surfaces as disclosed herein can be made of any one of a number of materials and take any one of a number of forms. In some embodiments, the surface is a filter. In some embodiments, the surface is a membrane. In some embodiments, the filter is a tangential flow filter. In some embodiments, the surface is a sponge or sponge-like matrix. In some embodiments, the surface is a matrix.

In some embodiments, the surface is a tortuous path surface. In some embodiments, the tortuous path surface comprises cellulose acetate. In some embodiments, the surface comprises a material selected from, without limitation, synthetic or natural polymers, polycarbonate, silicon, glass, metal, alloy, cellulose nitrate, silver, cellulose acetate, nylon, polyester, polyethersulfone, polyacrylonitrile (PAN), polypropylene, PVDF, polytetrafluorethylene, mixed cellulose ester, porcelain, and ceramic.

The surface disclosed herein can have any shape known in the art; e.g. a 3-dimensional shape. The 2-dimensional shape of the surface can be, without limitation, circular, elliptical, round, square, star-shaped, triangular, polygonal, pentagonal, hexagonal, heptagonal, or octagonal. In some embodiments, the surface is round in shape. In some embodiments, the surface 3-dimensional shape is cylindrical, conical, or cuboidal.

The surface can have various cross-sectional widths and thicknesses. In some embodiments, the surface cross-sectional width is between about 1 mm and about 1 m or any cross-sectional width or range of cross-sectional widths therebetween. In some embodiments, the surface has a defined thickness. In some embodiments, the surface thickness is uniform. In some embodiments, the surface thickness is variable. For example, in some embodiments, portions of the surface are thicker or thinner than other portions of the surface. In some embodiments, the surface thickness varies by about 1% to about 90% or any percentage or range of percentages therebetween. In some embodiments, the surface is between about 0.01 μm to about 5 mm thick or any thickness or range of thicknesses therebetween.

In some embodiments according to any of the methods described herein, the constriction is a pore or contained within a pore. In some embodiments, the pore is contained in a surface. In some embodiments, the surface is a filter. In some embodiments, the surface is a membrane. The cross-sectional width of the pores is related to the type of cell to be treated. In some embodiments, the pore size is a function of the diameter in suspension of the input anucleate cell or cluster of input anucleate cells to be treated. In some embodiments, the pore size is such that an input anucleate cell is perturbed upon passing through the pore. In some embodiments, the pore size is less than the diameter of the input anucleate cell. In some embodiments, the pore size is about 10% to about 99% of the diameter of the anucleate cell. In some embodiments, the pore size is about 10% to about 70% of the diameter of the input anucleate cell. In some embodiments, the pore size is about 10%, about 15%, about 20%, about 30%, about 40%, about 50%, about 60%, about 70%, about 80%, about 90%, or about 99% of the input anucleate cell diameter. Optimal pore size or pore cross-sectional width can vary based upon the application and/or cell type. In some applications, the pore size or pore cross-sectional width may be varied to modulate the relative amount of ghost formation from input anucleate cells. In some applications, the pore size or pore cross-sectional width may be reduced to increase the relative amount of ghost formation from input anucleate cells. In some embodiments, the pore size or pore cross-sectional width is about 0.1 μm to about 4 μm. In some embodiments, the pore size or pore cross-sectional width is about 0.25 μm to about 4 μm. In some embodiments, the pore size or pore cross-sectional width is about 4 μm, about 3.5 μm, about 3 μm, about 2.5 μm, about 2 μm, about 1.5 μm, about 1 μm, about 0.5 μm, about 0.25 μm, or about 0.1 μm. In some embodiments, the pore size or pore cross-sectional width is at or less than any of about 4 μm, about 3.5 μm, about 3 μm, about 2.5 μm, about 2 μm, about 1.5 μm, about 1 μm, about 0.5 μm, about 0.25 μm, or about 0.1 μm. In some embodiments, the pore size or pore cross-sectional width is any one of about 1.6 μm, about 1.8 μm, about 2.0 μm, about 2.2 μm, about 2.4 μm, about 2.6 μm, about 2.8 μm, or about 3.0 μm. In some embodiments, the pore size or pore cross-sectional width is about 2.2 μm. In certain embodiments, the input anucleate cells are passed through the pore under a pressure ranging from about 10 psi to about 90 psi. In certain embodiments, the input anucleate cells are passed through the constriction under a pressure ranging from about 5 psi to about 150 psi. In certain embodiments, the input anucleate cells are passed through the pore under a pressure ranging from any one of about 5 psi to about 10 psi, about 10 psi to about 20 psi, about 20 psi to about 30 psi, about 30 psi to about 40 psi, about 40 psi to about 50 psi, about 50 psi to about 60 psi, about 60 psi to about 70 psi, about 70 psi to about 80 psi, about 80 psi to about 90 psi, about 90 psi to about 100 psi, about 100 psi to about 110 psi, about 110 psi to about 120 psi, about 120 psi to about 130 psi, about 130 psi to about 140 psi, about 140 psi to about 150 psi, or about 150 psi to about 200 psi. In some embodiments, the pressure may be varied to modulate the relative amount of ghost formation from input anucleate cells. In some embodiments, the pressure may be increased to increase the relative amount of ghost formation from input anucleate cells. In some embodiments, the cell suspension is contacted with the antigen before, concurrently, and/or after passing through the pore.

The entrances and exits of the pore passage may have a variety of angles. The pore angle can be selected to minimize clogging of the pore while the anucleate cells are passing through. For example, the angle of the entrance or exit portion can be between about 0 and about 90 degrees. In some embodiments, the entrance or exit portion can be greater than 90 degrees. In some embodiments, the pores have identical entrance and exit angles. In some embodiments, the pores have different entrance and exit angles. In some embodiments, the pore edge is smooth, e.g. rounded or curved. A smooth pore edge has a continuous, flat, and even surface without bumps, ridges, or uneven parts. In some embodiments, the pore edge is sharp. A sharp pore edge has a thin edge that is pointed or at an acute angle. In some embodiments, the pore passage is straight. A straight pore passage does not contain curves, bends, angles, or other irregularities. In some embodiments, the pore passage is curved. A curved pore passage is bent or deviates from a straight line. In some embodiments, the pore passage has multiple curves, e.g. about 2, 3, 4, 5, 6, 7, 8, 9, 10 or more curves.

The pores can have any shape known in the art, including a 2-dimensional or 3-dimensional shape. The pore shape (e.g., the cross-sectional shape) can be, without limitation, circular, elliptical, round, square, star-shaped, triangular, polygonal, pentagonal, hexagonal, heptagonal, and octagonal. In some embodiments, the cross-section of the pore is round in shape. In some embodiments, the 3-dimensional shape of the pore is cylindrical or conical. In some embodiments, the pore has a fluted entrance and exit shape. In some embodiments, the pore shape is homogenous (i.e. consistent or regular) among pores within a given surface. In some embodiments, the pore shape is heterogeneous (i.e. mixed or varied) among pores within a given surface.

The surfaces described herein can have a range of total pore numbers. In some embodiments, the pores encompass about 10% to about 80% of the total surface area. In some embodiments, the surface contains about 1.0×10⁵ to about 1.0×10³⁰ total pores or any number or range of numbers therebetween. In some embodiments, the surface comprises between about 10 and about 1.0×10¹⁵ pores per mm² surface area.

The pores can be distributed in numerous ways within a given surface. In some embodiments, the pores are distributed in parallel within a given surface. In one such example, the pores are distributed side-by-side in the same direction and are the same distance apart within a given surface. In some embodiments, the pore distribution is ordered or homogeneous. In one such example, the pores are distributed in a regular, systematic pattern or are the same distance apart within a given surface. In some embodiments, the pore distribution is random or heterogeneous. In one such example, the pores are distributed in an irregular, disordered pattern or are different distances apart within a given surface. In some embodiments, multiple surfaces are distributed in series. The multiple surfaces can be homogeneous or heterogeneous in surface size, shape, and/or roughness. The multiple surfaces can further contain pores with homogeneous or heterogeneous pore size, shape, and/or number, thereby enabling the simultaneous delivery of a range of antigens and/or adjuvants into different types of anucleate cells.

In some embodiments, an individual pore has a uniform width dimension (i.e. constant width along the length of the pore passage). In some embodiments, an individual pore has a variable width (i.e. increasing or decreasing width along the length of the pore passage). In some embodiments, pores within a given surface have the same individual pore depths. In some embodiments, pores within a given surface have different individual pore depths. In some embodiments, the pores are immediately adjacent to each other. In some embodiments, the pores are separated from each other by a distance. In some embodiments, the pores are separated from each other by a distance of about 0.001 μm to about 30 mm or any distance or range of distances therebetween.

In some embodiments, the surface is coated with a material. The material can be selected from any material known in the art, including, without limitation, Teflon, an adhesive coating, surfactants, proteins, adhesion molecules, antibodies, anticoagulants, factors that modulate cellular function, nucleic acids, lipids, carbohydrates, nanoparticles, or transmembrane proteins. In some embodiments, the surface is coated with polyvinylpyrrolidone. In some embodiments, the material is covalently attached to the surface. In some embodiments, the material is non-covalently attached to the surface. In some embodiments, the surface molecules are released as the anucleate cells pass through the pores.

In some embodiments, the surface has modified chemical properties. In some embodiments, the surface is hydrophilic. In some embodiments, the surface is hydrophobic. In some embodiments, the surface is charged. In some embodiments, the surface is positively and/or negatively charged. In some embodiments, the surface can be positively charged in some regions and negatively charged in other regions. In some embodiments, the surface has an overall positive or overall negative charge. In some embodiments, the surface can be any one of smooth, electropolished, rough, or plasma treated. In some embodiments, the surface comprises a zwitterion or dipolar compound. In some embodiments, the surface is plasma treated.

In some embodiments, the surface is contained within a larger module. In some embodiments, the surface is contained within a syringe, such as a plastic or glass syringe. In some embodiments, the surface is contained within a plastic filter holder. In some embodiments, the surface is contained within a pipette tip.

In some embodiments according to any of the methods or any of the anucleate cell-derived vesicles described herein, a cell suspension comprising an input anucleate cell is passed through a constriction, wherein the constriction deforms the input anucleate cell thereby causing a perturbation of the cell such that an antigen and/or an adjuvant enters the input anucleate cell, wherein the perturbation in the input anucleate cell is a breach in the input anucleate cell that allows material from outside the cell to move into the input anucleate cell (e.g., a hole, tear, cavity, aperture, pore, break, gap, perforation). The deformation can be caused by, for example, pressure induced by mechanical strain and/or shear forces. In some embodiments, the perturbation is a perturbation within the anucleate cell membrane. In some embodiments, the perturbation is transient. In some embodiments, the cell perturbation lasts from about 1.0×10⁻⁹ seconds to about 24 hours, or any time or range of times therebetween. In some embodiments, the cell perturbation lasts for about 1.0×10⁻⁹ second to about 1 second, about 1 second to about 1 minute, about 1 minute to about 1 hour, about 1 hour to about 2 hours, about 2 hours to about 4 hours, about 4 hours to about 6 hours, about 6 hours to about 8 hours, about 8 hours to about 10 hours, about 10 hours to about 12 hours, about 12 hours to about 16 hours, about 16 hours to about 20 hours, or about 20 hours to about 24 hours. In some embodiments, the cell perturbation lasts for between any one of about 1.0×10⁻⁹ to about 1.0×10⁻¹, about 1.0×10⁻⁹ to about 1.0×10⁻², about 1.0×10⁻⁹ to about 1.0×10⁻³, about 1.0×10⁻⁹ to about 1.0×10⁻⁴, about 1.0×10⁻⁹ to about 1.0×10⁻⁵, about 1.0×10⁻⁹ to about 1.0×10⁻⁶, about 1.0×10⁻⁹ to about 1.0×10⁻⁷, or about 1.0×10⁻⁹ to about 1.0×10⁻⁸ seconds. In some embodiment, the cell perturbation lasts for any one of about 1.0×10⁻⁸ to about 1.0×10⁻¹, about 1.0×10⁻⁷ to about 1.0×10⁻¹, about 1.0×10⁻⁶ to about 1.0×10⁻¹, about 1.0×10⁻⁵ to about 1.0×10⁻¹, about 1.0×10⁻⁴ to about 1.0×10⁻¹, about 1.0×10⁻³ to about 1.0×10⁻¹, or about 1.0×10⁻² to about 1.0×10⁻¹ seconds. The cell perturbations (e.g., pores or holes) created by the methods described herein are not formed as a result of assembly of protein subunits to form a multimeric pore structure such as that created by complement or bacterial hemolysins.

In some embodiments, the passage of the antigen and/or the adjuvant into the anucleate cell-derived vesicle occurs simultaneously with the input anucleate cell passing through the constriction and/or the perturbation of the cell. In some embodiments, passage of the antigen and/or the adjuvant into the anucleate cell-derived vesicle occurs after the input anucleate cell passes through the constriction. In some embodiments, passage of the antigen and/or the adjuvant into the anucleate cell-derived vesicle occurs on the order of minutes after the input anucleate cell passes through the constriction. In some embodiments, the passage of the antigen and/or the adjuvant into the anucleate cell-derived vesicle occurs from about 1.0×10⁻² seconds to at least about 30 minutes after the input anucleate cell passes through the constriction. For example, the passage of the antigen and/or the adjuvant into the anucleate cell-derived vesicle occurs from about 1.0×10⁻² seconds to about 1 second, about 1 second to about 1 minute, or about 1 minute to about 30 minutes after the input anucleate cell passes through the constriction. In some embodiments, the passage of the antigen and/or the adjuvant into the anucleate cell-derived vesicle occurs about 1.0×10⁻² seconds to about 10 minutes, about 1.0×10⁻² seconds to about 5 minutes, about 1.0×10⁻² seconds to about 1 minute, about 1.0×10⁻² seconds to about 50 seconds, about 1.0×10⁻² seconds to about 10 seconds, about 1.0×10⁻² seconds to about 1 second, or about 1.0×10⁻² seconds to about 0.1 second after the input anucleate cell passes through the constriction. In some embodiments, the passage of the antigen and/or the adjuvant into the anucleate cell-derived vesicle occurs about 1.0×10⁻¹ seconds to about 10 minutes, about 1 second to about 10 minutes, about 10 seconds to about 10 minutes, about 50 seconds to about 10 minutes, about 1 minute to about 10 minutes, or about 5 minutes to about 10 minutes after the input anucleate cell passes through the constriction. In some embodiments, a perturbation in the resulting anucleate cell-derived vesicle after the input anucleate cell passes through the constriction is corrected within the order of about five minutes after the input anucleate cell passes through the constriction.

Ghost formation from an anucleate cell occurs when the entity shape is changed towards a more spherical morphology and may be accompanied by loss of some of its original cytoplasmic structures and contents. RBC ghost and erythrocyte ghost formations are phenomenon known in the art. In some embodiments, ghost formation after passing through a constriction is about 5% to about 100%. In some embodiments, ghost formation after passing through the constriction is at least about 5%, 10%, 20%, 30%, 40%, 50%, 60%, 70%, 75%, 80%, 85%, 90%, 95%, or 99%. In some embodiments, ghost formation is measured from about 1.0×10⁻² seconds to at least about 10 days after the cell passes through the constriction. For example, ghost formation is measured from about 1.0×10⁻² seconds to about 1 second, about 1 second to about 1 minute, about 1 minute to about 30 minutes, or about 30 minutes to about 2 hours after the cell passes through the constriction. In some embodiments, ghost formation is measured about 1.0×10⁻² seconds to about 2 hours, about 1.0×10⁻² seconds to about 1 hour, about 1.0×10⁻² seconds to about 30 minutes, about 1.0×10⁻² seconds to about 1 minute, about 1.0×10⁻² seconds to about 30 seconds, about 1.0×10⁻² seconds to about 1 second, or about 1.0×10⁻² seconds to about 0.1 second after the cell passes through the constriction. In some embodiments, ghost formation is measured about 1.5 hours to about 2 hours, about 1 hour to about 2 hours, about 30 minutes to about 2 hours, about 15 minutes to about 2 hours, about 1 minute to about 2 hours, about 30 seconds to about 2 hours, or about 1 second to about 2 hours after the cell passes through the constriction. In some embodiments, ghost formation is measured about 2 hours to about 5 hours, about 5 hours to about 12 hours, about 12 hours to about 24 hours, or about 24 hours to about 10 days after the cell passes through the constriction.

A number of parameters may influence the delivery of an antigen and/or an adjuvant to an anucleate cell-derived vesicle according to any of the methods or anucleate cell-derived vesicles described herein. In some embodiments, the cell suspension comprising the input anucleate cells is contacted with the antigen and/or the adjuvant before, concurrently, or after passing through the constriction. The input anucleate cell may pass through the constriction suspended in a solution that includes the antigen and/or the adjuvant to be delivered, although the antigen and/or the adjuvant can be added to the cell suspension after the input anucleate cells pass through the constriction to form anucleate cell-derived vesicles comprising antigen and/or adjuvant. In some embodiments, the antigen and/or the adjuvant to be delivered is coated on the constriction. In some embodiments, the antigen and/or the adjuvant to be delivered is coated on the surface. In some embodiments, the antigen and/or the adjuvant to be delivered is coated on the pore. In some embodiments, the antigen and/or the adjuvant to be delivered is coated on the filter.

Examples of parameters that may influence the delivery of the antigen and/or the adjuvant into the anucleate cell-derived vesicle include, but are not limited to, the dimensions of the constriction, the entrance angle of the constriction, the surface properties of the constrictions (e.g., roughness, chemical modification, hydrophilic, hydrophobic, etc.), the operating flow speeds (e.g., cell transit time through the constriction), the input anucleate cell concentration, the concentration of the antigen and/or the adjuvant in the cell suspension, and the amount of time that the anucleate cell-derived vesicles recovers or incubates after passing through the constrictions can affect the passage of the delivered antigen and/or adjuvant into the cell. Additional parameters influencing the delivery of the antigen and/or the adjuvant into the anucleate cell-derived vesicle can include the velocity of the input anucleate cell in the constriction, the shear rate in the constriction, the viscosity of the input anucleate cell suspension, the velocity component that is perpendicular to flow velocity, and time in the constriction. Such parameters can be designed to control delivery of the antigen and/or the adjuvant. In some embodiments, the anucleate cell-derived vesicle concentration ranges from about 10 to at least about 10¹² vesicles/mL or any concentration or range of concentrations therebetween. In some embodiments, concentrations of the antigen and/or the adjuvant to be delivered can range from about 10 ng/mL to about 1 g/mL or any concentration or range of concentrations therebetween. In some embodiments, concentrations of the antigen and/or the adjuvant to be delivered can range from about 1 pM to at least about 2 M or any concentration or range of concentrations therebetween. The composition of the cell suspension (e.g., osmolarity, salt concentration, serum content, cell concentration, pH, etc.) can impact delivery of the antigen and/or the adjuvant for stimulating and/or enhancing an immune response. In some embodiments, the aqueous solution is iso-osmolar or iso-tonic.

The temperature used in the methods of the present disclosure can be adjusted to affect antigen and/or adjuvant delivery and/or ghost formation in anucleate cell derived-vesicles. In some embodiments, the method is performed between about −5° C. and about 45° C. For example, the methods can be carried out at room temperature (e.g., about 20° C.), physiological temperature (e.g., about 37° C.), higher than physiological temperature (e.g., greater than about 37° C. to 45° C. or more), or reduced temperature (e.g., about −5° C. to about 4° C.), or temperatures between these exemplary temperatures.

Various methods can be utilized to drive the input anucleate cells in suspension through the constrictions. For example, pressure can be applied by a pump on the entrance side (e.g., gas cylinder, or compressor), a vacuum can be applied by a vacuum pump on the exit side, capillary action can be applied through a tube, and/or the system can be gravity fed. Displacement based flow systems can also be used (e.g., syringe pump, peristaltic pump, manual syringe or pipette, pistons, etc.). In some embodiments, the input anucleate cells are passed through the constrictions by positive pressure or negative pressure. Therefore in some embodiments according to any one of the methods or anucleate cell-derived vesicles described herein, the input anucleate cells are passed through the constrictions by positive pressure from the entrance side. In further embodiments, the positive pressure is applied using a pump. In some embodiments, the positive pressure is applied using a gas cylinder or compressor. In certain embodiments, the input anucleate cells are passed through the constriction under a pressure ranging from about 10 psi to about 90 psi. In certain embodiments, the input anucleate cells are passed through the constriction under a pressure ranging from about 5 psi to about 150 psi. In certain embodiments, the input anucleate cells are passed through the constriction under a pressure ranging from any one of about 5 psi to about 10 psi, about 10 psi to about 20 psi, about 20 psi to about 30 psi, about 30 psi to about 40 psi, about 40 psi to about 50 psi, about 50 psi to about 60 psi, about 60 psi to about 70 psi, about 70 psi to about 80 psi, about 80 psi to about 90 psi, about 90 psi to about 100 psi, about 100 psi to about 110 psi, about 110 psi to about 120 psi, about 120 psi to about 130 psi, about 130 psi to about 140 psi, about 140 psi to about 150 psi, or about 150 psi to about 200 psi. In some embodiments, the input anucleate cells are passed through the constrictions by constant pressure or variable pressure. In some embodiments, pressure is applied using a syringe. In some embodiments, pressure is applied using a pump. In some embodiments, the pump is a peristaltic pump or a diaphragm pump. In some embodiments, pressure is applied using a vacuum. In some embodiments, the input anucleate cells are passed through the constrictions by g-force. In some embodiments, the input anucleate cells are passed through the constrictions by centrifugal force. In some embodiments, the input anucleate cells are passed through the constrictions by capillary pressure. In some embodiments, the input anucleate cells are moved (e.g., pushed) through the constriction by application of pressure using a cell driver. As used herein, a cell driver is a device or component that applies a pressure or force to the suspension in order to drive an input anucleate cell through a constriction. In certain embodiments, the cell driver is selected from a group consisting of a pressure pump, a gas cylinder, a compressor, a vacuum pump, a syringe pump, a peristaltic pump, a pipette, a piston, a capillary actor, a human heart, human muscle, gravity, a microfluidic pumps, and a syringe.

In some embodiments, fluid flow directs the input anucleate cells through the constrictions. In some embodiments, the fluid flow is turbulent flow prior to the cells passing through the constriction. Turbulent flow is a fluid flow in which the velocity at a given point varies erratically in magnitude and direction. In some embodiments, the fluid flow through the constriction is laminar flow. Laminar flow involves uninterrupted flow in a fluid near a solid boundary in which the direction of flow at every point remains constant. In some embodiments, the fluid flow is turbulent flow after the cells pass through the constriction. The velocity at which the cells pass through the constrictions can be varied. In some embodiments, the cells pass through the constrictions at a uniform cell speed. In some embodiments, the cells pass through the constrictions at a fluctuating cell speed.

The cell suspension may be a mixed or purified population of cells. In some embodiments, the cell suspension is a mixed cell population, such as whole blood. In some embodiments, the cell suspension is a mixed cell population, such as a mixed population of anucleate cells. In some embodiments, the cell suspension is a purified cell population, such as a purified population of anucleate cells.

The composition of the cell suspension (e.g., osmolarity, salt concentration, serum content, cell concentration, pH, etc.) can impact delivery of the antigen and/or adjuvant for stimulating and/or enhancing an immune response. In some embodiments, the suspension comprises whole blood. Alternatively, the cell suspension is a mixture of cells in a physiological saline solution or physiological medium other than blood. In some embodiments, the cell suspension comprises an aqueous solution. In some embodiments, the aqueous solution comprises cell culture medium, PBS, salts, sugars, growth factors, animal derived products, bulking materials, surfactants, lubricants, vitamins, amino acids, proteins, cell cycle inhibitors, and/or an agent that impacts actin polymerization. In some embodiments, the cell culture medium is DMEM, Opti-MEM™, IMDM, or RPMI. Additionally, solution buffer can include one or more lubricants (pluronics or other surfactants) that can be designed, for example, to reduce or eliminate clogging of the surface and improve input cell viability. Exemplary surfactants include, without limitation, poloxamer, polysorbates, sugars or sugar alcohols such as mannitol, sorbitol, animal derived serum, and albumin protein. In some embodiments, the aqueous solution is iso-osmolar or isotonic. In some embodiments, the aqueous solution includes plasma.

In some configurations with certain types of cells, the input anucleate cells or the anucleate cell-derived vesicles can be incubated in one or more solutions that aid in the delivery of the compound to the interior of the anucleate cell-derived vesicle. In some embodiments, the anucleate cell-derived vesicle retains all or essentially all of the cytoskeletal structure compared to an unprocessed and untreated input anucleate cell. In some embodiments, the anucleate cell-derived vesicle retains about any one of 5%, 10%, 20%, 30%, 40%, 50%, 60%, 70%, 80%, 90%, 95% or 99.5% of the cytoskeletal structure of an unprocessed and untreated input anucleate cell. In some embodiments, the solution comprises an agent that impacts actin polymerization. In some embodiments, the agent that impacts actin polymerization is Latrunculin A, Cytochalasin, and/or Colchicine. For example, the input anucleate cells or the anucleate cell-derived vesicles can be incubated in a depolymerization solution such as Lantrunculin A (0.1 μg/ml) for 1 hour prior to delivery to depolymerize the actin cytoskeleton. As an additional example, the cells can be incubated in 10 μM Colchicine (Sigma) for 2 hours prior to delivery to depolymerize the microtubule network.

In some embodiments, the input anucleate cell population is enriched prior to use in the disclosed methods. For example, the input anucleate cells are obtained from a bodily fluid, e.g., peripheral blood, and optionally enriched or purified to concentrate anucleate cells. Cells may be enriched by any methods known in the art, including without limitation, magnetic cell separation, fluorescent activated cell sorting (FACS), or density gradient centrifugation.

The viscosity of the cell suspension can also impact the methods disclosed herein. In some embodiments, the viscosity of the cell suspension ranges from about 8.9×10⁻⁴ Pa·s to about 4.0×10⁻³ Pa·s or any value or range of values therebetween. In some embodiments, the viscosity ranges between any one of about 8.9×10⁻⁴ Pa·s to about 4.0×10⁻³ Pa·s, about 8.9×10⁻⁴ Pa·s to about 3.0×10⁻³ Pa·s, about 8.9×10 Pa·s to about 2.0×10⁻³ Pa·s, or about 8.9×10⁻³ Pa·s to about 1.0×10⁻³ Pa·s. In some embodiments, the viscosity ranges between any one of about 0.89 cP to about 4.0 cP, about 0.89 cP to about 3.0 cP, about 0.89 cP to about 2.0 cP, or about 0.89 cP to about 1.0 cP. In some embodiments, a shear thinning effect is observed, in which the viscosity of the cell suspension decreases under conditions of shear strain. Viscosity can be measured by any method known in the art, including without limitation, viscometers, such as a glass capillary viscometer, or rheometers. A viscometer measures viscosity under one flow condition, while a rheometer is used to measure viscosities which vary with flow conditions. In some embodiments, the viscosity is measured for a shear thinning solution such as blood. In some embodiments, the viscosity is measured between about −5° C. and about 45° C. For example, the viscosity is measured at room temperature (e.g., about 20° C.), physiological temperature (e.g., about 37° C.), higher than physiological temperature (e.g., greater than about 37° C. to 45° C. or more), reduced temperature (e.g., about −5° C. to about 4° C.), or temperatures between these exemplary temperatures.

In some embodiments, the cell suspension is contacted (such as first contacted) with the payload before passing through the constriction. In some embodiments, the cell suspension is contacted (such as first contacted) with the payload concurrently with passing through the constriction. In some embodiments, the cell suspension is contacted (such as first contacted) with the payload after passing through the constriction. In some embodiments, the cell suspension is at least contacted with the payload concurrently with passing through the constriction and after passing through the constriction. In some embodiments, the cell suspension is contacted with the payload before passing through the constriction, concurrently with passing through the constriction, and after passing through the constriction.

In some embodiments, the payload is a therapeutic payload. In some embodiments, the payload is an antigen. In some embodiments, the payload is an adjuvant. In some embodiments, the payload is a tolerogenic factor. In some embodiments, the payload is a polypeptide, a nucleic acid, a lipid, a carbohydrate, a small molecule, a complex (such as a protein-based complex, a nucleic acid complex, a protein-protein complex, nucleic acid-nucleic acid complex, or a protein-nucleic acid complex), or a nanoparticle.

In some embodiments, the cell suspension is contacted with an antigen, such as any antigen described herein. In some embodiments, the cell suspension is contacted with a plurality of different types of antigens (such as 2, 3, 4, or 5 different types of antigens), such as selected from any antigens described herein. In some embodiments, the cell suspension is contacted with an adjuvant, such as any adjuvant described herein. In some embodiments, the cell suspension is contacted with a plurality of different types of adjuvants (such as 2, 3, 4, or 5 different types of adjuvants), such as selected from any adjuvants described herein. In some embodiments, the cell suspension is contacted with a tolerogenic factor, such as any tolerogenic factor described herein. In some embodiments, the cell suspension is contacted with a plurality of different types of tolerogenic factors (such as 2, 3, 4, or 5 different types of tolerogenic factors), such as selected from any tolerogenic factors described herein. In some embodiments, the cell suspension is contacted with an antigen and an adjuvant. In some embodiments, the cell suspension is contacted with an adjuvant and a tolerogenic factor. In some embodiments, the cell suspension is contacted with an antigen and a tolerogenic factor. In some embodiments, the anucleate cell-derived vesicles comprise an antigen, an adjuvant, and a tolerogenic factor.

For example, in some embodiments, the antigen is capable of being processed into an MHC class I-restricted peptide. In some embodiments, the antigen is capable of being processed into an MHC class II-restricted peptide. In some embodiments, the antigen is capable of being processed into an MHC class I-restricted peptide and an MHC class II-restricted peptide. In some embodiments, the antigen is a disease-associated antigen. In some embodiments, the antigen is a tumor antigen. In some embodiments, the antigen is derived from a lysate. In some embodiments, the lysate is a tumor lysate. In some embodiments, the antigen is derived from a transplant lysate. In some embodiments, the antigen is a viral antigen, a bacterial antigen, or a fungal antigen. In some embodiments, the antigen is a microorganism. In some embodiments, the antigen is a polypeptide. In some embodiments, the antigen is a lipid antigen. In some embodiments, the antigen is a carbohydrate antigen. In some embodiments, a nucleic acid encoding the antigen is delivered to the cell. In some embodiments, the antigen is a modified antigen. In some embodiments, the modified antigen comprises an antigen fused with a polypeptide. In some embodiments, the modified antigen comprises an antigen fused with a targeting peptide. In some embodiments, the modified antigen comprises an antigen fused with a lipid. In some embodiments, the modified antigen comprises an antigen fused with a carbohydrate. In some embodiments, the modified antigen comprises an antigen fused with a nanoparticle. In some embodiments, a plurality of antigens is delivered to the anucleate cell. In some embodiments, the adjuvant is a CpG ODN, IFN-α, STING agonists, RIG-I agonists, poly I:C, imiquimod, resiquimod, and/or LPS.

### Additional Methods of Use

In some aspects, the present application provides methods of using anucleate cell-derived vesicles and/or compositions described herein.

In some embodiments, provided herein are methods for treating a disease or disorder in an individual in need thereof, the method comprising administering any anucleate cell-derived vesicle and/or composition described herein. In some embodiments, the anucleate cell-derived vesicles comprise a therapeutic payload. In some embodiments, the therapeutic payload comprises any one or more of an antigen, adjuvant, and tolerogenic factor. In some embodiments, the anucleate cell-derived vesicle of the composition comprises any or more of an antigen, adjuvant, and tolerogenic factor. In some embodiments, the composition comprises an anucleate cell and an adjuvant.

In some embodiments, the disease or disorder is a cancer, an infectious disease, or a viral-associated disease. In some embodiments, the disease is treatable by an enzyme replacement therapy (ERT); e.g., Gaucher disease. In some embodiments, the disease or disorder is Gaucher Type I disease, gout, hypophosphatasia, lysosomal acid lipase deficiency, Pompe disease, MPS IH (Hurler syndrome), MPS II (Hunter syndrome), MPS III A, B, C & D (Sanfilippo syndrome A, B, C, D), MPS IV A, B (Morquio syndrome), MPV VI (Maroteaux-Lamy syndrome), MPS VII (Sly syndrome), MPS IX (Natowicz syndrome), Fabry syndrome, PKU syndrome, medium-chain acyl-CoA dehydrogenase deficiency (MCADD), Celiac disease, myasthenia gravis, Graves' disease, pemphigus vulgaris, neruomyelitis optica (NMO), or Type I diabetes. In some embodiments, the cancer is a head and neck cancer, cervical cancer, uterine cancer, rectal cancer, penile cancer, ovarian cancer, testicular cancer, bone cancer, soft tissue cancer, skin cancer (e.g., melanoma), gastric cancer, intestinal cancer, colon cancer, prostate cancer, breast cancer, esophageal cancer, liver cancer, lung cancer, pancreatic cancer, brain cancer, or blood cancer.

In some embodiments, the disease or disorder is gout and the payload is an uricase, e.g., a semi-synthetic form, such as Pegloticase. In some embodiments, the disease or disorder is Gaucher Type I disease and the payload is a glucocerebrosidase, e.g., Imiglucerase, velaglucerase alfa, or β-glucosidase. In some embodiments, the disease or disorder is hypophosphatasia and the payload is a tissue non-specific alkaline phosphatase (TNSALP), e.g., Asfotase alfa. In some embodiments, the disease or disorder is lysosomal acid lipase deficiency and the payload is a lysosomal acid lipase, e.g., Sebelipase alfa. In some embodiments, the disease or disorder is Pompe disease and the payload is an alpha-glucosidase, e.g., alglucosidase alfa. In some embodiments, the disease or disorder is MPS IH (Hurler syndrome), IH/S (Hurler-Scheie syndrome), or IS (Scheie aka MPS V) and the payload is an α-L-iduronidase, e.g., laronidase. In some embodiments, the disease or disorder is MPS II (Hunter syndrome) and the payload is an iduronate sulfatase, e.g., Idursulfase. In some embodiments, the disease or disorder is MPS III A, B, C or D (Sanfilippo syndrome A, B, C, or D) and the payload is a heparan sulfate. In some embodiments, the disease or disorder is MPS IV A, B (Morquio syndrome) and the payload is a keratin sulfate or chondroitin 6-sulfate, e.g., elosulfase alfa. In some embodiments, the disease or disorder is MPV VI (Maroteaux-Lamy syndrome) and the payload is a N-acetylgalactosamine-4-sulfatase, e.g., galsulfase. In some embodiments, the disease or disorder is MPS VII (Sly syndrome) and the payload is a β-glucuronidase. In some embodiments, the disease or disorder is MPS IX (Natowicz syndrome) and the payload is a hyaluronidase. In some embodiments, the disease or disorder is Fabry syndrome and the payload is an α-galactosidase A, e.g., agalsidase beta. In some embodiments, the disease or disorder is PKU syndrome and the payload is a phenylalanine hydroxylase. In some embodiments, the disease or disorder is medium-chain acyl-CoA dehydrogenase deficiency (MCADD) and the payload is a medium-chain acyl-CoA dehydrogenase. In some embodiments, the disease or disorder is Celiac disease and the payload is a gliadin. In some embodiments, the disease or disorder is myasthenia gravis and the payload is an acetylcholine receptor and receptor-associated proteins. In some embodiments, the disease or disorder is Graves' disease and the payload is a thyroid stimulating hormone receptor (TSHR). In some embodiments, the disease or disorder is pemphigus vulgaris and the payload is a desmoglein 1 and 3. In some embodiments, the disease or disorder is neruomyelitis optica (NMO) and the payload is an aquaporin 4. In some embodiments, the disease or disorder is Type I diabetes and the payload is a GADD65, insulin, pro-insulin, or pre-pro-insulin.

In some embodiments, the viral-associated disease is an EBV-associated disease. In some embodiments, the viral-associated disease is an EBV-associated disease and the antigen is one or more of EBNA-1, EBNA-2, EBNA-3A, EBNA-3B, EBNA-3C, EBNA-LP, LMP-1, LMP-2A, LMP-2B, and EBER. In some embodiments, the EBV-associated disease is multiple sclerosis (MS). In some embodiments, the viral-associated disease is an HIV-associated disease. In some embodiments, the HIV-associated disease are opportunistic infections, which may include but are not limited to: candidiasis of bronchi, trachea, esophagus, or lungs; invasive cervical cancer; coccidioidomycosis; cryptococcosis; chronic intestinal cryptosporidiosis, Cytomegalovirus diseases; HIV-related encephalopathy; HSV-related chronic ulcers or bronchitis, pneumonitis, or esophagitis; histoplasmosis; chronic intestinal isosporiasis; Kaposi's sarcoma; lymphoma; tuberculosis; Mycobacterium avium complex (MAC); Pneumocystis carinii pneumonia (PCP); recurrent pneumonia; progressive multifocal leukoencephalopathy; recurrent Salmonella septicemia; Toxoplasmosis of brain; and wasting syndrome due to HIV. In some embodiments, the viral-associated disease is HPV. In some embodiments, the viral-associated disease is HPV and the antigen induces a response to E7. In some embodiments, the viral-associated disease is HPV and the antigen induces a response to E6. In some embodiments, the viral-associated disease is a HBV-associated disease. In some embodiments, the viral-associated disease is a RSV-associated disease. In some embodiments, the viral-associated disease is a KSHV-associated disease.

In some embodiments, the individual has cancer and the payload comprises an antigen. In some embodiments, the individual has cancer and the payload comprises an antigen and an adjuvant. In some embodiments, the antigen is a tumor antigen.

In some embodiments, the individual has an infectious disease or a viral-associated disease and wherein the payload comprises an antigen and an adjuvant. In some embodiments, the antigen is a viral antigen, a bacterial antigen or a fungal antigen. In some embodiments, the individual has an autoimmune disease and wherein the payload comprises an antigen. In some embodiments, the individual has an autoimmune disease and wherein the payload comprises an antigen and a tolerogenic factor.

In some embodiments, the individual has an infectious disease or a viral-associated disease and the payload comprises an antigen. In some embodiments, the individual has multiple sclerosis (MS) and the payload comprises an EBV antigen. In some embodiments, the individual has HIV and the payload comprises an antigen for treating an HIV-associated disease, such as an opportunistic infection.

In some embodiments, the methods described herein further comprise administering to the individual another therapeutic agent. In some embodiments, the method for treating further comprises administering to the individual one or more therapeutic agents. In some embodiments, the other therapeutic agent is administered prior to, concurrently with, or after administering to the individual anucleate cell-derived vesicles and/or compositions described herein. In some embodiments, the therapeutic agent is any one of an immune checkpoint inhibitor, or a cytokine. In some embodiments, the immune checkpoint inhibitor is targeted to any one of PD-1, PD-L1, CTLA-4, TIM-3, LAGS, TIGIT, VISTA, TIM1, B7-H4 (VTCN1) and BTLA. In some embodiments, the cytokine is IFN-γ, IFN-α, IL-10, IL-15, or IL-2 and modified forms thereof. In some embodiments, the cytokine is a tolerogenic cytokine, such as IL-10, TGF-B, and tolerance inducing forms of IL-2. In some embodiments, the therapeutic agent is a tolerogenic agent, such as rapamycin. In some embodiments, the methods described herein further comprise administering to the individual a radiotherapy.

In some embodiments, the anucleate cell-derived vesicle comprises an antigen and/or a tolerogenic factor to suppress an immune response and/or to induce tolerance. In some embodiments, the suppressed immune response and/or induced tolerance comprise a decreased autoimmune response. For example, the decreased autoimmune response can include, without limitation, a decreased immune response or induced tolerance against an antigen associated with Type I Diabetes, Rheumatoid arthritis, Psoriasis, Multiple Sclerosis, Neurodegenerative diseases which may have an immune component such as Neuromyelitis Optica (NMO) Alzheimer's disease, ALS, Huntington's Disease, and Parkinson's Disease, Systemic Lupus Erythromatosus, Sjogren's Disease, Crohn's disease, or Ulcerative Colitis. In some embodiments, the suppressed immune response and/or induced tolerance comprise a decreased allergic response. For example, the decreased allergic response can include a decreased immune response or induced tolerance against antigens associated with allergic asthma, atopic dermatitis, allergic rhinitis (hay fever), or food allergy. In some embodiments, the decreased allergic response can include a decreased immune response or induced tolerance against antigens associated with Celiac disease. In some embodiments, the antigen is an antigen associated with transplanted tissue. In some embodiments, the suppressed immune response and/or induced tolerance comprises a decreased immune response or induced tolerance against the transplanted tissue. In some embodiments, the antigen is associated with a virus. In some embodiments, the suppressed immune response and/or induced tolerance comprises a decreased pathogenic immune response or induced tolerance to the virus. For example, the pathogenic immune response can include the cytokine storm generated by certain viral infections. A cytokine storm is a potentially fatal immune reaction consisting of a positive feedback loop between cytokines and white blood cells.

In some embodiments, the suppressed immune response comprises a decreased immune response against a therapeutic agent. In some embodiments, the therapeutic agent is a clotting factor. Exemplary clotting factors include, without limitation, Factor VIII and Factor IX. In some embodiments, the therapeutic agent is an antibody. Exemplary therapeutic antibodies include, without limitation, anti-TNFα, anti-VEGF, anti-CD3, anti-CD20, anti-IL-2R, anti-Her2, anti-RSVF, anti-CEA, anti-IL-1β, anti-CD15, anti-myosin, anti-PSMA, anti-40 kDa glycoprotein, anti-CD33, anti-CD52, anti-IgE, anti-CD11a, anti-EGFR, anti-CS, anti-α-4 integrin, anti-IL-12/IL-23, anti-IL-6R, and anti-RANKL. In some embodiments, the therapeutic agent is a growth factor. Exemplary therapeutic growth factors include, without limitation, Erythropoietin (EPO) and megakaryocyte differentiation and growth factor (MDGF). In some embodiments, the therapeutic agent is a hormone. Exemplary therapeutic hormones include, without limitation, insulin, human growth hormone, and follicle stimulating hormone. In some embodiments, the therapeutic agent is a recombinant cytokine. Exemplary therapeutic recombinant cytokines include, without limitation, IFNβ, IFNα, and granulocyte-macrophage colony-stimulating factor (GM-CSF).

In some embodiments, the suppressed immune response comprises a decreased immune response against a therapeutic vehicle. In some embodiments, the therapeutic vehicle is a virus, such as an adenovirus, adeno-associated virus (AAV), baculovirus, herpes virus, or retrovirus used for gene therapy. In some embodiments, the therapeutic vehicle is a liposome. In some embodiments, the therapeutic vehicle is a nanoparticle. In some embodiments, the suppressed immune response comprises a decreased immune response against a viral capsid; e.g., an AAV capsid (e.g., AAV VP1, VP2 or VP3 capsid protein). In some embodiments, the decreased immune response against a viral therapeutic vehicle is directed against any serotype of the virus; for example but not limited to AAV1, AAV2, AAV3, AAV4, AAV5, AAV6, AAV7, AAV8, AAV9, AAV10, AAVrh8, or AAVrh10. In some embodiments, the decreased immune response, e.g., against a viral capsid, allows for one or more of higher initial dosage, repeat administration, longer half-life, and longer expression, e.g., repeat dosing of an AAV therapeutic vehicle.

In some embodiments, the suppressed immune response comprises a decreased immune response against a transgene product expressed by a therapeutic vehicle (e.g., a gene therapy vehicle). In some embodiments, the suppressed immune response comprises a decreased immune response against a transgene product expressed by an AAV gene therapy vector.

In some embodiments, the method for treating comprises administering to the individual one or more doses, such as any of 2, 3, 4, 5, 6, 7, 8, 9, 10, 11, or 12 doses, of anucleate cell-derived vesicles and/or compositions described herein. In some embodiments, the method for treating comprises administering to the individual up to 12 doses per year, such as any of 1, 2, 3, 4, 5, 6, 7, 8, 9, 10, 11, or 12 doses per year, of anucleate cell-derived vesicles and/or compositions described herein. In some embodiments, two or more doses are administered over a treatment course in uniform or non-uniform intervals, such as with spacing of any of, e.g., 1 day, 2 days, 3 days, 4, days, 5 days, 6 days, 1 week, or 2 weeks. In some embodiments, two or more doses are administered over a treatment course, wherein the interval between a first dose and a second dose is about 1 week to about 1 year, such as any of about 2 weeks to about 1 month, about 2 weeks to about 3 months, about 2 weeks to about 4 months, about 2 weeks to about 6 months, about 2 weeks to about 9 months, or about 2 weeks to about 12 months.

In some embodiments, provided herein are methods for preventing a disease or disorder in an individual in need thereof, the method comprising administering to the individual any of the anucleate cell-derived vesicles and/or compositions described herein. In some embodiments, the anucleate cell-derived vesicles comprise an antigen. In some embodiments, the individual has cancer and wherein the payload comprises an adjuvant. In some embodiments, the individual has cancer and wherein the payload comprises an antigen and an adjuvant. In some embodiments, the disease or disorder is cancer and the antigen is a tumor antigen. In some embodiments, the individual has an infectious disease and wherein the payload comprises an antigen. In some embodiments, the individual has an infectious disease and wherein the payload comprises an antigen and an adjuvant. In some embodiments, the antigen is a viral antigen, a bacterial antigen or a fungal antigen.

### Systems and Kits

In some aspects, the invention provides a system comprising the constriction, cell suspension, and compound for use in the methods disclosed herein. The system can include any embodiment described for the methods disclosed above, including microfluidic channels or a surface having pores to provide cell-deforming constrictions, cell suspensions, cell perturbations, delivery parameters, compounds, and/or applications etc. In some embodiment, the cell-deforming constrictions are sized for delivery of antigens and/or adjuvants to input anucleate cells. In some embodiments, the delivery parameters, such as operating flow speeds, cell concentration, antigen and/or adjuvant concentration, velocity of the cell in the constriction, and the composition of the cell suspension (e.g., osmolarity, salt concentration, serum content, cell concentration, pH, etc.) are optimized for maximum stimulation or enhancement of an immune response to the antigen and/or the adjuvant.

Also provided are kits or articles of manufacture for use in delivering an antigen and/or an adjuvant to anucleate cell-derived vesicles for stimulating or enhancing an immune response. In some embodiments, the kits comprise the compositions described herein (e.g. a microfluidic channel or surface containing pores, cell suspensions, and/or compounds) in suitable packaging. Suitable packaging materials are known in the art, and include, for example, vials (such as sealed vials), vessels, ampules, bottles, jars, flexible packaging (e.g., sealed Mylar or plastic bags), and the like. These articles of manufacture may further be sterilized and/or sealed.

The present disclosure also provides kits comprising components of the methods described herein and may further comprise instruction(s) for performing said methods to stimulate or enhance an immune response. The kits described herein may further include other materials, including other buffers, diluents, filters, needles, syringes, and package inserts with instructions for performing any methods described herein; e.g., instructions for stimulating or enhancing an immune response.

### Exemplary Embodiments

Embodiment 1. A method for delivering an antigen into an anucleate cell-derived vesicle, the method comprising: a) passing a cell suspension comprising an input anucleate cell through a cell-deforming constriction, wherein a diameter of the constriction is a function of a diameter of the input anucleate cell in the suspension, thereby causing perturbations of the input anucleate cell large enough for the antigen to pass through to form an anucleate cell-derived vesicle; and b) incubating the anucleate cell-derived vesicle with the antigen for a sufficient time to allow the antigen to enter the anucleate cell-derived vesicle.

Embodiment 2. The method of embodiment 1, wherein the input anucleate cell further comprises an adjuvant.

Embodiment 3. A method for delivering an adjuvant into an anucleate cell-derived vesicle, the method comprising: a) passing a cell suspension comprising an input anucleate cell through a cell-deforming constriction, wherein a diameter of the constriction is a function of a diameter of the input anucleate cell in the suspension, thereby causing perturbations of the input anucleate cell large enough for the adjuvant to pass through to form an anucleate cell-derived vesicle; and b) incubating the anucleate cell-derived vesicle with the adjuvant for a sufficient time to allow the adjuvant to enter the anucleate cell-derived vesicle.

Embodiment 4. The method of embodiment 3, wherein the input anucleate cell further comprises an antigen.

Embodiment 5. A method for delivering an antigen and an adjuvant into an anucleate cell-derived vesicle, the method comprising: a) passing a cell suspension comprising an input anucleate cell through a cell-deforming constriction, wherein a diameter of the constriction is a function of a diameter of the input anucleate cell in the suspension, thereby causing perturbations of the input anucleate cell large enough for the antigen and the adjuvant to pass through to form an anucleate cell-derived vesicle; and b) incubating the anucleate cell-derived vesicle with the antigen and the adjuvant for a sufficient time to allow the antigen and the adjuvant to enter the anucleate cell-derived vesicle.

Embodiment 6. A method for stimulating an immune response to an antigen in an individual, the method comprising administering to the individual an effective amount of an anucleate cell-derived vesicle comprising an antigen, wherein the anucleate cell-derived vesicle comprising the antigen is prepared by a process comprising the steps of: a) passing a cell suspension comprising an input anucleate cell through a cell-deforming constriction, wherein a diameter of the constriction is a function of a diameter of the input anucleate cell in the suspension, thereby causing perturbations of the input anucleate cell large enough for the antigen to pass through to form an anucleate cell-derived vesicle; and b) incubating the anucleate cell-derived vesicle with the antigen for a sufficient time to allow the antigen to enter the anucleate cell-derived vesicle.

Embodiment 7. The method of embodiment 6, wherein the method further comprises administering an adjuvant systemically to the individual.

Embodiment 8. The method of embodiment 7, wherein the adjuvant is administered systemically before, after or at the same time as the anucleate cell derived vesicle.

Embodiment 9. The method of any one of embodiments 6-8, wherein the input anucleate cell comprises an adjuvant.

Embodiment 10. A method for stimulating an immune response to an antigen in an individual, the method comprising administering to the individual an effective amount of an anucleate cell-derived vesicle comprising an antigen and an adjuvant, wherein the anucleate cell-derived vesicle comprising the antigen and the adjuvant is prepared by a process comprising the steps of: a) passing a cell suspension comprising an input anucleate cell through a cell-deforming constriction, wherein a diameter of the constriction is a function of a diameter of the input anucleate cell in the suspension, thereby causing perturbations of the input anucleate cell large enough for the antigen and the adjuvant to pass through to form an anucleate cell-derived vesicle; and b) incubating the anucleate cell-derived vesicle with the antigen and the adjuvant for a sufficient time to allow the antigen and the adjuvant to enter the anucleate cell-derived vesicle.

Embodiment 11. The method of embodiment 10, wherein the method further comprises administering an adjuvant systemically to the individual.

Embodiment 12. The method of embodiment 11, wherein the adjuvant is administered systemically before, after or at the same time as the anucleate cell-derived vesicle.

Embodiment 13. A method for treating a disease in an individual, comprising administering to the individual an anucleate cell-derived vesicle comprising a disease-associated antigen, wherein an immune response against the antigen ameliorates conditions of the disease, and wherein the anucleate cell-derived vesicle comprising the disease-associated antigen is prepared by a process comprising the steps of: a) passing a cell suspension comprising an input anucleate cell through a cell-deforming constriction, wherein a diameter of the constriction is a function of a diameter of the input anucleate cell in the suspension, thereby causing perturbations of the input anucleate cell large enough for the antigen to pass through to form an anucleate cell-derived vesicle; and b) incubating the anucleate cell-derived vesicle with the antigen for a sufficient time to allow the antigen to enter the anucleate cell-derived vesicle.

Embodiment 14. A method for preventing a disease in an individual, comprising administering to the individual an anucleate cell-derived vesicle comprising a disease-associated antigen, wherein an immune response against the antigen prevents development of the disease, and wherein the anucleate cell-derived vesicle comprising the disease-associated antigen is prepared by a process comprising the steps of: a) passing a cell suspension comprising an input anucleate cell through a cell-deforming constriction, wherein a diameter of the constriction is a function of a diameter of the input anucleate cell in the suspension, thereby causing perturbations of the input anucleate cell large enough for the antigen to pass through to form an anucleate cell-derived vesicle; and b) incubating the anucleate cell-derived vesicle with the antigen for a sufficient time to allow the antigen to enter the anucleate cell-derived vesicle.

Embodiment 15. A method for vaccinating an individual against an antigen, comprising administering to the individual an anucleate cell-derived vesicle comprising the antigen, wherein the anucleate cell-derived vesicle comprising the antigen is prepared by a process comprising the steps of: a) passing a cell suspension comprising an input anucleate cell through a cell-deforming constriction, wherein a diameter of the constriction is a function of a diameter of the input anucleate cell in the suspension, thereby causing perturbations of the input anucleate cell large enough for the antigen to pass through to form an anucleate cell-derived vesicle; and b) incubating the anucleate cell-derived vesicle with the antigen for a sufficient time to allow the antigen to enter the anucleate cell-derived vesicle.

Embodiment 16. The method of any one of embodiments 13-15, wherein the method further comprises administering an adjuvant systemically to the individual.

Embodiment 17. The method of embodiment 16, wherein the adjuvant is administered systemically before, after or at the same time as the anucleate cell derived vesicle.

Embodiment 18. The method of embodiment 13-17, wherein the input anucleate cell comprises an adjuvant.

Embodiment 19. A method for treating a disease in an individual, comprising administering to the individual an anucleate cell-derived vesicle comprising a disease-associated antigen and an adjuvant, wherein an immune response against the antigen ameliorates conditions of the disease, and wherein the anucleate cell-derived vesicle comprising the disease-associated antigen and the adjuvant is prepared by a process comprising the steps of: a) passing a cell suspension comprising an input anucleate cell through a cell-deforming constriction, wherein a diameter of the constriction is a function of a diameter of the input anucleate cell in the suspension, thereby causing perturbations of the input anucleate cell large enough for the antigen and an adjuvant to pass through to form an anucleate cell-derived vesicle; and b) incubating the anucleate cell-derived vesicle with the antigen and the adjuvant for a sufficient time to allow the antigen and the adjuvant to enter the anucleate cell-derived vesicle.

Embodiment 20. A method for preventing a disease in an individual, comprising administering to the individual an anucleate cell-derived vesicle comprising a disease-associated antigen and an adjuvant, wherein an immune response against the antigen prevents development of the disease, and wherein the anucleate cell-derived vesicle comprising a disease-associated antigen and an adjuvant is prepared by a process comprising the steps of: a) passing a cell suspension comprising an input anucleate cell through a cell-deforming constriction, wherein a diameter of the constriction is a function of a diameter of the input anucleate cell in the suspension, thereby causing perturbations of the input anucleate cell large enough for the antigen and an adjuvant to pass through to form an anucleate cell-derived vesicle; an b) incubating the anucleate cell-derived vesicle with the antigen and the adjuvant for a sufficient time to allow the antigen and the adjuvant to enter the anucleate cell-derived vesicle.

Embodiment 21. A method for vaccinating an individual against an antigen, comprising administering to the individual an anucleate cell-derived vesicle comprising the antigen and an adjuvant, wherein the anucleate cell-derived vesicle comprising the antigen and the adjuvant is prepared by a process comprising the steps of: a) passing a cell suspension comprising an input anucleate cell through a cell-deforming constriction, wherein a diameter of the constriction is a function of a diameter of the input anucleate cell in the suspension, thereby causing perturbations of the input anucleate cell large enough for the antigen and an adjuvant to pass through to form an anucleate cell-derived vesicle; and b) incubating the anucleate cell-derived vesicle with the antigen and the adjuvant for a sufficient time to allow the antigen and the adjuvant to enter the anucleate cell-derived vesicle.

Embodiment 22. A method for treating a disease in an individual, wherein an immune response against a disease-associated antigen ameliorates conditions of the disease, the method comprising: a) passing a cell suspension comprising an input anucleate cell through a cell-deforming constriction, wherein a diameter of the constriction is a function of a diameter of the input anucleate cell in the suspension, thereby causing perturbations of the input anucleate cell large enough for the antigen to pass through to form an anucleate cell-derived vesicle; b) incubating the anucleate cell-derived vesicle with the antigen for a sufficient time to allow the antigen to enter the anucleate cell-derived vesicle, thereby generating an anucleate cell-derived vesicle comprising the antigen; and c) administering the anucleate cell-derived vesicle comprising the antigen to the individual.

Embodiment 23. A method for preventing a disease in an individual, wherein an immune response against a disease-associated antigen prevents development of the disease, the method comprising: a) passing a cell suspension comprising an input anucleate cell through a cell-deforming constriction, wherein a diameter of the constriction is a function of a diameter of the input anucleate cell in the suspension, thereby causing perturbations of the input anucleate cell large enough for the antigen to pass through to form an anucleate cell-derived vesicle; b) incubating the anucleate cell-derived vesicle with the antigen for a sufficient time to allow the antigen to enter the anucleate cell-derived vesicle, thereby generating an anucleate cell-derived vesicle comprising the antigen; and c) administering the anucleate cell-derived vesicle comprising the antigen to the individual.

Embodiment 24. A method for vaccinating an individual against an antigen, the method comprising: a) passing a cell suspension comprising an input anucleate cell through a cell-deforming constriction, wherein a diameter of the constriction is a function of a diameter of the input anucleate cell in the suspension, thereby causing perturbations of the input anucleate cell large enough for the antigen to pass through to form an anucleate cell-derived vesicle; b) incubating the anucleate cell-derived vesicle with the antigen for a sufficient time to allow the antigen to enter the anucleate cell-derived vesicle, thereby generating an anucleate cell-derived vesicle comprising the antigen; and c) administering the anucleate cell-derived vesicle comprising the antigen to the individual.

Embodiment 25. The method of any one of embodiments 19-24, wherein the method further comprises administering an extravesicular adjuvant systemically to the individual.

Embodiment 26. The method of embodiment 25, wherein the extravesicular adjuvant is administered before, after or at the same time as the anucleate cell-derived vesicle.

Embodiment 27. The method of embodiment 19-24, wherein the input anucleate cell comprises an adjuvant.

Embodiment 28. A method for treating a disease in an individual, wherein an immune response against a disease-associated antigen ameliorates conditions of the disease, the method comprising: a) passing a cell suspension comprising an input anucleate cell through a cell-deforming constriction, wherein a diameter of the constriction is a function of a diameter of the input anucleate cell in the suspension, thereby causing perturbations of the input anucleate cell large enough for the disease-associated antigen and an adjuvant to pass through to form an anucleate cell-derived vesicle; b) incubating the anucleate cell-derived vesicle with the antigen and the adjuvant for a sufficient time to allow the antigen and the adjuvant to enter the anucleate cell-derived vesicle, thereby generating an anucleate cell-derived vesicle comprising the antigen and the adjuvant; and c) administering the anucleate cell-derived vesicle comprising the antigen and the adjuvant to the individual.

Embodiment 29. A method for preventing a disease in an individual, wherein an immune response against a disease-associated antigen prevents development of the disease, the method comprising: a) passing a cell suspension comprising an input anucleate cell through a cell-deforming constriction, wherein a diameter of the constriction is a function of a diameter of the input anucleate cell in the suspension, thereby causing perturbations of the input anucleate cell large enough for the antigen and an adjuvant to pass through to form an anucleate cell-derived vesicle; b) incubating the anucleate cell-derived vesicle with the antigen and the adjuvant for a sufficient time to allow the antigen and the adjuvant to enter the anucleate cell-derived vesicle, thereby generating an anucleate cell-derived vesicle comprising the antigen and the adjuvant; and c) administering the anucleate cell-derived vesicle comprising the antigen and the adjuvant to the individual.

Embodiment 30. A method for vaccinating an individual against an antigen, the method comprising, a) passing a cell suspension comprising an input anucleate cell through a cell-deforming constriction, wherein a diameter of the constriction is a function of a diameter of the input anucleate cell in the suspension, thereby causing perturbations of the input anucleate cell large enough for the antigen and an adjuvant to pass through to form an anucleate cell-derived vesicle; b) incubating the anucleate cell-derived vesicle with the antigen and the adjuvant for a sufficient time to allow the antigen and the adjuvant to enter the anucleate cell-derived vesicle, thereby generating an anucleate cell-derived vesicle comprising the antigen and the adjuvant; and c) administering the anucleate cell-derived vesicle comprising the antigen and the adjuvant to the individual.

Embodiment 31. The method of any one of embodiments 28-30, wherein the method further comprises administering an extravesicular adjuvant systemically to the individual.

Embodiment 32. The method of embodiment 31, wherein the extravesicular adjuvant is administered before, after or at the same time as the anucleate cell derived vesicle.

Embodiment 33. The method of any one of embodiments 13-32, wherein the disease is cancer, an infectious disease or a viral-associated disease.

Embodiment 34. The method of any one of embodiments 6-33 wherein the anucleate cell-derived vesicle is autologous to the individual.

Embodiment 35. The method of any one of embodiments 6-33, wherein the anucleate cell-derived vesicle is allogeneic to the individual.

Embodiment 36. The method of any one of embodiments 6-35, wherein the anucleate cell-derived vesicle is in a pharmaceutical formulation.

Embodiment 37. The method of any one of embodiments 6-36, wherein the anucleate cell-derived vesicle is administered systemically.

Embodiment 38. The method of any one of embodiments 6-37, wherein the anucleate cell-derived vesicle is administered intravenously, intraarterially, subcutaneously, intramuscularly, or intraperitoneally.

Embodiment 39. The method of any one of embodiments 6-38, wherein the anucleate cell-derived vesicle is administered to the individual in combination with a therapeutic agent.

Embodiment 40. The method of embodiment 39, wherein the therapeutic agent is administered before, after or at the same time as the anucleate cell-derived vesicle.

Embodiment 41. The method of embodiment 39 or 40, wherein the therapeutic agent is an immune checkpoint inhibitor and/or a cytokine.

Embodiment 42. The method of embodiment 41, wherein the cytokine is one or more of IFN-α, IFN-γ, IL-2 or IL-15.

Embodiment 43. The method of embodiment 41, wherein the immune checkpoint inhibitor is targeted to any one of PD-1, PD-L1, CTLA-4, TIM-3, LAG3, TIGIT, VISTA, TIM1, B7-H4 (VTCN1) and BTLA.

Embodiment 44. The method of any one of embodiments 1, 2, or 4-43, wherein the antigen is capable of being processed into an MHC class I-restricted peptide and/or an MHC class II-restricted peptide.

Embodiment 45. The method of any one of embodiments 1, 2, or 4-43, wherein the antigen is a CD-1 restricted antigen.

Embodiment 46. The method of any one of embodiments 1, 2, or 4-45, wherein the antigen is a disease-associated antigen.

Embodiment 47. The method of any one of embodiments 1, 2, or 4-46, wherein the antigen is a tumor antigen.

Embodiment 48. The method of any one of embodiments 1, 2, or 4-47, wherein the antigen is derived from a lysate.

Embodiment 49. The method of embodiment 48, wherein the lysate is a tumor lysate.

Embodiment 50. The method of any one of embodiments 1, 2, or 4-46, wherein the antigen is a viral antigen, a bacterial antigen or a fungal antigen.

Embodiment 51. The method of any one of embodiments 1, 2, or 4-46, wherein the antigen is a microorganism.

Embodiment 52. The method of any one of embodiments 1, 2, or 4-50, wherein the antigen is a polypeptide.

Embodiment 53. The method of any one of embodiments 1, 2, or 4-50, wherein the antigen is a lipid antigen.

Embodiment 54. The method of any one of embodiments 1, 2, or 4-50, wherein the antigen is a carbohydrate antigen.

Embodiment 55. The method of any one of embodiments 1, 2, or 4-54, wherein the antigen is a modified antigen.

Embodiment 56. The method of embodiment 55, wherein the modified antigen comprises an antigen fused with a polypeptide.

Embodiment 57. The method of embodiment 56, wherein the modified antigen comprises an antigen fused with a targeting peptide.

Embodiment 58. The method of embodiment 55, wherein the modified antigen comprises an antigen fused with a lipid.

Embodiment 59. The method of embodiment 55, wherein the modified antigen comprises an antigen fused with a carbohydrate.

Embodiment 60. The method of embodiment 55, wherein the modified antigen comprises an antigen fused with a nanoparticle.

Embodiment 61. The method of any one of embodiments 1-60, wherein a plurality of antigens is delivered to the anucleate cell-derived vesicle.

Embodiment 62. The method of any one of embodiments 2-5, 7-12, 16-21, 25-61 wherein the adjuvant is a CpG ODN, IFN-α, STING agonists, RIG-I agonists, poly I:C, polyinosinic-polycytidylic acid stabilized with polylysine and carboxymethylcellulose (HILTONOL®), imiquimod, resiquimod, and/or lipopolysaccharide (LPS).

Embodiment 63. The method of embodiment 62, wherein the adjuvant is low molecular weight poly I:C.

Embodiment 64. The method of any one of embodiments 1-63, wherein the input anucleate cell is a red blood cell.

Embodiment 65. The method of any one of embodiments 1-63, wherein the red blood cell is an erythrocyte.

Embodiment 66. The method of any one of embodiments 1-63, wherein the red blood cell is a reticulocyte.

Embodiment 67. The method of any one of embodiments 1-63, wherein the input anucleate cell is a platelet.

Embodiment 68. The method of any one of embodiments 1-67, wherein the input anucleate cell is a mammalian cell.

Embodiment 69. The method of any one of embodiments 1-68, wherein the input anucleate cell is a monkey, mouse, dog, cat, horse, rat, sheep, goat, pig, or rabbit cell.

Embodiment 70. The method of any one of embodiments 1-68, wherein the input anucleate cell is a human cell.

Embodiment 71. The method of any one of embodiments 1-70, wherein the constriction is contained within a microfluidic channel.

Embodiment 72. The method of embodiment 71, wherein the microfluidic channel comprises a plurality of constrictions.

Embodiment 73. The method of embodiment 72, wherein the plurality of constrictions are arranged in series and/or in parallel.

Embodiment 74. The method of any one of embodiments 1-73, wherein the constriction is between a plurality of micropillars; between a plurality of micropillars configured in an array; or between one or more movable plates.

Embodiment 75. The method of any one of embodiments 1-70, wherein the constriction is a pore or contained within a pore.

Embodiment 76. The method of embodiment 75, wherein the pore is contained in a surface.

Embodiment 77. The method of embodiment 76, wherein the surface is a filter.

Embodiment 78. The method of embodiment 76, wherein the surface is a membrane.

Embodiment 79. The method of any one of embodiments 1-76, wherein the constriction size is a function of the diameter of the input anucleate cell in suspension.

Embodiment 80. The method of any one of embodiments 1-79, wherein the constriction size is about 10%, about 20%, about 30%, about 40%, about 50%, about 60%, or about 70% of the diameter of the input anucleate cell in suspension.

Embodiment 81. The method of any one of embodiments 1-79, wherein the constriction has a width of about 0.25 μm to about 4 μm.

Embodiment 82. The method of any one of embodiments 1-79, wherein the constriction has a width of about 4 μm, 3.5 μm, about 3 μm, about 2.5 μm, about 2 μm, about 1.5 μm, about 1 μm, about 0.5 μm, or about 0.25 μm.

Embodiment 83. The method of any one of embodiments 1-79, wherein the constriction has a width of about 2.2 μm.

Embodiment 84. The method of any one of embodiments 1-83, wherein the input anucleate cells are passed through the constriction under a pressure ranging from about 10 psi to about 90 psi.

Embodiment 85. The method of any one of embodiments 1-84, wherein said cell suspension is contacted with the antigen before, concurrently, or after passing through the constriction.

Embodiment 86. An anucleate cell-derived vesicle comprising an antigen, wherein the anucleate cell-derived vesicle comprising the antigen is prepared by a process comprising the steps of: a) passing a cell suspension comprising an input anucleate cell through a cell-deforming constriction, wherein a diameter of the constriction is a function of a diameter of the input anucleate cell in the suspension, thereby causing perturbations of the input anucleate cell large enough for the antigen to pass through to form an anucleate cell-derived vesicle; and b) incubating the anucleate cell-derived vesicle with the antigen for a sufficient time to allow the antigen to enter the anucleate cell-derived vesicle; thereby generating the anucleate cell-derived vesicle comprising the antigen.

Embodiment 87. The anucleate cell-derived vesicle of embodiment 86, wherein the input anucleate cell comprises an adjuvant.

Embodiment 88. An anucleate cell-derived vesicle comprising an adjuvant, wherein the anucleate cell-derived vesicle comprising the adjuvant is prepared by a process comprising the steps of: a) passing a cell suspension comprising an input anucleate cell through a cell-deforming constriction, wherein a diameter of the constriction is a function of a diameter of the input anucleate cell in the suspension, thereby causing perturbations of the input anucleate cell large enough for the adjuvant to pass through to form an anucleate cell-derived vesicle; and b) incubating the anucleate cell-derived vesicle with the adjuvant for a sufficient time to allow the adjuvant to enter the anucleate cell-derived vesicle; thereby generating the anucleate cell-derived vesicle comprising the adjuvant.

Embodiment 89. The anucleate cell-derived vesicle of embodiment 88, wherein the input anucleate cell comprises an antigen.

Embodiment 90. An anucleate cell-derived vesicle comprising an antigen and an adjuvant, wherein the anucleate cell-derived vesicle comprising the antigen and the adjuvant is prepared by a process comprising the steps of: a) passing a cell suspension comprising an input anucleate cell through a cell-deforming constriction, wherein a diameter of the constriction is a function of a diameter of the input anucleate cell in the suspension, thereby causing perturbations of the input anucleate cell large enough for the antigen and the adjuvant to pass through to form an anucleate cell-derived vesicle; and b) incubating the anucleate cell-derived vesicle with the antigen and the adjuvant for a sufficient time to allow the antigen and the adjuvant to enter the anucleate cell-derived vesicle; thereby generating the anucleate cell-derived vesicle comprising the antigen and the adjuvant.

Embodiment 91. The anucleate cell-derived vesicle of any one of embodiments 86-90, wherein the anucleate cell-derived vesicle is a red blood cell-derived vesicle or a platelet-derived vesicle.

Embodiment 92. The anucleate cell-derived vesicle of embodiment 91, wherein the red blood cell-derived vesicle is an erythrocyte-derived vesicle, or a reticulocyte-derived vesicle.

Embodiment 93. The anucleate cell-derived vesicle of any one of embodiments 86, 87, or 89-92, wherein the antigen is capable of being processed into an MHC class I-restricted peptide and/or an MHC class II-restricted peptide.

Embodiment 94. The anucleate cell-derived vesicle of any one of embodiments 86, 87, or 89-92, wherein the antigen is a CD-1 restricted antigen.

Embodiment 95. The anucleate cell-derived vesicle of any one of embodiments 86, 87, or 89-94, wherein the antigen is a disease-associated antigen.

Embodiment 96. The anucleate cell-derived vesicle of any one of embodiments 86, 87, or 89-95, wherein the antigen is a tumor antigen.

Embodiment 97. The anucleate cell-derived vesicle of any one of embodiments 86, 87, or 89-96, wherein the antigen is derived from a lysate.

Embodiment 98. The anucleate cell-derived vesicle of embodiment 97, wherein the lysate is a tumor lysate.

Embodiment 99. The anucleate cell-derived vesicle of any one of embodiments 86, 87, or 89-95, wherein the antigen is a viral antigen, a bacterial antigen or a fungal antigen.

Embodiment 100. The anucleate cell-derived vesicle of any one of embodiments 86, 87, or 89-95, wherein the antigen is a microorganism.

Embodiment 101. The anucleate cell-derived vesicle of any one of embodiments 86, 87, or 89-99, wherein the antigen is a polypeptide.

Embodiment 102. The anucleate cell-derived vesicle of any one of embodiments 86, 87, or 89-99, wherein the antigen is a lipid antigen.

Embodiment 103. The anucleate cell-derived vesicle of any one of embodiments 86, 87, or 89-99, wherein the antigen is a carbohydrate antigen.

Embodiment 104. The anucleate cell-derived vesicle of any one of embodiments 86, 87, or 89-103, wherein the antigen is a modified antigen.

Embodiment 105. The anucleate cell-derived vesicle of embodiment 104, wherein the modified antigen comprises an antigen fused with a polypeptide.

Embodiment 106. The anucleate cell-derived vesicle of embodiment 105, wherein the modified antigen comprises an antigen fused with a targeting peptide.

Embodiment 107. The anucleate cell-derived vesicle of embodiment 104, wherein the modified antigen comprises an antigen fused with a lipid.

Embodiment 108. The anucleate cell-derived vesicle of embodiment 104, wherein the modified antigen comprises an antigen fused with a carbohydrate.

Embodiment 109. The anucleate cell-derived vesicle of embodiment 104, wherein the modified antigen comprises an antigen fused with a nanoparticle.

Embodiment 110. The anucleate cell-derived vesicle of any one of embodiments 86, 87, or 89-109, wherein a plurality of antigens is delivered to the anucleate cell-derived vesicle.

Embodiment 111. The anucleate cell-derived vesicle of any one of embodiments 87-110 wherein the adjuvant is a CpG ODN, IFN-α, STING agonists, RIG-I agonists, poly I:C, polyinosinic-polycytidylic acid stabilized with polylysine and carboxymethylcellulose (HILTONOL®), imiquimod, resiquimod and/or LPS.

Embodiment 112. The anucleate cell-derived vesicle of embodiment 111, wherein the adjuvant is low molecular weight poly I:C.

Embodiment 113. The anucleate cell-derived vesicle of any one of embodiments 86-112 wherein the input anucleate cell is a red blood cell.

Embodiment 114. The anucleate cell-derived vesicle of any one of embodiments 86-112, wherein the input anucleate cell is an erythrocyte.

Embodiment 115. The anucleate cell-derived vesicle of any one of embodiments 86-112, wherein the input anucleate cell is a reticulocyte.

Embodiment 116. The anucleate cell-derived vesicle of any one of embodiments 86-112, wherein the input anucleate cell is a platelet.

Embodiment 117. The anucleate cell-derived vesicle of any one of embodiments 86-116 wherein the input anucleate cell is a mammalian cell.

Embodiment 118. The anucleate cell-derived vesicle of any one of embodiments 86-117, wherein the input anucleate cell is a monkey, mouse, dog, cat, horse, rat, sheep, goat, pig, or rabbit cell.

Embodiment 119. The anucleate cell-derived vesicle of any one of embodiments 86-117, wherein the input anucleate cell is a human cell.

Embodiment 120. The anucleate cell-derived vesicle of any one of embodiments 86-119 wherein a half-life of the anucleate cell-derived vesicle following administration to a mammal is decreased compared to a half-life of the input anucleate cell following administration to the mammal.

Embodiment 121. The anucleate cell-derived vesicle of any one of embodiments 86-115, or 117-120, wherein a hemoglobin content of the anucleate cell-derived vesicle is decreased compared to the hemoglobin content of the input anucleate cell.

Embodiment 122. The anucleate cell-derived vesicle of any one of embodiments 86-120, wherein ATP production of the anucleate cell-derived vesicle is decreased compared to ATP production of the input anucleate cell.

Embodiment 123. The anucleate cell-derived vesicle of any one of embodiments 113, 114, 117-122 wherein the anucleate cell-derived vesicle exhibits one or more of the following properties: (a) a circulating half-life in a mammal that is decreased compared to the input anucleate cell; (b) decreased hemoglobin level compared to the input anucleate cell; (c) a spherical morphology; (d) increased surface phosphatidylserine levels compared to the input anucleate cell, (e) reduced ATP production compared to the input anucleate cell.

Embodiment 124. The anucleate cell-derived vesicle of any one of embodiments 113, 114, 117-122, wherein the input anucleate cell is an erythrocyte and wherein the anucleate cell-derived vesicle has a reduced biconcave shape compared to the input anucleate cell.

Embodiment 125. The anucleate cell-derived vesicle of embodiment 113, 114, 117-122 wherein the anucleate cell-derived vesicle is a red blood cell ghost.

Embodiment 126. The anucleate cell-derived vesicle of any one of embodiments 86-125, wherein the anucleate cell-derived vesicles prepared by the process have greater than about 1.5 fold more phosphatidylserine on its surface compared to the input anucleate cell.

Embodiment 127. The anucleate cell-derived vesicle of any one of embodiments 86-126, wherein a population profile of anucleate cell-derived vesicles prepared by the process exhibits higher average phosphatidylserine levels on the surface compared to the input anucleate cells.

Embodiment 128. The anucleate cell-derived vesicle of any one of embodiments 86-127, wherein at least 50% of the population profile of anucleate cell-derived vesicles prepared by the process exhibits higher phosphatidylserine levels on the surface compared to the input anucleate cells

Embodiment 129. The anucleate cell-derived vesicle of any one of embodiments 86-128, wherein the anucleate cell-derived vesicle exhibits enhanced uptake in a tissue or cell compared to the input anucleate cell.

Embodiment 130. The anucleate cell-derived vesicle of embodiment 129, wherein the anucleate cell-derived vesicle exhibits enhanced uptake in liver and/or spleen or by a phagocytic cell and/or an antigen-presenting cell compared to the uptake of the input anucleate cell.

Embodiment 131. The anucleate cell-derived vesicle of any one of embodiments 86-130, wherein the anucleate cell-derived vesicle is modified to enhance uptake in a tissue or cell compared to an unmodified anucleate cell-derived vesicle.

Embodiment 132. The anucleate cell-derived vesicle of embodiment 131, wherein the anucleate cell-derived vesicle is modified to enhance uptake in liver and/or spleen or by a phagocytic cell and/or an antigen-presenting cell compared to the uptake of the input anucleate cell.

Embodiment 133 The anucleate cell-derived vesicle of any one of embodiments 86-132, wherein the anucleate cell-derived vesicle comprises CD47 on its surface.

Embodiment 134. The anucleate cell-derived vesicle of any one of embodiments 86-133, wherein the anucleate cell-derived vesicle is not (a) heat processed, (b) chemically treated, and/or (c) subjected to hypotonic or hypertonic conditions during the preparation of the anucleate cell-derived vesicles.

Embodiment 135. The anucleate cell-derived vesicle of any one of embodiments 86-134, wherein the osmolarity of the cell suspension is maintained throughout the process.

Embodiment 136. The anucleate cell-derived vesicle of embodiments 86-135, wherein the osmolarity of the cell suspension is maintained between 200 mOsm and 400 mOsm throughout the process.

Embodiment 137. The anucleate cell-derived vesicle of any one of embodiments 86-136, wherein the constriction is contained within a microfluidic channel.

Embodiment 138. The anucleate cell-derived vesicle of embodiment 137, wherein the microfluidic channel comprises a plurality of constrictions.

Embodiment 139. The anucleate cell-derived vesicle of embodiment 138, wherein the plurality of constrictions are arranged in series and/or in parallel.

Embodiment 140. The anucleate cell-derived vesicle of any one of embodiments 86-139, wherein the constriction is between a plurality of micropillars; between a plurality of micropillars configured in an array; or between one or more movable plates.

Embodiment 141. The anucleate cell-derived vesicle of any one of embodiments 86-136, wherein the constriction is a pore or contained within a pore.

Embodiment 142. The anucleate cell-derived vesicle of embodiment 141, wherein the pore is contained in a surface.

Embodiment 143. The anucleate cell-derived vesicle of embodiment 142, wherein the surface is a filter.

Embodiment 144. The anucleate cell-derived vesicle of embodiment 142, wherein the surface is a membrane.

Embodiment 145. The anucleate cell-derived vesicle of any one of embodiments 86-144, wherein the constriction size is a function of the diameter of the input anucleate cell in suspension.

Embodiment 146. The anucleate cell-derived vesicle of any one of embodiments 86-144, wherein the constriction size is about 10%, about 20%, about 30%, about 40%, about 50%, about 60%, or about 70% of the diameter of the input anucleate cell in suspension.

Embodiment 147. The anucleate cell-derived vesicle of any one of embodiments 86-146, wherein the constriction has a width of about 0.25 μm to about 4 μm.

Embodiment 148. The anucleate cell-derived vesicle of any one of embodiments 86-147, wherein the constriction has a width of about 4 μm, 3.5 μm, about 3 μm, about 2.5 μm, about 2 μm, about 1.5 μm, about 1 μm, about 0.5 μm, or about 0.25 μm.

Embodiment 149. The anucleate cell-derived vesicle of any one of embodiments 86-147, wherein the constriction has a width of about 2.2 μm.

Embodiment 150. The anucleate cell-derived vesicle of any one of embodiments 86-149, wherein the input anucleate cells are passed through the constriction under a pressure ranging from about 10 psi to about 90 psi.

Embodiment 151. The anucleate cell-derived vesicle of any one of embodiments 86-150, wherein said cell suspension is contacted with the antigen before, concurrently, or after passing through the constriction.

Embodiment 152. A composition comprising a plurality of anucleate cell-derived vesicles of any one of embodiments 86-151.

Embodiment 153. The composition of embodiment 152, further comprising a pharmaceutically acceptable excipient.

Embodiment 154. A method for generating an anucleate cell-derived vesicle comprising an antigen, the method comprising: a) passing a cell suspension comprising an input anucleate cell through a cell-deforming constriction, wherein a diameter of the constriction is a function of a diameter of the input anucleate cell in the suspension, thereby causing perturbations of the input anucleate cell large enough for the antigen to pass through to form an anucleate cell-derived vesicle; b) incubating the anucleate cell-derived vesicle with the antigen for a sufficient time to allow the antigen to enter the anucleate cell-derived vesicle, thereby generating an anucleate cell-derived vesicle comprising the antigen.

Embodiment 155. The method of embodiment 154, wherein the input anucleate cell comprises an adjuvant.

Embodiment 156. A method for generating an anucleate cell-derived vesicle comprising an adjuvant, the method comprising: a) passing a cell suspension comprising an input anucleate cell through a cell-deforming constriction, wherein a diameter of the constriction is a function of a diameter of the input anucleate cell in the suspension, thereby causing perturbations of the input anucleate cell large enough for the adjuvant to pass through to form an anucleate cell-derived vesicle; b) incubating the anucleate cell-derived vesicle with the adjuvant for a sufficient time to allow the adjuvant to enter the anucleate cell-derived vesicle, thereby generating an anucleate cell-derived vesicle comprising the adjuvant.

Embodiment 157. The method of embodiment 156, wherein the input anucleate cell comprises an antigen.

Embodiment 158. A method for generating an anucleate cell-derived vesicle comprising an antigen and an adjuvant, the method comprising: a) passing a cell suspension comprising an input anucleate cell through a cell-deforming constriction, wherein a diameter of the constriction is a function of a diameter of the input anucleate cell in the suspension, thereby causing perturbations of the input anucleate cell large enough for the antigen and the adjuvant to pass through to form an anucleate cell-derived vesicle; b) incubating the anucleate cell-derived vesicle with the antigen and the adjuvant for a sufficient time to allow the antigen and the adjuvant to enter the anucleate cell-derived vesicle, thereby generating an anucleate cell-derived vesicle comprising the antigen and the adjuvant.

Embodiment 159. The method of any one of embodiments 154-158, wherein the anucleate cell-derived vesicle is a red blood cell-derived vesicle or a platelet derived vesicle.

Embodiment 160. The method of embodiment 159, wherein the red blood cell-derived vesicle is an erythrocyte-derived vesicle or a reticulocyte-derived vesicle.

Embodiment 161. The method of any one of embodiments 154, 155 or 157-160, wherein the antigen is capable of being processed into an MHC class I-restricted peptide and/or an MHC class II-restricted peptide.

Embodiment 162. The method of any one of embodiments 154, 155 or 157-160, wherein the antigen is a CD-1 restricted antigen.

Embodiment 163. The method of any one of embodiments 154, 155 or 157-162, wherein the antigen is a disease-associated antigen.

Embodiment 164. The method of any one of embodiments 154, 155 or 157-163, wherein the antigen is a tumor antigen.

Embodiment 165. The method of any one of embodiments 154, 155 or 157-164, wherein the antigen is derived from a lysate.

Embodiment 166. The method of embodiment 165, wherein the lysate is a tumor lysate.

Embodiment 167. The method of any one of embodiments 154, 155 or 157-163, wherein the antigen is a viral antigen, a bacterial antigen or a fungal antigen.

Embodiment 168. The method of any one of embodiments 154, 155 or 157-163, wherein the antigen is a microorganism.

Embodiment 169. The method of any one of embodiments 154, 155 or 157-167, wherein the antigen is a polypeptide.

Embodiment 170. The method of any one of embodiments 154, 155 or 157-167, wherein the antigen is a lipid antigen.

Embodiment 171. The method of any one of embodiments 154, 155 or 157-167, wherein the antigen is a carbohydrate antigen.

Embodiment 172. The method of any one of embodiments 154, 155 or 157-171, wherein the antigen is a modified antigen.

Embodiment 173. The method of embodiment 172, wherein the modified antigen comprises an antigen fused with a polypeptide.

Embodiment 174. The method of embodiment 173, wherein the modified antigen comprises an antigen fused with a targeting peptide.

Embodiment 175. The method of embodiment 174, wherein the modified antigen comprises an antigen fused with a lipid.

Embodiment 176. The method of embodiment 175, wherein the modified antigen comprises an antigen fused with a carbohydrate.

Embodiment 177. The method of embodiment 176, wherein the modified antigen comprises an antigen fused with a nanoparticle.

Embodiment 178. The method of any one of embodiments 154, 155 or 157-177, wherein a plurality of antigens is delivered to the anucleate cell-derived vesicle.

Embodiment 179. The method of any one of embodiments 155-178 wherein the adjuvant is a CpG ODN, IFN-α, STING agonists, RIG-I agonists, poly I:C, polyinosinic-polycytidylic acid stabilized with polylysine and carboxymethylcellulose (HILTONOL®), imiquimod, resiquimod, and/or LPS.

Embodiment 180. The method of embodiment 179, wherein the adjuvant is a low molecular weight poly I:C.

Embodiment 181. The method of any one of embodiments 154-180 wherein the input anucleate cell is a red blood cell.

Embodiment 182. The method of any one of embodiments 154-181, wherein the input anucleate cell is an erythrocyte.

Embodiment 183. The method of any one of embodiments 154-181, wherein the input anucleate cell is a reticulocyte.

Embodiment 184. The method of any one of embodiments 154-180, wherein the input anucleate cell is a platelet.

Embodiment 185. The method of any one of embodiments 154-184, wherein the input anucleate cell is a mammalian cell.

Embodiment 186. The method of any one of embodiments 154-185, wherein the input anucleate cell is a monkey, mouse, dog, cat, horse, rat, sheep, goat, pig, or rabbit cell.

Embodiment 187. The method of any one of embodiments 154-185, wherein the input anucleate cell is a human cell.

Embodiment 188. The method of any one of embodiments 154-187, wherein a half-life of the anucleate cell-derived vesicle following administration to a mammal is decreased compared to a half-life of the input anucleate cell following administration to the mammal.

Embodiment 189. The method of any one of embodiments 181-183, or 185-188, wherein a hemoglobin content of the anucleate cell-derived vesicle is decreased compared to the hemoglobin content of the input anucleate cell.

Embodiment 190. The method of any one of embodiments 181-189, wherein ATP production of the anucleate cell-derived vesicle is decreased compared to ATP production of the input anucleate cell.

Embodiment 191. The method of any one of embodiments 181-182 or 185-190, wherein the anucleate cell-derived vesicle exhibits one or more of the following properties: (a) a circulating half-life in a mammal that is decreased compared to the input anucleate cell; (b) decreased hemoglobin level compared to the input anucleate cell; (c) a spherical morphology; (d) increased surface phosphatidylserine levels compared to the input anucleate cell, (e) reduced ATP production compared to the input anucleate cell.

Embodiment 192. The method of any one of embodiments 181-182 or 185-191, wherein the input anucleate cell is an erythrocyte and wherein the anucleate cell-derived vesicle has a reduced biconcave shape compared to the input anucleate cell.

Embodiment 193. The method of embodiment 181-182 or 185-192, wherein the anucleate cell-derived vesicle is a red blood cell ghost.

Embodiment 194. The method of any one of embodiments 154-193, wherein the anucleate cell-derived vesicles prepared by the process have greater than about 1.5 fold more phosphatidylserine on its surface compared to the input anucleate cell.

Embodiment 195. The anucleate cell-derived vesicle of any one of embodiments 154-194, wherein a population profile of anucleate cell-derived vesicles prepared by the process exhibits higher average phosphatidylserine levels on the surface compared to the input anucleate cells.

Embodiment 196. The anucleate cell-derived vesicle of any one of embodiments 154-195, wherein at least 50% of the population profile of anucleate cell-derived vesicles prepared by the process exhibits higher phosphatidylserine levels on the surface compared to the input anucleate cells.

Embodiment 197. The anucleate cell-derived vesicle of any one of embodiments 154-196, wherein the anucleate cell-derived vesicle exhibits enhanced uptake in a tissue or cell compared to the input anucleate cell.

Embodiment 198. The anucleate cell-derived vesicle of embodiment 197, wherein the anucleate cell-derived vesicle exhibit enhanced uptake in liver and/or spleen or by a phagocytic cell and/or an antigen-presenting cell compared to the uptake of the input anucleate cell.

Embodiment 199. The anucleate cell-derived vesicle of any one of embodiments 154-198, wherein the anucleate cell-derived vesicle is modified to enhance uptake in a tissue or cell compared to the input anucleate cell.

Embodiment 200. The anucleate cell-derived vesicle of embodiment 199, wherein the anucleate cell-derived vesicle is modified to enhance uptake in liver and/or spleen or by a phagocytic cell and/or an antigen-presenting cell compared to the uptake of the input anucleate cell.

Embodiment 201. The anucleate cell-derived vesicle of any one of embodiments 154-200, wherein the anucleate cell-derived vesicle comprises CD47 on its surface.

Embodiment 202. The method of any one of embodiments 154-201, wherein the anucleate cell-derived vesicle is not (a) heat processed, (b) chemically treated, and/or (c) subjected to hypotonic or hypertonic conditions during the preparation of the anucleate cell-derived vesicles.

Embodiment 203. The method of any one of embodiments 154-202, wherein the osmolarity of the cell suspension is maintained throughout the process.

Embodiment 204. The method of embodiments 154-203, wherein the osmolarity of the cell suspension is maintained between about 200 mOsm and about 400 mOsm throughout the process.

Embodiment 205. The method of any one of embodiments 154-204, wherein the constriction is contained within a microfluidic channel.

Embodiment 206. The method of embodiment 205, wherein the microfluidic channel comprises a plurality of constrictions.

Embodiment 207. The method of embodiment 206, wherein the plurality of constrictions are arranged in series and/or in parallel.

Embodiment 208. The method of any one of embodiments 154-207, wherein the constriction is between a plurality of micropillars; between a plurality of micropillars configured in an array; or between one or more movable plates.

Embodiment 209. The method of any one of embodiments 154-208, wherein the constriction is a pore or contained within a pore.

Embodiment 210. The method of embodiment 209, wherein the pore is contained in a surface.

Embodiment 211. The method of embodiment 210, wherein the surface is a filter.

Embodiment 212. The method of embodiment 210, wherein the surface is a membrane.

Embodiment 213. The method of any one of embodiments 154-212, wherein the constriction size is a function of the diameter of the input anucleate cell in suspension.

Embodiment 214. The method of any one of embodiments 154-213, wherein the constriction size is about 10%, about 20%, about 30%, about 40%, about 50%, about 60%, or about 70% of the diameter of the input anucleate cell in suspension.

Embodiment 215. The method of any one of embodiments 154-214, wherein the constriction has a width of about 0.25 μm to about 4 μm.

Embodiment 216. The method of any one of embodiments 154-215, wherein the constriction has a width of about 4 μm, 3.5 μm, about 3 μm, about 2.5 μm, about 2 μm, about 1.5 μm, about 1 μm, about 0.5 μm, or about 0.25 μm.

Embodiment 217. The method of any one of embodiments 154-215, wherein the constriction has a width of about 2.2 μm.

Embodiment 218. The method of any one of embodiments 154-217, wherein the input anucleate cells are passed through the constriction under a pressure ranging from about 10 psi to about 90 psi.

Embodiment 219. The method of any one of embodiments 154-218, wherein said cell suspension is contacted with the antigen before, concurrently, or after passing through the constriction.

Embodiment 220. A composition comprising a population of anucleate cell-derived vesicles prepared by the method of any one of embodiments 154-219.

Embodiment 301. An anucleate cell-derived vesicle prepared from a parent anucleate cell, the anucleate cell-derived vesicle having one or more of the following properties: (a) a circulating half-life in a mammal is decreased compared to the parent anucleate cell, (b) decreased hemoglobin levels compared to the parent anucleate cell, (c) spherical morphology, (d) increased surface phosphatidylserine levels compared to the parent anucleate cell, or (e) reduced ATP production compared to the parent anucleate cell.

Embodiment 302. The anucleate cell-derived vesicle of embodiment 301, wherein the parent anucleate cell is a mammalian cell.

Embodiment 303. The anucleate cell-derived vesicle of embodiment 301 or 302, wherein the parent anucleate cell is human cell.

Embodiment 304. The anucleate cell-derived vesicle of any one of embodiments 301-303, wherein the parent anucleate cell is a red blood cell or a platelet.

Embodiment 305. The anucleate cell-derived vesicle of embodiment 304, wherein the red blood cell is an erythrocyte or a reticulocyte.

Embodiment 306. The anucleate cell-derived vesicle of any one of embodiments 301-305, wherein the circulating half-life of the anucleate cell-derived vesicle in a mammal is decreased compared to the parent anucleate cell.

Embodiment 307. The anucleate cell-derived vesicle of embodiment 306, wherein the circulating half-life in the mammal is decreased by more than about 50%, about 60%, about 70%, about 80%, about 90%, about 95%, about 96%, about 97%, about 98%, or about 99% compared to the parent anucleate cell.

Embodiment 308. The anucleate cell-derived vesicle of embodiment 307, wherein the parent anucleate cell is a human cell and wherein the circulating half-life of the anucleate cell-derived vesicle is less than about 1 minute, about 2 minutes, about 5 minutes, about 10 minutes, about 15 minutes, about 30 minutes, about 1 hour, about 6 hours, about 12 hours, about 1 day, about 2 days, about 3 days, about 4 days, about 5 days, about 10 days, about 25 days, about 50 days, about 75 days, about 100 days, about 120 days.

Embodiment 309. The anucleate cell-derived vesicle of any one of embodiments 301-308, wherein the parent anucleate cell is a red blood cell, wherein the hemoglobin levels in the anucleate cell-derived vesicle are decreased compared to the parent anucleate cell.

Embodiment 310. The anucleate cell-derived vesicle of embodiment 309, wherein the hemoglobin levels in the anucleate cell-derived vesicle are decreased by at least about 10%, about 20%, about 30%, about 40%, about 50%, about 60%, about 70%, about 80%, about 90%, about 99% or about 100% compared to the parent anucleate cell.

Embodiment 311. The anucleate cell-derived vesicle of embodiment 309, wherein the hemoglobin levels in the anucleate cell-derived vesicle are about 1%, about 5%, about 10%, about 20%, about 30%, about 40%, or about 50% the level of hemoglobin in the parent anucleate cell.

Embodiment 312. The anucleate cell-derived vesicle of any one of embodiments 301-311, wherein the parent anucleate cell is an erythrocyte and wherein the anucleate cell-derived vesicle is spherical in morphology.

Embodiment 313. The anucleate cell-derived vesicle of any one of embodiments 301-311, wherein the parent anucleate cell is an erythrocyte and wherein the anucleate cell-derived vesicle has a reduced biconcave shape compared to the parent anucleate cell.

Embodiment 314. The anucleate cell-derived vesicle of any one of embodiments 301-311, wherein the parent anucleate cell is a red blood cell or an erythrocyte and wherein the anucleate cell-derived vesicle is a red blood cell ghost (RBC ghost).

Embodiment 315. The anucleate cell-derived vesicle of any one of embodiments 301-312, wherein the anucleate cell-derived vesicle has increased surface phosphatidylserine levels compared to the parent anucleate cell.

Embodiment 316. The anucleate cell-derived vesicle of embodiments 315, wherein the anucleate cell-derived vesicles prepared by the process has greater than about 1.5 fold more phosphatidylserine on its surface compared to the parent anucleate cell.

Embodiment 317. The anucleate cell-derived vesicle of embodiment 315, wherein the anucleate cell-derived vesicle has about 10%, about 20%, about 30%, about 40%, about 50%, about 60%, about 70%, about 80%, about 90%, about 99%, about 100% or more than about 100% more phosphatidylserine on its surface compared to the parent anucleate cell.

Embodiment 318. The anucleate cell-derived vesicle of any one of embodiments 301-317, wherein the anucleate cell-derived vesicle has reduced ATP production compared to the parent anucleate cell.

Embodiment 319. The anucleate cell-derived vesicle of embodiment 318, wherein the anucleate cell-derived vesicle produces ATP at less than about 1%, about 5%, about 10%, about 20%, about 30%, about 40%, or about 50% the level of ATP produced by the parent anucleate cell.

Embodiment 320. The anucleate cell-derived vesicle of embodiment 319, wherein the anucleate cell-derived vesicle does not produce ATP.

Embodiment 321. The anucleate cell-derived vesicle of any one of embodiments 301-20, wherein the anucleate cell-derived vesicle is modified to enhance uptake in a tissue or cell compared to the parent anucleate cell.

Embodiment 322. The anucleate cell-derived vesicle of embodiment 321, wherein the anucleate cell-derived vesicle is modified to enhance uptake in liver or spleen or by a phagocytic cell or an antigen-presenting cell compared to the uptake of the parent anucleate cell.

Embodiment 323. The anucleate cell-derived vesicle of any one of embodiments 301-320, wherein the anucleate cell-derived vesicle comprises CD47 on its surface.

Embodiment 324. The anucleate cell-derived vesicle of any one of embodiments 301-319, wherein the parent anucleate cell was not (a) heat processed, (b) chemically treated, and/or (c) subjected to hypotonic or hypertonic conditions during the preparation of the anucleate cell-derived vesicles.

Embodiment 325. The anucleate cell-derived vesicle of any one of embodiments 301-324, wherein osmolarity was maintained during preparation of the anucleate cell-derived vesicle from the parent anucleate cell.

Embodiment 326. The anucleate cell-derived vesicle of embodiment 325, wherein the osmolarity was maintained between about 200 mOsm and about 600 mOsm.

Embodiment 327. The anucleate cell-derived vesicle of embodiment 325 or 326, wherein the osmolarity was maintained between about 200 mOsm and about 400 mOsm.

Embodiment 328. The anucleate cell-derived vesicle of any one of embodiments 301-327, wherein the anucleate cell-derived vesicle was prepared by a process comprising: passing a suspension comprising the input parent anucleate cells through a cell deforming constriction, wherein a diameter of the constriction is a function of a diameter of the input parent anucleate cell in the suspension, thereby causing perturbations of the anucleate cell large enough for a payload to pass through; thereby producing an anucleate cell-derived vesicle.

Embodiment 329. The anucleate cell-derived vesicle of any one of embodiments 301-328, wherein the anucleate cell-derived vesicle comprises a payload.

Embodiment 330. The anucleate cell-derived vesicle of embodiment 329, wherein the payload is a polypeptide, a nucleic acid, a lipid, a carbohydrate, a small molecule, a complex, a nanoparticle.

Embodiment 331. The anucleate cell-derived vesicle of embodiment 329, wherein the anucleate cell-derived vesicle was prepared by a process comprising: (a) passing a cell suspension comprising the input parent anucleate cell through a cell-deforming constriction, wherein a diameter of the constriction is a function of a diameter of the input parent anucleate cell in the suspension, thereby causing perturbations of the input parent anucleate cell large enough for the payload to pass through to form an anucleate cell-derived vesicle; and (b) incubating the anucleate cell-derived vesicle with the payload for a sufficient time to allow the payload to enter the anucleate cell-derived vesicle; thereby producing an anucleate cell-derived vesicle comprising the payload.

Embodiment 332. The anucleate cell-derived vesicle of any one of embodiments 301-331, wherein the anucleate cell-derived vesicle comprises an antigen.

Embodiment 333. The anucleate cell-derived vesicle of any one of embodiments 301-332, wherein the anucleate cell-derived vesicle comprises adjuvant.

Embodiment 334. The anucleate cell-derived vesicle of any one of embodiments 301-332, wherein the anucleate cell-derived vesicle comprises an antigen and/or a tolerogenic factor.

Embodiment 335. The anucleate cell-derived vesicle of embodiment 332, wherein the anucleate cell-derived vesicle was prepared by a process comprising: (a) passing a cell suspension comprising the input parent anucleate cell through a cell-deforming constriction, wherein a diameter of the constriction is a function of a diameter of the input parent anucleate cell in the suspension, thereby causing perturbations of the input parent anucleate cell large enough for the antigen to pass through to form an anucleate cell-derived vesicle; and (b) incubating the anucleate cell-derived vesicle with the antigen for a sufficient time to allow the antigen to enter the anucleate cell-derived vesicle; thereby producing an anucleate cell-derived vesicle comprising an antigen.

Embodiment 336. The anucleate cell-derived vesicle of embodiment 333, wherein the anucleate cell-derived vesicle was prepared by a process comprising: (a) passing a cell suspension comprising the input parent anucleate cell through a cell-deforming constriction, wherein a diameter of the constriction is a function of a diameter of the input parent anucleate cell in the suspension, thereby causing perturbations of the input parent anucleate cell large enough for the adjuvant to pass through to form an anucleate cell-derived vesicle; and (b) incubating the anucleate cell-derived vesicle with the adjuvant for a sufficient time to allow the adjuvant to enter the anucleate cell-derived vesicle; thereby producing an anucleate cell-derived vesicle comprising an adjuvant.

Embodiment 337. The anucleate cell-derived vesicle of embodiment 333, wherein the anucleate cell-derived vesicle comprises an antigen and an adjuvant, wherein the anucleate cell-derived vesicle was prepared by a process comprising: (a) passing a cell suspension comprising the input parent anucleate cell through a cell-deforming constriction, wherein a diameter of the constriction is a function of a diameter of the input parent anucleate cell in the suspension, thereby causing perturbations of the input parent anucleate cell large enough for the antigen and the adjuvant to pass through to form an anucleate cell-derived vesicle; and (b) incubating the anucleate cell-derived vesicle with the antigen and adjuvant for a sufficient time to allow the antigen and the adjuvant to enter the anucleate cell-derived vesicle; thereby producing an anucleate cell-derived vesicle comprising an antigen and an adjuvant.

Embodiment 338. The anucleate cell-derived vesicle of embodiment 334, wherein the anucleate cell-derived vesicle comprises an antigen and a tolerogenic factor, wherein the anucleate cell-derived vesicle was prepared by a process comprising: (a) passing a cell suspension comprising the input parent anucleate cell through a cell-deforming constriction, wherein a diameter of the constriction is a function of a diameter of the input parent anucleate cell in the suspension, thereby causing perturbations of the input parent anucleate cell large enough for the antigen and the tolerogenic factor to pass through to form an anucleate cell-derived vesicle; and (b) incubating the anucleate cell-derived vesicle with the antigen and the tolerogenic factor for a sufficient time to allow the antigen and the tolerogenic factor to enter the anucleate cell-derived vesicle; thereby producing an anucleate cell-derived vesicle comprising an antigen and an tolerogenic factor.

Embodiment 339. The anucleate cell-derived vesicle of any one of embodiments 328-338, wherein the constriction is contained within a microfluidic channel.

Embodiment 340. The anucleate cell-derived vesicle of embodiment 339, wherein the microfluidic channel comprises a plurality of constrictions.

Embodiment 341. The anucleate cell-derived vesicle of embodiment 340, wherein the plurality of constrictions are arranged in series and/or in parallel.

Embodiment 342. The anucleate cell-derived vesicle of any one of embodiments 328-341, wherein the constriction is between a plurality of micropillars, between a plurality of micropillars configured in an array, or between one or more movable plates.

Embodiment 343. The anucleate cell-derived vesicle of any one of embodiments 328-338, wherein the constriction is a pore or contained within a pore.

Embodiment 344. The anucleate cell-derived vesicle of embodiment 343, wherein the pore is contained in a surface.

Embodiment 345. The anucleate cell-derived vesicle of embodiment 344, wherein the surface is a filter.

Embodiment 346. The anucleate cell-derived vesicle of embodiment 344, wherein the surface is a membrane.

Embodiment 347. The anucleate cell-derived vesicle of any one of embodiments 328-346, wherein the constriction size is about 10%, about 20%, about 30%, about 40%, about 50%, about 60%, or about 70% of the cell diameter.

Embodiment 348. The anucleate cell-derived vesicle of any one of embodiments 328-346, wherein the constriction has a width of about 0.25 μm to about 4 μm.

Embodiment 349. The anucleate cell-derived vesicle of any one of embodiments 328-346, wherein the constriction has a width of about 4 μm, 3.5 μm, about 3 μm, about 2.5 μm, about 2 μm, about 1.5 μm, about 1 μm, about 0.5 μm, or about 0.25 μm.

Embodiment 350. The anucleate cell-derived vesicle of any one of embodiments 328-346, wherein the constriction has a width of about 2.2 μm.

Embodiment 351. The anucleate cell-derived vesicle of any one of embodiments 328-350, wherein the input parent anucleate cells are passed through the constriction under a pressure ranging from about 10 psi to about 150 psi.

Embodiment 352. The anucleate cell-derived vesicle of any one of embodiments 328-351, wherein said cell suspension is contacted with the payload before, concurrently, or after passing through the constriction.

Embodiment 353. The anucleate cell-derived vesicle of any one of embodiments 332-352, wherein the antigen is capable of being processed into an MHC class I-restricted peptide and/or an MHC class II-restricted peptide.

Embodiment 354. The anucleate cell-derived vesicle of any one of embodiments 332-353, wherein the antigen is a disease-associated antigen.

Embodiment 355. The anucleate cell-derived vesicle of any one of embodiments 332-354, wherein the antigen is a tumor antigen.

Embodiment 356. The anucleate cell-derived vesicle of any one of embodiments 332-354, wherein the antigen is derived from a lysate.

Embodiment 357. The anucleate cell-derived vesicle of embodiment 356, wherein the antigen is derived from a transplant lysate.

Embodiment 358. The anucleate cell-derived vesicle of embodiment 356, wherein the lysate is a tumor lysate.

Embodiment 359. The anucleate cell-derived vesicle of any one of embodiments 332-358, wherein the antigen is a viral antigen, a bacterial antigen or a fungal antigen.

Embodiment 360. The aculeate cell-derived vesicle of embodiment 359, wherein the viral antigen is a virus, a viral particle, or a viral capsid.

Embodiment 361. The anucleate cell-derived vesicle of any one of embodiments 332-354, wherein the antigen is a microorganism.

Embodiment 362. The anucleate cell-derived vesicle of any one of embodiments 332-361, wherein the antigen is a polypeptide.

Embodiment 363. The anucleate cell-derived vesicle of any one of embodiments 332-361, wherein the antigen is a lipid antigen.

Embodiment 364. The anucleate cell-derived vesicle of any one of embodiments 332-361, wherein the antigen is a carbohydrate antigen.

Embodiment 365. The anucleate cell-derived vesicle of any one of embodiments 332-361, wherein the antigen is a modified antigen.

Embodiment 366. The anucleate cell-derived vesicle of embodiment 365, wherein the modified antigen comprises an antigen fused with a polypeptide.

Embodiment 367. The anucleate cell-derived vesicle of embodiment 366, wherein the modified antigen comprises an antigen fused with a targeting peptide.

Embodiment 368. The anucleate cell-derived vesicle of embodiment 366, wherein the modified antigen comprises an antigen fused with a lipid.

Embodiment 369. The anucleate cell-derived vesicle of embodiment 366, wherein the modified antigen comprises an antigen fused with a carbohydrate.

Embodiment 370. The anucleate cell-derived vesicle of embodiment 366, wherein the modified antigen comprises an antigen fused with a nanoparticle.

Embodiment 371. The anucleate cell-derived vesicle of any one of embodiments 332-370, wherein a plurality of antigens is delivered to the anucleate cell.

Embodiment 372. The anucleate cell-derived vesicle of any one of embodiments 333, 336, 337, and 339 wherein the adjuvant is a CpG ODN, IFN-α, STING agonists, RIG-I agonists, poly I:C, imiquimod, resiquimod, and/or lipopolysaccharide (LPS).

Embodiment 373. A composition comprising a plurality of anucleate cell-derived vesicles of any one of embodiments 301-372.

Embodiment 374. A composition comprising a plurality of anucleate cell-derived vesicles prepared from parent anucleate cells, the composition having one or more of the following properties: (a) greater than about 20% of the anucleate cell-derived vesicles in the composition have a circulating half-life in a mammal that is decreased compared to the parent anucleate cell, (b) greater than 20% of the anucleate cell-derived vesicles in the composition have decreased hemoglobin levels compared to the parent anucleate cell, (c) greater than 20% of the anucleate cell-derived vesicles in the composition have spherical morphology, (d) greater than 20% of the anucleate cell-derived vesicles in the composition are RBC ghosts, (e) greater than 20% of the anucleate cell-derived vesicles in the composition vesicles in the composition have higher levels of phosphatidylserine compared to the population of parent anucleate cells, or (f) greater than 20% of the anucleate cell-derived vesicles in the composition have reduced ATP production compared to the parent anucleate cell.

Embodiment 375. A composition comprising a plurality of anucleate cell-derived vesicles prepared from a population of a parent anucleate cell, the composition having one or more of the following properties: (a) greater than about 20% of the anucleate cell-derived vesicles in the composition have a circulating half-life in a mammal that is decreased compared to the average of the population of the parent anucleate cell, (b) greater than 20% of the anucleate cell-derived vesicles in the composition have decreased hemoglobin levels compared to the average of the population of the parent anucleate cell, (c) greater than 20% of the anucleate cell-derived vesicles in the composition have spherical morphology, (d) greater than 20% of the anucleate cell-derived vesicles in the composition are RBC ghosts, (e) greater than 20% of the anucleate cell-derived vesicles in the composition vesicles in the composition have higher levels of phosphatidylserine compared to the average of the population of the parent anucleate cell, or (f) greater than 20% of the anucleate cell-derived vesicles in the composition have reduced ATP production compared to the average of the population of the parent anucleate cell.

Embodiment 376. The composition of embodiment 374 or 375, wherein the parent anucleate cell is a mammalian cell.

Embodiment 377. The composition of any one of embodiments 374-376, wherein the parent anucleate cell is human cell.

Embodiment 378. The composition of any one of embodiments 374-377, wherein the parent anucleate cell is a red blood cell or a platelet.

Embodiment 379. The composition of embodiment 378, where the red blood cell is an erythrocyte or a reticulocyte.

Embodiment 380. The composition of any one of embodiments 374-378, wherein the circulating half-life of 20% of the anucleate cell-derived vesicles in the composition in a mammal is decreased compared to the parent anucleate cell or the average of the population of the parent anucleate cell.

Embodiment 381. The composition of embodiment 380, wherein the circulating half-life of 20% of the anucleate cell-derived vesicles in the composition in the mammal is decreased by more than about 50%, about 60%, about 70%, about 80% or about 90% compared to the parent anucleate cell or the average of the population of the parent anucleate cell.

Embodiment 382. The composition of embodiment 381, wherein the parent anucleate cell is a human cell and wherein the circulating half-life of 20% of the anucleate cell-derived vesicles in the composition is less than about 5 minutes, about 10 minutes, about 15 minutes, about 30 minutes, about 1 hour, about 6 hours, about 12 hours, about 1 day, about 2 days, about 3 days, about 4 days, about 5 days, about 10 days.

Embodiment 383. The composition of any one of embodiments 374-382, wherein the parent anucleate cell is a red blood cell and wherein the hemoglobin levels of 20% of the anucleate cell-derived vesicles in the composition are decreased compared to the parent anucleate cell or the average of the population of the parent anucleate cell.

Embodiment 384. The composition of embodiment 383, wherein the hemoglobin levels of 20% of the anucleate cell-derived vesicles in the composition of the anucleate cell-derived vesicle are decreased by at least about 10%, about 20%, about 30%, about 40%, about 50%, about 60%, about 70%, about 80%, about 90%, about 99% or about 100% compared to the parent anucleate cell or the average of the population of the parent anucleate cell.

Embodiment 385. The composition of embodiment 384, wherein the hemoglobin levels of 20% of the anucleate cell-derived vesicles in the composition are about 1%, about 5%, about 10%, about 20%, about 30%, about 40%, or about 50% the level of hemoglobin in the parent anucleate cell or the average of the population of the parent anucleate cell.

Embodiment 386. The composition of any one of embodiments 374-385, wherein the parent anucleate cell is an erythrocyte and wherein greater than 20% of the anucleate cell-derived vesicles in the composition are spherical in morphology.

Embodiment 387. The composition of any one of embodiments 374-385, wherein the parent anucleate cell is an erythrocyte and wherein greater than 20% of the anucleate cell-derived vesicles in the composition have a reduced biconcave shape compared to the parent anucleate cell.

Embodiment 388. The composition of any one of embodiments 374-386, wherein the parent anucleate cell is a red blood cell or an erythrocyte and wherein greater than 20% of the anucleate cell-derived vesicles in the composition are red blood cell ghosts.

Embodiment 389. The composition of any one of embodiments 374-388, wherein greater than 20% of the anucleate cell-derived vesicles in the composition comprise surface phosphatidylserine.

Embodiment 390. The composition of any one of embodiments 374-389, wherein greater than 20% of the anucleate cell-derived vesicles in the composition comprise increased surface phosphatidylserine levels compared to the parent anucleate cells or the average of the population of the parent anucleate cell.

Embodiment 391. The composition of embodiment 390, wherein greater than 20% of the anucleate cell-derived vesicles in the composition have about 10%, about 20%, about 30%, about 40%, about 50%, about 60%, about 70%, about 80%, about 90%, about 99%, about 100% or more than about 100% higher surface phosphatidylserine levels compared to a composition comprising a plurality of parent anucleate cells.

Embodiment 392. The composition of any one of embodiments 374-391, wherein greater than 20% of the anucleate cell-derived vesicles in the composition have reduced ATP production compared to the parent anucleate cell or the average of the population of the parent anucleate cell.

Embodiment 393. The composition of embodiment 392, wherein greater than 20% of the anucleate cell-derived vesicles in the composition produce ATP at less than about 1%, about 5%, about 10%, about 20%, about 30%, about 40%, or about 50% the level of ATP produced by the parent anucleate cell or the average of the population of the parent anucleate cell.

Embodiment 394. The composition of embodiment 393, wherein the anucleate cell-derived vesicle does not produce ATP.

Embodiment 395. The composition of any one of embodiments 374-393, wherein the parent anucleate cell was not (a) heat processed, (b) chemically treated, and/or (c) subjected to hypotonic or hypertonic conditions during the preparation of the compositions.

Embodiment 396. The composition of any one of embodiments 301-324, wherein osmolarity was maintained during preparation of the anucleate cell-derived vesicles from the parent anucleate cell.

Embodiment 397. The composition of embodiment 396, wherein the osmolarity was maintained between about 200 mOsm and about 600 mOsm.

Embodiment 398. The composition of embodiment 396 or 397, wherein the osmolarity was maintained between about 200 mOsm and about 400 mOsm.

Embodiment 399. The composition of any one of embodiments 374-398, wherein the anucleate cell-derived vesicles of the composition were prepared by a process comprising: passing a suspension comprising the input parent anucleate cells through a cell deforming constriction, wherein a diameter of the constriction is a function of a diameter of the input parent anucleate cells in the suspension, thereby causing perturbations of the anucleate cells large enough for a payload to pass through; thereby producing the anucleate cell-derived vesicles.

Embodiment 400. The composition of any one of embodiments 374-399, wherein the anucleate cell-derived vesicles of the composition comprise a payload.

Embodiment 401. The composition of embodiment 400, wherein the payload is a therapeutic payload.

Embodiment 402. The composition of embodiment 400, wherein the payload is a polypeptide, a nucleic acid, a lipid, a carbohydrate a small molecule, a complex, a nanoparticle.

Embodiment 403. The composition of embodiment 402, wherein the anucleate cell-derived vesicles of the composition were prepared by a process comprising: (a) passing a cell suspension comprising the input parent anucleate cells through a cell-deforming constriction, wherein a diameter of the constriction is a function of a diameter of the input parent anucleate cells in the suspension, thereby causing perturbations of the input parent anucleate cells large enough for the payload to pass through to form an anucleate cell-derived vesicles; and (b) incubating the anucleate cell-derived vesicles with the payload for a sufficient time to allow the payload to enter the anucleate cell-derived vesicles; thereby producing an anucleate cell-derived vesicles comprising the payload.

Embodiment 404. The composition of any one of embodiments 374-403, wherein the anucleate cell-derived vesicles comprise an antigen.

Embodiment 405. The composition of any one of embodiments 374-404, wherein the anucleate cell-derived vesicles comprise an adjuvant.

Embodiment 406. The composition of any one of embodiments 374-404, wherein the anucleate cell-derived vesicles comprise an antigen and a tolerogenic factor.

Embodiment 407 The composition of embodiment 404, wherein the anucleate cell-derived vesicles of the composition were prepared by a process comprising: (a) passing a cell suspension comprising the input parent anucleate cell through a cell-deforming constriction, wherein a diameter of the constriction is a function of a diameter of the input parent anucleate cell in the suspension, thereby causing perturbations of the input parent anucleate cell large enough for the antigen to pass through to form an anucleate cell-derived vesicle; and (b) incubating the anucleate cell-derived vesicle with the antigen for a sufficient time to allow the antigen to enter the anucleate cell-derived vesicle; thereby producing an anucleate cell-derived vesicle comprising an antigen.

Embodiment 408. The composition of embodiment 405, wherein the anucleate cell-derived vesicles of the composition were prepared by a process comprising: (a) passing a cell suspension comprising the input parent anucleate cell through a cell-deforming constriction, wherein a diameter of the constriction is a function of a diameter of the input parent anucleate cell in the suspension, thereby causing perturbations of the input parent anucleate cell large enough for the adjuvant to pass through to form an anucleate cell-derived vesicle; and (b) incubating the anucleate cell-derived vesicle with the adjuvant for a sufficient time to allow the adjuvant to enter the anucleate cell-derived vesicle; thereby producing an anucleate cell-derived vesicle comprising an adjuvant.

Embodiment 409. The composition of embodiment 405, wherein the anucleate cell-derived vesicles of the composition comprises an antigen and an adjuvant, wherein the anucleate cell-derived vesicles of the composition were prepared by a process comprising: (a) passing a cell suspension comprising the input parent anucleate cell through a cell-deforming constriction, wherein a diameter of the constriction is a function of a diameter of the input parent anucleate cell in the suspension, thereby causing perturbations of the input parent anucleate cell large enough for the antigen and the adjuvant to pass through to form an anucleate cell-derived vesicle; and (b) incubating the anucleate cell-derived vesicle with the antigen and the adjuvant for a sufficient time to allow the antigen and the adjuvant to enter the anucleate cell-derived vesicle; thereby producing an anucleate cell-derived vesicle comprising an antigen and/or an adjuvant.

Embodiment 410. The composition of embodiment 406, wherein the composition comprises an antigen and a tolerogenic factor, wherein the anucleate cell-derived vesicles of the composition were prepared by a process comprising: (a) passing a cell suspension comprising the input parent anucleate cell through a cell-deforming constriction, wherein a diameter of the constriction is a function of a diameter of the input parent anucleate cell in the suspension, thereby causing perturbations of the input parent anucleate cell large enough for the antigen and the tolerogenic factor to pass through to form an anucleate cell-derived vesicle; and (b) incubating the anucleate cell-derived vesicle with the antigen and the tolerogenic factor for a sufficient time to allow the antigen and the tolerogenic factor to enter the anucleate cell-derived vesicle; thereby producing an anucleate cell-derived vesicle comprising an antigen and/or an tolerogenic factor.

Embodiment 411. The composition of any one of embodiments 399, 403, and 407-410, wherein the constriction is contained within a microfluidic channel.

Embodiment 412. The composition of embodiment 384, wherein the microfluidic channel comprises a plurality of constrictions.

Embodiment 413. The composition of embodiment 412, wherein the plurality of constrictions are arranged in series and/or in parallel.

Embodiment 414. The composition of any one of embodiments 400-413, wherein the constriction is between a plurality of micropillars, between a plurality of micropillars configured in an array, or between one or more movable plates.

Embodiment 415. The composition of any one of embodiments 399, 403, and 407-410, wherein the constriction is a pore or contained within a pore.

Embodiment 416. The composition of embodiment 415, wherein the pore is contained in a surface.

Embodiment 417. The composition of embodiment 416, wherein the surface is a filter.

Embodiment 418. The composition of embodiment 416, wherein the surface is a membrane.

Embodiment 419. The composition of any one of embodiments 399-418, wherein the constriction size is about 10%, about 20%, about 30%, about 40%, about 50%, about 60%, or about 70% of the cell diameter.

Embodiment 420. The composition of any one of embodiments 399-419, wherein the constriction has a width of about 0.25 μm to about 4 μm.

Embodiment 421. The composition of any one of embodiments 399-420, wherein the constriction has a width of about 4 μm, 3.5 μm, about 3 μm, about 2.5 μm, about 2 μm, about 1.5 μm, about 1 μm, about 0.5 μm, or about 0.25 μm.

Embodiment 422. The composition of any one of embodiments 399-420, wherein the constriction has a width of about 2.2 μm.

Embodiment 423. The composition of any one of embodiments 399-422, wherein the input parent anucleate cells are passed through the constriction under a pressure ranging from about 10 psi to about 150 psi.

Embodiment 424. The composition of any one of embodiments 399-423, wherein said cell suspension is contacted with the antigen before, concurrently, or after passing through the constriction.

Embodiment 425. The composition of any one of embodiments 404-424, wherein the antigen is capable of being processed into an MHC class I-restricted peptide and/or an MHC class II-restricted peptide.

Embodiment 426. The composition of any one of embodiments 404-425, wherein the antigen is a disease-associated antigen.

Embodiment 427. The composition of any one of embodiments 404-426, wherein the antigen is a tumor antigen.

Embodiment 428. The composition of any one of embodiments 404-427, wherein the antigen is derived from a lysate.

Embodiment 429. The composition of embodiment 428, wherein the lysate is a transplant lysate.

Embodiment 430. The composition of embodiment 428, wherein the lysate is a tumor lysate.

Embodiment 431. The composition of any one of embodiments 404-426, wherein the antigen is a viral antigen, a bacterial antigen or a fungal antigen.

Embodiment 432. The composition of any one of embodiments 404-426, wherein the antigen is a microorganism.

Embodiment 433. The composition of any one of embodiments 404-432, wherein the antigen is a polypeptide.

Embodiment 434. The composition of any one of embodiments 404-433, wherein the antigen is a lipid antigen.

Embodiment 435. The composition of any one of embodiments 404-433, wherein the antigen is a carbohydrate antigen.

Embodiment 436. The composition of any one of embodiments 404-433, wherein the antigen is a modified antigen.

Embodiment 437. The composition of embodiment 436, wherein the modified antigen comprises an antigen fused with a polypeptide.

Embodiment 438. The composition of embodiment 437, wherein the modified antigen comprises an antigen fused with a targeting peptide.

Embodiment 439. The composition of embodiment 437, wherein the modified antigen comprises an antigen fused with a lipid.

Embodiment 440. The composition of embodiment 437, wherein the modified antigen comprises an antigen fused with a carbohydrate.

Embodiment 441. The composition of embodiment 437, wherein the modified antigen comprises an antigen fused with a nanoparticle.

Embodiment 442. The composition of any one of embodiments 404-441, wherein a plurality of antigens is delivered to the anucleate cell.

Embodiment 443. The composition of any one of embodiments 405, 408, 409, and 411-442 wherein the adjuvant is a CpG ODN, IFN-α, STING agonists, RIG-I agonists, poly I:C, imiquimod, resiquimod, and/or LPS.

Embodiment 444. The composition of any one of embodiments 373-443, wherein the composition is a pharmaceutical composition.

Embodiment 445. A method of making a composition comprising a plurality of anucleate cell-derived vesicles prepared from parent anucleate cells, the composition having one or more of the following properties: (a) greater than 20% of the anucleate cell-derived vesicles in the composition have a circulating half-life in a mammal that is decreased compared to the parent anucleate cell, (b) greater than 20% of the anucleate cell-derived vesicles in the composition have decreased hemoglobin levels compared to the parent anucleate cell, (c) greater than 20% of the anucleate cell-derived vesicles in the composition have spherical morphology, (d) greater than 20% of the anucleate cell-derived vesicles in the composition are RBC ghosts, (e) greater than 20% of the anucleate cell-derived vesicles in the composition have higher levels of phosphatidylserine, or (f) greater than 20% of the anucleate cell-derived vesicles in the composition have reduced ATP production compared to the parent anucleate cell; the method comprising passing a cell suspension comprising the parent anucleate cell through a cell-deforming constriction, wherein a diameter of the constriction is a function of a diameter of the parent anucleate cell in the suspension, thereby causing perturbations of the parent anucleate cell large enough for a payload to pass through to form an anucleate cell-derived vesicle, thereby producing an anucleate cell-derived vesicle.

Embodiment 446. The method of embodiment 445, wherein the constriction is contained within a microfluidic channel.

Embodiment 447. The method of embodiment 446, wherein the microfluidic channel comprises a plurality of constrictions.

Embodiment 448. The method of embodiment 447, wherein the plurality of constrictions are arranged in series and/or in parallel.

Embodiment 449. The anucleate cell-derived vesicle of any one of embodiments 445-448, wherein the constriction is between a plurality of micropillars, between a plurality of micropillars configured in an array, or between one or more movable plates.

Embodiment 450. The method of embodiment 446 or 447, wherein the constriction is a pore or contained within a pore.

Embodiment 451. The method of embodiment 450, wherein the pore is contained in a surface.

Embodiment 452. The method of embodiment 451, wherein the surface is a filter.

Embodiment 453. The method of embodiment 451, wherein the surface is a membrane.

Embodiment 454. The method of any one of embodiments 445-453, wherein the constriction size is about 10%, about 20%, about 30%, about 40%, about 50%, about 60%, or about 70% of the cell diameter.

Embodiment 455. The method of any one of embodiments 445-454, wherein the constriction has a width of about 0.25 μm to about 4 μm.

Embodiment 456. The method of any one of embodiments 445-454, wherein the constriction has a width of about 4 μm, 3.5 μm, about 3 μm, about 2.5 μm, about 2 μm, about 1.5 μm, about 1 μm, about 0.5 μm, or about 0.25 μm.

Embodiment 457. The method of any one of embodiments 445-454, wherein the constriction has a width of about 2.2 μm.

Embodiment 458. The method of any one of embodiments 445-457, wherein the input parent anucleate cells are passed through the constriction under a pressure ranging from about 10 psi to about 150 psi.

Embodiment 459. The method of any one of embodiments 445-458, wherein said cell suspension is contacted with a payload before, concurrently, or after passing through the constriction such that the payload enters the cell.

Embodiment 460. The method of embodiment 459, wherein the payload is a therapeutic payload.

Embodiment 461. The method of embodiment 459 or 460, wherein the payload is a polypeptide, a nucleic acid, a lipid, a carbohydrate a small molecule, a complex, or a nanoparticle.

Embodiment 462. The method of any one of embodiments 459-461, wherein the payload is an antigen and/or an adjuvant.

Embodiment 463. The method of any one of embodiments 459-461, wherein the payload is an antigen and/or a tolerogenic factor.

Embodiment 464. A method for treating a disease or disorder in an individual in need thereof, the method comprising administering the anucleate cell-derived vesicle of any one of embodiments 301-372.

Embodiment 465. A method for treating a disease or disorder in an individual in need thereof, the method comprising administering the composition of any one of embodiments 373-444.

Embodiment 466. The method of embodiment 464 or 465, wherein the anucleate cell-derived vesicles comprise a therapeutic payload.

Embodiment 467. The method of embodiment 466, wherein the individual has cancer and wherein the payload comprises an antigen.

Embodiment 468. The method of embodiment 466 or 467, wherein the individual has cancer and wherein the payload comprises an antigen and an adjuvant.

Embodiment 469. The method of embodiment of embodiment 467 or 468, wherein the antigen is a tumor antigen.

Embodiment 470. The method of embodiment 466, wherein the individual has an infectious disease or a viral-associated disease and wherein the payload comprises an antigen.

Embodiment 471. The method of embodiment 466 or 470, wherein the individual has an infectious disease or a viral-associated disease and wherein the payload comprises an antigen and an adjuvant.

Embodiment 472. The method of embodiment 471 wherein the antigen is a viral antigen, a bacterial antigen or a fungal antigen.

Embodiment 473. The method of embodiment 466, wherein the individual has an autoimmune disease and wherein the payload comprises an antigen.

Embodiment 474. The method of embodiment 466 or 473, wherein the individual has an autoimmune disease and wherein the payload comprises an antigen and/or a tolerogenic factor.

Embodiment 475. A method for preventing a disease or disorder in an individual in need thereof, the method comprising administering the anucleate cell-derived vesicle of any one of embodiments 301-372.

Embodiment 476. A method for preventing a disease or disorder in an individual in need thereof, the method comprising administering the composition of any one of embodiments 373-444.

Embodiment 477. The method of embodiment 475 or 476, wherein the anucleate cell-derived vesicles comprise an antigen.

Embodiment 478. The method of embodiment 475 or 476, wherein the individual has cancer and wherein the payload comprises an antigen and an adjuvant.

Embodiment 479. The method of embodiment 477 or 478, wherein the disease or disorder is cancer and the antigen is a tumor antigen.

Embodiment 480. The method of embodiment 477 or 478, wherein the individual has an infectious disease and wherein the payload comprises an antigen.

Embodiment 481. The method of embodiment 477 or 478, wherein the individual has an infectious disease and wherein the payload comprises an antigen and an adjuvant

Embodiment 482. The method of embodiment 481, wherein the antigen is a viral antigen, a bacterial antigen or a fungal antigen.

### EXAMPLES

The following examples are given for the purpose of illustrating various embodiments of the disclosure and are not meant to limit the present disclosure in any fashion. One skilled in the art will appreciate readily that the present disclosure is well adapted to carry out the objects and obtain the ends and advantages mentioned, as well as those objects, ends and advantages inherent herein. Changes therein and other uses which are encompassed within the spirit of the disclosure as defined by the scope of the claims will occur to those skilled in the art.

### Example 1

This example demonstrates, in part, that anucleate cell-derived vesicles comprising loaded antigen and/or adjuvant can induce an in vivo antigen-specific immune response.

### Materials and Methods

To determine in vivo antigen-specific immune response, cell-derived vesicles treated according to the conditions in Table 1, such as red blood cell-derived vesicles loaded with a model antigen and/or adjuvant, were administered to mice and then the number of antigen-specific T cells and the levels of inflammatory cytokines, IFN-γ and IL-2, were measured by flow cytometry. Specifically, red blood cells (RBCs) were obtained from C57BL/6J donor mice, and loaded intracellularly with a fluorescently-tagged IgG antibody (IgG488, 20 μg/mL), Ova protein (200 μg/mL), and/or polyinosinic:polycytidylic acid (poly I:C) (300 μg/mL), with or without systemic treatment with free Ova (10 μg/mouse) and/or poly I:C (25 μg/mouse), according to Groups A-H (5 mice/group) detailed in Table 1. In Table 1, conditions in parentheses following RBC indicate intracellular cargo and conditions outside of parentheses following RBC were co-administered systemically (i.e., not intracellular cargo).

Groups administered RBCs received a dose of 150 million (M) RBCs. Negative control animals received 150 M RBCs either: incubated with Ova (200 μg/mL), washed and co-injected into mice with free poly I:C (Endo+poly I:C) (Group A); loaded with antibody alone (RBC (IgG488) (Group B); or loaded with antibody and antigen (RBC (IgG488+Ova) (Group D). Positive control animals received 150 M RBCs either: loaded with antibody alone and co-administered with free Ova and poly I:C (RBC (IgG488)+Ova+poly I:C) (Group C); or 1 M dendritic cells (DCs) pulsed with the minimal epitope of Ova (SIINFEKL—1 μg/mL) (Group H). On Day 0, compositions respective to each treatment conditions were adoptively transferred to recipient C57BL/6J mice. On Day 7, spleens were harvested, restimulated with SIINFEKL (1 μg/mL) for Ova-specific tetramer, and subjected to intracellular cytokine staining (ICS) for IFN-γ and IL-2 according to the description below.

TABLE 1
Treatment groups.

| | | Free OVA | Free Poly I:C |
|---|---|---|---|
| Group | Condition* | per animal | per animal |
| A | Endo + Poly I:C | 0 | 25 μg |
| B | RBC (IgG488) | 0 | 0 |
| C | RBC (IgG488) + OVA + | 10 μg | 25 μg |
| | Poly I:C | | |
| D | RBC (IgG488 + OVA) | 0 | 0 |
| E | RBC (IgG488 + Poly I:C) | 0 | 0 |
| F | RBC (IgG488 + OVA) + | 0 | 25 μg |
| | Poly I:C | | |
| G | RBC (IgG488 + OVA + | 0 | 0 |
| | Poly I:C) | | |
| H | Pulsed DCs | 0 | 0 |

*Conditions in parentheses following RBC indicate intracellular cargo; conditions outside parentheses following RBC were co-administered systematically.

### Results

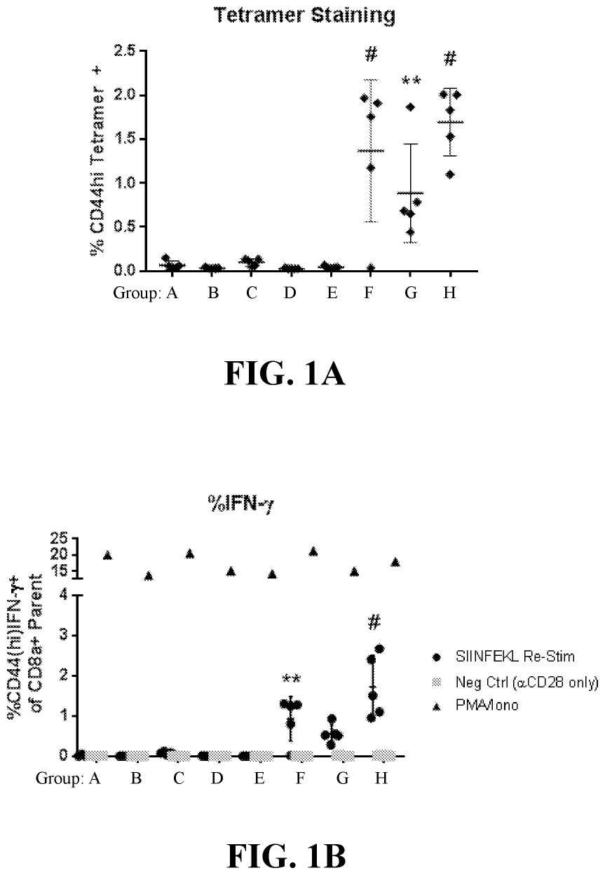

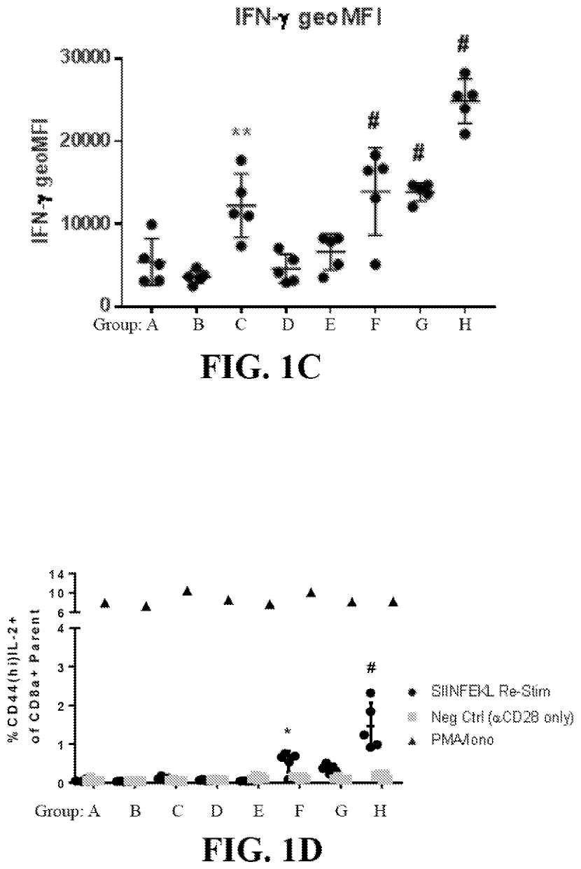

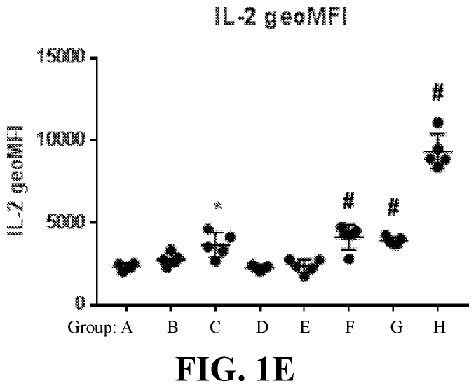

The number of antigen-specific T cells was measured via tetramer staining as discussed above. Group F (RBC (IgG488+Ova)+poly I:C), Group G (RBC (IgG488+Ova+poly I:C)), and the positive control pulsed DCs (Group H) induced statistically significant increases in Ova-reactive T cells (FIG. 1A). These data showed that there is a requirement for antigen and adjuvant, with the adjuvant either co-encapsulated or systemically administered, to induce an antigen-specific response as determined by tetramer staining. The percentage of cells having IFN-γ was significantly increased in Group F (RBC (IgG488+Ova)+poly I:C) and Group H (pulsed DCs), while there was also a slight statistically insignificant increase in Group G (RBC (IgG488+Ova+poly I:C)) (FIG. 1B). The amount of IFN-γ per cell increased in a statistically significant manner in the same groups as observed in the % of IFN-γ+ cells (Groups F and H), while there was also a significant increase in Group C (RBC (IgG488)+Ova+poly I:C) and Group G (RBC (IgG488+Ova+poly I:C) (FIG. 1C). Similar to the trend observed with IFN-γ, the percentage of IL-2+ cells was only significantly increased in pulsed DCs (Group H) and RBC (IgG488+Ova)+poly I:C (Group F) (FIG. 1D). The levels of IL-2 per cell was significantly increased in the pulsed DCs (Group H) and RBC (IgG488+Ova)+poly I:C (Group F), as well as the RBC (IgG488)+Ova+poly I:C (Group C) and RBC (IgG488+Ova+poly I:C) (Group G) conditions, which is the same trend observed with the amount of IFN-γ per cell (FIG. 1E). Taken together, these data show that anucleate cell-derived vesicles can induce an antigen-specific response, with a requirement for antigen and adjuvant (encapsulated or not) and the best response was observed in the RBC (IgG488+Ova)+poly I:C condition (Group F). All comparisons were made to Endo+poly I:C negative control (Group A) (5 mice/group, *P<0.05, **P<0.01, #P<0.005).

### Example 2

This example demonstrates, in part, that different doses of anucleate cell-derived vesicles comprising loaded antigen and/or adjuvant can induce varying levels of an in vivo antigen-specific immune response. Specifically, higher doses of anucleate cell-derived vesicles comprising loaded antigen and/or adjuvant can induced a greater in vivo antigen-specific immune response.

### Materials and Methods

To determine in vivo antigen-specific immune response, cell-derived vesicles treated according to the conditions in Table 2, such as red blood cell-derived vesicles loaded with a model antigen and/or adjuvant, were administered to mice and then the number of antigen-specific T cells and the levels of inflammatory cytokines, IFN-γ and IL-2, were measured by flow cytometry. Specifically, red blood cells (RBCs) were obtained from C57BL/6J donor mice, and loaded with a fluorescently-tagged IgG antibody (IgG488, 20 μg/mL), Ova protein (200 μg/mL) and/or poly I:C (300 μg/mL), with or without systemic treatment with free Ova (10 μg/mouse) and/or poly I:C (25 μg/mouse), according to the groups (5 mice/group) detailed in Table 2. In Table 2, conditions in parentheses following RBC indicate intracellular cargo and conditions outside of parentheses following RBC were co-administered systemically (i.e., not intracellular cargo).

Various combinations of loaded and systemically administered free antigen and adjuvant were compared. Negative control animals received 150 M red blood cells incubated with Ova (200 μg/mL), washed and co-injected into mice with free poly I:C (Endo+poly I:C) (Group I). Positive control animals received 1 M dendritic cells (DCs) pulsed with the minimal epitope of Ova (SIINFEKL—1 μg/mL) (Group N). On Day 0, loaded anucleate cell-derived vesicles, incubated red blood cells, or dendritic cells were adoptively transferred to recipient C57BL/6J mice. On Day 7, spleens were harvested, restimulated with SIINFEKL (1 μg/mL) for Ova-specific tetramer, and subjected to intracellular cytokine staining (ICS) for IFN-γ and IL-2.

TABLE 2
Treatment groups.

| | | Free OVA | Free Poly I:C |
|---|---|---|---|
| Group | Condition* | per animal | per animal |
| I | 150M Endo + Poly I:C | 0 | 25 μg |
| J | 150M RBC (IgG488) + OVA + | 10 μg | 25 μg |
| | Poly I:C | | |
| K | 150M RBC (IgG488 + OVA) + | 0 | 25 μg |
| | Poly I:C | | |
| L | 150M RBC (IgG488 + OVA + | 0 | 0 |
| | Poly I:C) | | |
| M | 1B RBC (IgG488 + OVA + | 0 | 0 |
| | Poly I:C) | | |
| N | Pulsed DCs | 0 | 0 |

*Conditions in parentheses following RBC indicate intracellular cargo; conditions outside parentheses following RBC were co-administered systematically.

### Results

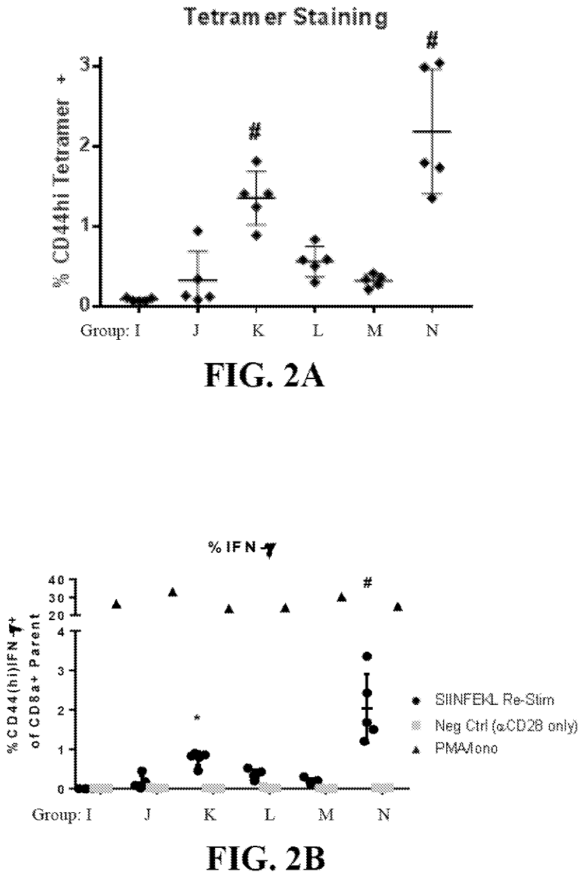

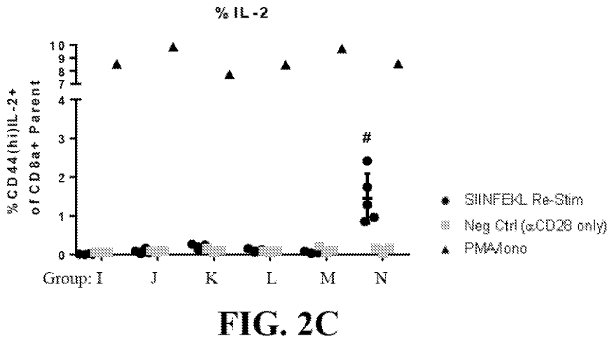

RBC (IgG488+Ova)+poly I:C (Group K) and the positive control pulsed DC (Group N) induced statistically significant increases in Ova-reactive T cells (FIG. 2A). The conditions where anucleate cell-derived vesicles contained only antibody and co-administered with free Ova and poly I:C (RBC (IgG488)+Ova+poly I:C; Group J), as well as the vesicles containing antibody, Ova, and poly I:C (RBC (IgG488+Ova+poly I:C)) at both the 150 million cells/animal (Group L) and the 1 billion (B) cell/animal (Group M) exhibited slight but statistically insignificant increases in the percentage of Ova-specific T cells (FIG. 2A). Interestingly, the higher dose of loaded vesicles (1 B RBC (IgG488+Ova+poly I:C) (Group M)) led to a lower endogenous response relative to the lower 150 M dose (Group L). The percentage of IFN-γ+ cells trended similarly to the tetramer data, with only the pulsed DC positive control (Group N) and RBC (IgG488+Ova)+poly I:C (Group K) conditions leading to significant increases in the proportion of IFN-γ+ cells (FIG. 2B). There was also a slight increase in the percentage of IFN-γ+ cells in the lower dose of RBC (IgG488+Ova+poly I:C) (Group L), but it was not significant (FIG. 2B). However, the percentage of IL-2+ cells only significantly increased in the positive control (FIG. 2C). Taken together, these data show that the best response was obtained with vesicles loaded with antigen and systemic co-administration of free poly I:C, and that unexpectedly the higher dose of antigen+adjuvant loaded vesicles (1 B) (Group M) led to a lower endogenous response. All comparisons were made to Endo+poly I:C negative control (Group I) (5 mice/group, *P<0.05, **P<0.01, #P<0.005).

### Example 3

This example demonstrates, in part, the effect of using different adjuvants or dosing strategies on in vivo antigen-specific immune response.

### Materials and Methods

To determine in vivo antigen-specific immune response, cell-derived vesicles treated according to the conditions in Table 3, such as red blood cell-derived vesicles loaded with a model antigen and/or adjuvant, were administered to mice and then the number of antigen-specific T cells and the levels of inflammatory cytokines, IFN-γ and IL-2, were measured by flow cytometry. Specifically, red blood cells were obtained from C57BL/6J donor mice, and loaded with a fluorescently-tagged IgG antibody (IgG488, 20 μg/mL), Ova protein (200 μg/mL) and/or an adjuvant (either poly I:C (300 or 3000 μg/mL), lipopolysaccharide (LPS, 300 μg/mL), or R848 (100 μg/mL)) at varying doses and prime-boost schedules, according to the groups (5 mice/group) as detailed in Table 3.

TABLE 3
Treatment groups.

| | | RBCs per | | Adjuvant SQZ |
|---|---|---|---|---|
| Group | Condition* | animal (M) | Adjuvant | conc (mg/mL) |
| O | Endo + poly I:C | 150 | Poly I:C | — |
| P | RBC (IgG488 + | 15 | Poly I:C | 0.3 |
| | Ova + poly I:C) | | | |
| Q | RBC (IgG488 + | 150 | Poly I:C | 0.3 |
| | Ova + poly I:C) | | | |
| R | RBC (IgG488 + | 150 | Poly I:C | 0.3 |
| | Ova + poly I:C) + | | | |
| | Boost | | | |
| S | RBC (IgG488 + | 150 | Poly I:C | 3 |
| | Ova + high dose | | | |
| | poly I:C) | | | |
| T | RBC (IgG488 + | 150 | LPS | 0.3 |
| | Ova + LPS) | | | |
| U | SQZ (IgG488 + | 150 | R-848 | 0.1 |
| | Ova + R848) | | | |

*Conditions in parentheses following RBC indicate intracellular cargo; conditions outside parentheses following RBC were co-administered systematically..

Negative control animals received red blood cells incubated with Ova (200 μg/mL), washed and co-injected into mice with free poly I:C (Endo+poly I:C) (Group 0). On Day 0, RBC-loaded anucleate cell-derived vesicles, incubated red blood cells or dendritic cells were adoptively transferred to recipient C57BL/6J mice. On Day 2, Group R (RBC (IgG488+Ova+poly I:C)+Boost) received a second (boost) dose of 150 M RBC (Ova+poly I:C) loaded vesicles. On Day 7, spleens were harvested, restimulated with SIINFEKL (1 μg/mL) for Ova-specific tetramer, and subjected to intracellular cytokine staining (ICS) for IFN-γ and IL-2.

### Results

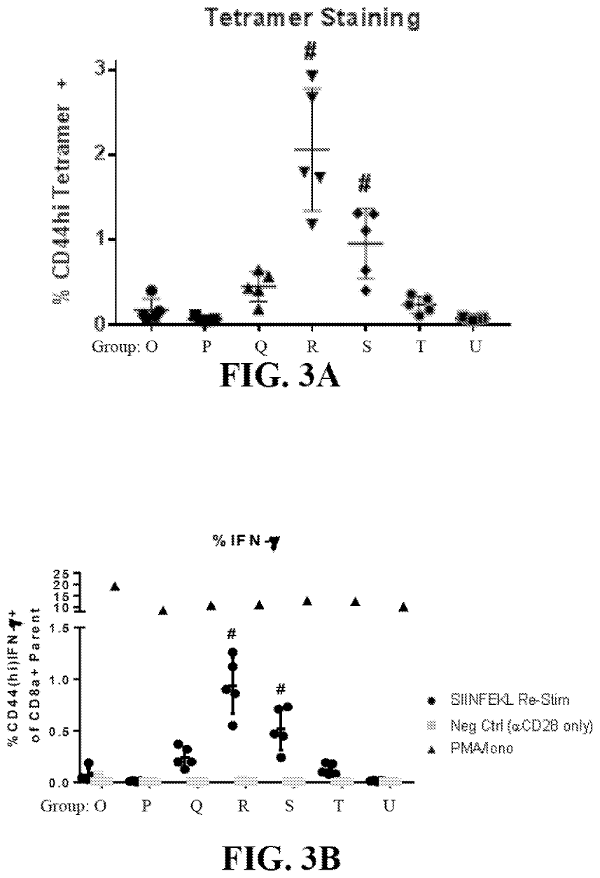

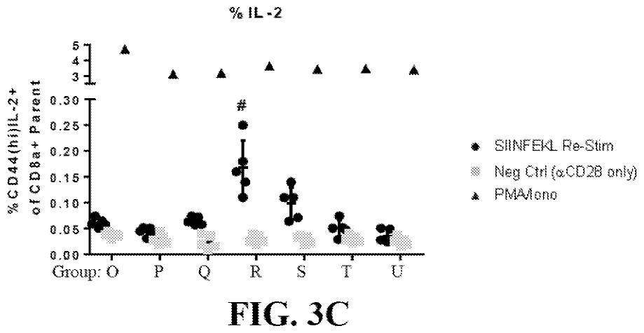

The number of antigen-specific T cells were measured by tetramer staining, with only Group R, the boosted condition (RBC (IgG488+Ova+poly I:C)+Boost) and Group S, the high dose adjuvant (RBC (IgG488+Ova+high dose poly I:C)) generating statistically significant increases in Ova-reactive T cells over the Endo control (FIG. 3A). The trend observed with tetramer staining was further supported by the percentage of cells positive for IFN-γ as measured by ICS, with the same conditions generating statistically significant increases in the % of IFN-γ relative to control (FIG. 3B). While this trend was also observed for the boosted condition via IL-2 ICS, the high dose poly I:C condition only led to a modest, statistically insignificant increase in the proportion of IL-2+ cells (FIG. 3C). Taken together, these data show that using the adjuvant poly I:C led to the highest endogenous response and that the use of a second booster on Day 2 led to much higher responses, even over a single administration of vesicles comprising Ova and a 10-fold higher dose of poly I:C. All comparisons were made to Endo+poly I:C negative control (Group 0) (5 mice/group, *P<0.05, **P<0.01, #P<0.005).

### Example 4

In order to determine the metabolic activity of normal anucleate cells compared to their anucleate cell-derived vesicle counterparts, the level of glycolysis of RBCs and RBC-derived vesicles can be indirectly measured over time by monitoring the level of lactate production using a fluorescent enzymatic assay. RBC metabolic activity can be measured by generation of lactate through glycolysis. Without mitochondria, glycolysis is the only way RBCs make ATP which is required to flip phosphatidylserine on the external membrane leaflet back to the intracellular membrane leaflet. Lack of ATP means RBCs cannot return cell surface phosphatidylserine back to basal levels.

### Materials & Methods

Human RBCs were obtained from whole blood by Ficoll separation, resuspended in citrate-phosphate-dextrose with adenine (dCPDA-1) buffer at 1 billion cells/mL and fluorescently-labeled Rat IgG (20 μg/mL) was delivered at room temperature via SQZ (2.2 μm constriction width at 50 psi) to generate RBC-derived vesicles comprising the IgG. Cells were then incubated at 37° C. for the indicated time points and supernatant was collected. To quantify the levels of lactate produced by RBCs and RBC-derived vesicles, the Lactate-Glo assay (Promega) was employed to assay supernatant from the respective time points. Briefly, the supernatants were subjected to inactivation and neutralization steps, prior to the addition of the fluorescent lactate detection reagent. Fluorescence was normalized to a blank control and the absolute lactate levels in the supernatant were determined using a lactate standard curve (0.1-10 μM).

### Results

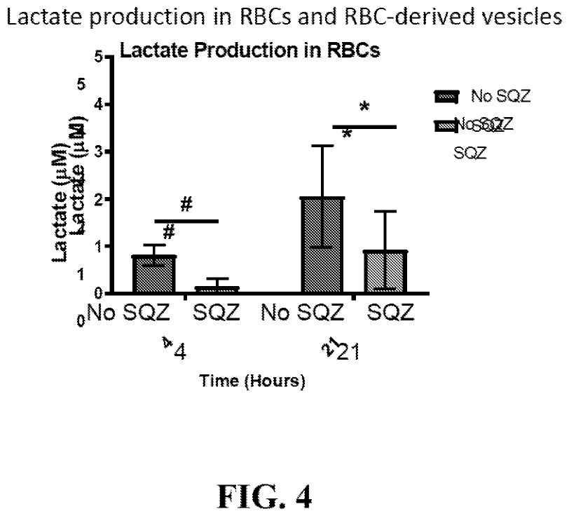

Human RBC-derived vesicles that were generated by SQZ exhibited a significantly lower level of lactate production at both 4 and 21 hours (FIG. 4). At 4 hours, RBC-derived vesicles generated by SQZ (SQZ) had a ˜5-fold reduction in lactate production relative to untreated RBCs (No SQZ), indicative of decreased metabolic activity and ATP generation (left bars, #P<0.005). Even after an extended recovery time (21h), RBC-derived vesicles generated by SQZ (SQZ) had a significantly lower (2-fold; right bars, *P<0.05) amount of lactate production relative to No SQZ. Taken together, these data show that RBC-derived vesicles that are loaded by SQZ have significantly altered metabolic potential.

### Example 5

This example demonstrates, in part, the effect of SQZ-mediated loading on the morphology and surface phosphatidylserine levels of anucleate cell-derived vesicles.

### Materials & Methods

To quantify the impact of SQZ-mediated delivery on the morphology and surface phosphatidylserine levels of anucleate cell-derived vesicles, red blood cell (RBC)-derived vesicles were loaded with a fluorescently-tagged antigen, injected into a recipient mouse followed by serial blood draws over time. The half-life of the anucleate cell-derived vesicles were then determined from the persistence of fluorescence signal in the blood. Specifically, red blood cells (RBCs) were obtained from C57BL/6J donor mice, and either left untreated (Untrtd), incubated in the presence of fluorescently-tagged (D-FITC) 3 kDa Dextran (200 μg/mL—No SQZ) or processed by SQZ-mediated loading to generate RBC-derived vesicles loaded with dextran (SQZ). Cells or vesicles generated by each condition were stained with CellTrace Violet® (CT), a membrane labeling dye. Samples from each condition were assessed by ImageStream® analysis to determine morphological changes.

To quantify the impact of SQZ-mediated delivery on the morphology and surface phosphatidylserine levels, the Dextran incubated RBCs prepared above were further incubated in RPMI containing CaCl₂) buffer (0.4 mM) at 37° C. for 2h, followed by treatment with ionomycin (8 μM) for 30 mins, conditions known to induce phosphatidylserine surface presentation (giving rise to Positive Control, +ctrl). Cells from Untrt, +ctrl and No SQZ samples, as well as SQZ-loaded vesicles (SQZ) were then stained with Annexin V and the levels of surface phosphatidylserine were measured in parallel by flow cytometry.

### Results

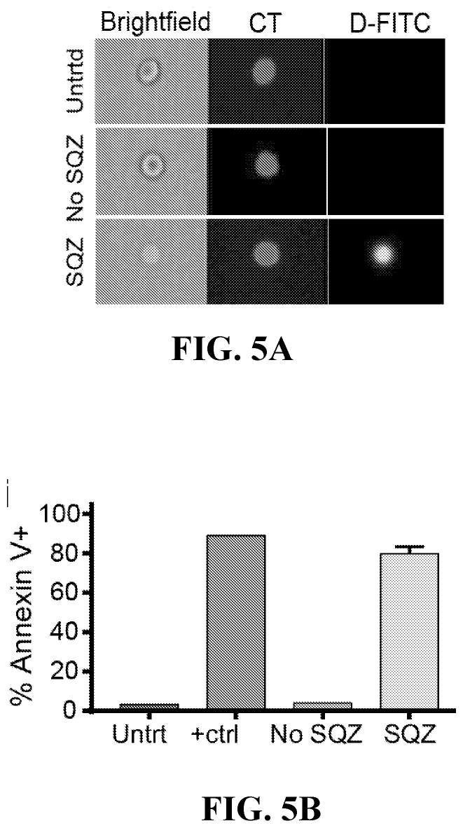

The results of the ImageStream analysis show that RBCs that were left untreated (Untrt) or incubated with dextran in the absence of SQZ (No SQZ) maintained the biconcave shape normally associated with RBCs when imaged under brightfield (FIG. 5A). SQZ-loaded RBC-derived vesicles, however, showed a distinct change in morphology, exhibiting generally more spherical shapes as shown by brightfield imaging. Additionally, only the SQZ-loaded vesicles (SQZ), but not the untreated (Untrtd) or incubated RBCs (No SQZ), showed fluorescence signal from the fluorescently-tagged dextran (D-FITC), indicating that delivery was only achieved with SQZ-mediated loading. Samples in all conditions were positive for CellTrace Violet®, showing the presence of a lipid bilayer.

The RBCs or RBC-derived vesicles described above were also tested for the levels of surface phosphatidylserine levels, a marker of membrane scrambling in RBC-derived vesicles (FIG. 5B). When compared to untreated (Untrt) and incubated RBCs (No SQZ), SQZ-loaded vesicles (SQZ) exhibited significantly higher levels of phosphatidylserine staining, with >80% of cells positive for Annexin V in the SQZ-loaded RBC-derived vesicles, relative to <5% for the RBCs without SQZ processing. The percentage of cells positive for higher levels of surface phosphatidylserine in the SQZ condition was similar to those seen with the positive control (+ctrl). Overall, these data indicate that SQZ-mediated delivery led to efficient loading of RBC-derived vesicles, noticeable modulation in morphology from the input RBCs, while significantly increasing surface phosphatidylserine levels.

### Example 6

This example demonstrates, in part, the effect of SQZ-mediated loading on the circulating half-life of anucleate cell-derived vesicles.

### Materials & Methods

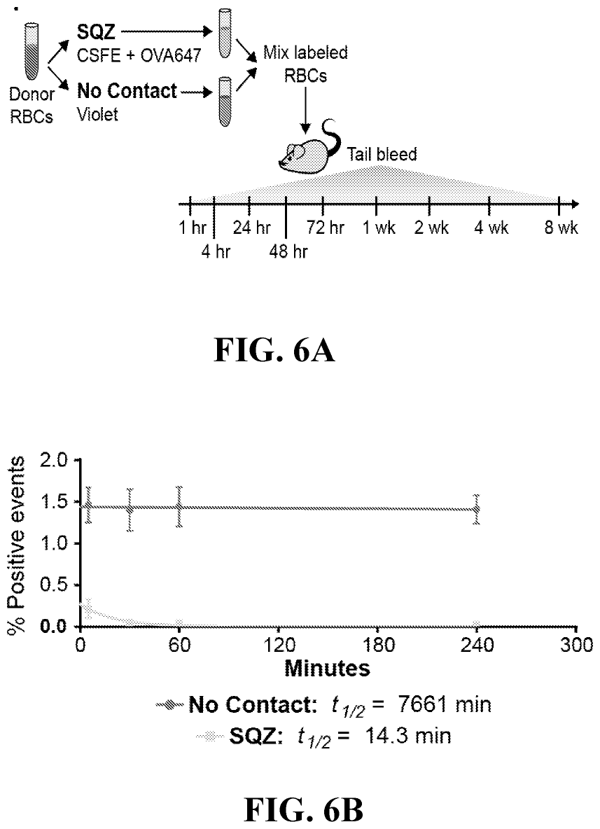

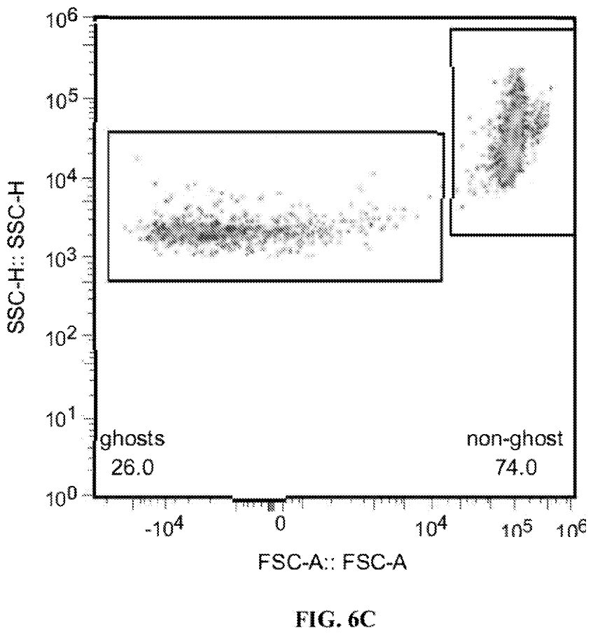

To quantify the impact of SQZ-mediated delivery on the circulating half-life of anucleate cell-derived vesicles, red blood cell-derived vesicles were loaded with a fluorescently-tagged antigen, injected into a recipient mouse followed by serial blood draws over time. The half-life of the anucleate cell-derived vesicles were then determined from the persistence of fluorescence signal in the blood. Specifically, red blood cells (RBCs) were obtained from C57BL/6J donor mice and processed with SQZ loading to generate RBC-derived vesicles with fluorescently-tagged Ova protein (Ova-647-200 μg/mL) as outlined in FIG. 6A (3 mice/group). At 0 minutes, Ova-loaded RBC-derived vesicles (200 million vesicles/animal) were stained with CFSE. An equal amount of RBCs, used as non-SQZ control, were stained with CellTrace Violet. The CFSE-stained, OVA-loaded RBC-derived vesicles and the Celltrace Violet-stained RBCs were mixed and were adoptively transferred to recipient C57BL/6J mice. At 5, 30, 60 and 240 minutes, blood was collected from the tail vein of each mouse, and the number of circulating fluorescently-tagged RBC-derived vesicles as well as Violet stained RBCs were quantified by flow cytometry.

### Results

The levels of CFSE positive RBC-derived vesicles (SQZ-loaded with antigen) in circulation had dropped significantly by the first time point (5 mins) and were nearly undetectable by 15 mins (FIG. 6B). However, RBCs that were not SQZ-processed, but were labeled with CellTrace Violet®, persisted at similar levels over the plotted time course (FIG. 6B) Repeated blood draws were performed beyond the plotted time courses showed that while the relative levels of both SQZ processed RBC-derived vesicles and the non-SQZ RBCs were generally stable after about 1 h, but using the data retrieved up to 8 weeks, the half-lives for both SQZ processed RBC-derived vesicles and the non-SQZ RBCs. The relatively short-lived RBC-derived vesicles had a half-life of 14.3 mins, while the labeled RBCs without SQZ-processing had a half-life of 7661 minutes (˜5 days). Flow plot diagrams of the mixture of RBCs/RBC-derived vesicles that were injected into recipient mice are displayed in FIG. 6C. This forward vs side scatter plot also provided quantitation of entities showing changes in morphology. The greater percentage of cells in the top right quadrant of the graph represented non SQZ-processed RBCs, while the smaller populations of SQZ-loaded RBC-derived vesicles were represented in the cluster on the left. This also illustrates the finding that the morphology of SQZ-loaded RBC-derived vesicles was significantly altered from that of RBCs that were not SQZ-processed. Taken together, these data showed that the RBC-derived vesicles cleared significantly faster than RBCs that were not SQZ-processed, and that the two were noticeably different morphologically by flow cytometry.

### Example 7

This example demonstrates, in part, the effect of SQZ-mediated loading on the hemoglobin content of anucleate cell-derived vesicles.

### Materials & Methods

To quantify the impact of SQZ-mediated delivery on the hemoglobin (Hb) content of anucleate cell-derived vesicles, RBC-derived vesicles were generated by SQZ-processing under various conditions and the amount of remaining hemoglobin, as compared to that of input RBCs, was quantified using the HemoCue® system. Specifically, RBCs were obtained from C57BL/6J donor mice, and either left untreated (NC—negative control), incubated in a solution that was diluted 1:20 in water (5 μL blood in 95 μL water—Lysis Control) or SQZ-processed at two different pressures (10 & 12 psi). After centrifugation, the amount of hemolysis (loss of Hb) was determined using a HemoCue® system by determining the concentration of Hb in the supernatant versus that in the full cell suspension according to the following equation: % hemolysis=([Free Hb]/[Total Hb])*100.

### Results

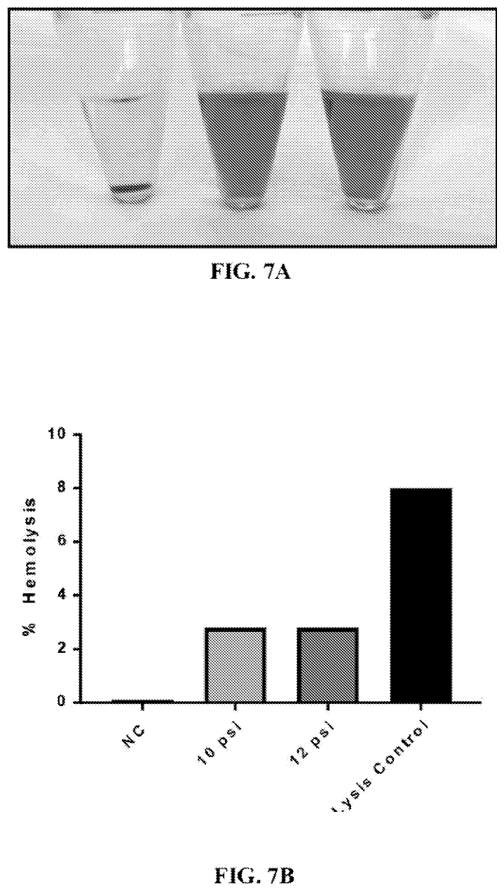

After centrifugation, it was visually apparent that SQZ-processed RBC-derived vesicles led to significant hemolysis (loss of Hb), which was readily observed from the diffuse red color of the RBC-derived vesicle supernatant, compared to the essentially clear supernatant with intensely red cell pellet as observed with non-SQZ processed RBCs (FIG. 7A). When quantifying using HemoCue® system, the Lysis Control displayed 8% hemolysis, while both conditions of SQZ-loaded RBC-derived vesicles (at 10 & 12 psi) exhibited approximately 3% hemolysis. In comparison, the RBCs not processed by SQZ merely registered 0.2% hemolysis (FIG. 7B). Taken together, these data show that the Hb levels decreased in SQZ-loaded RBC-derived vesicles relative to untreated input RBCs.

### Example 8

This example demonstrates, in part, the effect of SQZ-mediated loading on the hemoglobin content of anucleate cell-derived vesicles.

### Materials & Methods

To quantify the impact of SQZ-mediated delivery on the hemoglobin (Hb) content of anucleate cell-derived vesicles, RBC-derived vesicles were generated by SQZ-processing under various conditions and the amount of remaining hemoglobin, as compared to that of input RBCs, was quantified by LC/MS. Specifically, RBCs were obtained from NOD donor mice, and were either incubated with FAM-tagged insulin B9-23 peptide (75 μM) in PBS (Endo Control), or processed through SQZ-mediated loading to generate RBC-derived vesicles loaded with insulin B9-23 peptide (SQZ). Samples from Endo Control or SQZ (from 5E7-1.5E8 cells or vesicles/replicate sample) were then subjected to a standard peptide denaturation, reduction, alkylation and trypsinization (DRAT) procedure prior to liquid chromatography/mass spectrometry analysis (LC/MS). Following derivatization, samples were run using reverse-phase LC, and the levels of two known hemoglobin peptides (Hb peptide #1—FLASVSTVLTSK; Hb peptide #2—VGAHAGEYGAEALER) were quantified by calculating the area under the curve for the respective peak.

### Results

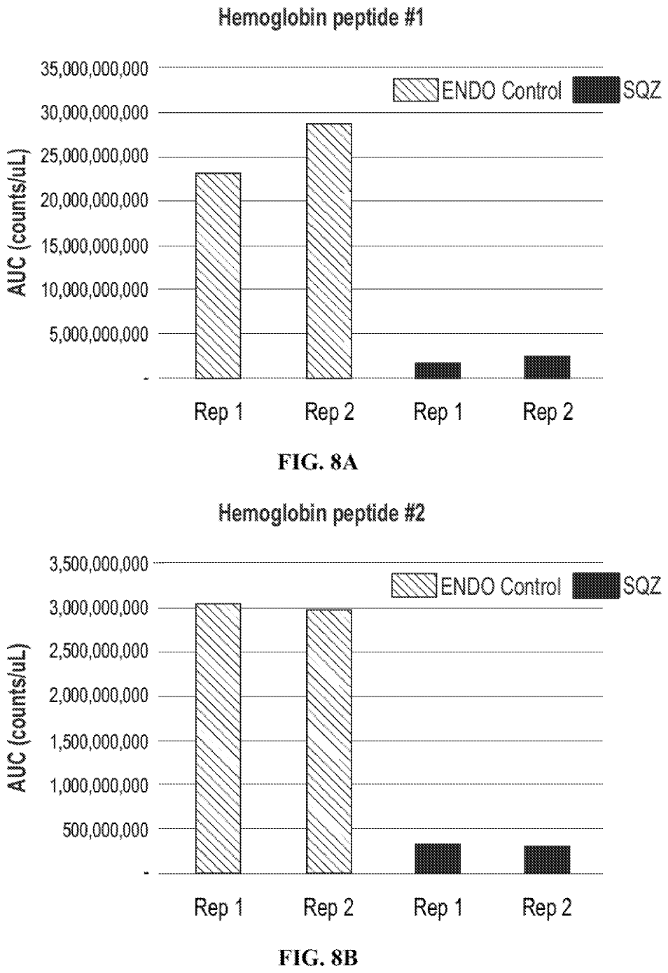

The LC/MS analysis showed that RBCs that were not SQZ-processed (Endo Control) had almost ten-fold more Hb content than the SQZ-loaded RBC-derived vesicles (2 technical replicates/sample; shown separately). The trend observed between Endo Control and SQZ was observed for both Hb peptides, with a >80% relative reduction of Hb content in the SQZ sample (FIG. 8A-8B). Taken together, these data show that a significant amount of Hb was lost when RBCs were SQZ-processed to become RBC-derived vesicles, as compared to their unprocessed RBC counterparts.

### Example 9

This example demonstrates, in part, the effect of constriction size and pressure in SQZ-mediated loading on the ghost formation in derivation of anucleate cell-derived vesicles.

### Materials & Methods

To quantify the impact of constriction width and pressure in SQZ-mediated loading on ghost formation in the derivation of anucleate cell-derived vesicles, RBC-derived vesicles were generated by SQZ-processing under various constriction widths and pressure conditions, and the amount of amount of ghost formation was quantified by flow cytometry. Specifically, red blood cells (RBCs; 100 M cells/mL) were obtained from C57BL/6J donor mice, and were either incubated with fluorescently-tagged Ova (10 μg/mL) in diluted CPDA-1 solution (Endo), or were SQZ-loaded with Ova using combinations of two different constriction diameters (2.2 or 2.5 μm) and two different driving pressures (30 and 50 psi). The amount of ghost formation was then determined by flow cytometry. The non-ghost and ghost vesicle profiles were previously illustrated in FIG. 6C.

### Results

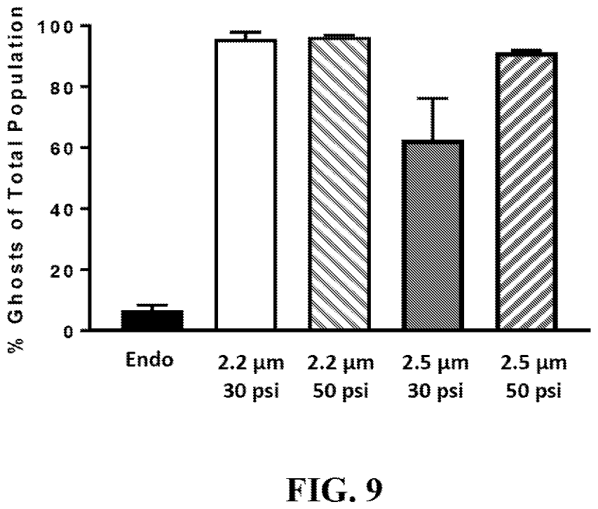

By altering the constriction width and/or the pressure in SQZ processing, the relative amount of ghost formation resulting from SQZ-loading of RBC-derived vesicles can be modulated. As expected, RBCs that were not SQZ-processed (Endo) displayed a very low percentage of ghost formation (˜5%). All SQZ conditions tested led to significantly higher percentages of ghost formation relative to Endo (P<0.005) (FIG. 9). With a 2.5 μm constriction width, SQZ-processing at 30 psi driving pressure led to ˜60% ghost formation, but ghost formation was increased to >90% when SQZ-processing at 50 psi driving pressure (P<0.05) (FIG. 8). Additionally, ghost formation in SQZ-processing using narrower constriction width (2.2 μm) also led to a higher percentage of ghost formation (>90% ghost formation) as compared to a wider constriction width (2.5-60% ghost formation) when the same driving pressure of 30 psi was applied (P<0.05) (FIG. 9). Taken together, these data show that by altering the driving pressure or the constriction width, the percentage of ghost formation from SQZ-loading anucleate cell-derived vesicles could be actively tuned.

### Example 10

This example demonstrates, in part, the effect of SQZ-mediated loading on the circulating half-life of anucleate cell-derived vesicles.

### Materials & Methods

In order to determine circulation kinetics of SQZ-processed anucleate cells, murine RBCs were first labeled with PKH-26 and then subjected to SQZ-processing to generate RBC-derived vesicles. The RBC-derived vesicles were then injected into mice (1 billion vesicles for each of 2 mice), and the fluorescently (PHK-26) labeled, SQZ-processed RBC-derived vesicles were tracked in the murine blood over the course of 24 hours post-administration. Specifically, the vesicles were measured via fluorescence at 0 min, 15 min, 30 min, 1 hour, 4 hour, and 24 hour post-administration.

Another set of mice were injected with fluorescently labeled RBCs that were not subjected to SQZ-processing (1 billion cells for each of 3 mice). The fluorescently (PHK-26) labeled, unprocessed RBCs were tracked in the murine blood over the course of 24 hours post-administration. Specifically, the unprocessed RBCs were measured via fluorescence at 0 min, 15 min, 30 min, 1 hour, 4 hour, and 24 hour post-administration.

The persistence of fluorescently (PKH-26) labeled RBC-derived vesicles and the unprocessed RBC counterparts in mouse blood stream was determined by fluorescence as assayed by flow cytometry.

### Results

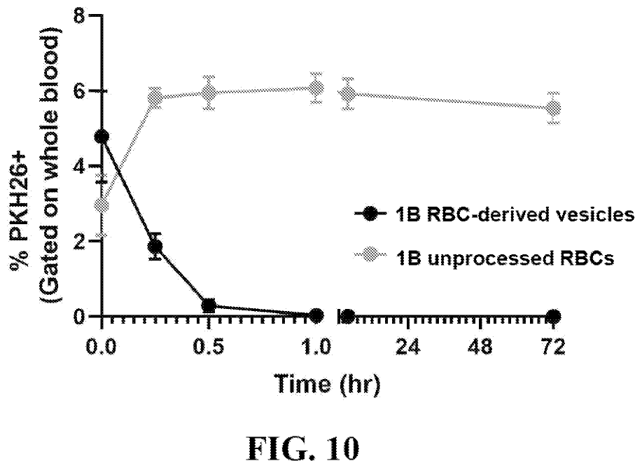

A shown in FIG. 10, SQZ-processed RBC derived-vesicles were rapidly cleared from blood. Specifically, most of the RBC-derived vesicles were cleared from blood within 60 minutes and had a circulating half-life of ˜10 minutes. In comparison, unprocessed RBCs persisted in the bloodstream for the duration of the experiment (72 hours). Taken together, these data showed that the RBC-derived vesicles cleared significantly faster than RBCs that were not SQZ-processed.

### Example 11

To evaluate the mechanism by which SQZ-processed anucleate cell-derived vesicles elicit CD8+ T-cell response, the cell types and organs involved in the uptake of the anucleate cell-derived vesicles in vivo were examined. This example demonstrates, in part, the cell types and organs involved in the immediate uptake of SQZ-processed anucleate cell-derived vesicles after intravenous injection.

### Materials & Methods

C57BL/6J mice were obtained from The Jackson Laboratory, from which 20 females were used as recipient mice for vaccination and 10 females were used as donor mice. RBCs extracted from donor mice were fluorescently labeled with PKH-26 and subsequently SQZ-processed in the presence of antigen (E7 SLP) and adjuvant (Poly I:C) to generate E7-loaded RBC-derived vesicles. A first group of recipient mice were injected with fluorescently labeled RBC-derived vesicles containing E7 and Poly I:C. A control group of recipient mice was injected with PBS (control).

One hour after the injection, liver, lung, spleen, and bone marrow of injected mice were harvested and examined for presence of labeled SQZ-processed RBC-derived vesicles. From the harvested liver and spleen, cells were extracted and the macrophages (MØ; CD45⁺, F4/80⁺, CD11b^{-/low}), dendritic cells (DCs; CD45+, F4/80⁻, CD11c^{hi}, MHC-II^{hi}) and B cells (CD45+, F4/80⁻,CD19⁺, FSC^{lo}, SSC^{lo}) were analyzed for uptake of RBC-derived vesicles, as assayed by their fluorescent labels via flow cytometry.

### Results

As shown in FIG. 11A, SQZ-processed RBC vesicles were primarily taken up in liver and spleen, and less so in bone marrow and lung. As shown in FIG. 11B, in the liver and spleen, the SQZ-processed RBC vesicles were primarily engulfed by macrophages (MO) and dendritic cells. Background signal observed in splenic or liver-derived cells from mice injected with PBS was below the threshold of detection.

### Example 12

This example demonstrates, in part, that anucleate cell-derived vesicles comprising loaded antigen and/or adjuvant can induce an in vivo antigen-specific immune response.

### Materials & Methods

On Day 0, 10 female OT-I mice and 10 female OT-II mice were sacrificed. Spleens and lymph nodes (inguinal, axillary, brachial, cervical, and mesenteric) were harvested, from which single cell suspensions were generated. Antigen-specific CD8+ T cells were immuno-magnetically separated from OT-I mice cells. Antigen-specific CD4+ T cells were immuno-magnetically separated from OT-II mice cells. The OVA-specific CD4+ and CD8+ T cells were stained with CFSE prior to injection into CD45.1 mice, where 2.5 M of either CD4+ T cells or CD8+ T cells were administered per mice, respectively.

On Day 1, RBCs were isolated from murine blood from three euthanatized B6 mice, and the murine RBCs were SQZ-processed in the presence of OVA (200 μM) and poly I:C (1 mg/ml) to generate RBC-derived vesicles containing antigen and adjuvant. Subsequently, the CD45.1 mice previously administered with CFSE-labeled OT-I CD8+ T cells or OT-II CD4+ T cells were injected with PBS (control) or with 250 M of the loaded RBC-derived vesicles.

On Day 4, mice administered with CFSE-labeled OVA-specific CD4+ and CD8+ T cells and subsequently injected with either (i) PBS; or (ii) OVA & Poly I:C-loaded RBC-derived vesicles were sacrificed, with their spleens harvested. The harvested spleens were manually dissociated by passage through a filter and the CD4+ and CD8+ T cell proliferation was measured by CSFE dilution via flow cytometry.

### Results

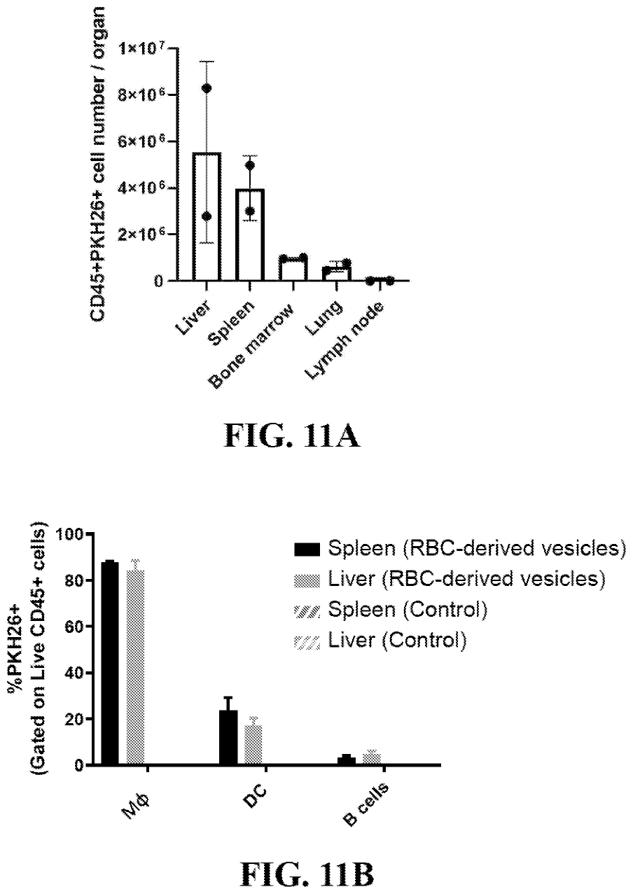

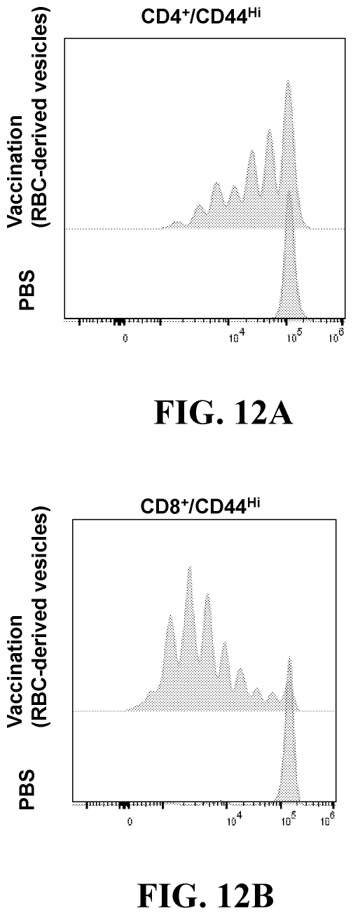

As shown in FIGS. 12A and 12B, mice receiving RBC-derived vesicles SQZ-loaded with OVA and Poly I:C exhibited robust OT-I CD4+ T cell and OT-II CD8+ T cell proliferation respectively, as shown by the significant CFSE dilution compared to control. These results indicate anucleate cell-derived vesicles comprising loaded antigen and adjuvant can induce an antigen-specific immune response in vivo.

### Example 13

This example demonstrates, in part, that anucleate cell-derived vesicles comprising loaded antigen and adjuvant can induce an endogenous antigen-specific T cell response.

### Materials & Methods

On Day 0, RBCs were isolated from murine blood from three euthanatized B6 mice, and the murine RBCs were partitioned and SQZ-processed in the presence of (i) OVA (200 μM); or (ii) poly I:C (1 mg/ml); or (iii) both OVA and poly I:C, to generate respective RBC-derived vesicles containing antigen and/or adjuvant. Subsequently, the CD45.1 mice were injected with PBS (control) or with 250 M of the respective RBC-derived vesicles loaded with antigen and/or adjuvant.

On Day 7, mice administered either with PBS, or with the respective RBC-derived vesicles, were sacrificed, and their splenocytes were harvested. The splenocytes were re-stimulated with SIINFEKL, and subjected to staining for CD44 and intracellular cytokine staining for IFN-gamma to detect any endogenous T cell activation.

### Results

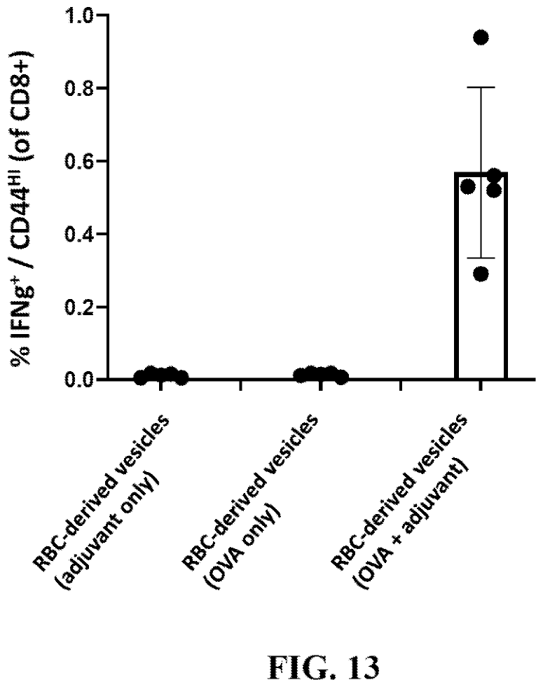

As shown in FIG. 13, mice receiving RBC-derived vesicles loaded with either only antigen (OVA) or only adjuvant (Poly I:C) exhibited no activation of CD8+ T cells, whereas mice receiving OVA & Poly I:C-loaded RBC-derived vesicles exhibited robust OVA-specific T cell proliferation, as shown by the significant increase in percentage of CD44^{hi} and IFNγ⁺ cells among total endogenous CD8+ T cell population upon re-stimulation with SIINFEKL. These results indicate anucleate cell-derived vesicles loaded with both antigen and adjuvant can induce an endogenous antigen-specific T cell response.

### Example 14

This example demonstrates, in part, that anucleate cell-derived vesicles comprising loaded antigen and adjuvant can induce an endogenous antigen-specific T cell response.

### Materials and Methods

On Day 0, RBCs were isolated using Ficoll gradient procedure from murine blood of thirteen euthanatized B6 donor mice, and a RBC suspension with a cell concentration of 1 billion/mL was prepared. The RBC suspension was partitioned into 4 groups, which were SQZ-processed at 50 psi with a 2.2 μm diameter constriction in the presence of (i) adjuvant (Poly I:C) only, (ii) antigen (E7 SLP) only, or (iii) antigen and adjuvant (E7 SLP+Poly I:C); to generate RBC-derived vesicles containing the respective payloads.

Subsequently, CD45.1 mice were injected retro-orbitally (RO) with PBS (control), or 250 million of the respective RBC-derived vesicles loaded with antigen and/or adjuvant.

On Day 7, mice were sacrificed, and their splenocytes were harvested. The splenocytes were re-stimulated with E7 peptide, and subjected to staining for CD44 and intracellular cytokine staining for IFN-gamma to detect any endogenous T cell activation.

### Results

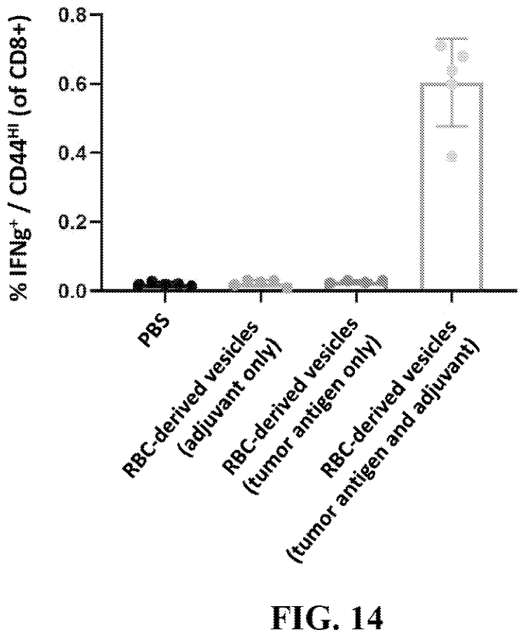

As shown in FIG. 14, mice receiving RBC-derived vesicles loaded with either only antigen (E7) or only adjuvant (Poly I:C) exhibited no activation of CD8+ T cells, whereas mice receiving E7 & Poly I:C-loaded RBC-derived vesicles exhibited robust E7-specific T cell proliferation, as shown by the significant increase in percentage of CD44^{hi} and IFNγ⁺ cells among total CD8+ T cell population upon re-stimulation with E7 peptide. These results indicate anucleate cell-derived vesicles loaded with both antigen and adjuvant can induce an endogenous antigen-specific T cell response.

### Example 15

This example demonstrates, in part, the effect of using different priming and boosting regimens of antigen-loaded anucleate cell-derived vesicles on in vivo antigen-specific immune response.

RBCs were isolated using Ficoll gradient procedure from murine blood of thirteen euthanatized B6 donor mice, and the resulting RBC suspension was SQZ-processed in the presence of antigen and adjuvant (100 μM E7 SLP+1 mg/mL Poly I:C) to generate RBC-derived vesicles containing antigen and adjuvant.

On Day 0, CD45.1 mice were injected retro-orbitally (RO), at 250 million vesicles per mouse, with PBS (control) or with RBC-derived vesicles loaded with antigen and adjuvant (Prime). Subsets of the mice having received the priming administration of the loaded RBC-derived vesicles further received (i) a boosting dose of the loaded RBC-derived vesicles on Day 2 (2 Day Boost); or (ii) two boosting doses of the loaded RBC-derived vesicles, on Day 2 and Day 7 (7 Day Boost).

At 7 days subsequent to the final immunization (Day 7 for Prime and PBS Control; Day 9 for 2 Day Boost, Day 14 for 7 Day Boost), the respective mice sacrificed, and their splenocytes were harvested. The splenocytes were subjected to CD44 and E7-specific tetramer staining, measured via flow cytometry, to detect activation of endogenous E7-specific T cells.

### Results

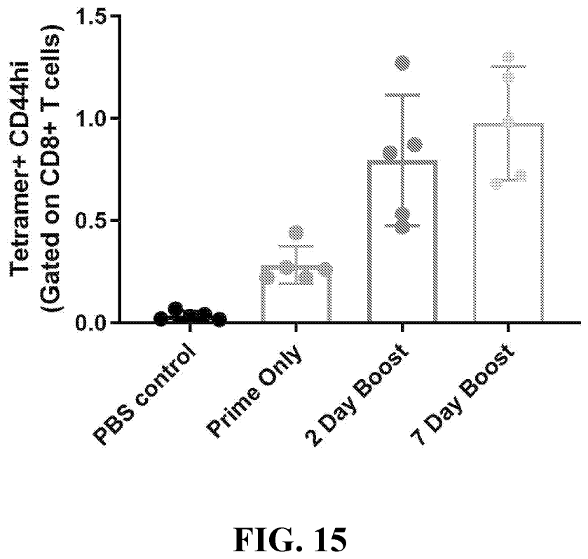

As shown in FIG. 15, mice receiving E7 & Poly I:C-loaded RBC-derived vesicles exhibited CD8+ T cell activation, as shown by the considerable increase in percentage of CD44^{hi} and tetramer⁺ cells among total CD8+ T cell population. In addition, the induction of endogenous T cell activation was observed to be increasingly robust as mice received additional boosting doses of the E7 & Poly I:C-loaded RBC-derived vesicles (FIG. 15). These results indicate anucleate cell-derived vesicles loaded with both antigen and adjuvant can induce an endogenous antigen-specific T cell response, and the induction could be enhanced by using a prime-and-boost dosing regimen.

### Example 16

This example demonstrates, in part, that anucleate cell-derived vesicles comprising loaded tumor antigen and adjuvant can be used as a prophylactic immunization against tumor.

### Materials & Methods

Female C57BL/6J mice were obtained from The Jackson Laboratory, and were used as recipient mice for vaccination and as well as donor mice. On Day 0, RBCs were extracted from donor mice and were SQZ-processed in the presence of 100 μM E7 SLP and 1 mg/mL Poly I:C to generate RBC-derived vesicles containing a tumor antigen and an adjuvant. Recipient mice were then administered with (i) PBS, or (ii) 250 million RBC-derived vesicles containing the tumor antigen and adjuvant.

7 days after immunization (Day 7), mice were subcutaneously implanted in the right rear flank with TC-1 tumor cells expressing HPV E7. TC-1 tumor growth was then measured two times per week and compared to tumor growth in untreated mice for 41 days. Tumor size was measured by the formula ((length×width²)/2). Mouse body weight and survival were recorded over 60 days.

### Results

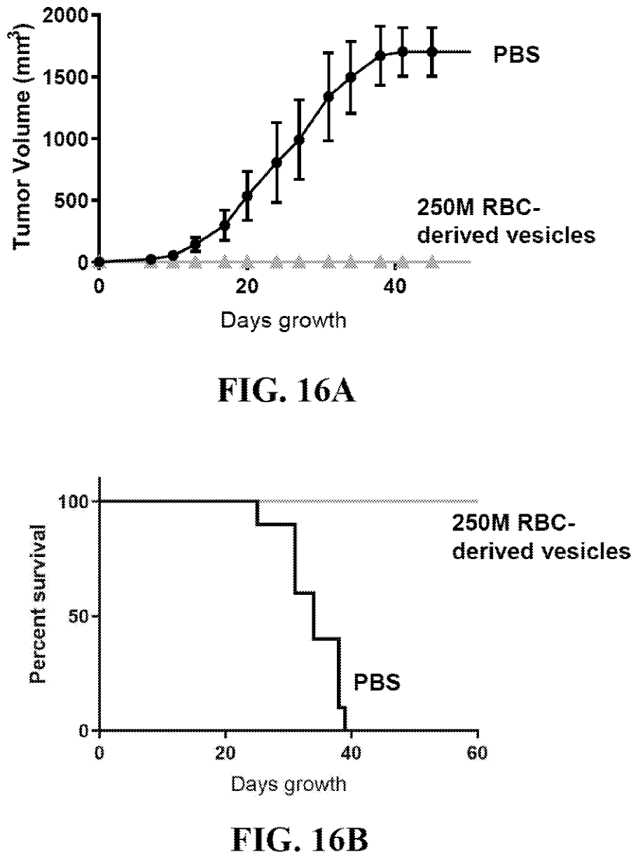

As shown in FIG. 16A, tumor growth was completely inhibited in mice treated with E7+Poly I:C-loaded vesicles, as compared to control mice where tumor grew unabated. As shown in FIG. 16B, none of the PBS-treated mice (0/10) survived past Day 41 (Median survival=34 days), whereas all mice prophylactically immunized with E7+Poly I:C-loaded vesicles (10/10) remained tumor free for at least 60 days. These data show that anucleate cell-derived vesicles loaded with tumor antigen and adjuvant can effectively prevent tumor growth and improve survival in a prophylactic model of HPV-associated cancer.

### Example 17

This example demonstrates, in part, that anucleate cell-derived vesicles comprising loaded tumor antigen and adjuvant can be used as a therapeutic immunization against tumor.

### Materials & Methods

Female C57BL/6J mice were obtained from The Jackson Laboratory, and were used as recipient mice for vaccination and as well as donor mice.

On Day 0, recipient mice were subcutaneously implanted with TC-1 tumor cells expressing HPV E7. On Day 10, RBCs were extracted from donor mice and were SQZ-processed at 50 psi with a 2.2 μm diameter constriction in the presence of 100 μM E7 SLP and 1 mg/mL LMW Poly I:C to generate RBC-derived vesicles containing a tumor antigen and an adjuvant. Recipient mice were subsequently (i) left untreated/administered with PBS (control); or (ii) administered a dose of either 1 billion, 250 million, or 100 million E7+Poly I:C-loaded RBC-derived vesicles.

TC-1 tumor growth was measured beginning 1 week post-tumor implantation two times per week and compared to tumor growth in untreated mice for 41 days. Tumor size was measured by the formula ((length×width²)/2). Mouse body weight and survival were recorded over 50 days.

### Materials & Methods

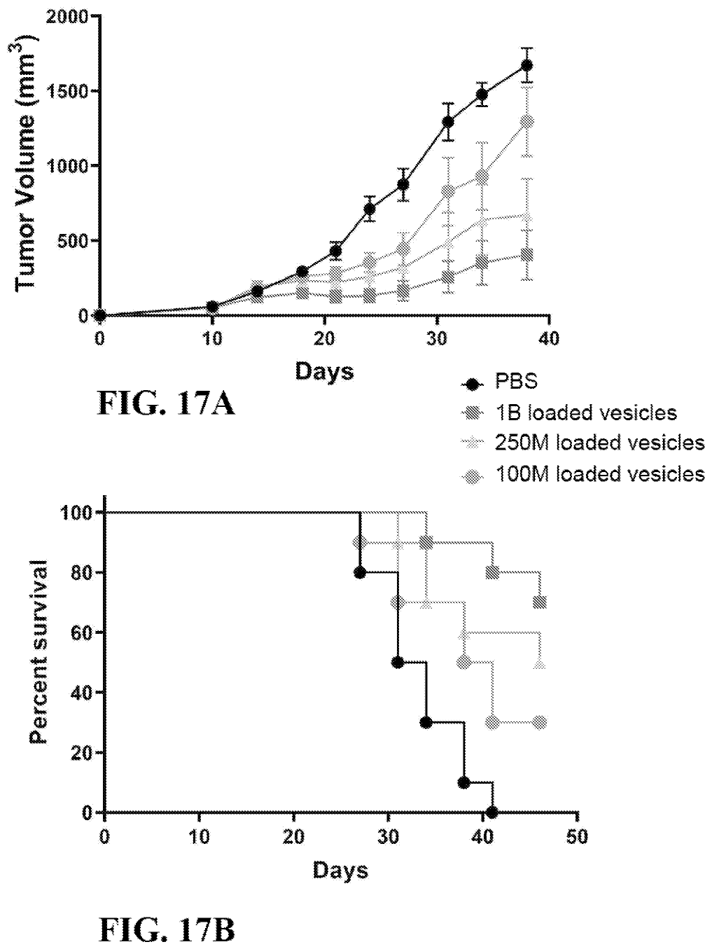

As shown in FIG. 17A, tumor growth was significantly inhibited in mice treated with 1 billion or 250 million E7+Poly I:C-loaded RBC-derived vesicles, and also noticeably in mice treated with 100 million E7+Poly I:C-loaded RBC-derived vesicles, as compared to control mice where tumor grew unabated. As shown in FIG. 17B, none of the control mice survived past Day 41 (median survival=32.5 days), whereas more than half of the mice immunized therapeutically with 1 billion or 250 million E7+Poly I:C-loaded vesicles were viable for at least 41 days, and the therapeutic efficacy correlated with dose of vesicles administered (median survival=39.5 days for 100 million vesicles administered; 46 days for 250 million vesicles administered; did not reach median survival at 46 days for 1 billion vesicles administered). These data show that anucleate cell-derived vesicles loaded with tumor antigen and adjuvant can induce tumor regression and enhance survival in a therapeutic model of HPV-associated cancer.

### Example 18

This example demonstrates, in part, the effect of using different priming and boosting regimens of antigen-loaded anucleate cell-derived vesicles on efficacy as a therapeutic immunization against tumor.

### Materials & Methods

Female C57BL/6J mice were obtained from The Jackson Laboratory, and were used as recipient mice for vaccination and as well as donor mice.

On Day 0, recipient mice were subcutaneously implanted with TC-1 tumor cells expressing HPV E7.

RBCs were extracted from donor mice and were SQZ-processed at 50 psi with a 2.2 μm diameter constriction in the presence of 100 μM E7 SLP and 1 mg/mL LMW Poly I:C to generate RBC-derived vesicles containing a tumor antigen and an adjuvant. Recipient mice were subsequently (i) left untreated/administered with PBS (control); or (ii) administered a dose of 100 million E7+Poly I:C-loaded RBC-derived vesicles on Day 10 (Prime); (iii) administered doses of 100 million E7+Poly I:C-loaded RBC-derived vesicles each on Day 10 and Day 12 (Prime/Boost); or (iv) administered doses of 100 million E7+Poly I:C-loaded RBC-derived vesicles each on Day 10, Day 12 and Day 14 (Prime/Boost/Boost).

TC-1 tumor growth was measured beginning 1 week post-tumor implantation two times per week and compared to tumor growth in untreated mice for 41 days. Tumor size was measured by the formula ((length×width²)/2). Mouse body weight and survival were recorded over 50 days.

### Results

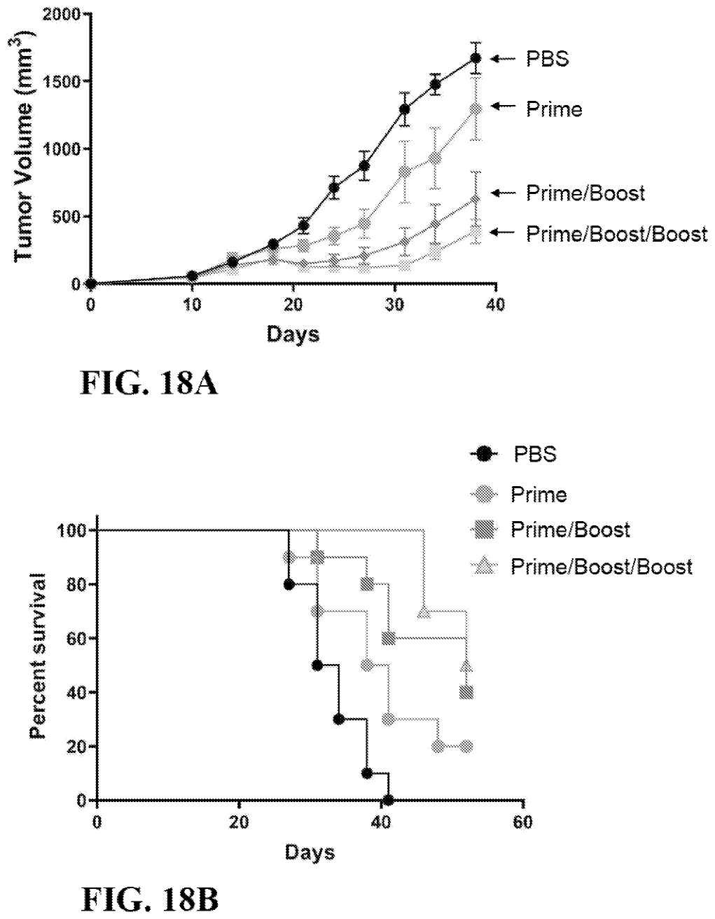

As shown in FIG. 18A, while tumor growth was noticeably inhibited in mice treated with 1 dose of 100 million E7+Poly I:C-loaded RBC-derived vesicles (Prime) as compared to control mice; the tumor regression was more significant when mice were treated with additional boosting doses of the E7+Poly I:C-loaded RBC-derived vesicles (Prime/Boost, Prime/Boost/Boost). As shown in FIG. 18B, all control mice expired at 41 days (median survival=32.5 days), while ˜30% of mice survived with 1 treatment dose of 100 million E7+Poly I:C-loaded RBC-derived vesicles (Prime) at Day 41 (median survival=39.5 days). Further boosting doses significantly increased survival at Day 41, with more than 50% survival for mice receiving 2 doses of the loaded vesicles (Prime/Boost) and 100% survival for mice receiving 3 doses of the loaded vesicles (Prime/Boost/Boost) (median survival=52 days for both). These data show that anucleate cell-derived vesicles loaded with tumor antigen and adjuvant can induce tumor regression and enhance survival in a therapeutic model of HPV-associated cancer, and the tumor regression and survival can be improved with further boosting regimen.

### Example 19

This example demonstrates, in part, the quantity of E7-specific CD8+ T cells in the tumor microenvironment of TC-1 tumors post immunization with SQZ-loaded anucleate cell-derived vesicles and correlation of the E7-specific CD8+ T cells with tumor clearance in a tumor growth model.

### Materials & Methods

Female C57BL/6J mice were obtained from The Jackson Laboratory, and were used as recipient mice for vaccination and as well as donor mice.

On Day 0, recipient mice were subcutaneously implanted with TC-1 tumor cells expressing HPV E7 (50 k/mouse in 100 μL of PBS).

On Day 14, RBCs were extracted from donor mice and were SQZ-processed at 50 psi with a 2.2 μm diameter constriction in the presence of (i) 1 mg/mL Poly I:C only; or (ii) 100 μM E7 SLP and 1 mg/mL Poly I:C to generate RBC-derived vesicles containing a tumor antigen and/or an adjuvant. Recipient mice were subsequently (i) administered with PBS (control); or (ii) administered 250 million Poly I:C-loaded RBC-derived vesicles; or (iii) administered 250 million E7+Poly I:C-loaded RBC-derived vesicles.

On Day 21 and 26 (7 and 12 days post-immunization), tumors were excised and weighed, and from which respective single-cell suspensions were generated. The single-cell suspension was assessed for total CD8+ T cells, regulatory T cells (CD45⁺, B220⁻, CD11b⁻, CD4⁺, FoxP3⁺), and antigen-specific tumor-infiltrating lymphocytes (TILs) by flow cytometry.

### Results

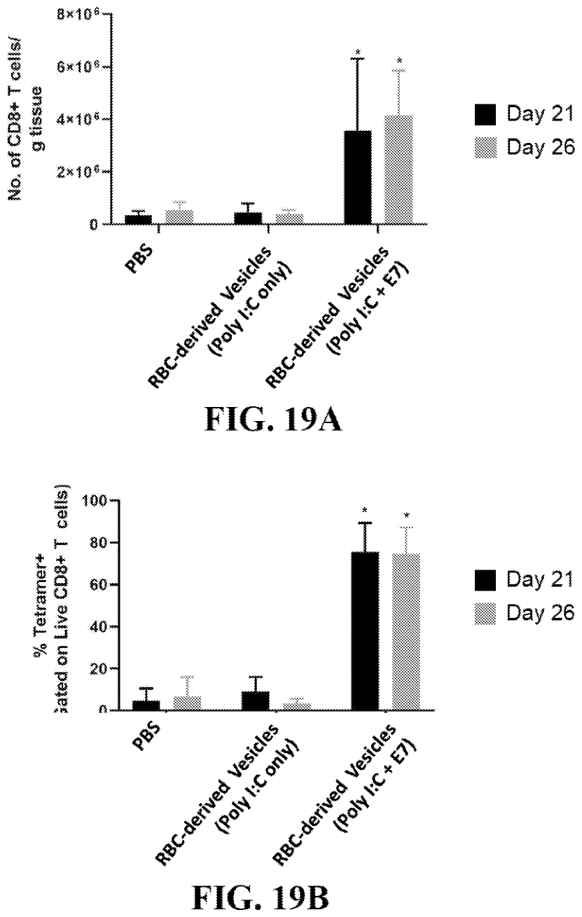

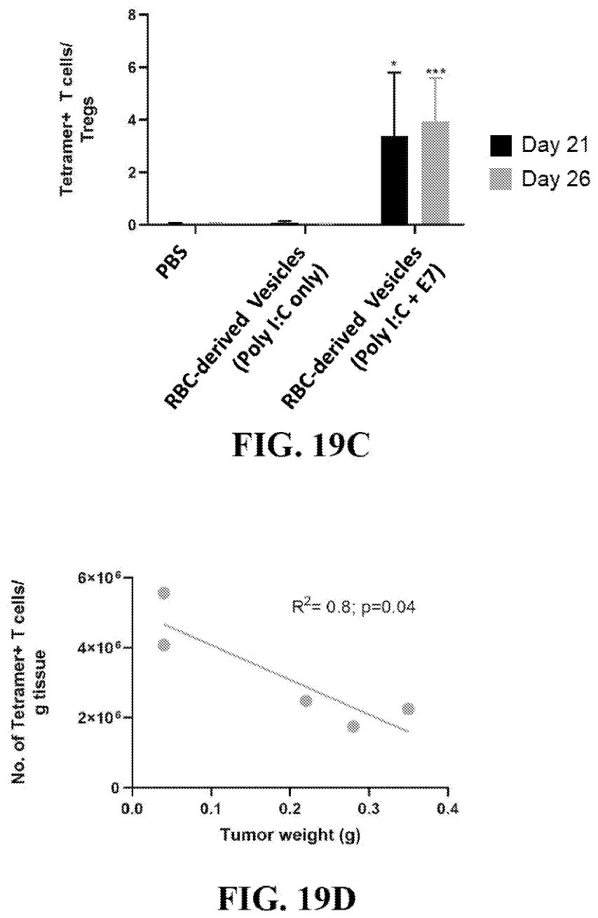

As seen in FIG. 19A, mice immunized with E7+Poly I:C-loaded RBC-derived vesicles had a significant increase in the percentage of CD8+ T cells in the tumor compared to mice receiving Poly I:C-loaded RBC-derived vesicles and control mice on both 7 and 12 days after immunization (i.e. 21^{st} and 26^{th} day post tumor implant). In mice receiving E7+Poly I:C-loaded RBC-derived vesicles, the majority of these CD8+ T cells were specific for the E7 antigen as determined by tetramer staining (>70% of the CD8+ population) (FIG. 19B). To investigate the relative amount of regulatory T cell activation by the respective vesicles, the percentage of cells positive for E7-specific tetramer staining in the tumor was also normalized to the amount of regulatory T cells in the tumor (FIG. 19C), and the results demonstrate that immunization with E7+Poly I:C-loaded RBC-derived vesicles more significantly increased the presence of E7-specific CD8+ T cells (TILs) compared to regulatory T cells in the tumor microenvironment post immunization, in comparison to administration with Poly I:C-loaded RBC-derived vesicles or PBS. As shown in FIG. 19D, the amount in E7 specific CD8+ T cells (TILs) was also observed to inversely correlate with tumor weight, demonstrating tumor regression would correlate with an influx of E7-specific CD8+ T cells. These data demonstrate that immunization with E7+Poly I:C-loaded RBC-derived vesicles in the TC-1 mouse tumor model led to a significant increase in E7-specific CD8+ T cells infiltrating the tumor. The increase in E7-specific CD8+ T cells (FIGS. 19A-19C) coupled with a correlating decrease in tumor volume (FIG. 19D) supported that E7+Poly I:C-loaded RBC-derived vesicles reduced tumor burden by expanding E7-specific effector CD8+ T cells.

### Example 20

This example demonstrates, in part, the effect of SQZ-mediated processing in payload delivery, ghost formation and surface phosphatidylserine levels in human anucleate cell-derived vesicles.

### Materials & Methods

Human red blood cell (RBC) were obtained from healthy donors, and the resulting RBC suspension (2 billion/mL) was either left untreated (No SQZ-Cells), or SQZ-processed (60 psi, 2.2 μm diameter constriction) in the presence of a fluorescently-tagged E7 SLP (antigen) and Poly I:C (adjuvant) to generate human RBC-derived vesicles loaded with antigen and adjuvant (H-SQZ-Vesicles).

To quantify the efficacy of SQZ-mediated delivery, No-SQZ-Cells and H-SQZ-Vesicles were measured for the presence of fluorescently-tagged E7 SLP by flow cytometry.

To quantify for ghost formation, No-SQZ-Cells and H-SQZ-Vesicles were subjected to flow cytometry and analyzed for forward scatter and side scatter, using procedures as described in Example 6 and FIG. 6C.

To quantify the impact of SQZ-mediated processing on the surface phosphatidylserine levels, No-SQZ-Cells and H-SQZ-Vesicles were stained with Annexin V and the levels of surface phosphatidylserine were measured in by flow cytometry, using procedures as described in Example 5.

### Results

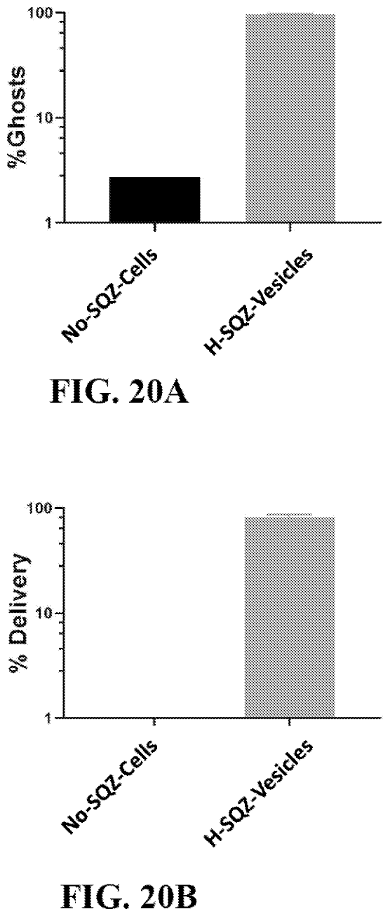

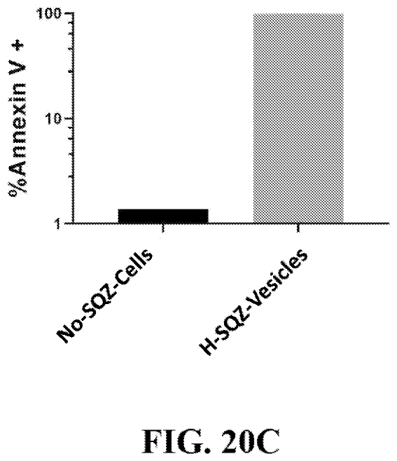

As shown in FIG. 20A, SQZ-processing of human RBCs resulted in RBC-derived vesicles (H-SQZ-Vesicles) of which a large majority displayed a ghost profile. In contrast, a very low proportion of untreated human RBCs (No SQZ-Cells) displayed a ghost profile. Furthermore, as shown in FIG. 20B, SQZ-processing of human RBCs in the presence of a payload showed effective delivery to a high percentage of the resulting RBC-derived vesicles (H-SQZ-Vesicles). As shown in FIG. 20C, SQZ-processing of human RBCs resulted in RBC-derived vesicles (H-SQZ-vesicles) of which a large majority displayed surface phosphatidylserine (Annexin V+). In contrast, a very low proportion of untreated human RBCs (No SQZ-Cells) displayed surface phosphatidylserine.

### Example 21

The mechanism by which SQZ-processed human anucleate cell-derived vesicles leads to antigen presentation and activation of CD8+ T-cell response was evaluated. It is important to understand the kinetics of the uptake of the anucleate cell-derived vesicles by antigen presenting cells. This example demonstrates, in part, the efficiency of human monocyte-derived dendritic cells (MoDCs) in internalizing SQZ-processed human anucleate cell-derived vesicles in vitro.

### Materials & Methods

Human red blood cell (RBC) were obtained from healthy donors, and the resulting RBC suspension (2 billion/mL) was fluorescently labeled with PKH-26, and either left untreated, or SQZ-processed (60 psi, 2.2 μm diameter constriction) in the presence of E7 SLP to generate human RBC-derived vesicles loaded with antigen (H-SQZ-Vesicles). To quantify the efficacy of MoDCs in internalizing SQZ-processed human anucleate cell-derived vesicles, MoDCs were seeded in 96-well plates, incubated overnight, at 37° C. or 0° C. (on ice), with H-SQZ-Vesicles at a spectrum of vesicle concentration. The MoDCs were subsequently isolated and analyzed for increase in fluorescence by flow cytometry.

### Results

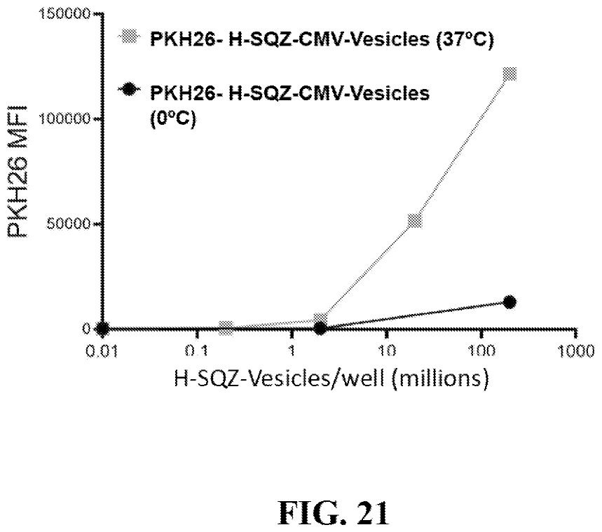

As shown in FIG. 21, when incubated with H-SQZ Vesicles, MoDCs were significantly more efficient in internalization of H-SQZ-Vesicles at 37° C. than at 0° C. In addition, the internalization of H-SQZ-Vesicles was observed to be dependent on H-SQZ Vesicle concentration up to at least 100 million vesicles per well of seeded MoDCs.

### Example 22

This example demonstrates, in part, that human anucleate cell-derived vesicles comprising loaded antigen and adjuvant can induce an antigen-specific immune response in vitro.

### Materials and Methods

Human red blood cell (RBC) were obtained from healthy donors, and the resulting RBC suspension (2 billion/mL) was SQZ-processed in the presence of CMV antigen (pp65) to generate human RBC-derived vesicles loaded with CMV antigen pp65 (H-SQZ-CMV-Vesicles). pp65-specific CD8+ responder T cell were co-cultured with an exogenous adjuvant (10 μg/mL Poly I:C) and either (i) medium (negative control), (ii) pp65 peptide (positive control), or (iii) H-SQZ-CMV-Vesicles; and incubated for 24h at 37° C. After 24h, supernatant was harvested from each condition and the level of IFN-γ production was assessed by IFN-γ ELISA.

### Results

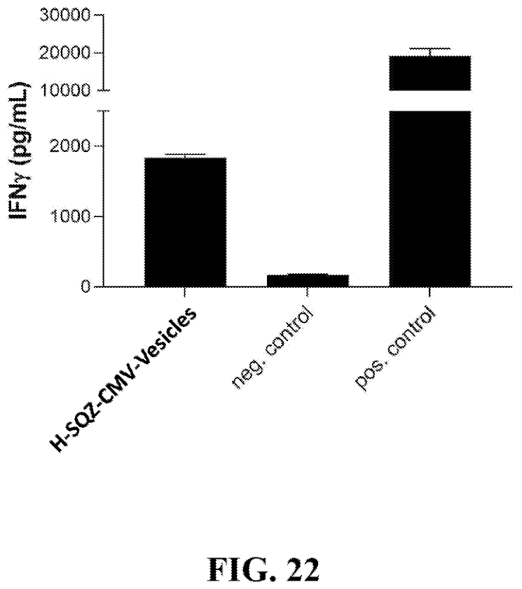

As shown in FIG. 22, IFN-γ production and secretion by CMV antigen-specific CD8+ responder T cell was significantly increased when co-cultured with H-SQZ-CMV-Vesicles or CMV antigen peptide (positive control), as compared to the minimal IFN-γ secretion for responder T cells incubated with media (negative control). These results indicate human anucleate cell-derived vesicles comprising loaded antigen and adjuvant can induce an antigen-specific immune response in vitro.

### Example 23

This example demonstrates, in part, the effect of SQZ-mediated processing in payload delivery, ghost formation and surface phosphatidylserine levels in murine anucleate cell-derived vesicles.

### Materials & Methods

RBCs were isolated using Ficoll gradient procedure from murine blood of euthanatized B6 donor mice, and a RBC suspension with a cell concentration of 1 billion/mL was prepared. The resulting RBC suspension was either (i) incubated with fluorescently-labeled OVA or fluorescently-labeled IgG (unprocessed RBCs; No SQZ), or (ii) SQZ-processed the presence of fluorescently-labeled OVA or fluorescently-labeled IgG to generate human RBC-derived vesicles loaded with respective payloads (SQZ).

To quantify the efficacy of SQZ-mediated delivery, unprocessed RBCs (No SQZ) and SQZ-processed RBC vesicles (SQZ) were measured for the presence of fluorescently-tagged payloads by flow cytometry.

To quantify for ghost formation, unprocessed RBCs (No SQZ) and SQZ-processed RBC vesicles (SQZ) were subjected to flow cytometry and analyzed for forward scatter and side scatter, using procedures as described in Example 6 and FIG. 6C.

To quantify the impact of SQZ-mediated processing on the surface phosphatidylserine levels, unprocessed RBCs (No SQZ) and SQZ-processed RBC vesicles (SQZ) were stained with Annexin V and the levels of surface phosphatidylserine were measured by flow cytometry, using procedures as described in Example 5.

### Results

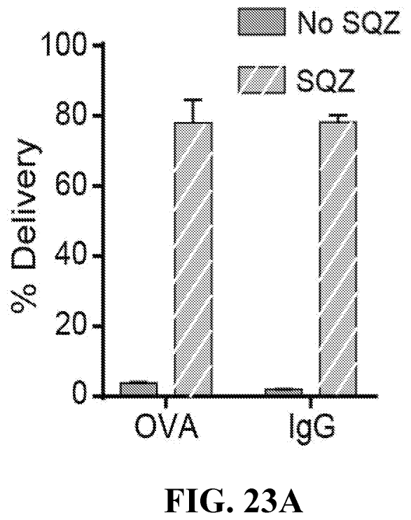

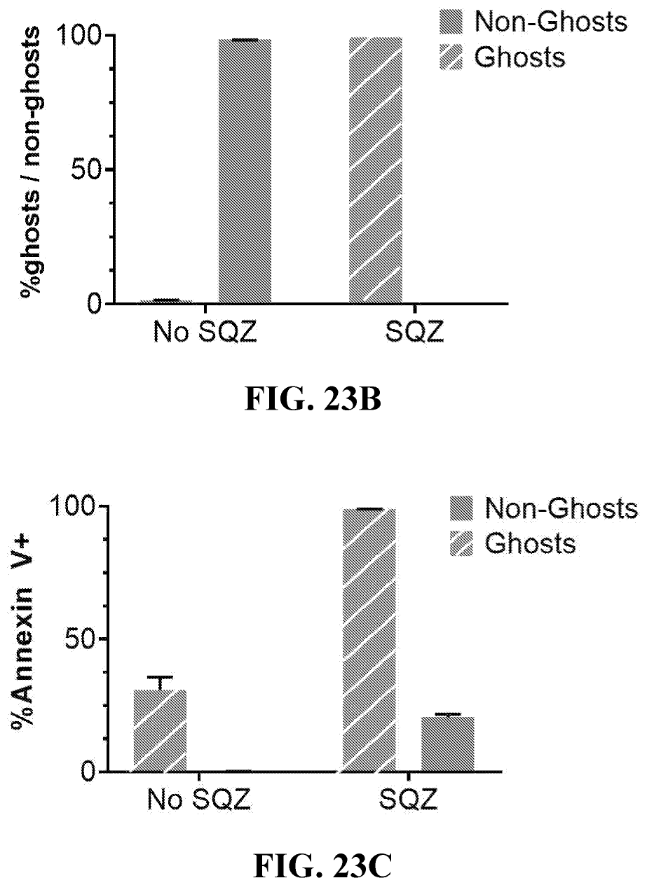

As shown in FIG. 23A, SQZ-processing of murine RBCs in the presence of a payload showed effective delivery of either OVA or IgG to a high percentage (˜80%) of the resulting RBC-derived vesicles (SQZ). As shown in FIG. 23B, SQZ-processing of murine RBCs resulted in RBC-derived vesicles of which a large majority displayed a ghost profile. In contrast, a very low proportion of unprocessed RBCs (No SQZ) displayed a ghost profile. Furthermore, as shown in FIG. 23C, the ghost and non-ghost populations from unprocessed RBCs (No SQZ) or RBC-derived vesicles (SQZ) were analyzed for surface phosphatidylserine levels. While approximately 25% of ghost population in unprocessed RBCs displayed surface phosphatidylserine, almost all of the ghost population within RBC-derived vesicles displayed surface phosphatidylserine. On the other hand, virtually none of the non-ghost population in unprocessed RBCs displayed surface phosphatidylserine, whereas about 15% of the non-ghost population in RBC-derived vesicles displayed surface phosphatidylserine.

### Example 24

This example demonstrates, in part, the ability of anucleate cell-derived vesicles containing antigen delivered by SQZ to induce in vivo antigen-dependent tolerance towards a viral capsid.

### Materials and Methods

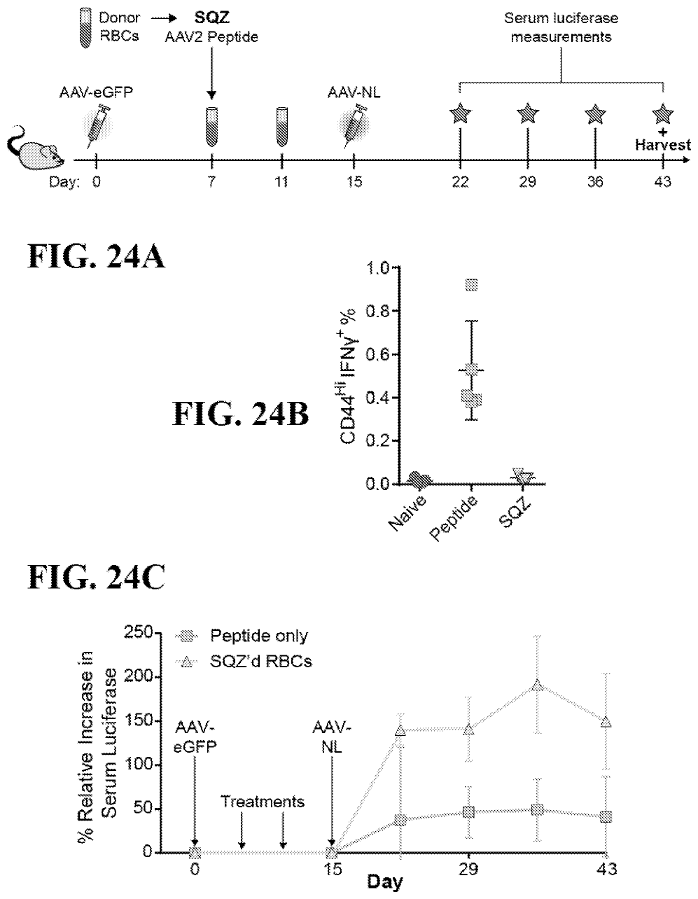

To determine the ability of anucleate cell-derived vesicles containing antigen delivered by SQZ to induce in vivo antigen-dependent tolerance towards a viral capsid, the responses of splenocytes from animals that were treated with virus and RBC-derived vesicles SQZ-loaded with AAV2 minimal epitope were measured by IFN-γ ICS. Specifically, on Day 0, C57BL/6J recipient mice were injected with AAV2 GFP virus (GFP-expressing AAV2 virus; 1E12 viral particles/mL in 100 μL PBS/mouse; 20 mice/group) or PBS alone (5 mice/group), all administered retro-orbitally (RO). On Days 7 & 11, mice were either injected (100 M/mouse) with RBCs incubated with immunogenic peptide for AAV2 capsid (SNYNKSVNV, 200 μg/mL) (Peptide) or RBC-derived vesicles SQZ-loaded with SNYNKSVNV (SQZ). On Day 15, mice were injected with AAV2 NanoLuc virus (Soluble luciferase expressing AAV2 virus; 1E12 viral particles/mL in 100 μL PBS/mouse; 20 mice/group) or PBS alone (5 mice) RO, respectively. The second virus administered was labeled with a different transgene to avoid any immune responses to the GFP transgene from the initial virus administration. On Days 22, 29, 36, 43, 100-200 μL of blood was collected via cheek bleed and the luciferase levels in serum were measured by spectrophotometry. Additionally, on Day 43, mice were sacrificed, with their spleens harvested, isolated and re-stimulated with the antigenic peptide; and the levels of the cytokine IFN-γ were measured by intracellular cytokine staining (ICS) and flow cytometry (FIG. 24A).

### Results

While there was no IFN-γ response observed in naïve animals upon stimulation with AAV2-NL, a significant increase in IFN-γ levels was observed in mice treated with SNYNKSVNV-incubated RBCs (Peptide) (P<0.005, compared to naïve), while mice treated with RBC-derived vesicles SQZ-loaded with SNYNKSVNV (SQZ) exhibited minimal immune response to the AAV2 peptide similar to that of naïve animals (FIG. 24B), indicating that the SQZ-loaded RBC-derived vesicles could reduce cytokine response specific to the loaded antigen. Furthermore, the serum luciferase measurements showed that mice treated with incubated RBCs (Peptide) did not exhibit measurable levels of luciferase over the time course tested, suggesting that no tolerance to AAV2 was induced in these animals and that the repeat dosing of AAV2-NL did not lead to transgene expression. In comparison, mice treated with SQZ-loaded RBC vesicles exhibited a 100-200% increase in serum luciferase levels (#P<0.005 compared to Peptide) indicating that tolerance to AAV2 allowed for successful expression of AAV-NL upon repeat dosing (FIG. 24C). Taken together, these data support the observation that anucleate cell-derived vesicles loaded with a viral capsid antigen can induce viral antigen-specific immune tolerance, allowing for repeated dosing of therapeutics AAV vectors.

### Example 25

This example demonstrates, in part, the ability of anucleate cell-derived vesicles containing antigen delivered by SQZ to induce in vivo antigen-dependent tolerance towards an antibody.

### Materials and Methods

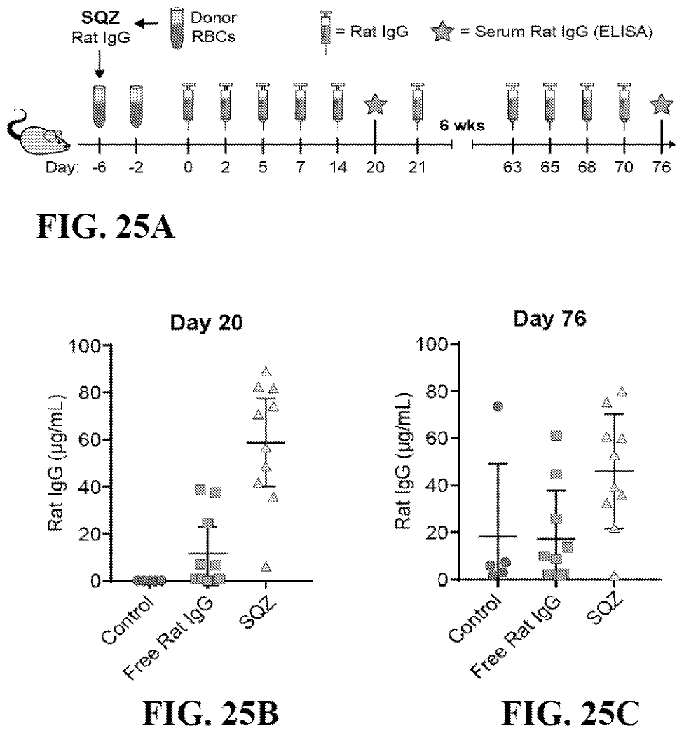

To determine the ability of anucleate cell-derived vesicles containing antigen delivered by SQZ to induce in vivo antigen-dependent tolerance towards an antibody, a rat antibody (IgG2b) was repeatedly administered to mice that were treated with RBC-derived vesicles SQZ-loaded with IgG2b, and the level of circulating antibody was quantified over time. Specifically, at Day −6 & Day −2, C57BL/6J recipient mice were injected (i) with PBS (Control; 5 mice/group), (ii) with free rat IgG2b (200 μg/mL; 5 mice/group) or (iii) with RBC-derived vesicles loaded with rat IgG2b by SQZ (100 M/mouse; 10 mice/group). Subsequent IV injection of rat IgG2b was repeatedly conducted from Day 0 to 14 and from Day 63 to 70 according to the schedule illustrated in FIG. 25A, and levels of circulating rat IgG2b in serum were assessed on Days 20 and 76 by ELISA.

### Results

As shown in FIGS. 25B and 25C, only the mice treated with SQZ-loaded RBC-derived vesicles exhibited reduced immune reactions to rat IgG, as observed from statistically significant increases in detectable levels of rat IgG in circulation. This increased circulating rat IgG was observed at the earlier time point (20 days; #P<0.005) (FIG. 25B), and was maintained even after the longer treatment regime over 70 days (76 days; *P<0.05) (FIG. 25C). Taken together, the data shows that SQZ-loaded anucleate cell-derived vesicles can be used to induce in vivo antigen-dependent tolerance to overcome anti-drug antibody reactions for a prolonged period, making repeated administration of potentially immunogenic biologics possible.

### Example 26

This example demonstrates, in part, the ability of anucleate cell-derived vesicles containing antigen delivered by SQZ to induce in vivo antigen-dependent tolerance towards an antigen associated with Type 1 diabetes (T1D).

### Materials and Methods

To determine the ability of anucleate cell-derived vesicles containing a SQZ-loaded antigen to induce in vivo antigen-dependent tolerance towards a diabetes associated antigen, two different animal models of T1D were employed: the Insulin B9-23 (Ins B9-23) model and the BDC2.5 transfer model.

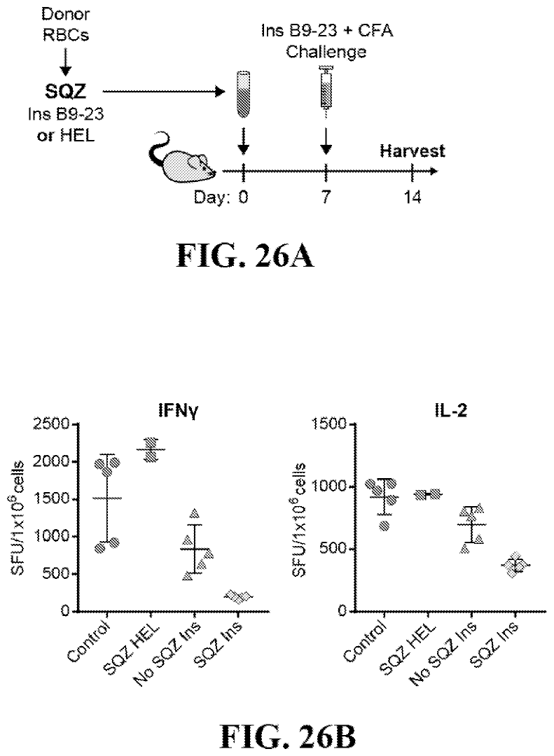

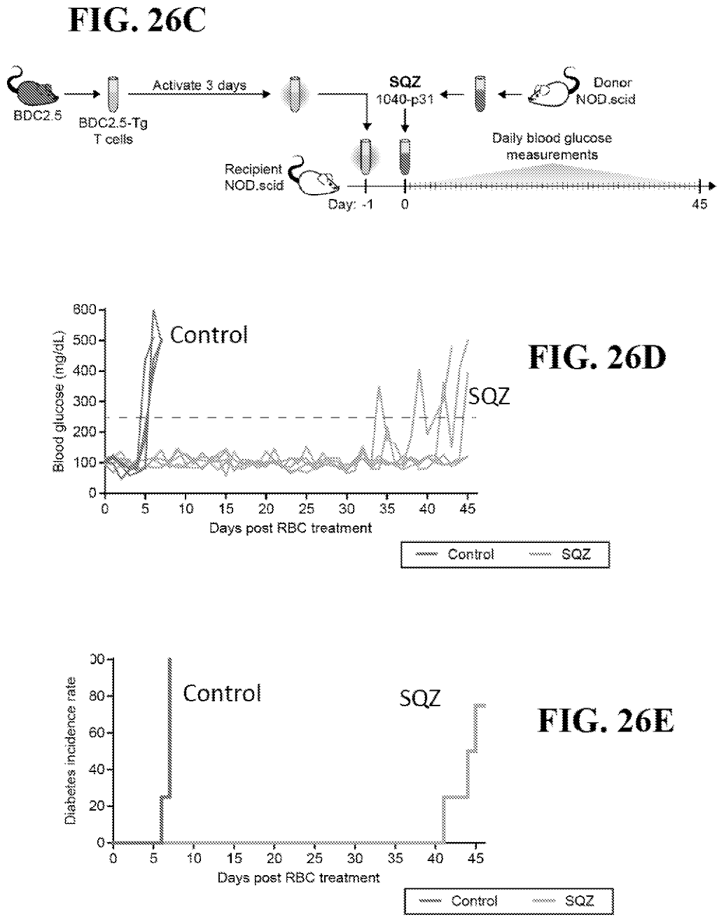

For the Ins B9-23 model, the magnitude of tolerization to Ins B9-23 was determined by cytokine production following re-stimulation with Ins B9-23 peptide in splenocytes of mice that were treated with RBC-derived vesicles SQZ-loaded with Ins B9-23 peptide. Specifically, on Day 0, NOD mice were treated with either (i) vehicle (Control), (ii) RBC-derived vesicles (1 B cells/animal in 200 μL) loaded with control peptide (SQZ HEL; 80 μM) or (iii) RBC-derived vesicles SQZ-loaded with fluorescently-tagged Ins B9-23 (SQZ FAM; 75 μM). On Day 7, mice were challenged with a 1:1 emulsion of Ins B9-23 and complete Freund's adjuvant (1 mg/mL-100 μL). On Day 14, inguinal lymph nodes were harvested, re-challenged with Ins B9-23, and intracellular cytokine staining (ICS) was conducted for IFN-γ and IL-2 and measured by flow cytometry (FIG. 26A).

For the BDC2.5 transfer model, the magnitude of tolerization was measured by delay in T1D onset in animals treated with RBC-derived vesicles SQZ-loaded with the peptide mimeotope 1040-p31. Specifically, to induce rapid onset of T1D, lymph nodes were first harvested from female BDC2.5 donor mice (3 M cells/mL) and activated by culturing with acetylated p31 peptide (500 nM in D10v2) for 3 days. Then, on Day −1, BDC2.5 T cells were harvested from the cultured lymph nodes and adoptively transferred into NOD/SCID recipient mice (1E6 cells/mouse; 5 mice/group). On Day 0, red blood cells were harvested from NOD/SCID donor mice and SQZ-loaded with 1040-p31 mimeotope peptide, and the loaded RBC-derived vesicles (SQZ; 1 B cells/mouse) or vehicle (Control; 200 μL PBS) were injected into the recipient NOD/SCID mice. Blood was drawn daily from mice and the circulating amount of blood glucose was quantified (FIG. 26C). Diabetes onset was defined by 2 consecutive measurements with >250 mg/dL of blood glucose registered. The animals were monitored for a period of 45 days, or until disease onset, whichever came first.

### Results

For the Ins B9-23 model, the results showed statistically significant reductions in levels of inflammatory cytokines IFN-γ and IL-2 in re-stimulated splenocytes from mice that were treated with RBC-derived vesicles SQZ-loaded with Ins B9-23 (SQZ FAM), compared with either SQZ HEL mice (#P<0.005) or naïve mice (Control) (#P<0.005) (FIG. 26B). This result indicated a significant reduction of antigen-specific cytokine response in mice treated with RBC-derived vesicles SQZ-loaded with a T1D relevant antigen.

For the BDC2.5 transfer model, onset of disease was on average delayed by about 35 days in mice treated with RBC-derived vesicles SQZ-loaded with 1040-p31, relative to control treated animals (FIG. 26D, FIG. 26E). This delayed onset indicated an increased tolerance to 1040-p31 in mice treated with RBC-derived vesicles SQZ-loaded with 1040-p31.

Taken together, these data support the finding that SQZ-mediated delivery of autoimmune-relevant autoantigens to RBC-derived vesicles can be used to prevent immune responses and onset of autoimmune diseases.

What is claimed is:

1. A method for delivering an antigen into an anucleate cell-derived vesicle, the method comprising:
a) passing a cell suspension comprising an input anucleate cell through a cell-deforming constriction, wherein a diameter of the constriction is a function of a diameter of the input anucleate cell in the suspension, thereby causing perturbations of the input anucleate cell large enough for the antigen to pass through to form an anucleate cell-derived vesicle; and
b) incubating the anucleate cell-derived vesicle with the antigen for a sufficient time to allow the antigen to enter the anucleate cell-derived vesicle.

2. The method of claim 1, wherein the input anucleate cell further comprises an adjuvant.

3. A method for delivering an adjuvant into an anucleate cell-derived vesicle, the method comprising:
a) passing a cell suspension comprising an input anucleate cell through a cell-deforming constriction, wherein a diameter of the constriction is a function of a diameter of the input anucleate cell in the suspension, thereby causing perturbations of the input anucleate cell large enough for the adjuvant to pass through to form an anucleate cell-derived vesicle; and
b) incubating the anucleate cell-derived vesicle with the adjuvant for a sufficient time to allow the adjuvant to enter the anucleate cell-derived vesicle.

4. The method of claim 3, wherein the input anucleate cell further comprises an antigen.

5. A method for delivering an antigen and an adjuvant into an anucleate cell-derived vesicle, the method comprising:
a) passing a cell suspension comprising an input anucleate cell through a cell-deforming constriction, wherein a diameter of the constriction is a function of a diameter of the input anucleate cell in the suspension, thereby causing perturbations of the input anucleate cell large enough for the antigen and the adjuvant to pass through to form an anucleate cell-derived vesicle; and
b) incubating the anucleate cell-derived vesicle with the antigen and the adjuvant for a sufficient time to allow the antigen and the adjuvant to enter the anucleate cell-derived vesicle.

6. A method for stimulating an immune response to an antigen in an individual, the method comprising administering to the individual an effective amount of an anucleate cell-derived vesicle comprising an antigen, wherein the anucleate cell-derived vesicle comprising the antigen is prepared by a process comprising the steps of:
a) passing a cell suspension comprising an input anucleate cell through a cell-deforming constriction, wherein a diameter of the constriction is a function of a diameter of the input anucleate cell in the suspension, thereby causing perturbations of the input anucleate cell large enough for the antigen to pass through to form an anucleate cell-derived vesicle; and
b) incubating the anucleate cell-derived vesicle with the antigen for a sufficient time to allow the antigen to enter the anucleate cell-derived vesicle.

7. The method of claim 6, wherein the method further comprises administering an adjuvant systemically to the individual.

8. The method of claim 7, wherein the adjuvant is administered systemically before, after or at the same time as the anucleate cell derived vesicle.

9. The method of any one of claims 6-8, wherein the input anucleate cell comprises an adjuvant.

10. A method for stimulating an immune response to an antigen in an individual, the method comprising administering to the individual an effective amount of an anucleate cell-derived vesicle comprising an antigen and an adjuvant, wherein the anucleate cell-derived vesicle comprising the antigen and the adjuvant is prepared by a process comprising the steps of:
a) passing a cell suspension comprising an input anucleate cell through a cell-deforming constriction, wherein a diameter of the constriction is a function of a diameter of the input anucleate cell in the suspension, thereby causing perturbations of the input anucleate cell large enough for the antigen and the adjuvant to pass through to form an anucleate cell-derived vesicle; and
b) incubating the anucleate cell-derived vesicle with the antigen and the adjuvant for a sufficient time to allow the antigen and the adjuvant to enter the anucleate cell-derived vesicle.

11. The method of claim 10, wherein the method further comprises administering an adjuvant systemically to the individual.

12. The method of claim 11, wherein the adjuvant is administered systemically before, after or at the same time as the anucleate cell-derived vesicle.

13. A method for treating a disease in an individual, comprising administering to the individual an anucleate cell-derived vesicle comprising a disease-associated antigen, wherein an immune response against the antigen ameliorates conditions of the disease, and wherein the anucleate cell-derived vesicle comprising the disease-associated antigen is prepared by a process comprising the steps of:
a) passing a cell suspension comprising an input anucleate cell through a cell-deforming constriction, wherein a diameter of the constriction is a function of a diameter of the input anucleate cell in the suspension, thereby causing perturbations of the input anucleate cell large enough for the antigen to pass through to form an anucleate cell-derived vesicle; and
b) incubating the anucleate cell-derived vesicle with the antigen for a sufficient time to allow the antigen to enter the anucleate cell-derived vesicle.

14. A method for preventing a disease in an individual, comprising administering to the individual an anucleate cell-derived vesicle comprising a disease-associated antigen, wherein an immune response against the antigen prevents development of the disease, and wherein the anucleate cell-derived vesicle comprising the disease-associated antigen is prepared by a process comprising the steps of:
a) passing a cell suspension comprising an input anucleate cell through a cell-deforming constriction, wherein a diameter of the constriction is a function of a diameter of the input anucleate cell in the suspension, thereby causing perturbations of the input anucleate cell large enough for the antigen to pass through to form an anucleate cell-derived vesicle; and
b) incubating the anucleate cell-derived vesicle with the antigen for a sufficient time to allow the antigen to enter the anucleate cell-derived vesicle.

15. A method for vaccinating an individual against an antigen, comprising administering to the individual an anucleate cell-derived vesicle comprising the antigen, wherein the anucleate cell-derived vesicle comprising the antigen is prepared by a process comprising the steps of:
a) passing a cell suspension comprising an input anucleate cell through a cell-deforming constriction, wherein a diameter of the constriction is a function of a diameter of the input anucleate cell in the suspension, thereby causing perturbations of the input anucleate cell large enough for the antigen to pass through to form an anucleate cell-derived vesicle; and
b) incubating the anucleate cell-derived vesicle with the antigen for a sufficient time to allow the antigen to enter the anucleate cell-derived vesicle.

16. The method of any one of claims 13-15, wherein the method further comprises administering an adjuvant systemically to the individual.

17. The method of claim 16, wherein the adjuvant is administered systemically before, after or at the same time as the anucleate cell derived vesicle.

18. The method of claim 13-17, wherein the input anucleate cell comprises an adjuvant.

19. A method for treating a disease in an individual, comprising administering to the individual an anucleate cell-derived vesicle comprising a disease-associated antigen and an adjuvant, wherein an immune response against the antigen ameliorates conditions of the disease, and wherein the anucleate cell-derived vesicle comprising the disease-associated antigen and the adjuvant is prepared by a process comprising the steps of:
a) passing a cell suspension comprising an input anucleate cell through a cell-deforming constriction, wherein a diameter of the constriction is a function of a diameter of the input anucleate cell in the suspension, thereby causing perturbations of the input anucleate cell large enough for the antigen and an adjuvant to pass through to form an anucleate cell-derived vesicle; and
b) incubating the anucleate cell-derived vesicle with the antigen and the adjuvant for a sufficient time to allow the antigen and the adjuvant to enter the anucleate cell-derived vesicle.

20. A method for preventing a disease in an individual, comprising administering to the individual an anucleate cell-derived vesicle comprising a disease-associated antigen and an adjuvant, wherein an immune response against the antigen prevents development of the disease, and wherein the anucleate cell-derived vesicle comprising a disease-associated antigen and an adjuvant is prepared by a process comprising the steps of:
a) passing a cell suspension comprising an input anucleate cell through a cell-deforming constriction, wherein a diameter of the constriction is a function of a diameter of the input anucleate cell in the suspension, thereby causing perturbations of the input anucleate cell large enough for the antigen and an adjuvant to pass through to form an anucleate cell-derived vesicle; and
b) incubating the anucleate cell-derived vesicle with the antigen and the adjuvant for a sufficient time to allow the antigen and the adjuvant to enter the anucleate cell-derived vesicle.

21. A method for vaccinating an individual against an antigen, comprising administering to the individual an anucleate cell-derived vesicle comprising the antigen and an adjuvant, wherein the anucleate cell-derived vesicle comprising the antigen and the adjuvant is prepared by a process comprising the steps of:
a) passing a cell suspension comprising an input anucleate cell through a cell-deforming constriction, wherein a diameter of the constriction is a function of a diameter of the input anucleate cell in the suspension, thereby causing perturbations of the input anucleate cell large enough for the antigen and an adjuvant to pass through to form an anucleate cell-derived vesicle; and
b) incubating the anucleate cell-derived vesicle with the antigen and the adjuvant for a sufficient time to allow the antigen and the adjuvant to enter the anucleate cell-derived vesicle.

22. A method for treating a disease in an individual, wherein an immune response against a disease-associated antigen ameliorates conditions of the disease, the method comprising
a) passing a cell suspension comprising an input anucleate cell through a cell-deforming constriction, wherein a diameter of the constriction is a function of a diameter of the input anucleate cell in the suspension, thereby causing perturbations of the input anucleate cell large enough for the antigen to pass through to form an anucleate cell-derived vesicle;
b) incubating the anucleate cell-derived vesicle with the antigen for a sufficient time to allow the antigen to enter the anucleate cell-derived vesicle, thereby generating an anucleate cell-derived vesicle comprising the antigen; and
c) administering the anucleate cell-derived vesicle comprising the antigen to the individual

23. A method for preventing a disease in an individual, wherein an immune response against a disease-associated antigen prevents development of the disease, the method comprising
a) passing a cell suspension comprising an input anucleate cell through a cell-deforming constriction, wherein a diameter of the constriction is a function of a diameter of the input anucleate cell in the suspension, thereby causing perturbations of the input anucleate cell large enough for the antigen to pass through to form an anucleate cell-derived vesicle;
b) incubating the anucleate cell-derived vesicle with the antigen for a sufficient time to allow the antigen to enter the anucleate cell-derived vesicle, thereby generating an anucleate cell-derived vesicle comprising the antigen; and
c) administering the anucleate cell-derived vesicle comprising the antigen to the individual.

24. A method for vaccinating an individual against an antigen, the method comprising,
a) passing a cell suspension comprising an input anucleate cell through a cell-deforming constriction, wherein a diameter of the constriction is a function of a diameter of the input anucleate cell in the suspension, thereby causing perturbations of the input anucleate cell large enough for the antigen to pass through to form an anucleate cell-derived vesicle;
b) incubating the anucleate cell-derived vesicle with the antigen for a sufficient time to allow the antigen to enter the anucleate cell-derived vesicle, thereby generating an anucleate cell-derived vesicle comprising the antigen; and
c) administering the anucleate cell-derived vesicle comprising the antigen to the individual.

25. The method of any one of claims 19-24, wherein the method further comprises administering an extravesicular adjuvant systemically to the individual.

26. The method of claim 25, wherein the extravesicular adjuvant is administered before, after or at the same time as the anucleate cell-derived vesicle.

27. The method of claim 19-24, wherein the input anucleate cell comprises an adjuvant.

28. A method for treating a disease in an individual, wherein an immune response against a disease-associated antigen ameliorates conditions of the disease, the method comprising
a) passing a cell suspension comprising an input anucleate cell through a cell-deforming constriction, wherein a diameter of the constriction is a function of a diameter of the input anucleate cell in the suspension, thereby causing perturbations of the input anucleate cell large enough for the disease-associated antigen and an adjuvant to pass through to form an anucleate cell-derived vesicle;
b) incubating the anucleate cell-derived vesicle with the antigen and the adjuvant for a sufficient time to allow the antigen and the adjuvant to enter the anucleate cell-derived vesicle, thereby generating an anucleate cell-derived vesicle comprising the antigen and the adjuvant; and
c) administering the anucleate cell-derived vesicle comprising the antigen and the adjuvant to the individual

29. A method for preventing a disease in an individual, wherein an immune response against a disease-associated antigen prevents development of the disease, the method comprising
a) passing a cell suspension comprising an input anucleate cell through a cell-deforming constriction, wherein a diameter of the constriction is a function of a diameter of the input anucleate cell in the suspension, thereby causing perturbations of the input anucleate cell large enough for the antigen and an adjuvant to pass through to form an anucleate cell-derived vesicle;
b) incubating the anucleate cell-derived vesicle with the antigen and the adjuvant for a sufficient time to allow the antigen and the adjuvant to enter the anucleate cell-derived vesicle, thereby generating an anucleate cell-derived vesicle comprising the antigen and the adjuvant; and
c) administering the anucleate cell-derived vesicle comprising the antigen and the adjuvant to the individual.

30. A method for vaccinating an individual against an antigen, the method comprising,
a) passing a cell suspension comprising an input anucleate cell through a cell-deforming constriction, wherein a diameter of the constriction is a function of a diameter of the input anucleate cell in the suspension, thereby causing perturbations of the input anucleate cell large enough for the antigen and an adjuvant to pass through to form an anucleate cell-derived vesicle;
b) incubating the anucleate cell-derived vesicle with the antigen and the adjuvant for a sufficient time to allow the antigen and the adjuvant to enter the anucleate cell-derived vesicle, thereby generating an anucleate cell-derived vesicle comprising the antigen and the adjuvant; and
c) administering the anucleate cell-derived vesicle comprising the antigen and the adjuvant to the individual.

31. The method of any one of claims 28-30, wherein the method further comprises administering an extravesicular adjuvant systemically to the individual.

32. The method of claim 31, wherein the extravesicular adjuvant is administered before, after or at the same time as the anucleate cell derived vesicle.

33. The method of any one of claims 13-32, wherein the disease is cancer, an infectious disease or a viral-associated disease.

34. The method of any one of claims 6-33 wherein the anucleate cell-derived vesicle is autologous to the individual.

35. The method of any one of claims 6-33, wherein the anucleate cell-derived vesicle is allogeneic to the individual.

36. The method of any one of claims 6-35, wherein the anucleate cell-derived vesicle is in a pharmaceutical formulation.

37. The method of any one of claims 6-36, wherein the anucleate cell-derived vesicle is administered systemically.

38. The method of any one of claims 6-37, wherein the anucleate cell-derived vesicle is administered intravenously, intraarterially, subcutaneously, intramuscularly, or intraperitoneally.

39. The method of any one of claims 6-38, wherein the anucleate cell-derived vesicle is administered to the individual in combination with a therapeutic agent.

40. The method of claim 39, wherein the therapeutic agent is administered before, after or at the same time as the anucleate cell-derived vesicle.

41. The method of claim 39 or 40, wherein the therapeutic agent is an immune checkpoint inhibitor and/or a cytokine.

42. The method of claim 41, wherein the cytokine is one or more of IFN-α, IFN-γ, IL-2 or IL-15.

43. The method of claim 41, wherein the immune checkpoint inhibitor is targeted to any one of PD-1, PD-L1, CTLA-4, TIM-3, LAGS, TIGIT, VISTA, TIM1, B7-H4 (VTCN1) and BTLA.

44. The method of any one of claim 1, 2, or 4-43, wherein the antigen is capable of being processed into an MHC class I-restricted peptide and/or an MHC class II-restricted peptide.

45. The method of any one of claim 1, 2, or 4-43, wherein the antigen is a CD-1 restricted antigen.

46. The method of any one of claim 1, 2, or 4-45, wherein the antigen is a disease-associated antigen.

47. The method of any one of claim 1, 2, or 4-46, wherein the antigen is a tumor antigen.

48. The method of any one of claim 1, 2, or 4-47, wherein the antigen is derived from a lysate.

49. The method of claim 48, wherein the lysate is a tumor lysate.

50. The method of any one of claim 1, 2, or 4-46, wherein the antigen is a viral antigen, a bacterial antigen or a fungal antigen.

51. The method of any one of claim 1, 2, or 4-46, wherein the antigen is a microorganism.

52. The method of any one of claim 1, 2, or 4-50, wherein the antigen is a polypeptide.

53. The method of any one of claim 1, 2, or 4-50, wherein the antigen is a lipid antigen.

54. The method of any one of claim 1, 2, or 4-50, wherein the antigen is a carbohydrate antigen.

55. The method of any one of claim 1, 2, or 4-54, wherein the antigen is a modified antigen.

56. The method of claim 55, wherein the modified antigen comprises an antigen fused with a polypeptide.

57. The method of claim 56, wherein the modified antigen comprises an antigen fused with a targeting peptide.

58. The method of claim 55, wherein the modified antigen comprises an antigen fused with a lipid.

59. The method of claim 55, wherein the modified antigen comprises an antigen fused with a carbohydrate.

60. The method of claim 55, wherein the modified antigen comprises an antigen fused with a nanoparticle.

61. The method of any one of claims 1-60, wherein a plurality of antigens is delivered to the anucleate cell-derived vesicle.

62. The method of any one of claims 2-5, 7-12, 16-21, 25-61 wherein the adjuvant is a CpG ODN, IFN-α, STING agonists, RIG-I agonists, poly I:C, polyinosinic-polycytidylic acid stabilized with polylysine and carboxymethylcellulose (HILTONOL®), imiquimod, resiquimod, and/or lipopolysaccharide (LPS).

63. The method of claim 62, wherein the adjuvant is low molecular weight poly I:C.

64. The method of any one of claims 1-63, wherein the input anucleate cell is a red blood cell.

65. The method of any one of claims 1-63, wherein the red blood cell is an erythrocyte.

66. The method of any one of claims 1-63, wherein the red blood cell is a reticulocyte.

67. The method of any one of claims 1-63, wherein the input anucleate cell is a platelet.

68. The method of any one of claims 1-67, wherein the input anucleate cell is a mammalian cell.

69. The method of any one of claims 1-68, wherein the input anucleate cell is a monkey, mouse, dog, cat, horse, rat, sheep, goat, pig, or rabbit cell.

70. The method of any one of claims 1-68, wherein the input anucleate cell is a human cell.

71. The method of any one of claims 1-70, wherein the constriction is contained within a microfluidic channel.

72. The method of claim 71, wherein the microfluidic channel comprises a plurality of constrictions.

73. The method of claim 72, wherein the plurality of constrictions are arranged in series and/or in parallel.

74. The method of any one of claims 1-73, wherein the constriction is between a plurality of micropillars; between a plurality of micropillars configured in an array; or between one or more movable plates.

75. The method of any one of claims 1-70, wherein the constriction is a pore or contained within a pore.

76. The method of claim 75, wherein the pore is contained in a surface.

77. The method of claim 76, wherein the surface is a filter.

78. The method of claim 76, wherein the surface is a membrane.

79. The method of any one of claims 1-76, wherein the constriction size is a function of the diameter of the input anucleate cell in suspension.

80. The method of any one of claims 1-79, wherein the constriction size is about 10%, about 20%, about 30%, about 40%, about 50%, about 60%, or about 70% of the diameter of the input anucleate cell in suspension.

81. The method of any one of claims 1-79, wherein the constriction has a width of about 0.25 μm to about 4 μm.

82. The method of any one of claims 1-79, wherein the constriction has a width of about 4 μm, 3.5 μm, about 3 μm, about 2.5 μm, about 2 μm, about 1.5 μm, about 1 μm, about 0.5 μm, or about 0.25 μm.

83. The method of any one of claims 1-79, wherein the constriction has a width of about 2.2 μm.

84. The method of any one of claims 1-83, wherein the input anucleate cells are passed through the constriction under a pressure ranging from about 10 psi to about 90 psi.

85. The method of any one of claims 1-84, wherein said cell suspension is contacted with the antigen before, concurrently, or after passing through the constriction.

86. An anucleate cell-derived vesicle comprising an antigen, wherein the anucleate cell-derived vesicle comprising the antigen is prepared by a process comprising the steps of:
a) passing a cell suspension comprising an input anucleate cell through a cell-deforming constriction, wherein a diameter of the constriction is a function of a diameter of the input anucleate cell in the suspension, thereby causing perturbations of the input anucleate cell large enough for the antigen to pass through to form an anucleate cell-derived vesicle; and
b) incubating the anucleate cell-derived vesicle with the antigen for a sufficient time to allow the antigen to enter the anucleate cell-derived vesicle;
thereby generating the anucleate cell-derived vesicle comprising the antigen.

87. The anucleate cell-derived vesicle of claim 86, wherein the input anucleate cell comprises an adjuvant.

88. An anucleate cell-derived vesicle comprising an adjuvant, wherein the anucleate cell-derived vesicle comprising the adjuvant is prepared by a process comprising the steps of:
a) passing a cell suspension comprising an input anucleate cell through a cell-deforming constriction, wherein a diameter of the constriction is a function of a diameter of the input anucleate cell in the suspension, thereby causing perturbations of the input anucleate cell large enough for the adjuvant to pass through to form an anucleate cell-derived vesicle; and
b) incubating the anucleate cell-derived vesicle with the adjuvant for a sufficient time to allow the adjuvant to enter the anucleate cell-derived vesicle;
thereby generating the anucleate cell-derived vesicle comprising the adjuvant.

89. The anucleate cell-derived vesicle of claim 88, wherein the input anucleate cell comprises an antigen.

90. An anucleate cell-derived vesicle comprising an antigen and an adjuvant, wherein the anucleate cell-derived vesicle comprising the antigen and the adjuvant is prepared by a process comprising the steps of:
a) passing a cell suspension comprising an input anucleate cell through a cell-deforming constriction, wherein a diameter of the constriction is a function of a diameter of the input anucleate cell in the suspension, thereby causing perturbations of the input anucleate cell large enough for the antigen and the adjuvant to pass through to form an anucleate cell-derived vesicle; and
b) incubating the anucleate cell-derived vesicle with the antigen and the adjuvant for a sufficient time to allow the antigen and the adjuvant to enter the anucleate cell-derived vesicle; thereby generating the anucleate cell-derived vesicle comprising the antigen and the adjuvant.

91. The anucleate cell-derived vesicle of any one of claims 86-90, wherein the anucleate cell-derived vesicle is a red blood cell-derived vesicle or a platelet-derived vesicle.

92. The anucleate cell-derived vesicle of claim 91, wherein the red blood cell-derived vesicle is an erythrocyte-derived vesicle, or a reticulocyte-derived vesicle.

93. The anucleate cell-derived vesicle of any one of claim 86, 87, or 89-92, wherein the antigen is capable of being processed into an MHC class I-restricted peptide and/or an MHC class II-restricted peptide.

94. The anucleate cell-derived vesicle of any one of claim 86, 87, or 89-92, wherein the antigen is a CD-1 restricted antigen.

95. The anucleate cell-derived vesicle of any one of claim 86, 87, or 89-94, wherein the antigen is a disease-associated antigen.

96. The anucleate cell-derived vesicle of any one of claim 86, 87, or 89-95, wherein the antigen is a tumor antigen.

97. The anucleate cell-derived vesicle of any one of claim 86, 87, or 89-96, wherein the antigen is derived from a lysate.

98. The anucleate cell-derived vesicle of claim 97, wherein the lysate is a tumor lysate.

99. The anucleate cell-derived vesicle of any one of claim 86, 87, or 89-95, wherein the antigen is a viral antigen, a bacterial antigen or a fungal antigen.

100. The anucleate cell-derived vesicle of any one of claim 86, 87, or 89-95, wherein the antigen is a microorganism.

101. The anucleate cell-derived vesicle of any one of claim 86, 87, or 89-99, wherein the antigen is a polypeptide.

102. The anucleate cell-derived vesicle of any one of claim 86, 87, or 89-99, wherein the antigen is a lipid antigen.

103. The anucleate cell-derived vesicle of any one of claim 86, 87, or 89-99, wherein the antigen is a carbohydrate antigen.

104. The anucleate cell-derived vesicle of any one of claim 86, 87, or 89-103, wherein the antigen is a modified antigen.

105. The anucleate cell-derived vesicle of claim 104, wherein the modified antigen comprises an antigen fused with a polypeptide.

106. The anucleate cell-derived vesicle of claim 105, wherein the modified antigen comprises an antigen fused with a targeting peptide.

107. The anucleate cell-derived vesicle of claim 104, wherein the modified antigen comprises an antigen fused with a lipid.

108. The anucleate cell-derived vesicle of claim 104, wherein the modified antigen comprises an antigen fused with a carbohydrate.

109. The anucleate cell-derived vesicle of claim 104, wherein the modified antigen comprises an antigen fused with a nanoparticle.

110. The anucleate cell-derived vesicle of any one of claim 86, 87, or 89-109, wherein a plurality of antigens is delivered to the anucleate cell-derived vesicle.

111. The anucleate cell-derived vesicle of any one of claims 87-110 wherein the adjuvant is a CpG ODN, IFN-α, STING agonists, RIG-I agonists, poly I:C, polyinosinic-polycytidylic acid stabilized with polylysine and carboxymethylcellulose (HILTONOL®), imiquimod, resiquimod and/or LPS.

112. The anucleate cell-derived vesicle of claim 111, wherein the adjuvant is low molecular weight poly I:C.

113. The anucleate cell-derived vesicle of any one of claims 86-112 wherein the input anucleate cell is a red blood cell.

114. The anucleate cell-derived vesicle of any one of claims 86-112, wherein the input anucleate cell is an erythrocyte.

115. The anucleate cell-derived vesicle of any one of claims 86-112, wherein the input anucleate cell is a reticulocyte.

116. The anucleate cell-derived vesicle of any one of claims 86-112, wherein the input anucleate cell is a platelet.

117. The anucleate cell-derived vesicle of any one of claims 86-116 wherein the input anucleate cell is a mammalian cell.

118. The anucleate cell-derived vesicle of any one of claims 86-117, wherein the input anucleate cell is a monkey, mouse, dog, cat, horse, rat, sheep, goat, pig, or rabbit cell.

119. The anucleate cell-derived vesicle of any one of claims 86-117, wherein the input anucleate cell is a human cell.

120. The anucleate cell-derived vesicle of any one of claims 86-119 wherein a half-life of the anucleate cell-derived vesicle following administration to a mammal is decreased compared to a half-life of the input anucleate cell following administration to the mammal.

121. The anucleate cell-derived vesicle of any one of claim 86-115, or 117-120, wherein a hemoglobin content of the anucleate cell-derived vesicle is decreased compared to the hemoglobin content of the input anucleate cell.

122. The anucleate cell-derived vesicle of any one of claims 86-120, wherein ATP production of the anucleate cell-derived vesicle is decreased compared to ATP production of the input anucleate cell.

123. The anucleate cell-derived vesicle of any one of claims 113, 114, 117-122 wherein the anucleate cell-derived vesicle exhibits one or more of the following properties:
(a) a circulating half-life in a mammal that is decreased compared to the input anucleate cell;
(b) decreased hemoglobin level compared to the input anucleate cell;
(c) a spherical morphology;
(d) increased surface phosphatidylserine levels compared to the input anucleate cell,
(e) reduced ATP production compared to the input anucleate cell.

124. The anucleate cell-derived vesicle of any one of claims 113, 114, 117-122, wherein the input anucleate cell is an erythrocyte and wherein the anucleate cell-derived vesicle has a reduced biconcave shape compared to the input anucleate cell.

125. The anucleate cell-derived vesicle of claim 113, 114, 117-122 wherein the anucleate cell-derived vesicle is a red blood cell ghost.

126. The anucleate cell-derived vesicle of any one of claims 86-125, wherein the anucleate cell-derived vesicles prepared by the process have greater than about 1.5 fold more phosphatidylserine on its surface compared to the input anucleate cell.

127. The anucleate cell-derived vesicle of any one of claims 86-126, wherein a population profile of anucleate cell-derived vesicles prepared by the process exhibits higher average phosphatidylserine levels on the surface compared to the input anucleate cells.

128. The anucleate cell-derived vesicle of any one of claims 86-127, wherein at least 50% of the population profile of anucleate cell-derived vesicles prepared by the process exhibits higher phosphatidylserine levels on the surface compared to the input anucleate cells

129. The anucleate cell-derived vesicle of any one of claims 86-128, wherein the anucleate cell-derived vesicle exhibits enhanced uptake in a tissue or cell compared to the input anucleate cell.

130. The anucleate cell-derived vesicle of claim 129, wherein the anucleate cell-derived vesicle exhibits enhanced uptake in liver and/or spleen or by a phagocytic cell and/or an antigen-presenting cell compared to the uptake of the input anucleate cell.

131. The anucleate cell-derived vesicle of any one of claims 86-130, wherein the anucleate cell-derived vesicle is modified to enhance uptake in a tissue or cell compared to an unmodified anucleate cell-derived vesicle.

132. The anucleate cell-derived vesicle of claim 131, wherein the anucleate cell-derived vesicle is modified to enhance uptake in liver and/or spleen or by a phagocytic cell and/or an antigen-presenting cell compared to the uptake of the input anucleate cell.

133. The anucleate cell-derived vesicle of any one of claims 86-132, wherein the anucleate cell-derived vesicle comprises CD47 on its surface.

134. The anucleate cell-derived vesicle of any one of claims 86-133, wherein the anucleate cell-derived vesicle is not (a) heat processed, (b) chemically treated, and/or (c) subjected to hypotonic or hypertonic conditions during the preparation of the anucleate cell-derived vesicles.

135. The anucleate cell-derived vesicle of any one of claims 86-134, wherein the osmolarity of the cell suspension is maintained throughout the process.

136. The anucleate cell-derived vesicle of claims 86-135, wherein the osmolarity of the cell suspension is maintained between 200 mOsm and 400 mOsm throughout the process.

137. The anucleate cell-derived vesicle of any one of claims 86-136, wherein the constriction is contained within a microfluidic channel.

138. The anucleate cell-derived vesicle of claim 137, wherein the microfluidic channel comprises a plurality of constrictions.

139. The anucleate cell-derived vesicle of claim 138, wherein the plurality of constrictions are arranged in series and/or in parallel.

140. The anucleate cell-derived vesicle of any one of claims 86-139, wherein the constriction is between a plurality of micropillars; between a plurality of micropillars configured in an array; or between one or more movable plates.

141. The anucleate cell-derived vesicle of any one of claims 86-136, wherein the constriction is a pore or contained within a pore.

142. The anucleate cell-derived vesicle of claim 141, wherein the pore is contained in a surface.

143. The anucleate cell-derived vesicle of claim 142, wherein the surface is a filter.

144. The anucleate cell-derived vesicle of claim 142, wherein the surface is a membrane.

145. The anucleate cell-derived vesicle of any one of claims 86-144, wherein the constriction size is a function of the diameter of the input anucleate cell in suspension.

146. The anucleate cell-derived vesicle of any one of claims 86-144, wherein the constriction size is about 10%, about 20%, about 30%, about 40%, about 50%, about 60%, or about 70% of the diameter of the input anucleate cell in suspension.

147. The anucleate cell-derived vesicle of any one of claims 86-146, wherein the constriction has a width of about 0.25 μm to about 4 μm.

148. The anucleate cell-derived vesicle of any one of claims 86-147, wherein the constriction has a width of about 4 μm, 3.5 μm, about 3 μm, about 2.5 μm, about 2 μm, about 1.5 μm, about 1 μm, about 0.5 μm, or about 0.25 μm.

149. The anucleate cell-derived vesicle of any one of claims 86-147, wherein the constriction has a width of about 2.2 μm.

150. The anucleate cell-derived vesicle of any one of claims 86-149, wherein the input anucleate cells are passed through the constriction under a pressure ranging from about 10 psi to about 90 psi.

151. The anucleate cell-derived vesicle of any one of claims 86-150, wherein said cell suspension is contacted with the antigen before, concurrently, or after passing through the constriction.

152. A composition comprising a plurality of anucleate cell-derived vesicles of any one of claims 86-151.

153. The composition of claim 152, further comprising a pharmaceutically acceptable excipient.

154. A method for generating an anucleate cell-derived vesicle comprising an antigen, the method comprising:
a) passing a cell suspension comprising an input anucleate cell through a cell-deforming constriction, wherein a diameter of the constriction is a function of a diameter of the input anucleate cell in the suspension, thereby causing perturbations of the input anucleate cell large enough for the antigen to pass through to form an anucleate cell-derived vesicle;
b) incubating the anucleate cell-derived vesicle with the antigen for a sufficient time to allow the antigen to enter the anucleate cell-derived vesicle, thereby generating an anucleate cell-derived vesicle comprising the antigen.

155. The method of claim 154, wherein the input anucleate cell comprises an adjuvant.

156. A method for generating an anucleate cell-derived vesicle comprising an adjuvant, the method comprising:
a) passing a cell suspension comprising an input anucleate cell through a cell-deforming constriction, wherein a diameter of the constriction is a function of a diameter of the input anucleate cell in the suspension, thereby causing perturbations of the input anucleate cell large enough for the adjuvant to pass through to form an anucleate cell-derived vesicle;
b) incubating the anucleate cell-derived vesicle with the adjuvant for a sufficient time to allow the adjuvant to enter the anucleate cell-derived vesicle, thereby generating an anucleate cell-derived vesicle comprising the adjuvant.

157. The method of claim 156, wherein the input anucleate cell comprises an antigen.

158. A method for generating an anucleate cell-derived vesicle comprising an antigen and an adjuvant, the method comprising:
a) passing a cell suspension comprising an input anucleate cell through a cell-deforming constriction, wherein a diameter of the constriction is a function of a diameter of the input anucleate cell in the suspension, thereby causing perturbations of the input anucleate cell large enough for the antigen and the adjuvant to pass through to form an anucleate cell-derived vesicle;
b) incubating the anucleate cell-derived vesicle with the antigen and the adjuvant for a sufficient time to allow the antigen and the adjuvant to enter the anucleate cell-derived vesicle, thereby generating an anucleate cell-derived vesicle comprising the antigen and the adjuvant.

159. The method of any one of claims 154-158, wherein the anucleate cell-derived vesicle is a red blood cell-derived vesicle or a platelet derived vesicle.

160. The method of claim 159, wherein the red blood cell-derived vesicle is an erythrocyte-derived vesicle or a reticulocyte-derived vesicle.

161. The method of any one of claim 154, 155 or 157-160, wherein the antigen is capable of being processed into an MHC class I-restricted peptide and/or an MHC class II-restricted peptide.

162. The method of any one of claim 154, 155 or 157-160, wherein the antigen is a CD-1 restricted antigen.

163. The method of any one of claim 154, 155 or 157-162, wherein the antigen is a disease-associated antigen.

164. The method of any one of claim 154, 155 or 157-163, wherein the antigen is a tumor antigen.

165. The method of any one of claim 154, 155 or 157-164, wherein the antigen is derived from a lysate.

166. The method of claim 165, wherein the lysate is a tumor lysate.

167. The method of any one of claim 154, 155 or 157-163, wherein the antigen is a viral antigen, a bacterial antigen or a fungal antigen.

168. The method of any one of claim 154, 155 or 157-163, wherein the antigen is a microorganism.

169. The method of any one of claim 154, 155 or 157-167, wherein the antigen is a polypeptide.

170. The method of any one of claim 154, 155 or 157-167, wherein the antigen is a lipid antigen.

171. The method of any one of claim 154, 155 or 157-167, wherein the antigen is a carbohydrate antigen.

172. The method of any one of claim 154, 155 or 157-171, wherein the antigen is a modified antigen.

173. The method of claim 172, wherein the modified antigen comprises an antigen fused with a polypeptide.

174. The method of claim 173, wherein the modified antigen comprises an antigen fused with a targeting peptide.

175. The method of claim 174, wherein the modified antigen comprises an antigen fused with a lipid.

176. The method of claim 175, wherein the modified antigen comprises an antigen fused with a carbohydrate.

177. The method of claim 176, wherein the modified antigen comprises an antigen fused with a nanoparticle.

178. The method of any one of claim 154, 155 or 157-177, wherein a plurality of antigens is delivered to the anucleate cell-derived vesicle.

179. The method of any one of claims 155-178 wherein the adjuvant is a CpG ODN, IFN-α, STING agonists, RIG-I agonists, poly I:C, polyinosinic-polycytidylic acid stabilized with polylysine and carboxymethylcellulose (HILTONOL®), imiquimod, resiquimod, and/or LPS.

180. The method of claim 179, wherein the adjuvant is a low molecular weight poly I:C.

181. The method of any one of claims 154-180 wherein the input anucleate cell is a red blood cell.

182. The method of any one of claims 154-181, wherein the input anucleate cell is an erythrocyte.

183. The method of any one of claims 154-181, wherein the input anucleate cell is a reticulocyte.

184. The method of any one of claims 154-180, wherein the input anucleate cell is a platelet.

185. The method of any one of claims 154-184, wherein the input anucleate cell is a mammalian cell.

186. The method of any one of claims 154-185, wherein the input anucleate cell is a monkey, mouse, dog, cat, horse, rat, sheep, goat, pig, or rabbit cell.

187. The method of any one of claims 154-185, wherein the input anucleate cell is a human cell.

188. The method of any one of claims 154-187, wherein a half-life of the anucleate cell-derived vesicle following administration to a mammal is decreased compared to a half-life of the input anucleate cell following administration to the mammal.

189. The method of any one of claim 181-183, or 185-188, wherein a hemoglobin content of the anucleate cell-derived vesicle is decreased compared to the hemoglobin content of the input anucleate cell.

190. The method of any one of claims 181-189, wherein ATP production of the anucleate cell-derived vesicle is decreased compared to ATP production of the input anucleate cell.

191. The method of any one of claim 181-182 or 185-190, wherein the anucleate cell-derived vesicle exhibits one or more of the following properties:
(a) a circulating half-life in a mammal that is decreased compared to the input anucleate cell;
(b) decreased hemoglobin level compared to the input anucleate cell;
(c) a spherical morphology;
(d) increased surface phosphatidylserine levels compared to the input anucleate cell,
(e) reduced ATP production compared to the input anucleate cell.

192. The method of any one of claim 181-182 or 185-191, wherein the input anucleate cell is an erythrocyte and wherein the anucleate cell-derived vesicle has a reduced biconcave shape compared to the input anucleate cell.

193. The method of claim 181-182 or 185-192, wherein the anucleate cell-derived vesicle is a red blood cell ghost.

194. The method of any one of claims 154-193, wherein the anucleate cell-derived vesicles prepared by the process have greater than about 1.5 fold more phosphatidylserine on its surface compared to the input anucleate cell.

195. The anucleate cell-derived vesicle of any one of claims 154-194, wherein a population profile of anucleate cell-derived vesicles prepared by the process exhibits higher average phosphatidylserine levels on the surface compared to the input anucleate cells.

196. The anucleate cell-derived vesicle of any one of claims 154-195, wherein at least 50% of the population profile of anucleate cell-derived vesicles prepared by the process exhibits higher phosphatidylserine levels on the surface compared to the input anucleate cells.

197. The anucleate cell-derived vesicle of any one of claims 154-196, wherein the anucleate cell-derived vesicle exhibits enhanced uptake in a tissue or cell compared to the input anucleate cell.

198. The anucleate cell-derived vesicle of claim 197, wherein the anucleate cell-derived vesicle exhibit enhanced uptake in liver and/or spleen or by a phagocytic cell and/or an antigen-presenting cell compared to the uptake of the input anucleate cell.

199. The anucleate cell-derived vesicle of any one of claims 154-198, wherein the anucleate cell-derived vesicle is modified to enhance uptake in a tissue or cell compared to the input anucleate cell.

200. The anucleate cell-derived vesicle of claim 199, wherein the anucleate cell-derived vesicle is modified to enhance uptake in liver and/or spleen or by a phagocytic cell and/or an antigen-presenting cell compared to the uptake of the input anucleate cell.

201. The anucleate cell-derived vesicle of any one of claims 154-200, wherein the anucleate cell-derived vesicle comprises CD47 on its surface.

202. The method of any one of claims 154-201, wherein the anucleate cell-derived vesicle is not (a) heat processed, (b) chemically treated, and/or (c) subjected to hypotonic or hypertonic conditions during the preparation of the anucleate cell-derived vesicles.

203. The method of any one of claims 154-202, wherein the osmolarity of the cell suspension is maintained throughout the process.

204. The method of claims 154-203, wherein the osmolarity of the cell suspension is maintained between about 200 mOsm and about 400 mOsm throughout the process.

205. The method of any one of claims 154-204, wherein the constriction is contained within a microfluidic channel.

206. The method of claim 205, wherein the microfluidic channel comprises a plurality of constrictions.

207. The method of claim 206, wherein the plurality of constrictions are arranged in series and/or in parallel.

208. The method of any one of claims 154-207, wherein the constriction is between a plurality of micropillars; between a plurality of micropillars configured in an array; or between one or more movable plates.

209. The method of any one of claims 154-208, wherein the constriction is a pore or contained within a pore.

210. The method of claim 209, wherein the pore is contained in a surface.

211. The method of claim 210, wherein the surface is a filter.

212. The method of claim 210, wherein the surface is a membrane.

213. The method of any one of claims 154-212, wherein the constriction size is a function of the diameter of the input anucleate cell in suspension.

214. The method of any one of claims 154-213, wherein the constriction size is about 10%, about 20%, about 30%, about 40%, about 50%, about 60%, or about 70% of the diameter of the input anucleate cell in suspension.

215. The method of any one of claims 154-214, wherein the constriction has a width of about 0.25 μm to about 4 μm.

216. The method of any one of claims 154-215, wherein the constriction has a width of about 4 μm, 3.5 μm, about 3 μm, about 2.5 μm, about 2 μm, about 1.5 μm, about 1 μm, about 0.5 μm, or about 0.25 μm.

217. The method of any one of claims 154-215, wherein the constriction has a width of about 2.2 μm.

218. The method of any one of claims 154-217, wherein the input anucleate cells are passed through the constriction under a pressure ranging from about 10 psi to about 90 psi.

219. The method of any one of claims 154-218, wherein said cell suspension is contacted with the antigen before, concurrently, or after passing through the constriction.

220. A composition comprising a population of anucleate cell-derived vesicles prepared by the method of any one of claims 154-219.

221. An anucleate cell-derived vesicle prepared from a parent anucleate cell, the anucleate cell-derived vesicle having one or more of the following properties:
(a) a circulating half-life in a mammal is decreased compared to the parent anucleate cell,
(b) decreased hemoglobin levels compared to the parent anucleate cell,
(c) spherical morphology,
(d) increased surface phosphatidylserine levels compared to the parent anucleate cell, or
(e) reduced ATP production compared to the parent anucleate cell.

222. A composition comprising a plurality of anucleate cell-derived vesicles prepared from parent anucleate cells, the composition having one or more of the following properties:
(a) greater than about 20% of the anucleate cell-derived vesicles in the composition have a circulating half-life in a mammal that is decreased compared to the parent anucleate cell,
(b) greater than 20% of the anucleate cell-derived vesicles in the composition have decreased hemoglobin levels compared to the parent anucleate cell,
(c) greater than 20% of the anucleate cell-derived vesicles in the composition have spherical morphology,
(d) greater than 20% of the anucleate cell-derived vesicles in the composition are RBC ghosts,
(e) greater than 20% of the anucleate cell-derived vesicles in the composition vesicles in the composition have higher levels of phosphatidylserine compared to the population of parent anucleate cells, or
(f) greater than 20% of the anucleate cell-derived vesicles in the composition have reduced ATP production compared to the parent anucleate cell.

223. A composition comprising a plurality of anucleate cell-derived vesicles prepared from a population of a parent anucleate cell, the composition having one or more of the following properties:
(a) greater than about 20% of the anucleate cell-derived vesicles in the composition have a circulating half-life in a mammal that is decreased compared to the average of the population of the parent anucleate cell,
(b) greater than 20% of the anucleate cell-derived vesicles in the composition have decreased hemoglobin levels compared to the average of the population of the parent anucleate cell,
(c) greater than 20% of the anucleate cell-derived vesicles in the composition have spherical morphology,
(d) greater than 20% of the anucleate cell-derived vesicles in the composition are RBC ghosts,
(e) greater than 20% of the anucleate cell-derived vesicles in the composition vesicles in the composition have higher levels of phosphatidylserine compared to the average of the population of the parent anucleate cell, or
(f) greater than 20% of the anucleate cell-derived vesicles in the composition have reduced ATP production compared to the average of the population of the parent anucleate cell.

224. A method of making a composition comprising a plurality of anucleate cell-derived vesicles prepared from parent anucleate cells, the composition having one or more of the following properties:
(a) greater than 20% of the anucleate cell-derived vesicles in the composition have a circulating half-life in a mammal that is decreased compared to the parent anucleate cell,
(b) greater than 20% of the anucleate cell-derived vesicles in the composition have decreased hemoglobin levels compared to the parent anucleate cell,
(c) greater than 20% of the anucleate cell-derived vesicles in the composition have spherical morphology,
(d) greater than 20% of the anucleate cell-derived vesicles in the composition are RBC ghosts,
(e) greater than 20% of the anucleate cell-derived vesicles in the composition have higher levels of phosphatidylserine, or
(f) greater than 20% of the anucleate cell-derived vesicles in the composition have reduced ATP production compared to the parent anucleate cell;
the method comprising passing a cell suspension comprising the parent anucleate cell through a cell-deforming constriction, wherein a diameter of the constriction is a function of a diameter of the parent anucleate cell in the suspension, thereby causing perturbations of the parent anucleate cell large enough for a payload to pass through to form an anucleate cell-derived vesicle, thereby producing an anucleate cell-derived vesicle.

225. A method for treating a disease or disorder in an individual in need thereof, the method comprising administering the anucleate cell-derived vesicle of claim 221.

226. A method for treating a disease or disorder in an individual in need thereof, the method comprising administering the composition of claim 222.

227. A method for preventing a disease or disorder in an individual in need thereof, the method comprising administering the anucleate cell-derived vesicle of claim 221.

228. A method for preventing a disease or disorder in an individual in need thereof, the method comprising administering the composition of claim 222.
